(19) United States
(12) Patent Application Publication
(10) Pub. No.: US 2004/0166544 A1
(43) Pub. Date: Aug. 26, 2004

(54) Antibodies to c-Met for the treatment of cancers

(76) Inventors: Phillip A. Morton, Chesterfield, MO (US); John A. Arbuckle, Brentwood, MO (US); Michelle L. Evans, Carmel, IN (US); William D. Joy, St. Louis, MO (US); Larry E. Kahn, St. Louis, MO (US); Jeng-Jong Shieh, Chesterfield, MO (US)

Correspondence Address: Pharmacia Corporation, Global Patent Department, Mail Zone MC5, P. O. Box 1027, St. Louis, MO, 63141, US

(21) Appl. No.: 10/779,461

(22) Filed: Feb. 13, 2004

Related U.S. Application Data
(60) Provisional application No. 60/447,073, filed on Feb. 13, 2003.

(51) Int. Cl. A61K 39/395; G01N 33/574; C07K 16/30

(52) U.S. Cl. 435/7.23; 530/388.8; 424/155.1

(57) ABSTRACT

Antibodies specific for c-Met, a protein tyrosine kinase whose ligand is hepatocyte growth factor (HGF), are provided. The antibodies and fragments thereof may block binding of HGF to c-Met. Antagonist antibodies can be employed to block binding of HGF to c-Met or substantially inhibit c-Met activation. The c-Met antibodies may be included in pharmaceutical compositions, articles of manufacture, or kits. Methods of treating cancer, pathological liver conditions, and ophthalmic diseases using the c-Met antibodies are also provided.

The present application claims priority under Title 35, United States Code, §119 to U.S. Provisional application Serial No. 60/447,073, filed Feb. 13, 2003, which is incorporated by reference in its entirety as if written herein.

### FIELD OF THE INVENTION

This application relates to c-Met protein tyrosine kinase antibodies, particularly antagonists of HGF binding to c-Met. The application also relates to the use of the antibodies in therapy or diagnosis of particular pathological conditions in mammals, including cancer.

### BACKGROUND OF THE INVENTION

Hepatocyte growth factor (HGF) functions as a growth factor for particular tissues and cell types. HGF was identified initially as a mitogen for hepatocytes [Michalopoulos et al., Cancer Res., 44:4414-4419 (1984); Russel et al., J. Cell. Physiol., 119:183-192 (1984); Nakamura et al., Biochem. Biophys. Res. Comm., 122:1450-1459 (1984)]. Nakamura et al., supra, reported the purification of HGF from the serum of partially hepatectomized rats. Subsequently, HGF was purified from rat platelets, and its subunit structure was determined [Nakamura et al., Proc. Natl. Acad. Sci. USA, 83:6489-6493 (1986); Nakamura et al., FEBS Letters, 224:311-316 (1987)]. The purification of human HGF from human plasma was first described by Gohda et al., J. Clin. Invest., 81:414-419 (1988).

Both rat HGF and human HGF have been molecularly cloned, including the cloning and sequencing of a naturally occurring variant lacking 5 amino acids designated “delta5 HGF” [Miyazawa et al., Biochem. Biophys. Res. Comm., 163:967-973 (1989); Nakamura et al., Nature, 342:440-443 (1989); Seki et al., Biochem. Biophys. Res. Commun. 172:321-327 (1990); Tashiro et al., Proc. Natl. Acad. Sci. USA, 87:3200-3204 (1990); Okajima et al., Eur. J. Biochem., 193:375-381 (1990)].

The mature form of human HGF, corresponding to the major form purified from serum, is a disulfide-linked heterodimer derived by proteolytic cleavage of the pro-hormone between amino acids R494 and V495. This cleavage generates a molecule composed of an α-subunit of 440 amino acids (Mᵣ 69 kDa) and a β-subunit of 234 amino acids (Mᵣ 34 kDa). The nucleotide sequence of human HGF cDNA reveals that both the α-and the β-chains are contained in a single open reading frame coding for a pre-pro precursor protein. In the predicted primary structure of mature human HGF, an interchain disulfide bridge is formed between Cys 487 of the α-chain and Cys 604 in the β-chain [see Nakamura et al., Nature, supra]. The N-terminus of the α chain is preceded by 54 amino acids, starting with a methionine. This segment includes a characteristic hydrophobic leader (signal) sequence of 31 residues and the prosequence. The α-chain starts at amino acid (aa) 55, and contains four kringle domains. The kringle 1 domain extends from about aa 128 to about aa 206, the kringle 2 domain is between about aa 211 and about aa 288, the kringle 3 domain is defined as extending from about aa 303 to about aa 383, and the kringle 4 domain extends from about aa 391 to about aa 464 of the α-chain.

The definition of the various kringle domains is based on their homology with kringle-like domains of other proteins (such as prothrombin and plasminogen); therefore, the above limits are only approximate. To date, the function of these kringles has not been determined. The β-chain of human HGF shows 38% homology to the catalytic domain of serine protease plasminogen. However, two of the three residues which form the catalytic triad of serine proteases requisite for enzymatic activity are not conserved in human HGF. Therefore, despite its serine protease-like domain, human HGF appears to have no proteolytic activity, and the precise role of the β-chain remains unknown. HGF contains four putative glycosylation sites, which are located at positions 294 and 402 of the α-chain and at positions 566 and 653 of the β-chain.

In a portion of cDNA isolated from human leukocytes, in-frame deletion of 15 base pairs was observed. Transient expression of the cDNA sequence in COS-1 cells revealed that the encoded HGF molecule (delta5 HGF) lacking 5 amino acids in the kringle 1 domain was fully functional [Seki et al., supra].

A naturally occurring human HGF variant has been identified which corresponds to an alternative spliced form of the transcript containing the coding sequences for the N-terminal finger and first two kringle domains of mature HGF [Chan et al., Science, 254:1382-1385 (1991); Miyazawa et al., Eur. J. Biochem. 197:15-22 (1991)]. This variant, designated HGF/NK2, has been proposed to be a competitive antagonist of mature HGF. Comparisons of the amino acid sequence of rat HGF with that of human HGF have revealed that the two sequences are highly conserved and have the same characteristic structural features. The length of the four kringle domains in rat HGF is exactly the same as in human HGF. Furthermore, the cysteine residues are located in exactly the same positions, an indication of similar three-dimensional structures [Okajima et al., supra; Tashiro et al., supra].

HGF and HGF variants are described further in U.S. Pat. Nos. 5,227,158, 5,316,921, and 5,328,837.

The HGF receptor has been identified as the product of the c-Met proto-oncogene [Bottaro et al., Science, 251:802-804 (1991); Naldini et al., Oncogene, 6:501-504 (1991); WO 92/13097 published Aug. 6, 1992; WO 93/15754 published Aug. 19, 1993]. The receptor is usually referred to as “c-Met” or “p190^{MET}” and typically comprises, in its native form, a 190-kDa heterodimeric (a disulfide-linked 50-kDa α-chain and a 145-kDa β-chain) membrane-spanning tyrosine kinase protein [Park et al., Proc. Natl. Acad. Sci. USA, 84:6379-6383 (1987)]. Several truncated forms of the c-Met receptor have also been described [WO 92/20792; Prat et al., Mol. Cell. Biol., 11:5954-5962 (1991)].

The binding activity of HGF to c-Met is believed to be conveyed by a functional domain located in the N-terminal portion of the HGF molecule, including the first two kringles [Matsumoto et al., Biochem. Biophys. Res. Commun. 181:691-699 (1991); Hartmann et al., Proc. Natl. Acad. Sci., 89:11574-11578 (1992); Lokker et al., EMBO J., 11:2503-2510 (1992); Lokker and Godowski, J. Biol. Chem., 268:17145-117150 (1991)]. The c-Met protein tyrosine kinase becomes phosphorylated on several tyrosine residues of the 145-kDa β-subunit upon HGF binding.

Certain antibodies to HGF receptor have been reported in the literature. Several such antibodies are described below.

Prat et al., Mol. Cell. Biol., supra, describe several monoclonal antibodies specific for the extracellular domain of the β-chain encoded by the c-Met gene [see also, WO 92/20792]. The monoclonal antibodies were selected following immunization of Balb/c mice with whole living GTL-16 cells (human gastric carcinoma cell line) overexpressing the c-Met protein. The spleen cells obtained from the immunized mice were fused with Ag8.653 myeloma cells, and hybrid supernatants were screened for binding to GTL-16 cells. Four monoclonal antibodies, referred to as DL-21, DN-30, DN-31, and DO-24, were selected.

Prat et al., Int. J. Cancer, 49:323-328 (1991) describe using c-Met monoclonal antibody DO-24 for detecting distribution of the c-Met protein in human normal and neoplastic tissues [see, also, Yamada et al., Brain Research, 637:308-312 (1994)]. The murine monoclonal antibody DO-24 was reported to be an IgG2a isotype antibody.

Crepaldi et al., J. Cell Biol., 125: 313-320 (1994) report using monoclonal antibodies DO-24 and DN-30 [described in Prat et al., Mol. Cell. Biol., supra] and monoclonal antibody DQ-13 to delineate the subcellular distribution of c-Met in epithelial tissues and in MDCK cell monolayers. According to Crepaldi et al., monoclonal antibody DQ-13 was raised against a peptide corresponding to nineteen carboxy-terminal amino acids (from Ser¹³⁷² to Ser¹³⁹⁰⁾ of the human c-Met sequence.

A monoclonal antibody specific for the cytoplasmic domain of human c-Met has also been described [Bottaro et al., supra].

Monovalent c-Met antibodies, including 1A3.3.13 antibody (ATCC deposit No. HB-11894) and 5D5.11.6 antibody (ATCC deposit No. HB-11895), and methods of treating cancers using such are disclosed in U.S. Pat. No. 5,686,292: US and U.S. Pat. No. 6,207,152.

Several of the monoclonal antibodies referenced above are commercially available from Upstate Biotechnology Incorporated, Lake Placid, N.Y. Monoclonal antibodies DO-24 and DL-21, specific for the extracellular epitope of c-Met, are available from Upstate Biotechnology Incorporated. Monoclonal antibody DQ-13, specific for the intracellular epitope of c-Met, is also available from Upstate Biotechnology Incorporated.

Various biological activities have been described for HGF and its receptor [see, generally, Chan et al., Hepatocyte Growth Factor—Scatter Factor (HGF—SF) and the C-Met Receptor, Goldberg and Rosen, eds., Birkhauser Verlag-Basel (1993), pp. 67-79]. It has been observed that levels of HGF increase in the plasma of patients with hepatic failure [Gohda et al., supra] and in the plasma [Lindroos et al., Hepatol. 13:734-750 (1991)] or serum [Asami et al., J. Biochem. 109:8-13 (1991)] of animals with experimentally induced liver damage. The kinetics of this response are usually rapid, and precedes the first round of DNA synthesis during liver regeneration. HGF has also been shown to be a mitogen for certain cell types, including melanocytes, renal tubular cells, keratinocytes, certain endothelial cells and cells of epithelial origin [Matsumoto et al., Biochem. Biophys. Res. Commun. 176:45-51 (1991); Igawa et al., Biochem. Biophys. Res. Commun. 174:831-838 (1991); Han et al., Biochem., 30:9768-9780 (1991); Rubin et al., Proc. Natl. Acad. Sci. USA, 88:415-419 (1991)]. Both HGF and the c-Met protooncogene have been postulated to play a role in microglial reactions to CNS injuries [DiRenzo et al., Oncogene, 8:219-222 (1993)].

HGF can also act as a “scatter factor”, an activity that promotes the dissociation and motility of epithelial and vascular endothelial cells in vitro [Stoker et al., Nature, 327:239-242 (1987); Weidner et al., J. Cell Biol., 111:2097-2108 (1990); Naldini et al., EMBO J., 10:2867-2878 (1991); Giordano et al., Proc. Natl. Acad. Sci. USA, 90:649-653 (1993)]. Moreover, HGF has recently been described as an epithelial morphogen [Montesano et al., Cell, 67:901-908 (1991)]. Therefore, HGF has been postulated to be important in tumor invasion [Comoglio, Hepatocyte Growth Factor—Scatter Factor (HGF—SF) and the C-Met Receptor, Goldberg and Rosen, eds., Birkhauser Verlag-Basel (1993), pp. 131-165]. Bellusci et al., Oncogene, 9:1091-1099 (1994) report that HGF can promote motility and invasive properties of NBT-II bladder carcinoma cells.

c-Met RNA has been detected in several murine myeloid progenitor tumor cell lines [Iyer et al., Cell Growth and Differentiation, 1:87-95 (1990)]. Further, c-Met is expressed in various human solid tumors [Prat et al., Int. J. Cancer, supra]. Overexpression of the c-Met oncogene has also been suggested to play a role in the pathogenesis and progression of thyroid tumors derived from follicular epithelium [DiRenzo et al., Oncogene, 7:2549-2553 (1992)]. Chronic c-Met/HGF receptor activation has also been observed in certain malignancies [Cooper et al., EMBO J., 5:2623 (1986); Giordano et al., Nature, 339:155 (1989)].

In view of the role of HGF and/or c-Met in potentiating or promoting such diseases or pathological conditions, it would be useful to have a means of substantially reducing or inhibiting one or more of the biological effects elicited by binding of HGF to c-Met.

### BRIEF DESCRIPTION OF THE DRAWINGS

FIGS. 1a-g show alignments of the amino acid sequences of the light and heavy regions of PGIA-01-08, PGIA-03-A9, PGIA-03-A11, PGIA-03-B2, PGIA-04-A5, PGIA-04-A8, and PGIA-05-A1 c-Met scFv antibodies to the germline sequence. C-met scFv alignments to germnine. Differences between query sequence and the first germline sequence are bolded and underlined. CDR sequences are highlighted in gray boxes.

FIG. 2 shows inhibition of HGF binding to recombinant c-Met protein by c-Met IgG antibodies 11978, 11994, 12075, and 12119.

FIG. 3 shows inhibition of HGF-dependent cellular proliferation in 184B5 cells by c-Met IgG antibodies 11978, 11994, and 12075.

FIG. 4 shows enhanced tyrosine phosphorylation of the c-Met kinase domain in HCT-116 human colon carcinoma cells following treatment with c-Met IgG antibodies 11978, 11994, 12075, 12119, 12123, 12133, and 12136 determined by Western blot and ELISA.

FIG. 5 shows blocking of HGF binding to c-Met by Fab fragments derived from c-Met antibodies 11978, 11994, 12075, and 12123.

FIG. 6 shows enhanced tyrosine phosphorylation of the c-Met kinase domain by Fab fragments derived from c-Met antibodies 11978, 11994, 12075, 12119, 12123, 12133, and 12136.

FIG. 7 shows inhibition of HGF dependent cellular proliferation of 184B5 cells by Fab fragment derived from c-Met antibody 11994.

FIG. 8 is a representative graph testing the antagonistic and agonistic potential of c-Met IgG antibodyl 1978 in a scatter assay.

FIG. 9 is a graph created from the determination of the wound areas from a H441 cell wound healing (scratch) assay. c-Met IgG antibodies 12133, 12136, 11994, and 12119 show a dose dependent inhibition of cell migration into the scratch.

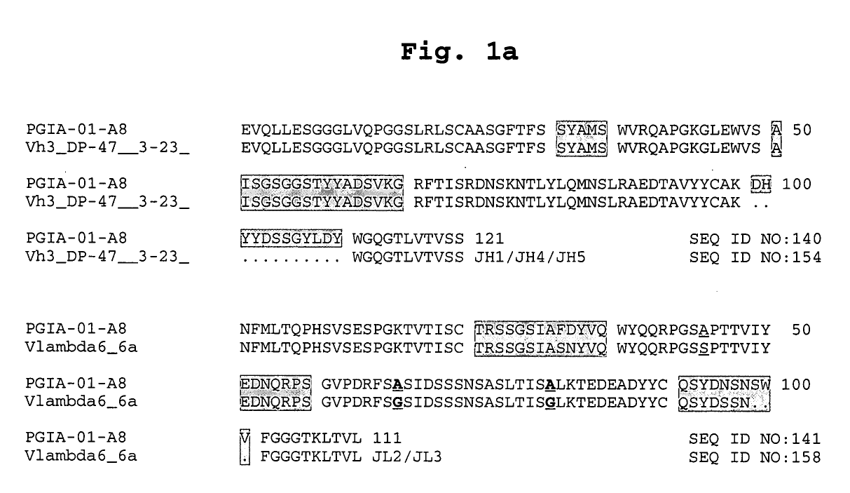

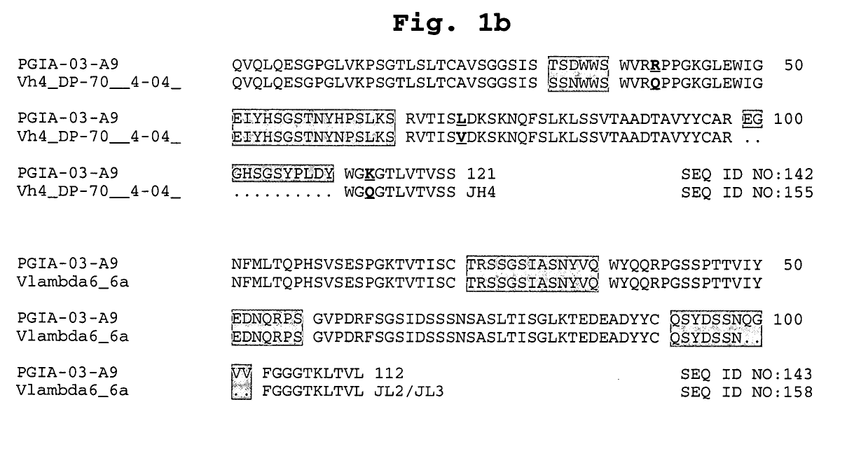

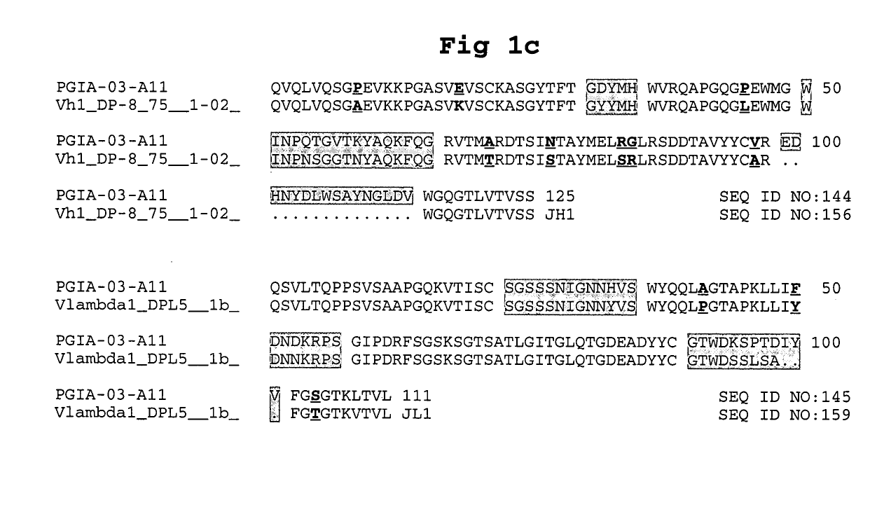

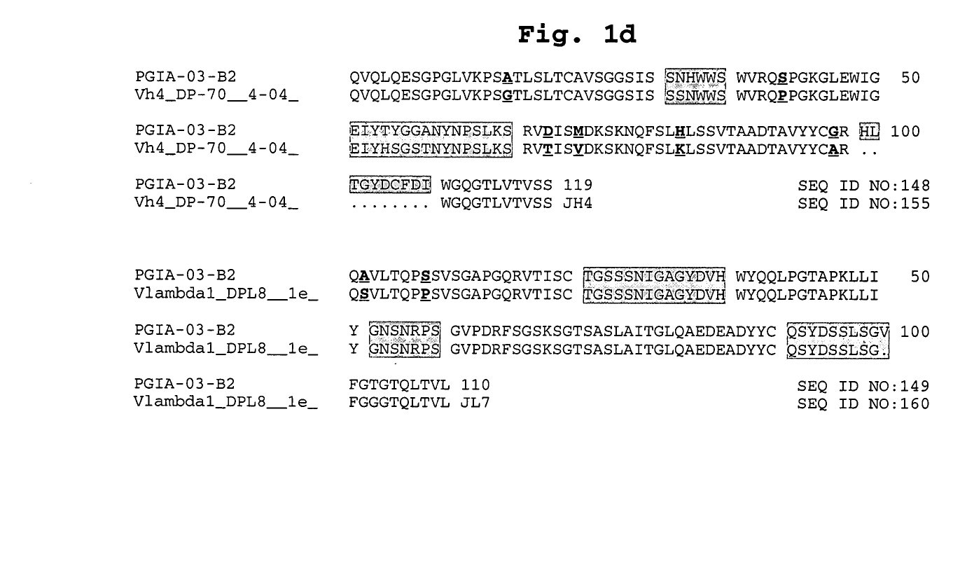

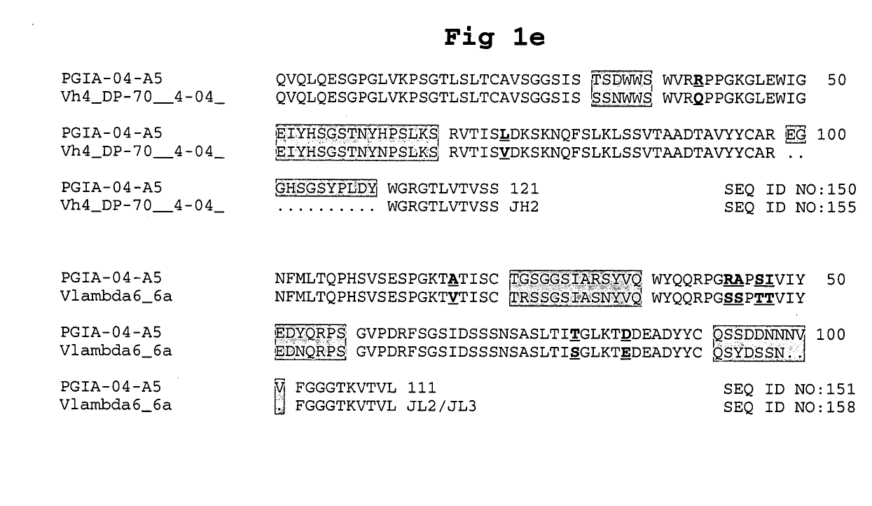

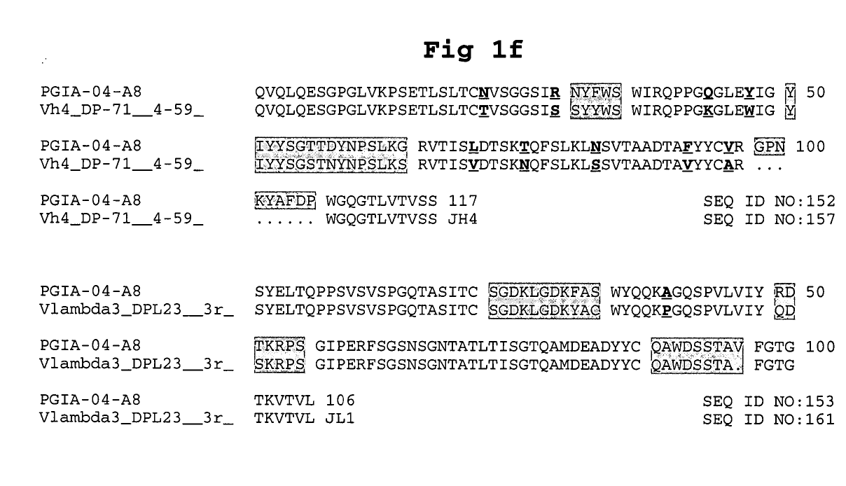

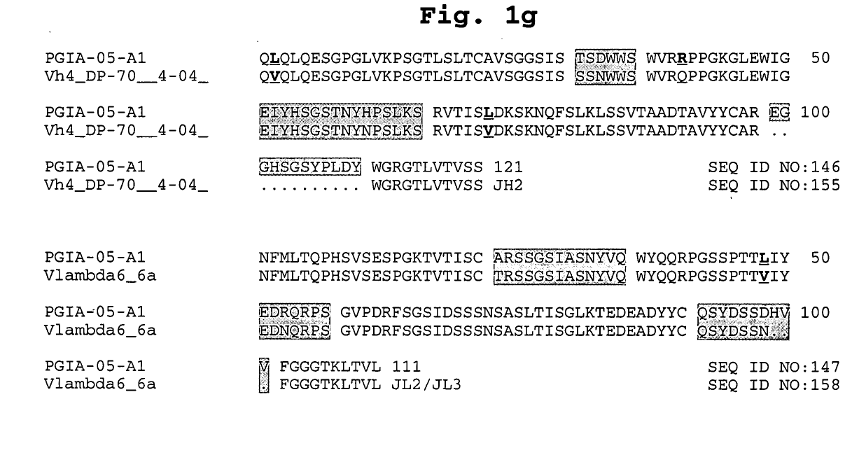

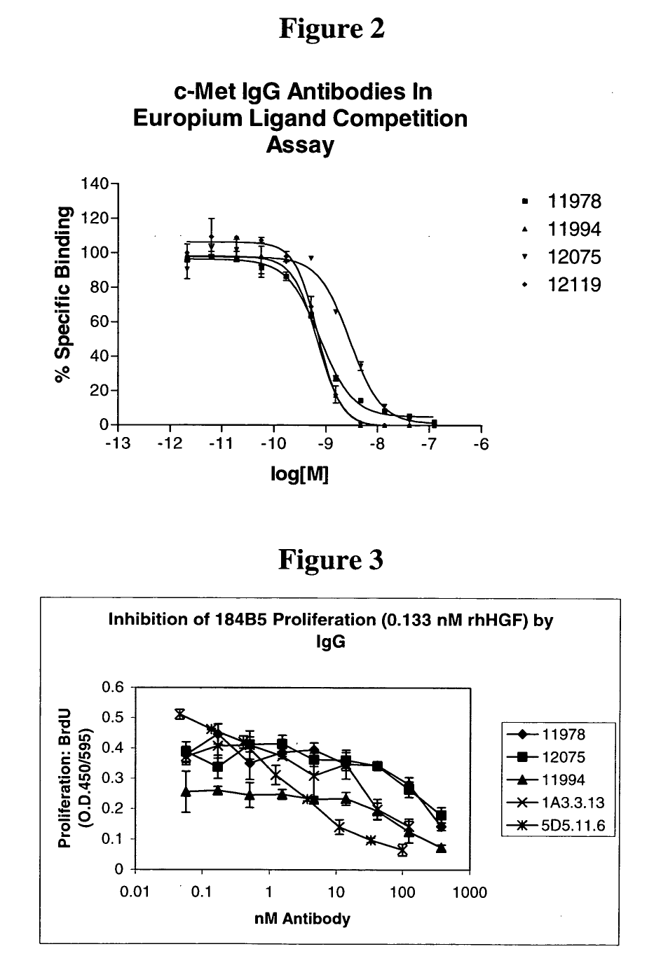

### SUMMARY OF THE INVENTION

The present invention provides an isolated antibody or antigen-binding portion thereof that binds c-Met, preferably one that binds to primate and human c-Met, and more preferably one that is a human antibody. The invention provides c-Met antibodies that inhibit the binding of HGF to c-Met, and also provides c-Met antibodies that activate c-Met tyrosine phosphorylation.

The invention provides a pharmaceutical composition comprising the antibody and a pharmaceutically acceptable carrier. The pharmaceutical composition may further comprise another component, such as an anti-tumor agent or an imaging reagent.

Diagnostic and therapeutic methods are also provided by the invention. Diagnostic methods include a method for diagnosing the presence or location of a c-Met-expressing tissue using a c-Met antibody. A therapeutic method comprises administering the antibody to a subject in need thereof, preferably in conjunction with administration of another therapeutic agent.

The invention provides an isolated cell line, such as a hybridoma, that produces a c-Met antibody.

The invention also provides nucleic acid molecules encoding the heavy and/or light chain or antigen-binding portions thereof of a c-Met antibody.

The invention provides vectors and host cells comprising the nucleic acid molecules, as well as methods of recombinantly producing the polypeptides encoded by the nucleic acid molecules.

Non-human transgenic animals that express the heavy and/or light chain or antigen-binding portions thereof of a c-Met antibody are also provided. The invention also provides a method for treating a subject in need thereof with an effective amount of a nucleic acid molecule encoding the heavy and/or light chain or antigen-binding portions thereof of a c-Met antibody.

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Using Antibodies: A Laboratory Manual Cold Spring Harbor Laboratory Press, Cold Spring Harbor, N.Y. (1999), which are incorporated herein by reference.

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

As used herein, the terms “hepatocyte growth factor” and “HGF” refer to a growth factor typically having a structure with six domains (finger, Kringle 1, Kringle 2, Kringle 3, Kringle 4 and serine protease domains). Fragments of HGF constitute HGF with fewer domains and variants of HGF may have some of the domains of HGF repeated; both are included if they still retain their respective ability to bind a HGF receptor. The terms “hepatocyte growth factor” and “HGF” include hepatocyte growth factor from humans and any non-human mammalian species, and in particular rat HGF. The terms as used herein include mature, pre, pre-pro, and pro forms, purified from a natural source, chemically synthesized or recombinantly produced. Human HGF is encoded by the cDNA sequence published by Miyazawa et al., 1989, supra, or Nakamura et al., 1989, supra. The sequences reported by Miyazawa et al. and Nakamura et al. differ in 14 amino acids. The reason for the differences is not entirely clear; polymorphism or cloning artifacts are among the possibilities. Both sequences are specifically encompassed by the foregoing terms. It will be understood that natural allelic variations exist and can occur among individuals, as demonstrated by one or more amino acid differences in the amino acid sequence of each individual. The terms “hepatocyte growth factor” and “HGF” specifically include the delta5 huHGF as disclosed by Seki et al., supra.

The terms “HGF receptor” and “c-Met” when used herein refer to a cellular receptor for HGF, which typically includes an extracellular domain, a transmembrane domain and an intracellular domain, as well as variants and fragments thereof which retain the ability to bind HGF. The terms “HGF receptor” and “c-Met” include the polypeptide molecule that comprises the full-length, native amino acid sequence encoded by the gene variously known as p 190^{MET.} The present definition specifically encompasses soluble forms of c-Met, and c-Met from natural sources, synthetically produced in vitro or obtained by genetic manipulation including methods of recombinant DNA technology. The c-Met variants or fragments preferably share at least about 65% sequence homology, and more preferably at least about 75% sequence homology with any domain of the human c-Met amino acid sequence published in Rodrigues et al., Mol. Cell. Biol., 11:2962-2970 (1991); Park et al., Proc. Natl. Acad. Sci., 84:6379-6383 (1987); or Ponzetto et al., Oncogene, 6:553-559 (1991).

The term “HGF biological activity” when used herein refers to any mitogenic, motogenic, or morphogenic activities of HGF or any activities occurring as a result of HGF binding to c-Met. The term “c-Met activation” refers to c-Met dimerization or HGF-induced tyrosine kinase activity within c-Met. Activation of c-Met may occur as a result of HGF binding to c-Met, but may alternatively occur independent of any HGF binding to c-Met. In addition “c-Met activation” may occur following the binding of a c-Met monoclonal antibody to c-Met. HGF biological activity may, for example, be determined in an in vitro or in vivo assay of HGF-induced cell proliferation, cell scattering, or cell migration. The effect of a HGF receptor antagonist can be determined in an assay suitable for testing the ability of HGF to induce DNA synthesis in cells expressing c-Met such as mink lung cells or human mammary epithelial cells (described in Example 5). DNA synthesis can, for example, be assayed by measuring incorporation of ³H-thymidine into DNA. The effectiveness of the c-Met antagonist can be determined by its ability to block proliferation and incorporation of the ³H-thymidine into DNA. The effect of c-Met antagonists can also be tested in vivo in animal models.

The term “polypeptide” encompasses native or artificial proteins, protein fragments, and polypeptide analogs of a protein sequence. A polypeptide may be monomeric or polymeric.

The term “isolated protein” or “isolated polypeptide” is a protein or polypeptide that by virtue of its origin or source of derivation, (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein separation and purification techniques well known in the art.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally occurring sequence. Fragments typically are at least 5, 6, 8, or amino acids long, preferably at least 14 amino acids long, more preferably at least amino acids long, usually at least 20 amino acids long, even more preferably at least 70, 80, 90, 100, 150 or 200 amino acids long.

The term “polypeptide analog” as used herein refers to a polypeptide that is comprised of a segment of at least amino acids that has substantial identity to a portion of an amino acid sequence and that has at least one of the following properties: (1) specific binding to c-Met under suitable binding conditions, (2) ability to block HGF binding to c-Met, or (3) ability to reduce c-Met cell surface expression or tyrosine phosphorylation in vitro or in vivo. Typically, polypeptide analogs comprise a conservative amino acid substitution (or insertion or deletion) with respect to the naturally occurring sequence. Analogs typically are at least 20 amino acids long, preferably at least 50, 60, 70, 80, 90, 100, 150 or 200 amino acids long or longer, and can often be as long as a full-length naturally occurring polypeptide.

Preferred amino acid substitutions are those which, (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference. Non-peptide analogs are commonly used in the pharmaceutical industry as drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 1 or more amino acids primarily responsible for antigen recognition. The carboxy-terminal portion of each chain defines a constant region primarily responsible for effector function. Human light chains are classified as either kappa or lambda chains. Heavy chains are classified as μ, Δ, γ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarily determining regions or CDRs. The CDRs from the two chains of each pair are aligned by the framework regions, enabling binding to a specific epitope. From N-terminus to C-terminus, both light and heavy chains comprise the domains FRI, CDR1, FR2, CDR2, FR3, CDR3 and FR4. The assignment of amino acids to each domain is in accordance with the definitions of Kabat, et al., Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarily determining region (CDR) fragments, single-chain antibodies (scFv), chimeric antibodies, diabodies and polypeptides that contain at least a portion of an immunoglobulin that is sufficient to confer specific antigen binding to the polypeptide.

An Fab fragment is a monovalent fragment consisting of the VL, VH, CL and CH1 domains; a F(ab′)2 fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain.

A single-chain antibody (scFv) is an antibody in which a VL and VH regions are paired to form a monovalent molecule via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:64446448, 1993, and Poljak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally occurring immunoglobulin has two identical binding sites; a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature.

Examples of isolated antibodies include an c-Met antibody that has been affinity purified using c-Met is an antigen, an anti- c-Met antibody that has been synthesized by a hybridoma or other cell line in vitro, and a human c-Met antibody derived from a transgenic mouse.

The term “human antibody” includes all antibodies that have one or more variable and constant regions derived from human immunoglobulin sequences.

In a preferred embodiment, all of the variable and constant domains are derived from human immunoglobulin sequences (a fully human antibody). These antibodies may be prepared in a variety of ways, as described below.

A “humanized antibody” is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152, and 5,877,293.

The term “chimeric antibody” refers to an antibody that contains one or more regions from one antibody and one or more regions from one or more other antibodies. In a preferred embodiment, one or more of the CDRs are derived from a human c-Met antibody. In a more preferred embodiment, all of the CDRs are derived from a human c-Met antibody. In another preferred embodiment, the CDRs from more than one human c-Met antibody are mixed and matched in a chimeric antibody. For instance, a chimeric antibody may comprise a CDR1 from the light chain of a first human c-Met antibody may be combined with CDR2 and CDR3 from the light chain of a second human c-Met antibody, and the CDRs from the heavy chain may be derived from a third c-Met antibody. Further, the framework regions may be derived from one of the same c-Met antibodies, from one or more different antibodies, such as a human antibody, or from a humanized antibody. A “neutralizing antibody” or “an inhibitory antibody” is an antibody that inhibits the binding of c-Met to HGF when an excess of the c-Met antibody reduces the amount of HGF bound to c-Met by at least about 20%. In a preferred embodiment, the antibody reduces the amount of HGF bound to c-Met by at least 40%, more preferably 60%, even more preferably 80%, or even more preferably 85%. The binding reduction may be measured by any means known to one of ordinary skill in the art, for example, as measured in an in vitro competitive binding assay. An example of measuring the reduction in binding of HGF to c-Met is presented below in Example 4.

An “activating antibody” is an antibody that activates c-Met by at least about 20% when added to a cell, tissue, or organism expressing c-Met, when compared to the activation achieved by an equivalent molar amount of HGF. In a preferred embodiment, the antibody activates c-Met activity by at least 40%, more preferably 60%, even more preferably 80%, or even more preferably 85% of the level of activation achieved by an equivalent molar amount of HGF. In a more preferred embodiment, the activating antibody is added in the presence of HGF. In another preferred embodiment, the activity of the activating antibody is measured by determining the amount of tyrosine phosphorylation and activation of c-Met.

Fragments or analogs of antibodies can be readily prepared by those of ordinary skill in the art following the teachings of this specification. Preferred amino and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure have been described by Bowie et al. Science 253:164(1991).

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnsson, B., et al. (1991) Anal. Biochem. 198:268-277.

The term “K_{off}” refers to the off rate constant for dissociation of an antibody from the antibody/antigen complex.

The term “K_{d}” refers to the dissociation constant of a particular antibody-antigen interaction.

The term “epitope” includes any molecular determinant capable of specific binding to an immunoglobulin or T-cell receptor. Epitopes usually consist of chemically active surface groupings of molecules such as amino acids or sugar side chains and usually have specific three-dimensional structural characteristics, as well as specific charge characteristics. An antibody is said to specifically bind an antigen when the dissociation constant is <1 M, preferably <100 nM, preferably <10 nM, and most preferably <1 nM.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass.(1991)), which is incorporated herein by reference. Stereoisomers (e.g., D-amino acids) of the twenty conventional amino acids, unnatural amino acids such as α-, α-2,5 disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methyl arginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide”, (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotides” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like.

The term “oligonucleotide linkages” referred to herein includes Oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

“Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences, and fusion partner sequences. The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked.

Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e. g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides, and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×SSPE or SSC, 50% formamide, S× Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art that can be used to measure nucleotide sequence identity. For instance, polynucleotide sequences can be compared using FASTA, Gap, or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183: 63-98 (1990); Pearson, Methods Mol. Biol. 132: 185-219 (2000); Pearson, Methods Enzymol. 266: 227-258 (1996); Pearson, J. Mol. Biol. 276: 71-84 (1998; herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleic acid sequence encompasses its complement unless otherwise specified. Thus, a reference to a nucleic acid molecule having a particular sequence should be understood to encompass its complementary strand, with its complementary sequence.

In the molecular biology art, researchers use the terms “percent sequence identity”, “percent sequence similarity” and “percent sequence homology” interchangeably. In this application, these terms shall have the same meaning with respect to nucleic acid sequences only.

The term “substantial similarity” or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, share at least 75% or 80% sequence identity, preferably at least 90% or 95% sequence identity, even more preferably at least 98% or 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e. g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. See, e.g., Pearson, Methods Mol. Biol. 24: 307-31 (1994), herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256: 1443-45 (1992), herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions, and other modifications, including conservative amino acid substitutions. For instance, GCG contains programs such as “Gap” and “Bestfit” which can be used with default parameters to determine sequence homology or sequence identity between closely related polypeptides, such as homologous.

Polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (1990); Pearson (2000). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215: 403410 (1990); Altschul et al., Nucleic Acids Res. 25:3389-402 (1997); herein incorporated by reference.

The length of polypeptide sequences compared for homology will generally be at least about 16 amino acid residues, usually at least about residues, more usually at least about 24 residues, typically at least about 28 residues, and preferably more than about 35 residues. When searching a database containing sequences from a large number of different organisms, it is preferable to compare amino acid sequences.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B. gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

In some embodiments, labels are attached by spacer arms of various lengths to reduce potential steric hindrance.

The term “agent” is used herein to denote a chemical compound, a mixture of chemical compounds, a biological macromolecule, or an extract made from biological materials. The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

The term “patient” includes human and veterinary subjects.

### Human c-Met Antibodies and Characterization Thereof

Human antibodies avoid certain of the problems associated with antibodies that possess mouse or rat variable and/or constant regions. The presence of such mouse or rat derived sequences can lead to the rapid clearance of the antibodies or can lead to the generation of an immune response against the antibody by a patient.

Therefore, in one embodiment, the invention provides humanized anti-c-Met antibodies. In a preferred embodiment, the invention provides fully human c-Met antibodies by introducing human immunoglobulin genes into a rodent so that the rodent produces fully human antibodies. More preferred are fully human anti-human c-Met antibodies. Fully human c-Met antibodies directed against human c-Met are expected to minimize the immunogenic and allergic responses intrinsic to mouse or mouse-derivatized monoclonal antibodies (Mabs) and thus to increase the efficacy and safety of the administered antibodies. The use of fully human antibodies can be expected to provide a substantial advantage in the treatment of chronic and recurring human diseases, such as inflammation and cancer, which may require repeated antibody administrations. In another embodiment, the invention provides a c-Met antibody that does not bind complement.

In a preferred embodiment, the c-Met antibody is selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1 or a fragment of any one thereof. In a preferred embodiment, the c-Met antibody is selected from PGIA-01-A8, PGIA-03-A9, PGIA-03-A11, PGIA-03-B2, PGIA-04-A5, PGIA-04-A8, and PGIA-05-A1 or a fragment of any one thereof. In a preferred embodiment the c-Met antibody is selected from PGIA-03-A9, PGIA-04-A5, and PGIA-04-A8 or a fragment of any one thereof.

Table 1 shows the amino acid sequences of the scFvs PGIA-01-A1 through PGIA-05-A1 above.

TABLE 1

| | |
|---|---|
| PGIA-1-A1 | |
| EVQLLESGRGLVQPGGSLRLSCAASGFTFSSYAMSWVRQAPGKGLEWVSAISGSGGS | SEQ ID NO:1 |
| TYYADSVKGRFTISRDNSKNTLYLQMNSLRAEDTAVYYCARWGQGTTVTVSSGGGGS | |
| GGGGSGGGGSAQAVLTQPSSVSGAPGQRVTISCTGSSSNIGADYDVHWYQQLPGTAP | |
| KLLIYGNNNRPSGVPDRFSGSKSGTSASLAITGLQAEDEADYYCQSYDNSPDAYVVF | |
| GGGTKLTVLS, | |
| PGIA-1-A2 | |
| QVQLVQSGAEVRKPGASVKVSCKTSGYTFIDYYIHWVRQAPGQGLEWMGWVNPVTGT | SEQ ID NO:2 |
| SGSSPNFRGRVTMTTDTSGNTAYMELRSLRSDDTAVFYCARRHQQSLDYWGQGTLVT | |
| VSSGGGGSGGGGSGGGGSAQSVLTQPPSVSAPPGQKVTISCSGSSSNIGTNYVSWYQ | |
| QLPGTAPKLLIYDNHKRPSVIPDRFSGSKSGTSATLGISGLQTGDEADYYCGTWDYS | |
| LSTWVFGGGTKLTVLG, | |
| PGIA-1-A3 | |
| QLQLQESGPGLVKPSGTLSLTCAVSGDSVSSYYWWSWVRQPPGKGLEWIGEIERDGS | SEQ ID NO:3 |
| SNYNRSLKSRVTISPDKPKNQFSLRLSSVTAADTAIYYCARHIRGYDAFDIWGRGTL | |
| VTVSSGGGGSGGGGSGGGGSAQSVLTQPPSVSGAPGQRVTISCTGSSSNIGAGYDVH | |
| WYQQFPGRAPKLLIYGNTNRPSGVPDRFSGSKSDISASLAITGLQAEDEADYYCQSY | |
| DSNLTGVFGGGT, | |
| PGIA-1-A4 | |
| QVQLVQSGAEVRKPGASVKVSCKTSGYTFMDYYIHWVRQAPGQGLEWMGWSNPVTGT | SEQ ID NO:4 |
| SGSSPKFRGRVTLTTDTSGNTAYLDLRSLRSDDTAVFYCARRHQQSLDYWGQGTMVT | |
| VSSGGGGSGGGGSGGGGSAQSVLTQPPSVSAAPGQKVTISCSGSSSNIGNNYVSWYQ | |
| QLPGTAPKLLMYENSKRPSGIPDRFSGSKSGTSGTLGITGLQTGDEADYYCGTWDTS | |
| LRAWVFGGGTKVTVLG, | |
| PGIA-1-A5 | |
| QVQLQQSGAEVRKPGASAKVSCKTSGYTFIDYYIHWVRQAPGQGLEWMGWINPVTGA | SEQ ID NO:5 |
| SGSSPNFRGRVTLTTDTSGNTAYMELRSLRSDDTAVFYCARRHQQSLDYWGRGTTVT | |
| VSSGGGGSGGGGSGGGGSAQSVVTQPPSVSAAPGQKVTISCSGRTSNIGNNYVSWYQ | |
| QVPGAPPKLLIFDNNKRPSGTPARFSGSKSGTSATLAISGLQTGDEADYYCGTWDTT | |
| LRGFVFGPGTKVTVLG, | |
| PGIA-1-A6 | |
| QLQLQESGPGLVKPSGTLSLTCAVSGGSISSTNWWSWVRQPPGKGLEWIGEIYHSGS | SEQ ID NO:6 |
| TNYNPSLKSRVTISVDKSKNHFSLNLSSVTAADTAVYYCARDSMGSTGWHYGMDLWG | |
| RGTLVTVSSGGGGSGGGGSGGGGSAQSALTQPPSASGSPGQSVTISCSGSSSDIGDY | |
| NHVSWYQQHPGKAPKLMIYDVNKWPSGVPDRFSGSKSGNTASLTVSGLQAEDEADYY | |
| CSSYSGIYNLVFGGGTKVTVLG, | |
| PGIA-1-A7 | |
| EVQLVQSGAEVKKPGSSVKVSCKASGGTFKTYAINWVRQAPGQGLEWMGGIIPVLGT | SEQ ID NO:7 |
| ANYVQKFQGRVTITADESTTTAYMELRGLRSEDTAVYYCARGEGSGWYDHYYGLDVW | |
| GQGTLVTVSSGGGGSGGGGSGGGGSAQSVLTQPPSASGTRGQRVTISCSGSSSNIGS | |
| NTVNWYRQLPGTAPKLLIFGDDQRPSGVPDRFSGSRSGTSVSLAISGLQSEDEADYY | |
| CAAWDDSLNGGVFGGGTKLTVLG, | |
| PGIA-1-A8 | |
| EVQLLESGGGLVQPGGSLRLSCAASGFTFSSYAMSWVRQAPGKGLEWVSAISGSGGS | SEQ ID NO:8 |
| TYYADSVKGRFTISRDNSKNTLYLQMNSLRAEDTAVYYCAKDHYYDSSGYLDYWGQG | |
| TLVTVSSGGGGSGGGGSGGGGSALNFMLTQPHSVSESPGKTVTISCTRSSGSIAFDY | |
| VQWYQQRPGSAPTTVIYEDNQRPSGVPDRFSASIDSSSNSASLTISALKTEDEADYY | |
| CQSYDNSNSWVFGGGTKLTVLG, | |
| PGIA-1-A9 | |
| KVQLLESGGGLVQPGGSLRLSCAASGFTFSSYAMSWVRQAPGKGLEWVSAISGSGGS | SEQ ID NO:9 |
| TYYADSVKGRFTISRDNSKNTLYLQMNSLRAEDTAVYYCAKDDVRNAFDIWGRGTTV | |
| TVSSGGGGSGGGGSGGGGSAQSVLTQPPSVSVSPGQTTSITCSRDKLGEQYVYWYQQ | |
| RPGQSPILLLYQDSRRPSWIPERFSGSNSGDTATLTISGTQALDEADYYCQAWDNSS | |
| YVAFGGGTKVTVLG, | |
| PGIA-1-A10 | |
| EVQLLESGGGLVQPGGSLRLSCAASGFTFSSYAMSWVRQAPGKGLEWVSAISGSGGS | SEQ ID NO:10 |
| TYYADSVKGRFTISRDNSKNTLYLQMNSLRAEDTAVYYCARGGELWNPYLDYWGQGT | |
| LVTVSSGGGGSGGGGSGGGGSALPVLTQPPSVSVAPGKTARITCGGNDIASKSVQWF | |
| QQKPGQAPVLVIYYDSDRPSGIPERFSGSNSENTATLTISRVEAGDEADYYCQVWDS | |
| SSDHPVFGGGTKLTVLG, | |
| PGIA-1-A11 | |
| QVQLVQSGAEVKKPGESLKISCKGSGYTFTNYWIAWVRQMPGKGLEWMGIIYPDDSD | SEQ ID NO:11 |
| TRYNPSFQGQVTMSADKSIDTAYLQWSSLKASDTAIYYCARPSGWNDNGYFDYWGRG | |
| TTVTVSSGGGGSGGGGSGGGGSALNFMLTQPHSVSASPGKTVTLSCTGSSGSIASNY | |
| VQWYRQRPGSAPTTVIYDDNQRPSGVPDRFSGSIDSSSNSASLTISGLKTEDEADYY | |
| CQSFDNDNHWVFGGGTKLTVLG, | |
| PGIA-1-A12 | |
| QVQLQESGPGLVRSSGILSLTCSVSGVSVSSNNWWSWVRQTPGKGLEWIGEIYQTGT | SEQ ID NO:12 |
| TNYNPSLKSRVAISLDKSRNQFSLILKSVTAADTAVYYCARTSSAWSNADWGKGTMV | |
| TVSSGGGGSGGGGSGGGGSALSSELTQDPSASGSPGQSVSISCTGTSSDVGGYNYVS | |
| WYQQHPGKAPKLMISEVTKRPSGVPDRFSGSKSGNTASLTVSGLQAEDEADYYCSSF | |
| GANNNYLVFGGGTKLTVLG, | |
| PGIA-1-B1 | |
| QVQLQESGPRLVKPSQTLSLTCTVSNDSIISGDYFWSWIRQPPGKGLEWIGNIFYTG | SEQ ID NO:13 |
| STSYNPSLKSRLTMSLDTSKNQFSLRLSSVTAADTAVYFCARGRQGMNWNSGTYFDS | |
| WGRGTLVTVSSGGGGSGGGGSGGGGSALSYVLTQPPSVSVAPGKTANITCGGKNIGN | |
| KSVQWYQQKPGQAPVVVMYYDSDRPSGIPERFSGSNAGNTATLTIDRVEAGDEADYY | |
| CQVWDKSSDRPVFGGGTKLTVLG, | |
| PGIA-1B2 | |
| QVQLVQSGAEVKKPGASVKVSCKTSGYTFMEYYIHWVRQAPGQGLEWMGWSNPVTGT | SEQ ID NO:14 |
| SGSSPKFRGRVTLTTDTSGNTAYLDLRSLRSDDTAVFYCARRHQQSLDYWGQGTLVT | |
| VSSGGGGSGGGGSGGGGSAQSVVTQPPSASGSPGQSVTISCSGYSSSNIGNNAVSWY | |
| QQLPGTAPKLLIFDNNKRPSGIPARFSGSQSGTTATLGITGLQTGDEADYFCGTWDS | |
| SLSAFVFGSGTKVTVLG, | |
| PGIA-2-A1 | |
| EVQLVQSGAEVKKPGSSVKVSCKASGGSFSNYDFSWVRQAPGQGLEWMGEIINAFGS | SEQ ID NO:15 |
| SRYAQKFQDRVTITADESASTAYMELRGLTSEDTATYYCARAERWELNMAFDMWGRG | |
| TLVTVSSGGGGSGGGGSGGGGSAQSVLTQPPSVSVAPGQTARITCGGDNIGRKNVHW | |
| YQQRPGLAPVLVVYDDTDRPSGIPERFSGSNSGDTATLTITWVEAGDEADYYCQLWD | |
| SDTYDVLFGGGTKLTVLG, | |
| PGIA-2-A2 | |
| EVQLVQSGAEVKKPGSSVKVSCKSSGGPFSSYGISWVRQAPGQGLEWMGGISPIFGT | SEQ ID NO:16 |
| ANYAQKFQGRVTITADESTETAYMELSSLRSEDTAVYYCARDESPVGFYALDIWGRG | |
| TTVTVSSGGGGSGGGGSGGGGSALSYELTQPPSVSVAPGQTARINCGGDKIGSRSVH | |
| WYQQKPGQAPVMVVYDDSDRPSGIPERFSGSNSGNTATLTISSVEAGDEADYYCQVW | |
| DGSTDPWVFGGGTKVTVLG, | |
| PGIA-2-A3 | |
| EVQLVQSGAEMKKPGSSVKVSCKASGGTFSSYAVNWVRQAPGQGLEWMGGIIPIFDT | SEQ ID NO:17 |
| SNYAQKFQGRLTMTADDSTNTAYMELRSLRSEDTAVYYCARGAPRGTVMAFSSYYFD | |
| LWGQGTLVTVSSGGGGSGGGGSGGGGSALNFMLTQPHSVSESPGKTVIISCAGSGGN | |
| IATNYVQWYQHRPGSAPITVIYEDNQRPSGVPDRFSGSVDSSSNSASLTISGLQTED | |
| EADYYCHSYDNTDQGVFGTGTKVTVLG, | |
| PGIA-2-A4 | |
| EVQLVESGGGLVQPGRSLRLSCAASGFTFDDYDMHWVRQAPGKGLEWVSSISWSGGT | SEQ ID NO:18 |
| IGYADSVKGRFTVSRDNAKNSLYLQMNSVRAEDTALYYCAKDRGAVAALPDYQYGMD | |
| VWGRGTLVTVSSGGGGSGGGGSGGGGSAQSALTQPASVSGSPGQSITISCTGTSSDI | |
| GSYNLVSWYQQHPGKAPKLMIYEDYKRASGVSNHFSGSKSGNTASLTISGLQAEDEA | |
| DYYCSSYAGSSAWVFGGGTKVTVLG, | |
| PGIA-2-A5 | |
| EVQLVQSGAEVRKPGSSMKVSCKASGDTFRNFAFSWVRQAPGQGLEWMGGVIPLVGP | SEQ ID NO:19 |
| PKYAQKFQGRLTITADESTSTSYMDLTSLTLEDTAVYFCARGGVYAPFDKWGQGTLV | |
| TVSSGGGGSGGGGSGGGGSAQSVVTQPPSVSEAPRQRVTISCSGSSSNIGNNAVNWY | |
| QQLPGKAPKLLIYYNDLLPSGVSDRFSGSKSGTSASLAISGLQSEDEADYYCAAWDD | |
| SLNGWVFGGGTKVTVLG, | |
| PGIA-2-A6 | |
| EVQLVQSGAEVKKPGSSVKVSCKASGGTFKTYAINWVRQAPGQGLEWMGGIIPVLGT | SEQ ID NO:20 |
| ANYVQKFQGRVTITADESTTTAYMELRGLRSEDTAVYYCARGEGSGWYDHYYGLDVW | |
| GQGTLVTVSSGGGGSGGGGSGGGGSAQSVLTQPPSASGTPGQRVTISCSGSSSNIGS | |
| NTVNWYRQLPGTAPKLLIFGDDQRPSGVPDRFSGSRSGTSVSLAISGLQSEDEADYY | |
| CAAWDDSLNGGVFGGGTKLTVLG, | |
| PGIA-2-A7 | |
| QLQLQESGPGLVKPSGTLSLTCAVSGGSISTSDWWSWVRRPPGKGLEWIGEIYHSGS | SEQ ID NO:21 |
| TNYHPSLKSRVTISLDKSKNQFSLKLSSVTAADTAVYYCAREGGHSGSYPLDYWGKG | |
| TLVTVSSGGGGSGGGGSGGGGSAQAVLTQPSSVSAAPGQKVTISCSGSSSNIGNNYV | |
| SWYQQLPGTAPKLLIYDNNKRPSGIPDRFSGSRSGTSATLGITGLQTGDEADYYCGT | |
| WDSSLSAVVFGTGTKLTVLG, | |
| PGIA-2-A8 | |
| QLQLQESGPGLVKPSGTLSLTCAVSGGSISSTNWWSWVRQPPGKGLEWIGEIYHSGS | SEQ ID NO:22 |
| TNYNPSLKSRVTISVDKSKNHFSLNLSSVTAADTAVYYCARDSMGSTGWHYGMDLWG | |
| KGTLVTVSSGGGGSGGGGSGGGGSAQSALTQPASVSGSPGQSIAISCTGTSSDVGGY | |
| NYVSWYQQHPGKAPKLMIYAVTNRPSGVSDRFSGSKSGNTASLTISGLQADDEADYY | |
| CSSYTSSSSLVFGGGTKLTVLG, | |
| PGIA-2-A9 | |
| GVQLVESGGGLVKPGGSLRLSCAASGFTFSSYTMNWVRQAPGKGLEWVSYISSSGSA | SEQ ID NO:23 |
| TYYADSVKGRFTISRDNANNSLYLQMNSLRAEDTAVYYCARGYRYGMDVWGQGTLVT | |
| VSSGGGGSGGGGSGGGGSGIVMTQSPSTLSASVGDRVTITCRASQGISSWLAWYQQK | |
| PGRAPKVLIYKASTLESGVPSRFSGSGSGTDFTLTISSLQPEDFATYYCQQSYSTPW | |
| TFGQGTKLEIKR, | |
| PGIA-2-A10 | |
| EVQLLESGGGLVQPGGSLRLTCAASGFTFSSYAMSWVRQAPGKGLEWVSAISGSGGS | SEQ ID NO:24 |
| TYYADSVKGRFTISRDNSKNTLYLQMNSLRAEDTAVYYCARDLAVAGIDYWGRGTMV | |
| TVSSGGGGSGGGGSGGGGSAQSVLTQPPSASGTPGQRVTISCSGSSSNIRSNYVYWY | |
| QQFPGTAPKLLIYRNNQRPSGVPDRFSGSKSGTSASLAISGLRSEDEADYYCAAWDD | |
| TLDAYVFAAGTKLTVLG, | |
| PGIA-2-A11 | |
| QVQLQESGPGLVKPSGTLSLTCAVSGGSISTSDWWSWVRRPPGKGLEWIGEIYHSGS | SEQ ID NO:25 |
| TNYHPSLKSRVTISLDKSKNQFSLKLSSVTAADTAVYYCAREGGHSGSYPLDYWGQG | |
| TLVTVSSGGGGSGGGGSGGGGSALNFMLTQPHSVSGSPGRTVTISCTRSSGSIATNY | |
| VQWYQQRPGSSPTIVIYEDNQRPSGVPDRFSGSIDTSSNSASLTISGLKTEDEADYY | |
| CQSYDSNNLGVVFGGGTQLTVLS, | |
| PGIA-2-A12 | |
| QVQLQQSGAEVRKPGASVKISCKTSGYTFMDYYIHWVRQAPGQGLEWMGWSNPVTGT | SEQ ID NO:26 |
| SGSSPKFRGRVTLTTDTSGNTAYLDLRSLRSDDTAVFYCARRHQQSLDYWGQGTLVT | |
| VSSGGGGSGGGGSGGGGSAQAVLTQPSSLSASPGASASLTCTLRSDINVGSYSINWY | |
| QQKPGSPPQYLLNYRSDSDKQQGSGVPSRFSGSKDASANAGILLISGLQSEDEADYY | |
| CMIWYRTAWVFGGGTKVTVLG, | |
| PGIA-2-B1 | |
| QVQLVQSGAEVRKPGASVKVSCKTSGYTFIEYYIHWVRQAPGQGLEWMGWSNPVTGT | SEQ ID NO:27 |
| SGSSPKFRGRVTLTTDTSGNTAYLDLRSLRSDDTAVFYCARRHQQSLDYWGRGTTVT | |
| VSSGGGGSGGGGSGGGGSAQSVLTQPPSVSAAPGQKVTISCSGTNSNIGNYYVSWYQ | |
| QLPGTAPKLLIYDNNKRPSGVPDRFSGSKSGTSASLVISGLRSEDEADYYCAAWDGS | |
| LTAWVFGGGTKVTVLG, | |
| PGIA-3-A1 | |
| QVQLQESGPGLVKPSGTLSLTCAVSGDSISSSNWWTWVRQPPGKGLEWIGEIFHSGT | SEQ ID NO:28 |
| TNYNPSLNNRVTISLDESRNQFSLELSSVTAADTAIYYCARDSGNYDDNRGYDYWGR | |
| GTLVTVSSGGGGSGGGGSGGGGSAQSVLTQPPSVSGAPGQRVTISCAGTSSNIGAGF | |
| DVHWYQLLPGRAPKLLIYGNNNRPSGVPDRFSGSKSGTSASLAISGLQSEDEGDYYC | |
| AAWDDTVGGPVFGGGTKLTVLG, | |
| PGIA-3-A2 | |
| QVQLQESGPGLVKPSGTLSLTCAVSGGSISSTNWWSWVRQPPGKGLEWIGEIYHSGS | SEQ ID NO:29 |
| TNYNPSLKSRVTISVDKSKNHFSLNLSSVTAADTAVYYCARDSMGSTGWHYGMDLWG | |
| RGTLVTVSSGGGGSGGGGSGGGGSAQSALTQPAAVSGSPGQSITISCTGSSSDVGGY | |
| NYVSWYQQHPGKAPKLLIYDVSDRPSGVSYRFSGSKSGNTASLTISGLQAEDEADYY | |
| CSSYTATGTLVFGGGTKLTVLG, | |
| PGIA-3-A3 | |
| QVQLQESGPGLVKPSGTLSLTCAVSGGSISSTNWWSWVRQPPGKGLEWIGEIYHSGS | SEQ ID NO:30 |
| TNYNPSLKSRVTISVDKSKNHFSLNLSSVTAADTAVYYCARDSMGSTGWHYGMDLWG | |
| QGTTVTVSSGGGGSGGGGSGGGGSAQSALTQPASVSGSPGQSITISCTGTSSDVGGY | |
| NYVSWYQQHPGKAPKLMIYEVSNRPLGVSNRFSGSKSGNTASLTISGLQAEDEGDYY | |
| CSSYTSSTTLIVFGGGTKLTVLG, | |
| PGIA-3-A4 | |
| QVQLQESGPGLVKPSGTLSLTCAVSGGSISTSDWWSWVRRPPGKGLEWIGEIYHSGS | SEQ ID NO:31 |
| TNYHPSLKSRVTISLDKSKNQFSLKLSSVTAADTAVYYCAREGGHSGSYPLDYWGQG | |
| TLVTVSSGGGGSGGGGSGGGGSAQSVLTQPPSVSGTTGQRVILSCSGGNSNIGYNSV | |
| NWYQQLPGTAPKLLIYTDDQRPSGVPDRFSGSRSGTSASLAISGLQSEDEADYYCAT | |
| WDDSLNAGVFGGGTKLTVLG, | |
| PGIA-3-A5 | |
| QVQLVQSGAEVRKPGASVRVSCKTSGYTFLEYYIHWVRQAPGQGLEWMAWSNPVTGT | SEQ ID NO:32 |
| SGSSPKFRGRVTLTADTSGNTAYLDLKSLTSDDTAIFYCARRHQQSLDYWGQGTLVT | |
| VSSGGGGSGGGGSGGGGSAQSVLTQPPSVSAAPGQTVTISCSGSNSNIGNNHVSWYR | |
| QLPETAPKLLIYDNNKRPSGIPDRFSGSKSGTSATLDITGLQTGDEADYYCATWDNS | |
| LSAPWVFGGGTKLTVLG, | |
| PGIA-3-A6 | |
| QVQLQESGAEVKKPGSSVKVSCKASGGTFSSSAISWVRQAPGQGLEWMGGIIPVFGT | SEQ ID NO:33 |
| ANYAQKFQDRVTITADESTSTAYLELSRLTSEDTAVYYCASRGEYDYGDYDVYYYYM | |
| EVWGQGTLVTVSSGGGGSGGGGSGGGGSAQSVLTQPPSVSVAPGQTARLTCGANNIG | |
| STSVHWYQQKPGQAPVLVIYDDTDRPSGIPERFSGSNSGNTATLTIRRVEAGDEADY | |
| YCQVWDTNSDHVIFGGGTKLTVLG, | |
| PGIA-3-A7 | |
| EVQLVQSGAEVKKPGSSVKVSCQASGGTFTSHAMYWVRQAPGQGLEWMGGIIPIFGR | SEQ ID NO:34 |
| TNYAQKFQGRVTFTADMSTSTAYMEMTSLRSDDTAVYYCARGDNWNDLYPIDYWGRG | |
| TLVTVSSGGGGSGGGGSGGGGSALNFMLTQPHSVSESPGKTVTISCTRSSGSIATTY | |
| VQWFQQRPGSSPTTVIYDDDQRPSGVPDRFSGSIDSSSNSASLTISGLMPEDEADYY | |
| CQSYDNTDLVFGGGTQLTVLS, | |
| PGIA-3-A8 | |
| EVQLVQSGAEVKKPGASVKVSCKVSGYSLSELSMHWVRQAPGKGLEWMGGFDPQNGY | SEQ ID NO:35 |
| TIYAQEFQGRITMTEDTSTDTVYMELGSLRSEDTAVYFCAAIEITGVNWYFDLWGKG | |
| TLVTVSSGGGGSGGGGSGGGGSALSSELTQDPDVSVALGQTVRITCQGDSLKKFYPG | |
| WYQQKPGQAPLLVLYGENIRPSRIPDRFSGSSSGNTATLTITGAQAEDEAVYYCNSR | |
| EASVHHVRVFGGGTKLTVLG, | |
| PGIA-3-A9 | |
| QVQLQESGPGLVKPSGTLSLTCAVSGGSISTSDWWSWVRRPPGKGLEWIGEIYHSGS | SEQ ID NO:36 |
| TNYHPSLKSRVTISLDKSKNQFSLKLSSVTAADTAVYYCAREGGHSGSYPLDYWGKG | |
| TLVTVSSGGGGSGGGGSGGGGSALNFMLTQPHSVSESPGKTVTISCTRSSGSIASNY | |
| VQWYQQRPGSSPTTVIYEDNQRPSGVPDRFSGSIDSSSNSASLTISGLKTEDEADYY | |
| CQSYDSSNQGVVFGGGTKLTVLG, | |
| PGIA-3-A10 | |
| QLQLQESGPGLVKPSGTLSLTCAVSGGSISTSDWWSWVRRPPGKGLEWIGEIYHSGS | SEQ ID NO:37 |
| TNYHPSLKSRVTISLDKSKNQFSLKLSSVTAADTAVYYCAREGGHSGSYPLDYWGQG | |
| TLVTVSSGGGGSGGGGSGGGGSALNFMLTQPHSVSESPGKTVTISCTGSSGSIASNY | |
| VQWYQQRPGSAPTTLIYEDDQRPSGVPDRFSGSVDSSSNSASLTISGLKTEDEADYY | |
| CQSYDRSNQAVVFGGGTKLTVLG, | |
| PGIA-3-A11 | |
| QVQLVQSGPEVKKPGASVEVSCKASGYTFTGDYMHWVRQAPGQGPEWMGWINPQTGV | SEQ ID NO:38 |
| TKYAQKFQGRVTMARDTSINTAYMELRGLRSDDTAVYYCVREDHNYDLWSAYNGLDV | |
| WGQGTLVTVSSGGGGSGGGGSGGGGSAQSVLTQPPSVSAAPGQKVTISCSGSSSNIG | |
| NNHVSWYQQLAGTAPKLLIFDNDKRPSGIPDRFSGSKSGTSATLGITGLQTGDEADY | |
| YCGTWDKSPTDIYVFGSGTKLTVLG, | |
| PGIA-3-A12 | |
| QVQLQESGPGLVKPSGTLSLTCAVSGGSISSSNWWSWVRQAPGKGLEWIGEIYYGGS | SEQ ID NO:39 |
| TNYNPSLKSRVTLSVDKSKNQFSLRLISVTAADTAVYYCARSSGLYGDYGNLWGRGT | |
| LVTVSSGGGGSGGGGSGGGGSAQSVVTQPPSVSAAPGQKVTISCSGSASNIGDHYIS | |
| WYQQFPGTAPKLLISDNDQRPSGIPDRFSGSKSGTSATLGITGLQTGDEADYYCGTW | |
| DSNLSSWVFGSGTKVTVLG, | |
| PGIA-3-B1 | |
| EVQLVQSGAEVKKPGATLKVSCKVSAYTFTDYSMHWVQQAPGKGLKWMGLIDLEDGN | SEQ ID NO:40 |
| TIYAEEFQDRVTITADTSTDTAYMDLSSLRSEDTAVFYCAISPLRGLTADVFDVWGQ | |
| GTLVTVSSGGGGSGGGGSGGGGSAQSALTQPASASGSPGQSITISCTGTSSDIGRYD | |
| FVSWYQRQPGKAPKLMIYDVINRPSGVSSRFSGSKSGNTASLTISGLQAEDEADYYC | |
| SSYAGSTTLYVFGTGTKLTVLG, | |
| PGIA-3-B2 | |
| QVQLQESGPGLVKPSATLSLTCAVSGGSISSNHWWSWVRQSPGKGLEWIGEIYTYGG | SEQ ID NO:41 |
| ANYNPSLKSRVDISMDKSKNQFSLHLSSVTAADTAVYYCGRHLTGYDCFDIWGQGTL | |
| VTVSSGGGGSGGGGSGGGGSAQAVLTQPSSVSGAPGQRVTISCTGSSSNIGAGYDVH | |
| WYQQLPGTAPKLLIYGNSNRPSGVPDRFSGSKSGTSASLAITGLQAEDEADYYCQSY | |
| DSSLSGVFGTGTQLTVLS, | |
| PGIA-3-B3 | |
| QVQLQESGPGLVKPSGTLSLTCAVSGGSISTSDWWSWVRRPPGKGLEWIGEIYHSGS | SEQ ID NO:42 |
| TNYHPSLKSRVTISLDKSKNQFSLKLSSVTAADTAVYYCAREGGHSGSYPLDYWGQG | |
| TLVTVSSGGGGSGGGGSGGGGSALNFMLTQPHSVSESPGKTVTISCTRSSGSIASKY | |
| VQWYQQRPGSAPTSVIYEDNQRPSGVPDRFSGSIDSASNSASLTISGLKTEDEADYY | |
| CQSDDGSSVVFGGGTKVTVLG, | |
| PGIA-3-B4 | |
| EVQLVQSGAEVKKPGASVKVSCKASGYSFPSSGLSWVRQAPGQGPEWMGWIGIYNGN | SEQ ID NO:43 |
| TDYAQKFQGRVTMTTDKSTSTAYMELRSLRSDDTAVYYCARDSVGSISVAGTMQYYY | |
| FAMDVWGQGTLVTVSSGGGGSGGGGSGGGGSAQSVLTQPPSASGSPGQSVTTSCAGT | |
| RYDIGTYNYVSWYQQHPAKGPKLIIYAVSERPSGVPNRFSGSKSGNTASLTVSGLRA | |
| EDEAHYYCSSYAGNNNVIFGGGTKVTVLG, | |
| PGIA-3-B5 | |
| QVQLQESGPGLVKPSGTLSLTCAVSGGSISTSDWWSWVRRPPGKGLEWIGEIYHSGS | SEQ ID NO:44 |
| TNYHPSLKSRVTISLDKSKNQFSLKLSSVTAADTAVYYCAREGGHSGSYPLDYWGRG | |
| TMVTVSSGGGGSGGGGSGGGGSAQSVLTQPPSASGTPGQRVTISCSGSFSNIGGNYV | |
| NWYQQLPGTAPKLLIYGNNQRPSGVPDRFSSFKSGTSASLAISGLRSEDEADYYCAT | |
| WDDSQTVLFGGGTKLTVLG, | |
| PGIA-3-B6 | |
| EVQLLESGGGLVQPGGSLRLSCAASGFTFSSYAMSWVRQAPGKGLEWVSAISGSGGS | SEQ ID NO:45 |
| TYYADSVKGRFTISRDNSKNTLYLQMNSLRAEDTAVYYCARWNGFLTAHDSWGRGTM | |
| VTVSSGGGGSGGGGSGGGGSAQSVLTQPPSASGTPGQRVTISCSGSSSNIGTNYVYW | |
| YQQFPGTAPKLLIYRSNRRPSGVPDRFSASKSGTSASLVISGLRSEDEADYYCAAWD | |
| DRLNGEMFGGGTKVTVLG, | |
| PGIA-3-B7 | |
| EVQLLESGGGLVQPGGSLRLSCAASGFTFSSYAMSWVRQAPGKGLEWVSAISGSGGS | SEQ ID NO:46 |
| TYYADSVKGRFTISRDNSKNTLYLQMNSLRAEDTAVYYCARWSGRFYDFWGQGTTVT | |
| VSSGGGGSGGGGSGGGGSAQSVLTQPPSASGTPGQRITISCSGSSSNIGSNYVYWYQ | |
| QLPGTAPKILIYRNNQRPSGVPERFSGSKSGTSASLAISGLRSEDEADYYCAAWDDS | |
| LSEVFGGGTKVTVLG, | |
| PGIA-3-B8 | |
| EVQLLESGGGLVQPGGSLRLSCAASGFTFSSYAMSWVRQAPGKGLEWVSAISGSGGS | SEQ ID NO:47 |
| TYYADSVKGRFTISRDNSKNTLYLQMNSLRAEDTAVYYCARDKGYSGFDYWGRGTLV | |
| TVSSGGGGSGGGGSGGGGSAQSVLTQPPSASGTPGQRVTISCSGSSSNIGRHTVNWY | |
| QQLPGTAPKLLIYSNNQRPSGVPDRFSGSKSGTSASLAISGLQSEDEGHYHCAAWDD | |
| TLNGDVVFGGGTKVTVLG | |
| PGIA-4-A1 | |
| QLQLQESGPGLVKPSGTLSLTCAVSGGSISTSDWWSWVRRPPGKGLEWIGEIYHSGS | SEQ ID NO:48 |
| TNYHPSLKSRVTISLDKSKNQFSLKLSSVTAADTAVYYCAREGGHSGSYPLDYWGKG | |
| TLVTVSSGGGGSGGGGSGGGGSALNFMLTQPHSVSESPGKTVTISCTRSSGSIASNY | |
| VQWYQQRPGSSPTTVIYEDNQRPSGVPDRFSGSIDSSSNSASLTISGLKTEDEADYY | |
| CQSYDSSNPYVVFGGGTKLTVLG, | |
| PGIA-4-A2 | |
| QVQLQESGPGLVKPSGTLSLTCAVSGGSISTSDWWSWVRRPPGKGLEWIGEIYHSGS | SEQ ID NO:49 |
| TNYHPSLKSRVTISLDKSKNQFSLKLSSVTAADTAVYYCAREGGHSGSYPLDYWGQG | |
| TLVTVSSGGGGSGGGGSGGGGSALNFMLTQPHSVSGSPGRTVTISCTRSSGSIATNY | |
| VQWYQQRPGSSPTIVIYEDNQRPSGVPDRFSGSIDTSSNSASLTISGLKTEDEADYY | |
| CQSYDSNNLGVVFGGGTQLTVLS, | |
| PGIA-4-A3 | |
| QLQLQESGPGLVKPSGTLSLTCAVSGGSISTSDWWSWVRRPPGKGLEWIGEIYHSGS | SEQ ID NO:50 |
| TNYHPSLKSRVTISLDKSKNQFSLKLSSVTAADTAVYYCAREGGHSGSYPLDYWGQG | |
| TLVTVSSGGGGSGGGGSGGGGSAQSVVTQPPSVSAAPGQKVTISCSGSSSNIGNNYV | |
| SWYKQLPGTAPKLLIYDNNKRPSGIPDRFSGSKSGTSATLGITGLQTGDEADYYCGT | |
| WDSSLSGVVFGGGTKLTVLG, | |
| PGIA-4-A4 | |
| QLQLQESGPGLVKPSGTLSLTCAVSGGSISTSDWWSWVRRPPGKGLEWIGEIYHSGS | SEQ ID NO:51 |
| TNYHPSLKSRVTISLDKSKNQFSLKLSSVTAADTAVYYCAREGGHSGSYPLDYWGRG | |
| TLVTVSSGGGGSGGGGSGGGGSALNFMLTQPHSVSESPGKTVTISCTRSSGSIASNY | |
| VQWYQQRPGSSPTTLIYDDNQRPSGVPDRFSGSIDSSSNSASLTISGLKTEDEADYY | |
| CQSYDSSNLGVVFGGGTKLTVLG, | |
| PGIA-4-A5 | |
| QVQLQESGPGLVKPSGTLSLTCAVSGGSISTSDWWSWVRRPPGKGLEWIGEIYHSGS | SEQ ID NO:52 |
| TNYHPSLKSRVTISLDKSKNQFSLKLSSVTAADTAVYYCAREGGHSGSYPLDYWGRG | |
| TLVTVSSGGGGSGGGGSGGGGSALNFMLTQPHSVSESPGKTATISCTGSGGSIARSY | |
| VQWYQQRPGRAPSIVIYEDYQRPSGVPDRFSGSIDSSSNSASLTITGLKTDDEADYY | |
| CQSSDDNNNVVFGGGTKVTVLG, | |
| PGIA-4-A6 | |
| QVQLQESGPGLVKPSGTLSLTCAVSGGSISTSDWWSWVRRPPGKGLEWIGEIYHSGS | SEQ ID NO:53 |
| TNYHPSLKSRVTISLDKSKNQFSLKLSSVTAADTAVYYCAREGGHSGSYPLDYWGRG | |
| TLVTVSSGGGGSGGGGSGGGGSAQAVLTQPSSVSAAPGQKVTISCSGSSSNIGNNYV | |
| SWYQQLPGTAPKLLIYDNNERPSGIPDRFSGSKSGTSATLGITGLQTGDEADYYCGT | |
| WDSSLSTVVFGTGTKVTVLG, | |
| PGIA-4-A7 | |
| QLQLQESGPGLVKPSGTLSLTCAVSGGSISTSDWWSWVRRPPGKGLEWIGEIYHSGS | SEQ ID NO:54 |
| TNYHPSLKSRVTISLDKSKNQFSLKLSSVTAADTAVYYCAREGGHSGSYPLDYWGQG | |
| TLVTVSSGGGGSGGGGSGGGGSALNFMLTQPHSVSESPGKTVTVSCTGSGGNIASNY | |
| VQWYQQRPDSAPTLVIFEDTQRPSGVPARFSGSIDSSSNSASLIISSLRTEDEADYY | |
| CQSSDSNRVVFGGGTKVTVLG, | |
| PGIA-4-A8 | |
| QVQLQESGPGLVKPSETLSLTCNVSGGSIRNYFWSWIRQPPGQGLEYIGYIYYSGTT | SEQ ID NO:55 |
| DYNPSLKGRVTISLDTSKTQFSLKLNSVTAADTAFYYCVRGPNKYAFDPWGQGTLVT | |
| VSSGGGGSGGGGSGGGGSALSYELTQPPSVSVSPGQTASITCSGDKLGDKFASWYQQ | |
| KAGQSPVLVIYRDTKRPSGIPERFSGSNSGNTATLTISGTQAMDEADYYCQAWDSST | |
| AVFGTGTKVTVLG, | |
| PGIA-4-A9 | |
| QLQLQESGPGLVKPSGTLSLTCAVSGGSISTSDWWSWVRRPPGKGLEWIGEIYHSGS | SEQ ID NO:56 |
| TNYHPSLKSRVTISLDKSKNQFSLKLSSVTAADTAVYYCAREGGHSGSYPLDYWGQG | |
| TLVTVSSGGGGSGGGGSGGGGSALNFMLTQPHSVSESPGKTVTISCTRSSGSTDNNY | |
| VQWYQQRPGSSPTTVIFEDNQRPSGVPDRFSGSIDSSSNSASLTISGLKTEDEADYY | |
| CQSYDSHNQGVVFGGGTKLTVLG | |
| PGIA-4-A10 | |
| QLQLQESGPGLVKPSGTLSLTCAVSGGSISTSDWWSWVRRPPGKGLEWIGEIYHSGS | SEQ ID NO:57 |
| TNYHPSLKSRVTISLDKSKNQFSLKLSSVTAADTAVYYCAREGGHSGSYPLDYWGRG | |
| TLVTVSSGGGGSGGGGSGGGGSAQSVLTQPPSVSAAPGQKVTISCSGSSSNIGNSYV | |
| SWYKQLPGTAPKVLIYDNQKRSSGIPDRFSASKSGTSATLGITGLRTEDEADYYCGT | |
| WDTSLSAVVFGGGTKLTVLG, | |
| PGIA-4-A11 | |
| EVQLVESGPGLVKPSGTLSLTCAVSGGSISTSDWWSWVRRPPGKGLEWIGEIYHSGS | SEQ ID NO:58 |
| TNYHPSLKSRVTISLDKSKNQFSLKLSSVTAADTAVYYCAREGGHSGSYPLDYWGRG | |
| TLVTVSSGGGGSGGGGSGGGGSAQSVVTQPPSVSAAPGQKVTISCSGNFSNIEYNYV | |
| SWYQHLPGTAPKLLIFDNNQRPSWIPDRFSGSKSGTSATLGITGLQTGDEADYYCGT | |
| WDSSLNAGVFGGGTKVTVLG, | |
| PGIA-4-A12 | |
| EVQLLESGGGLVRPGGSLRLSCAASGFTFSSYAMSWVRQAPGKGLEWVSAISGSGGS | SEQ ID NO:59 |
| TYYADSVKGRFTISRDNSKNTLYLQMNSLRAEDTAVYYCAKDRRGVLDPWGKGTMVT | |
| VSSGGGGSGGGGSGGGGSAQSVLTQPPSVSGAPGQRVTISCTGSSSNIGAGYDVHWY | |
| QHLPGTAPRLLIYGNSNRPSGVPDRFSGSKSGTSASLAISGLQAEDEADYYCQSYDS | |
| SLSDWVFGGGTKVTVLG, and | |
| PGIA-5A1 | |
| QLQLQESGPGLVKPSGTLSLTCAVSGGSISTSDWWSWVRRPPGKGLEWIGEIYHSGS | SEQ ID NO:60 |
| TNYHPSLKSRVTISLDKSKNQFSLKLSSVTAADTAVYYCAREGGHSGSYPLDYWGRG | |
| TLVTVSSGGGGSGGGGSGGGGSALNFMLTQPHSVSESPGKTVTISCARSSGSIASNY | |
| VQWYQQRPGSSPTTLIYEDRQRPSGVPDRFSGSIDSSSNSASLTISGLKTEDEADYY | |
| CQSYDSSDHVVFGGGTKLTVLG. | |

In another preferred embodiment, the c-Met antibody comprises a light chain amino acid sequence from SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60, or one or more CDRs from these amino acid sequences. In another preferred embodiment, the c-Met antibody comprises a heavy chain amino acid sequence from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60 or one or more CDRs from these amino acid sequences.

Class and Subclass of C-Met Antibodies

The antibody may be an IgG, an IgM, an IgE, an IgA, or an IgD molecule. In a preferred embodiment, the antibody is an IgG and is an IgG1, IgG2, IgG3, or IgG4 subtype. In a more preferred embodiment, the c-Met antibody is subclass IgG1. In another preferred embodiment, the c-Met antibody is the same class and subclass as antibody PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1, which is IgG1.

The class and subclass of c-Met antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot, as well as other techniques.

Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

Molecule Selectivity

In another embodiment, the c-Met antibody has a selectivity for c-Met that is at least 50 times greater than its selectivity for IGF-1R, insulin, Ron, Axl, and Mer receptors. In a preferred embodiment, the selectivity of the c-Met antibody is more than 100 times greater than for IGF-1R, insulin, Ron, Axl, and Mer receptors. In an even more preferred embodiment, the c-Met antibody does not exhibit any appreciable specific binding to any other protein than c-Met. One may determine the selectivity of the c-Met antibody for c-Met using methods well known in the art following the teachings of the specification. For instance, one may determine the selectivity using Western blot, FACS, ELISA, or RIA. In a preferred embodiment, one may determine the molecular selectivity using Western blot.

Binding Affinity of c-Met Antibody to c-Met

In another aspect of the invention, the c-Met antibodies bind to c-Met with high affinity. In one embodiment, the c-Met antibody binds to c-Met with a K_{d} of 1×10⁻⁸ M or less. In a more preferred embodiment, the antibody binds to c-Met with a K_{d} or 1×10⁻⁹ M or less. In an even more preferred embodiment, the antibody binds to c-Met with a K_{d} or 5×10⁻¹⁰ M or less. In another preferred embodiment, the antibody binds to c-Met with a K_{d} of 1×10⁻¹⁰ M or less. In another preferred embodiment, the antibody binds to c-Met with substantially the same K_{d} as an antibody selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another preferred embodiment, the antibody binds to c-Met with substantially the same K_{d} as an antibody that comprises one or more CDRs from an antibody selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In still another preferred embodiment, the antibody binds to c-Met with substantially the same K_{d} as an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. In another preferred embodiment, the antibody binds to c-Met with substantially the same K_{d} as an antibody that comprises one or more CDRs from an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60.

In another aspect of the invention, the c-Met antibody has a low dissociation rate. In one embodiment, the c-Met antibody has a K_{off} of 1×10⁻¹ s⁻¹ or lower. In a preferred embodiment, the K_{off} is 5×10⁻⁵ s⁻¹ or lower. In another preferred embodiment, the K_{off} is substantially the same as an antibody selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another preferred embodiment, the antibody binds to c-Met with substantially the same K_{off} as an antibody that comprises one or more CDRs from an antibody selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In still another preferred embodiment, the antibody binds to c-Met with substantially the same K_{off} as an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. In another preferred embodiment, the antibody binds to c-Met with substantially the same K_{off} as an antibody that comprises one or more CDRs from an antibody that comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60 or a fragment thereof.

The binding affinity and dissociation rate of a c-Met antibody to c-Met may be determined by any method known in the art. In one embodiment, the binding affinity can be measured by competitive ELISAs, RIAs, or surface plasmon resonance, such as BIAcore. The dissociation rate can also be measured by surface plasmon resonance. In a more preferred embodiment, the binding affinity and dissociation rate is measured by surface plasmon resonance. In an even more preferred embodiment, the binding affinity and dissociation rate is measured using a BIAcore. An example of determining binding affinity and dissociation rate for binding of c-Met antibodies to the extracellular domain of human c-Met using BIAcore is described below in Example 10.

Half-Life c-Met Antibodies

According to another object of the invention, the c-Met antibody has a half-life of at least one day in vitro or in vivo. In a preferred embodiment, the antibody or portion thereof has a half-life of at least three days. In a more preferred embodiment, the antibody or portion thereof has a half-life of four days or longer. In another embodiment, the antibody or portion thereof has a half-life of eight days or longer. In another embodiment, the antibody or antigen-binding portion thereof is derivatized or modified such that it has a longer half-life, as discussed below.

In another preferred embodiment, the antibody may contain point mutations to increase serum half-life, such as described WO 00/09560, published Feb. 24, 2000.

The antibody half-life may be measured by any means known to one having ordinary skill in the art. For instance, the antibody half-life may be measured by Western blot, ELISA or RIA over an appropriate period of time. The antibody half-life may be measured in any appropriate animals, e.g., a monkey, such as a cynomolgus monkey, a primate or a human.

The invention also provides a c-Met antibody that binds the same antigen or epitope as a human c-Met antibody of the present invention. Further, the invention provides a c-Met antibody that cross-competes with a c-Met antibody known to block HGF binding. In a highly preferred embodiment, the known c-Met antibody is another human antibody. In a preferred embodiment, the human c-Met antibody has the same antigen or epitope of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another preferred embodiment, the human c-Met antibody comprises one or more CDRs from an antibody that binds the same antigen or epitope selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In still another preferred embodiment, the human c-Met antibody that binds the same antigen or epitope comprises one of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60 or a fragment thereof. In another preferred embodiment, the human c-Met antibody that binds the same antigen or epitope comprises one or more CDRs from an antibody of the amino acid sequences selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60.

One may determine whether a c-Met antibody binds to the same antigen using a variety of methods known in the art. For instance, one may determine whether a test c-Met antibody binds to the same antigen by using a c-Met antibody to capture an antigen that is known to bind to the c-Met antibody, such as c-Met, eluting the antigen from the antibody, and determining whether the test antibody will bind to the eluted antigen. One may determine whether the antibody binds to the same epitope as a c-Met antibody by binding the c-Met antibody to c-Met under saturating conditions, and then measuring the ability of the test antibody to bind to c-Met. If the test antibody is able to bind to the c-Met at the same time as the c-Met antibody, then the test antibody binds to a distinct epitope from the c-Met antibody. However, if the test antibody is not able to bind to the c-Met at the same time, then the test antibody binds to the same epitope, or shares an overlapping epitope binding site, as the human c-Met antibody. This experiment may be performed using ELISA, RIA, or surface plasmon resonance. In a preferred embodiment, the experiment is performed using surface plasmon resonance. In a more preferred embodiment, BIAcore is used. One may also determine whether a c-Met antibody cross-competes with another c-Met antibody. In a preferred embodiment, one may determine whether a c-Met antibody cross-competes with another by using the same method that is used to measure whether the c-Met antibody is able to bind to the same epitope as another c-Met antibody.

Light and Heavy Chain Usage

The invention also provides a c-Met antibody that comprises variable sequences encoded by a human λ or κ gene. In a preferred embodiment, the light chain variable sequences are encoded by the Vλ 1e, 1b, 3r, or 6a gene family. In one embodiment, the variable sequences are encoded by the Vκ A27, A30, or O12 gene family. In a more preferred embodiment, the light chain comprises no more than ten amino acid substitutions from the germline, preferably no more than six amino acid substitutions, and more preferably no more than three amino acid substitutions. In a preferred embodiment, the amino acid substitutions are conservative substitutions.

SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60 provide the amino acid sequences of the variable regions of c-Met antibody λ light chains. Following the teachings of this specification, one of ordinary skill in the art could determine the encoded amino acid sequence of the c-Met antibody light chains and the germline light chains and determine the differences between the germline sequences and the antibody sequences.

In a preferred embodiment, the VL of the c-Met antibody contains the same amino acid substitutions, relative to the germline amino acid sequence, as any one or more of the VL of antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. For example, the VL of the c-Met antibody may contain one or more amino acid substitutions that are the same as those present in antibody PGIA-03-A9, another amino acid substitution that is the same as that present in antibody PGIA-03-B2, and another amino acid substitution that is the same as antibody PGIA-01-A8. In this manner, one can mix and match different features of antibody binding in order to alter, e.g., the affinity of the antibody for c-Met or its dissociation rate from the antigen. In another embodiment, the amino acid substitutions are made in the same position as those found in any one or more of the VL of antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, but conservative amino acid substitutions are made rather than using the same amino acid. For example, if the amino acid substitution compared to the germline in one of the antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1 is glutamate, one may conservatively substitute aspartate.

Similarly, if the amino acid substitution is serine, one may conservatively substitute threonine. In another preferred embodiment, the light chain comprises an amino acid sequence that is the same as the amino acid sequence of the VL of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A1, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another highly preferred embodiment, the light chain comprises amino acid sequences that are the same as the CDR regions of the light chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another preferred embodiment, the light chain comprises an amino acid sequence from at least one CDR region of the light chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another preferred embodiment, the light chain comprises amino acid sequences from CDRs from different light chains. In a more preferred embodiment, the CDRs from different light chains are obtained from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A1, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another preferred embodiment, the light chain comprises a VL amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. In another embodiment, the light chain comprises an amino acid sequence encoded by a nucleic acid sequence selected from SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO: 102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120, fragments thereof, or a nucleic acid sequence that encodes an amino acid sequence having 1-10 amino acid insertions, deletions or substitutions therefrom. Preferably, the amino acid substitutions are conservative amino acid substitutions. In another embodiment, the antibody or portion thereof comprises a lambda light chain.

The present invention also provides a c-Met antibody or portion thereof, which comprises a human heavy chain or a sequence derived from a human heavy chain. In one embodiment, the heavy chain amino acid sequence is derived from a human V_{H} DP-35, DP-47, DP-70, DP-71, or VIV-4/4.35 gene family. In a more preferred embodiment, the heavy chain comprises no more than eight amino acid changes from germline, more preferably no more than six amino acid changes, and even more preferably no more than three amino acid changes.

SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60 provide the amino acid sequences of the variable regions of c-Met antibody heavy chains. Following the teachings of this specification, one of ordinary skill in the art could determine the encoded amino acid sequence of the c-Met antibody heavy chains and the germline heavy chains and determine the differences between the germline sequences and the antibody sequences.

In a preferred embodiment, the VH of the c-Met antibody contains the same amino acid substitutions, relative to the germline amino acid sequence, as any one or more of the VH of antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. Similar to what was discussed above, the VH of the c-Met antibody may contain one or more amino acid substitutions that are the same as those present in antibody PGIA-03-A9, another amino acid substitution that is the same as that present in antibody PGIA-03-B2, and another amino acid substitution that is the same as antibody PGIA-01-A8. In this manner, one can mix and match different features of antibody binding in order to alter, e.g., the affinity of the antibody for c-Met or its dissociation rate from the antigen. In another embodiment, the amino acid substitutions are made in the same position as those found in any one or more of the VH of antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, but conservative amino acid substitutions are made rather than using the same amino acid.

In another preferred embodiment, the heavy chain comprises an amino acid sequence that is the same as the amino acid sequence of the VH of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another highly preferred embodiment, the heavy chain comprises amino acid sequences that are the same as the CDR regions of the heavy chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another preferred embodiment, the heavy chain comprises an amino acid sequence from at least one CDR region of the heavy chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another preferred embodiment, the heavy chain comprises amino acid sequences from CDRs from different heavy chains. In a more preferred embodiment, the CDRs from different heavy chains are obtained from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another preferred embodiment, the heavy chain comprises a VH amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. In another embodiment, the heavy chain comprises a VH amino acid sequence encoded by a nucleic acid sequence selected from SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ If NO:105, SEQ ID NO:106, SEQ ID NO:107, SEQ ID NO:108, SEQ ID NO:109, SEQ ID NO:100, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120, a fragment thereof, or a nucleic acid sequence that encodes an amino acid sequence having 1-10 amino acid insertions, deletions or substitutions therefrom. In another embodiment, the substitutions are conservative amino acid substitutions.

Table 2 shows a nucleic acid sequences encoding the scFvs PGIA-01-A1 through PGIA-05-A1.

TABLE 2

| | |
|---|---|
| PGIA-01-A1 | |
| GAGGTGCAGCTGTTGGAGTCTGGGCGAGGCTTGGTACAGCCTGGGGGGTCCCTGAGA | SEQ ID NO:61 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAGATTTGCCGTAACTGGGGAGTTTGACTACTGGGGGCAGGGGACCACGGTC | |
| ACCGTCTCGAGTGGAGGCGGCGGTTCAGGCGGAGGTGGCTCTGGCGGTGGCGGAAGT | |
| GCACAGGCTGTGCTGACTCAGCCGTCCTCAGTGTCTGGGGCCCCAGGGCAGAGGGTC | |
| ACCATCTCCTGCACTGGGAGCAGCTCCAACATCGGGGCAGATTATGATGTACACTGG | |
| TACCAGCAGCTTCCAGGAACAGCCCCCAAACTCCTCATCTATGGTAACAACAATCGG | |
| CCCTCAGGGGTCCCTGACCGATTCTCTGGCTCCAAGTCTGGCACCTCAGCCTCCCTG | |
| GCCATCACTGGGCTCCAGGCTGAGGATGAGGCTGATTATTACTGCCAGTCCTATGAC | |
| AACAGCCCGGATGCCTATGTGGTCTTCGGCGGAGGGACCAAGCTGACCGTCCTAAGT, | |
| PGIA-01-A2 | |
| CAGGTGCAGCTGGTGCAGTCTGGGGCTGAGGTGAGAAAGCCTGGGGCCTCAGTGAAG | SEQ ID NO:62 |
| GTCTCCTGCAAGACTTCTGGATACACCTTCATCGACTACTATATACACTGGGTGCGA | |
| CAGGCCCCTGGACAAGGGCTTGAGTGGATGGGCTGGGTCAACCCTGTCACTGGAACC | |
| TCAGGCTCTTCACCCAACTTTCGGGGCAGGGTCACCATGACCACCGACACGTCCGGC | |
| AACACAGCCTATATGGAACTGAGGAGCCTTAGATCTGACGACACGGCCGTATTTTAC | |
| TGTGCGAGGCGTCACCAACAGAGCTTGGATTATTGGGGCCAGGGAACCCTGGTCACC | |
| GTCTCGAGTGGAGGCGGCGGTTCAGGCGGAGGTGGCTCTGGCGGTGGCGGAAGTGCA | |
| CAGTCTGTGTTGACGCAGCCGCCCTCAGTGTCTGCGCCCCCGGGACAGAAGGTCACC | |
| ATCTCCTGCTCTGGAAGCAGCTCCAACATTGGGACTAATTATGTATCCTGGTACCAG | |
| CAGCTCCCAGGAACAGCCCCCAAACTCCTCATTTATGACAATCATAAGCGACCCTCA | |
| GTGATTCCTGACCGCTTCTCTGGCTCCAAGTCTGGCACGTCAGCCACCCTGGGCATC | |
| TCCGGACTCCAGACTGGGGACGAGGCCGATTATTACTGCGGAACATGGGATTACAGC | |
| CTGAGTACTTGGGTGTTCGGCGGAGGGACCAAGCTGACCGTCCTAGGT, | |
| PGIA-01-A3 | |
| CAGTTGCAGCTGCAGGAGTCCGGCCCAGGACTGGTGAAGCCTTCGGGGACCCTGTCC | SEQ ID NO:63 |
| CTCACCTGCGCTGTCTCTGGAGACTCCGTCAGCAGTTATTACTGGTGGAGTTGGGTC | |
| CGCCAGCCCCCAGGGAAGGGGCTGGAGTGGATTGGAGAAATCTTTCGTGATGGGAGC | |
| TCCAACTACAACCGGTCCCTCAAGAGTCGGGTCACCATATCCCCAGACAAGCCCAAG | |
| AATCAGTTCTCTCTGAGGCTGAGCTCTGTGACCGCCGCGGACACGGCCATTTACTAC | |
| TGTGCGAGGCATATACGCGGTTATGATGCTTTTGACATCTGGGGCCGGGGAACCCTG | |
| GTCACCGTCTCGAGTGGAGGCGGCGGTTCAGGCGGAGGTGGCTCTGGCGGTGGCGGA | |
| AGTGCACAGTCTGTGTTGACGCAGCCGCCCTCAGTGTCTGGGGCCCCAGGGCAGAGG | |
| GTCACCATCTCCTGTACTGGGAGCAGCTCCAACATCGGGGCAGGTTATGATGTACAC | |
| TGGTACCAGCAGTTTCCAGGAAGAGCCCCCAAGCTCCTCATCTATGGTAACACCAAT | |
| CGGCCCTCAGGGGTCCCTGACCGATTCTCTGGCTCCAAGTCTGACATCTCAGCCTCC | |
| CTGGCCATCACTGGGCTCCAGGCTGAGGATGAGGCTGATTATTACTGTCAGTCCTAT | |
| GACAGCAACCTGACTGGGGTGTTCGGCGGAGGGACC, | |
| PGIA-01-A4 | |
| CAGGTGCAGCTGGTGCAGTCTGGGGCTGAGGTGAGGAAGCCTGGGGCCTCAGTGAAG | SEQ ID NO:64 |
| GTCTCCTGCAAGACTTCTGGATACACCTTCATGGACTACTACATACACTGGGTGCGA | |
| CAGGCCCCTGGACAAGGGCTTGAGTGGATGGGCTGGAGCAACCCTGTCACTGGTACG | |
| TCAGGCTCTTCACCTAAATTTCGGGGCAGGGTCACCTTGACCACTGACACGTCCGGC | |
| AACACAGCCTATTTGGACCTGAGGAGCCTTAGATCTGACGACACGGCCGTATTTTAC | |
| TGTGCGAGGCGTCACCAACAGAGCTTGGATTATTGGGGCCAAGGGACAATGGTCACC | |
| GTCTCGAGTGGAGGCGGCGGTTCAGGCGGAGGTGGCTCTGGCGGTGGCGGAAGTGCA | |
| CAGTCTGTGTTGACGCAGCCGCCCTCAGTGTCTGCGGCCCCAGGACAGAAGGTCACC | |
| ATCTCCTGCTCTGGAAGCAGCTCCAACATTGGGAATAATTATGTATCCTGGTACCAG | |
| CAACTCCCAGGAACAGCCCCCAAACTCCTCATGTATGAAAATAGTAAGCGACCCTCA | |
| GGGATTCCTGACCGGTTCTCTGGCTCCAAGTCTGGCACGTCAGGCACCCTGGGCATC | |
| ACCGGACTCCAGACTGGGGACGAGGCCGATTATTACTGCGGAACATGGGATACCAGC | |
| CTGAGAGCTTGGGTGTTCGGCGGAGGGACCAAGGTCACCGTCCTAGGT, | |
| PGIA-01-A5 | |
| CAGGTACAGCTGCAGCAGTCAGGGGCTGAGGTGAGGAAGCCTGGGGCCTCGGCGAAG | SEQ ID NO:65 |
| GTCTCCTGCAAGACTTCTGGATACACCTTCATCGACTACTATATACACTGGGTGCGA | |
| CAGGCCCCTGGACAAGGGCTTGAGTGGATGGGCTGGATCAACCCTGTCACTGGTGCC | |
| TCAGGCTCTTCACCTAACTTTCGGGGCAGGGTCACCTTGACCACCGACACGTCCGGC | |
| AACACAGCCTATATGGAGCTGAGGAGCCTTAGATCTGACGACACGGCCGTGTTTTAC | |
| TGTGCGAGGCGTCACCAACAGAGCTTGGATTATTGGGGGCGGGGGACCACGGTCACC | |
| GTCTCGAGTGGAGGCGGCGGTTCAGGCGGAGGTGGCTCTGGCGGTGGCGGAAGTGCA | |
| CAGTCTGTCGTGACGCAGCCGCCCTCAGTGTCTGCGGCTCCAGGACAGAAGGTCACC | |
| ATCTCCTGCTCTGGGAGGACATCCAACATTGGGAACAATTATGTATCCTGGTATCAG | |
| CAAGTCCCAGGAGCGCCCCCCAAACTACTCATTTTTGACAATAATAAGCGACCCTCA | |
| GGGACTCCTGCCCGATTCTCTGGCTCCAAGTCTGGCACGTCAGCCACCCTGGCCATC | |
| TCCGGACTCCAGACCGGGGACGAGGCCGATTATTACTGCGGAACATGGGATACTACC | |
| CTGCGTGGTTTTGTCTTCGGGCCCGGGACCAAGGTCACCGTCCTAGGT, | |
| PGIA-01-A6 | |
| CAGCTGCAGCTGCAGGAGTCGGGCCCAGGACTGGTGAAGCCTTCGGGGACCCTGTCC | SEQ ID NO:66 |
| CTCACCTGCGCTGTCTCTGGTGGCTCCATCAGCAGTACTAACTGGTGGAGTTGGGTC | |
| CGCCAGCCCCCAGGGAAGGGGCTGGAGTGGATTGGGGAAATCTATCATAGTGGGAGC | |
| ACCAACTACAACCCGTCCCTCAAGAGTCGAGTCACCATATCAGTAGACAAGTCCAAG | |
| AACCACTTCTCCCTGAACCTGAGCTCTGTGACCGCCGCGGACACGGCCGTGTATTAC | |
| TGTGCGAGAGATTCTATGGGAAGCACTGGCTGGCATTACGGTATGGACCTCTGGGGC | |
| CGGGGAACCCTGGTCACCGTCTCGAGTGGAGGCGGCGGTTCAGGCGGAGGTGGCTCT | |
| GGCGGTGGCGGAAGTGCACAATCTGCCCTGACTCAGCCTCCCTCCGCGTCCGGGTCT | |
| CCTGGACAGTCAGTCACCATCTCCTGCAGTGGAAGCAGTAGTGACATTGGTGATTAT | |
| AACCATGTCTCCTGGTACCAACAGCACCCAGGCAAAGCCCCCAAACTCATGATTTAT | |
| GACGTCAATAAGTGGCCCTCAGGGGTCCCTGATCGCTTCTCTGGCTCCAAGTCTGGC | |
| AACACGGCCTCCCTGACCGTCTCTGGGCTCCAGGCTGAGGATGAGGCTGATTATTAT | |
| TGCAGCTCATATTCAGGCATCTACAATTTGGTTTTCGGCGGAGGGACCAAGGTCACC | |
| GTCCTAGGT, | |
| PGIA-01-A7 | |
| GAGGTGCAGCTGGTGCAGTCTGGGGCTGAAGTGAAGAAGCCTGGGTCCTCGGTGAAG | SEQ ID NO:67 |
| GTCTCCTGTAAGGCCTCTGGAGGCACCTTCAAGACCTATGCTATCAATTGGGTGCGA | |
| CAGGCCCCTGGACAAGGGCTTGAGTGGATGGGAGGAATCATCCCTGTCCTGGGAACA | |
| GCAAATTACGTTCAGAAGTTCCAGGGCAGAGTCACGATTACCGCGGACGAATCGACG | |
| ACCACAGCCTACATGGAGCTGAGGGGCCTGAGATCTGAGGACACGGCCGTTTATTAT | |
| TGTGCGAGAGGAGAGGGCAGTGGCTGGTACGATCACTACTACGGATTGGACGTCTGG | |
| GGCCAAGGAACCCTGGTCACCGTCTCGAGTGGAGGCGGCGGTTCAGGCGGAGGTGGC | |
| TCTGGCGGTGGCGGAAGTGCACAGTCTGTGCTGACGCAGCCGCCCTCAGCGTCTGGG | |
| ACCCCCGGGCAGAGGGTCACCATCTCTTGTTCTGGAAGCAGCTCCAACATCGGAAGT | |
| AATACTGTAAACTGGTACCGGCAGCTCCCAGGAACGGCCCCCAAACTCCTCATCTTT | |
| GGTGATGATCAGCGGCCCTCAGGGGTCCCTGACCGATTCTCTGGCTCCAGGTCTGGC | |
| ACCTCAGTCTCCCTGGCCATCAGTGGGCTCCAGTCTGAGGATGAGGCTGACTATTAC | |
| TGTGCAGCATGGGATGACAGCCTGAATGGCGGGGTGTTCGGCGGAGGGACCAAGCTG | |
| ACCGTCCTAGGT, | |
| PGIA-01-A8 | |
| GAGGTGCAGCTGTTGGAGTCTGGGGGAGGCTTGGTACAGCCTGGGGGGTCCCTGAGA | SEQ ID NO:68 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAAAGATCATTACTATGATAGTAGTGGTTATCTTGACTACTGGGGCCAAGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGCCTTCGACTAT | |
| GTGCAGTGGTACCAGCAGCGCCCGGGCAGTGCCCCCACCACTGTGATCTATGAGGAT | |
| AATCAAAGACCCTCTGGGGTCCCTGATCGGTTCTCTGCCTCCATCGACAGCTCCTCC | |
| AACTCTGCCTCCCTCACCATCTCTGCACTGAAGACTGAGGACGAGGCTGACTACTAC | |
| TGTCAGTCTTATGATAACAGCAATTCTTGGGTCTTCGGCGGAGGGACCAAGCTGACC | |
| GTCCTAGGT, | |
| PGIA-01-A9 | |
| AAGGTGCAGCTGTTGGAGTCTGGGGGAGGCTTGGTACAGCCTGGGGGGTCCCTGAGA | SEQ ID NO:69 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAAAGATGATGTTCGGAATGCTTTTGATATCTGGGGGAGGGGGACCACGGTC | |
| ACCGTCTCGAGTGGAGGCGGCGGTTCAGGCGGAGGTGGCTCTGGCGGTGGCGGAAGT | |
| GCACAGTCTGTGCTGACTCAGCCACCCTCAGTGTCCGTGTCCCCAGGACAGACAACC | |
| AGCATCACCTGCTCTAGAGATAAGTTGGGAGAACAATATGTTTACTGGTATCAACAG | |
| AGGCCAGGCCAGTCCCCTATTCTACTCCTCTATCAAGATTCCAGGCGGCCCTCATGG | |
| ATCCCTGAGCGATTCTCTGGCTCCAACTCTGGGGACACAGCCACTCTGACCATCAGC | |
| GGGACCCAGGCTCTGGATGAGGCTGACTACTACTGTCAGGCGTGGGACAACAGTTCC | |
| TATGTAGCATTCGGCGGAGGGACCAAGGTCACCGTCCTAGGT, | |
| PGIA-01-A10 | |
| GAGGTGCAGCTGTTGGAGTCTGGGGGAGGCTTGGTACAGCCTGGGGGGTCCCTGAGA | SEQ ID NO:70 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAGAGGAGGGGAGCTGTGGAATCCATATTTAGACTACTGGGGCCAGGGCACC | |
| CTGGTCACCGTCTCGAGTGGAGGCGGCGGTTCAGGCGGAGGTGGCTCTGGCGGTGGC | |
| GGAAGTGCACTGCCTGTGCTGACTCAGCCCCCCTCAGTGTCAGTGGCCCCAGGGAAG | |
| ACGGCCAGGATTACCTGTGGGGGAAACGACATTGCAAGTAAAAGTGTGCAGTGGTTT | |
| CAGCAGAAGCCAGGCCAGGCCCCTGTACTGGTCATCTATTATGATAGCGACCGGCCC | |
| TCAGGGATCCCTGAGCGATTCTCTGGCTCCAACTCTGAGAACACGGCCACCCTGACC | |
| ATCAGCAGGGTCGAAGCGGGGGATGAGGCCGACTATTATTGTCAGGTGTGGGATAGC | |
| AGTAGTGATCATCCGGTGTTCGGCGGAGGGACCAAGCTGACCGTCCTAGGT, | |
| PGIA-01-A11 | |
| CAGGTCCAGCTGGTGCAGTCTGGGGCAGAGGTGAAAAAGCCCGGGGAGTCTCTGAAA | SEQ ID NO:71 |
| ATCTCCTGTAAGGGTTCTGGATACACTTTTACCAATTACTGGATCGCCTGGGTGCGC | |
| CAGATGCCCGGAAAAGGCCTGGAGTGGATGGGAATCATTTATCCTGATGACTCTGAT | |
| ACCAGATACAACCCGTCCTTCCAAGGCCAGGTCACCATGTCAGCCGACAAGTCCATC | |
| GACACCGCCTATCTGCAGTGGAGCAGCCTGAAGGCCTCGGACACCGCCATATATTAC | |
| TGTGCGAGACCCTCGGGCTGGAACGACAATGGCTACTTTGACTACTGGGGGCGAGGG | |
| ACCACGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGCGTCTCCG | |
| GGGAAGACGGTCACCCTCTCCTGCACCGGCTCCAGTGGCAGCATTGCCAGCAACTAT | |
| GTGCAGTGGTACCGGCAGCGCCCGGGCAGTGCCCCCACCACTGTGATCTATGACGAT | |
| AATCAAAGACCCTCTGGGGTCCCTGATCGTTTCTCTGGCTCCATCGACAGCTCCTCC | |
| AACTCTGCCTCCCTCACCATCTCTGGACTGAAGACTGAGGACGAGGCTGACTACTAC | |
| TGTCAGTCTTTTGATAACGACAATCATTGGGTGTTTGGCGGAGGGACCAAGCTGACC | |
| GTCCTAGGT, | |
| PGIA-01-A12 | |
| CAGGTGCAGCTGCAGGAGTCGGGCCCAGGACTGGTGAGGTCTTCGGGGATCCTGTCC | SEQ ID NO:72 |
| CTCACCTGCTCTGTCTCTGGTGTCTCCGTCAGCAGTAATAACTGGTGGAGTTGGGTC | |
| CGCCAGACCCCAGGGAAGGGGCTGGAGTGGATCGGGGAAATCTATCAGACCGGGACC | |
| ACCAACTACAACCCGTCTCTCAAGAGCCGAGTCGCCATATCACTAGACAAGTCCAGG | |
| AATCAGTTCTCCCTGATTTTGAAGTCTGTGACCGCCGCGGACACGGCCGTATATTAC | |
| TGCGCGAGAACTAGCAGCGCCTGGTCTAACGCTGATTGGGGCAAAGGGACAATGGTC | |
| ACCGTCTCGAGTGGAGGCGGCGGTTCAGGCGGAGGTGGCTCTGGCGGTGGCGGAAGT | |
| GCACTTTCTTCTGAGCTGACTCAGGACCCCTCCGCGTCCGGGTCTCCTGGACAGTCA | |
| GTCAGCATCTCTTGCACTGGAACCAGCAGTGACGTTGGTGGTTATAATTATGTCTCC | |
| TGGTACCAACAGCACCCAGGCAAAGCCCCCAAACTCATGATTTCTGAGGTCACTAAG | |
| CGGCCCTCAGGGGTCCCTGATCGCTTCTCTGGCTCCAAGTCTGGCAACACGGCCTCC | |
| CTGACCGTCTCTGGGCTCCAGGCTGAAGATGAGGCTGATTATTACTGCAGCTCATTT | |
| GGAGCCAACAACAATTATCTCGTATTCGGCGGAGGGACCAAGCTGACCGTCCTAGGT, | |
| PGIA-01-B1 | |
| CAGGTGCAGCTGCAGGAGTCGGGCCCAAGACTGGTGAAGCCTTCACAGACCCTGTCC | SEQ ID NO:73 |
| CTCACCTGCACTGTCTCTAATGACTCCATCATCAGTGGCGATTACTTCTGGAGTTGG | |
| ATCCGCCAGCCCCCAGGGAAGGGCCTGGAGTGGATTGGGAACATCTTTTATACTGGG | |
| AGCACCTCTTACAATCCGTCCCTCAAGAGTCGACTTACCATGTCCCTAGACACGTCC | |
| AAGAACCAGTTCTCCCTGAGATTGAGCTCTGTGACTGCCGCAGACACGGCCGTATAT | |
| TTTTGTGCCAGAGGTCGACAGGGGATGAACTGGAATTCCGGGACCTACTTCGACTCC | |
| TGGGGCAGAGGAACCCTGGTCACCGTCTCGAGTGGAGGCGGCGGTTCAGGCGGAGGT | |
| GGCTCTGGCGGTGGCGGAAGTGCACTTTCCTATGTGCTGACTCAGCCACCCTCTGTG | |
| TCCGTGGCCCCAGGAAAGACGGCCAATATAACTTGTGGGGGAAAGAACATTGGAAAT | |
| AAAAGTGTGCAGTGGTATCAGCAGAAGCCAGGCCAGGCCCCTGTGGTAGTCATGTAT | |
| TATGACAGCGACCGGCCCTCAGGGATTCCTGAGCGATTCTCTGGCTCCAACGCTGGG | |
| AACACGGCCACCCTGACCATCGACAGGGTCGAGGCCGGGGATGAGGCCGATTATTAC | |
| TGTCAGGTGTGGGATAAAAGTAGTGATCGTCCGGTCTTCGGCGGAGGGACCAAGCTG | |
| ACCGTCCTAGGT | |
| PGIA-01-B2 | |
| CAGGTCCAGCTGGTGCAGTCTGGGGCTGAGGTGAAGAAGCCTGGGGCCTCAGTGAAG | SEQ ID NO:74 |
| GTCTCCTGCAAGACTTCTGGATACACCTTCATGGAATACTACATACACTGGGTGCGA | |
| CAGGCCCCTGGACAAGGGCTTGAGTGGATGGGCTGGAGCAATCCTGTCACTGGTACG | |
| TCAGGCTCTTCACCTAAGTTTCGGGGCAGGGTCACCTTGACCACTGACACGTCCGGC | |
| AACACAGCCTATTTGGACCTGAGGAGCCTTAGATCTGACGACACGGCCGTTTTTTAC | |
| TGCGCGAGGCGTCATCAACAGAGCTTGGATTATTGGGGCCAAGGCACCCTGGTCACC | |
| GTCTCGAGTGGAGGCGGCGGTTCAGGCGGAGGTGGCTCTGGCGGTGGCGGAAGTGCA | |
| CAGTCTGTCGTGACGCAGCCGCCCTCCGCGTCCGGGTCTCCTGGACAGTCAGTCACC | |
| ATCTCCTGCTCTGGATACAGCTCCTCCAACATCGGGAATAATGCTGTCTCCTGGTAC | |
| CAACAACTCCCAGGAACAGCCCCCAAACTCCTCATTTTTGACAATAATAAGCGACCC | |
| TCAGGGATTCCTGCCCGATTCTCTGGCTCCCAGTCTGGCACGACAGCCACCCTGGGC | |
| ATCACCGGACTCCAGACTGGGGACGAGGCCGATTATTTCTGCGGAACATGGGATAGC | |
| AGCCTGAGTGCTTTTGTCTTCGGATCCGGGACCAAGGTCACCGTCCTAGGT, | |
| PGIA-02-A1 | |
| ATGGCCGAGGTGCAGCTGGTGCAGTCTGGGGCTGAGGTGAAGAAGCCTGGGTCCTCG | SEQ ID NO:75 |
| GTGAAGGTCTCCTGCAAGGCTTCTGGAGGCAGCTTCAGCAACTATGATTTCAGTTGG | |
| GTGCGGCAGGCCCCCGGACAAGGGCTTGAGTGGATGGGAGAGATCATCAATGCCTTT | |
| GGTTCATCAAGATACGCACAGAAATTCCAGGACAGAGTCACCATTACCGCGGACGAA | |
| TCCGCGAGCACAGCCTACATGGAACTAAGAGGCCTGACATCTGAGGACACGGCCACT | |
| TATTACTGTGCGAGGGCGGAAAGGTGGGAACTTAATATGGCTTTTGATATGTGGGGC | |
| AGAGGAACCCTGGTCACCGTCTCGAGTGGAGGCGGCGGTTCAGGCGGAGGTGGCTCT | |
| GGCGGTGGCGGAAGTGCACAGTCTGTGCTGACTCAGCCACCCTCGGTGTCAGTGGCC | |
| CCAGGGCAGACGGCCAGGATCACCTGTGGGGGAGACAATATAGGGAGAAAAAATGTC | |
| CACTGGTACCAGCAGCGGCCAGGCCTGGCCCCTGTTTTAGTCGTCTATGATGACACC | |
| GACCGGCCCTCAGGGATCCCTGAGCGATTCTCTGGCTCCAACTCTGGGGACACGGCC | |
| ACCCTGACCATCACCTGGGTCGAGGCCGGGGATGAAGCCGACTATTACTGTCAACTT | |
| TGGGATAGTGACACCTATGATGTTTTATTCGGCGGAGGGACCAAGCTGACCGTCCTA | |
| GGT, | |
| PGIA-02-A2 | |
| GAGGTGCAGCTGGTGCAGTCTGGGGCTGAGGTGAAGAAGCCTGGGTCCTCCGTGAAG | SEQ ID NO:76 |
| GTCTCCTGCAAGTCTTCTGGAGGCCCCTTCAGCAGCTATGGTATCAGCTGGGTGCGA | |
| CAGGCCCCCGGACAAGGGCTTGAGTGGATGGGAGGGATCAGCCCTATCTTTGGTACA | |
| GCAAACTACGCACAGAAGTTCCAGGGCAGAGTCACCATTACCGCGGACGAATCCACA | |
| CAGACAGCCTACATGGAGCTGAGTAGCCTGAGGTCTGAGGACACGGCCGTGTATTAC | |
| TGTGCGAGAGACGAGTCACCGGTCGGGTTTTATGCTTTGGATATCTGGGGGCGAGGG | |
| ACCACGGTCACCGTCTCGAGTGGAGGCGGCGGTTCAGGCGGAGGTGGCTCTGGCGGT | |
| GGCGGAAGTGCACTTTCCTATGAGCTGACTCAGCCACCCTCGGTGTCAGTGGCCCCA | |
| GGACAGACGGCCAGGATTAACTGTGGGGGAGACAAAATTGGAAGTAGAAGTGTACAC | |
| TGGTACCAGCAGAAGCCAGGCCAGGCCCCTGTGATGGTCGTCTATGATGATAGCGAC | |
| CGGCCCTCAGGGATCCCTGAGCGATTCTCTGGCTCCAACTCTGGGAACACGGCAACC | |
| CTGACCATCAGCAGTGTCGAAGCCGGGGATGAGGCCGACTATTATTGTCAGGTGTGG | |
| GATGGTAGTACTGATCCCTGGGTATTCGGCGGAGGGACCAAGGTCACCGTCCTAGGT, | |
| PGIA-02-A3 | |
| GAAGTGCAGCTGGTGCAGTCTGGGGCTGAGATGAAGAAGCCTGGGTCCTCGGTGAAG | SEQ ID NO:77 |
| GTCTCCTGCAAGGCATCTGGAGGCACCTTCAGCAGCTATGCTGTCAACTGGGTGCGA | |
| CAGGCCCCTGGACAAGGGCTTGAATGGATGGGAGGAATCATCCCTATTTTTGATACT | |
| TCGAACTACGCACAGAAGTTCCAGGGCAGACTCACGATGACCGCGGACGACTCCACG | |
| AACACAGCCTACATGGAACTGAGGAGCCTGAGATCTGAGGACACGGCCGTATATTAC | |
| TGTGCGAGAGGGGCCCCGAGGGGAACAGTTATGGCATTCAGCTCTTACTACTTTGAC | |
| TTATGGGGCCAGGGCACCCTGGTCACCGTCTCGAGTGGAGGCGGCGGTTCAGGCGGA | |
| GGTGGCTCTGGCGGTGGCGGAAGTGCACTTAATTTTATGCTGACTCAGCCCCACTCT | |
| GTGTCGGAGTCTCCGGGGAAGACAGTAATTATCTCCTGCGCCGGCAGCGGTGGCAAC | |
| ATTGCCACCAACTATGTGCAGTGGTACCAACATCGCCCGGGCAGTGCCCCCATTACT | |
| GTGATCTATGAGGATAATCAAAGACCCTCTGGAGTCCCTGATCGCTTCTCTGGCTCC | |
| GTCGACAGCTCCTCCAACTCTGCCTCCCTCACCATCTCTGGACTGCAGACTGAGGAC | |
| GAAGCTGACTACTACTGTCACTCTTATGACAACACCGATCAGGGGGTCTTCGGAACT | |
| GGGACCAAGGTCACCGTCCTAGGT, | |
| PGIA-02-A4 | |
| GAGGTGCAGCTGGTGGAGTCCGGGGGAGGCTTGGTACAGCCTGGCAGGTCCCTGAGA | SEQ ID NO:78 |
| CTCTCCTGTGCAGCCTCTGGATTCACCTTTGATGATTACGACATGCACTGGGTCCGG | |
| CAAGCTCCAGGGAAGGGCCTGGAGTGGGTCTCAAGTATTAGTTGGAGTGGTGGAACT | |
| ATAGGGTATGCGGACTCTGTGAAGGGCCGATTCACCGTCTCCAGAGACAACGCCAAG | |
| AACTCCCTGTATCTGCAAATGAACAGTGTGAGAGCTGAGGACACGGCCTTATATTAC | |
| TGTGCAAAAGACAGGGGCGCTGTAGCAGCTCTCCCCGACTATCAGTACGGTATGGAC | |
| GTCTGGGGCAGGGGCACCCTGGTCACCGTCTCGAGTGGAGGCGGCGGTTCAGGCGGA | |
| GGTGGCTCTGGCGGTGGCGGAAGTGCACAGTCTGCCCTGACTCAGCCTGCCTCCGTG | |
| TCTGGGTCTCCTGGACAGTCGATCACCATCTCCTGCACTGGAACCAGCAGTGATATT | |
| GGGAGTTATAACCTTGTCTCCTGGTACCAACAACACCCAGGCAAAGCCCCCAAACTC | |
| ATGATTTATGAGGACTATAAGCGGGCCTCAGGGGTTTCTAATCACTTCTCTGGCTCC | |
| AAGTCTGGCAACACGGCCTCCCTGACAATCTCTGGGCTCCAGGCTGAGGACGAGGCT | |
| GATTATTACTGCTCCTCATATGCAGGTAGTAGCGCTTGGGTGTTCGGCGGAGGGACC | |
| AAGGTCACCGTCCTAGGT, | |
| PGIA-02-A5 | |
| GAAGTGCAGCTGGTGCAGTCTGGGGCTGAGGTGAGGAAGCCTGGATCCTCGATGAAG | SEQ ID NO:79 |
| GTCTCCTGCAAGGCCTCTGGCGACACCTTCAGGAACTTTGCTTTCAGTTGGGTGCGA | |
| CAGGCCCCTGGACAAGGACTTGAATGGATGGGGGGAGTCATCCCTTTGGTTGGTCCA | |
| CCAAAGTACGCTCAGAAGTTCCAGGGCAGACTCACCATTACCGCGGACGAGTCCACG | |
| AGCACCTCCTACATGGACTTGACCAGCCTGACACTCGAAGACACGGCCGTCTATTTC | |
| TGTGCGCGAGGGGGGGTTTATGCTCCCTTTGACAAATGGGGCCAAGGAACCCTGGTC | |
| ACCGTCTCGAGTGGAGGCGGCGGTTCAGGCGGAGGTGGCTCTGGCGGTGGCGGAAGT | |
| GCACAGTCTGTCGTGACGCAGCCGCCCTCAGTGTCTGAAGCCCCCAGGCAGAGGGTC | |
| ACCATCTCCTGTTCTGGAAGCAGCTCCAACATCGGAAATAATGCTGTAAACTGGTAC | |
| CAGCAGCTCCCAGGAAAGGCTCCCAAACTCCTCATCTATTATAATGATCTGCTGCCC | |
| TCAGGGGTCTCTGACCGATTCTCTGGCTCCAAGTCTGGCACCTCAGCCTCCCTGGCC | |
| ATCAGTGGGCTCCAGTCTGAGGATGAGGCTGATTATTACTGTGCAGCATGGGATGAC | |
| AGCCTGAATGGCTGGGTGTTCGGCGGAGGGACCAAGGTCACCGTCCTAGGT, | |
| PGIA-02-A6 | |
| GAGGTGCAGCTGGTGCAGTCTGGGGCTGAAGTGAAGAAGCCTGGGTCCTCGGTGAAG | SEQ ID NO:80 |
| GTCTCCTGTAAGGCCTCTGGAGGCACCTTCAAGACCTATGCTATCAATTGGGTGCGA | |
| CAGGCCCCTGGACAAGGGCTTGAGTGGATGGGAGGAATCATCCCTGTCCTGGGAACA | |
| GCAAATTACGTTCAGAAGTTCCAGGGCAGAGTCACGATTACCGCGGACGAATCGACG | |
| ACCACAGCCTACATGGAGCTGAGGGGCCTGAGATCTGAGGACACGGCCGTTTATTAT | |
| TGTGCGAGAGGAGAGGGCAGTGGCTGGTACGATCACTACTACGGATTGGACGTCTGG | |
| GGCCAAGGAACCCTGGTCACCGTCTCGAGTGGAGGCGGCGGTTCAGGCGGAGGTGGC | |
| TCTGGCGGTGGCGGAAGTGCACAGTCTGTGCTGACGCAGCCGCCCTCAGCGTCTGGG | |
| ACCCCCGGGCAGAGGGTCACCATCTCTTGTTCTGGAAGCAGCTCCAACATCGGAAGT | |
| AATACTGTAAACTGGTACCGGCAGCTCCCAGGAACGGCCCCCAAACTCCTCATCTTT | |
| GGTGATGATCAGCGGCCCTCAGGGGTCCCTGACCGATTCTCTGGCTCCAGGTCTGGC | |
| ACCTCAGTCTCCCTGGCCATCAGTGGGCTCCAGTCTGAGGATGAGGCTGACTATTAC | |
| TGTGCAGCATGGGATGACAGCCTGAATGGCGGGGTGTTCGGCGGAGGGACCAAGCTG | |
| ACCGTCCTAGGT, | |
| PGIA-02-A7 | |
| CAGCTGCAGCTGCAGGAGTCGGGCCCAGGACTGGTGAAGCCTTCGGGGACCCTGTCC | SEQ ID NO:81 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCAAAGGA | |
| ACCCTGGTCACCGTCTCGAGTGGAGGCGGCGGTTCAGGCGGAGGTGGCTCTGGCGGT | |
| GGCGGAAGTGCACAGGCTGTGCTGACTCAGCCGTCCTCAGTGTCTGCGGCCCCAGGA | |
| CAGAAGGTCACCATCTCCTGCTCTGGAAGCAGCTCCAACATTGGGAATAATTATGTA | |
| TCCTGGTACCAGCAGCTCCCAGGAACAGCCCCCAAACTCCTCATTTATGACAATAAT | |
| AAGCGACCCTCAGGGATTCCTGACCGATTCTCTGGCTCCAGGTCTGGCACGTCAGCC | |
| ACCCTGGGCATCACCGGACTCCAGACTGGGGACGAGGCCGATTATTACTGCGGAACA | |
| TGGGATAGCAGCCTGAGTGCTGTAGTCTTCGGAACTGGGACCAAGCTGACCGTCCTA | |
| GGT, | |
| PGIA-02-A8 | |
| CAGCTGCAGCTGCAGGAGTCGGGCCCAGGACTGGTGAAGCCTTCGGGGACCCTGTCC | SEQ ID NO:82 |
| CTCACCTGCGCTGTCTCTGGTGGCTCCATCAGCAGTACTAACTGGTGGAGTTGGGTC | |
| CGCCAGCCCCCAGGGAAGGGGCTGGAGTGGATTGGGGAAATCTATCATAGTGGGAGC | |
| ACCAACTACAACCCGTCCCTCAAGAGTCGAGTCACCATATCAGTAGACAAGTCCAAG | |
| AACCACTTCTCCCTGAACCTGAGCTCTGTGACCGCCGCGGACACGGCCGTGTATTAC | |
| TGTGCGAGAGATTCTATGGGAAGCACTGGCTGGCATTACGGTATGGACCTCTGGGGC | |
| AAAGGCACCCTGGTCACCGTCTCGAGTGGAGGCGGCGGTTCAGGCGGAGGTGGCTCT | |
| GGCGGTGGCGGAAGTGCACAGTCTGCCCTGACTCAGCCTGCCTCCGTGTCTGGGTCT | |
| CCTGGACAGTCGATCGCCATCTCCTGCACTGGAACCAGCAGTGACGTTGGTGGTTAT | |
| AACTATGTCTCGTGGTACCAACAGCACCCAGGCAAAGCCCCCAAACTCATGATTTAT | |
| GCTGTCACTAATCGGCCCTCAGGGGTTTCTGATCGCTTCTCTGGCTCCAAGTCTGGC | |
| AACACGGCCTCCCTGACCATCTCTGGGCTCCAGGCTGACGACGAGGCTGATTATTAC | |
| TGCAGCTCATATACAAGCAGCAGCTCTCTGGTGTTCGGCGGAGGGACCAAGCTGACC | |
| GTCCTAGGT, | |
| PGIA-02-A9 | |
| GGGGTGCAGCTGGTGGAGTCTGGGGGAGGCCTGGTCAAGCCTGGGGGGTCCCTGAGA | SEQ ID NO:83 |
| CTCTCCTGTGCAGCCTCTGGATTCACCTTCAGTAGTTATACCATGAACTGGGTCCGC | |
| CAGGCTCCAGGGAAGGGGCTGGAGTGGGTTTCATACATTAGTAGTAGTGGTAGTGCC | |
| ACATACTACGCAGACTCTGTGAAGGGCCGATTCACCATCTCCAGGGACAACGCCAAC | |
| AACTCACTGTATCTGCAAATGAACAGCCTGAGAGCCGAGGACACGGCCGTGTATTAC | |
| TGTGCGAGAGGGTACCGCTACGGCATGGACGTCTGGGGCCAAGGAACCCTGGTCACC | |
| GTCTCGAGTGGTGGAGGCGGTTCAGGCGGAGGTGGCAGCGGCGGTGGCGGATCGGGC | |
| ATCGTGATGACCCAGTCTCCTTCCACCCTGTCTGCATCTGTAGGAGACAGAGTCACC | |
| ATCACTTGCCGGGCCAGTCAGGGTATTAGTAGCTGGTTGGCCTGGTATCAGCAGAAA | |
| CCAGGGAGAGCCCCTAAGGTCTTGATCTATAAGGCATCTACTTTAGAAAGTGGGGTC | |
| CCATCAAGGTTCAGCGGCAGTGGATCTGGGACAGATTTCACTCTCACCATCAGCAGT | |
| CTGCAACCTGAAGATTTTGCAACTTACTACTGTCAACAGAGTTACAGTACCCCGTGG | |
| ACGTTCGGCCAAGGGACCAAGCTGGAGATCAAACGT | |
| PGIA-02-A10 | |
| GAGGTGCAGCTGTTGGAGTCTGGGGGAGGCTTGGTACAGCCTGGGGGGTCCCTGAGA | SEQ ID NO:84 |
| CTCACCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAGAGATTTAGCAGTGGCAGGTATTGACTACTGGGGCCGGGGGACAATGGTC | |
| ACCGTCTCGAGTGGAGGCGGCGGTTCAGGCGGAGGTGGCTCTGGCGGTGGCGGAAGT | |
| GCACAGTCTGTGCTGACGCAGCCGCCCTCAGCGTCTGGGACCCCCGGGCAGAGGGTC | |
| ACCATATCTTGTTCTGGGAGCAGTTCCAACATCAGAAGTAATTATGTTTACTGGTAC | |
| CAGCAGTTCCCAGGAACGGCCCCCAAACTCCTCATTTATAGAAATAATCAGCGGCCC | |
| TCAGGGGTCCCTGACCGATTCTCTGGCTCCAAGTCTGGCACCTCAGCCTCCCTGGCC | |
| ATCAGTGGGCTCCGGTCCGAGGATGAGGCTGATTATTATTGTGCAGCATGGGATGAC | |
| ACCCTGGATGCTTATGTCTTCGCAGCTGGGACCAAGCTGACCGTCCTAGGT, | |
| PGIA-02-A11 | |
| CAGGTGCAGCTGCAGGAGTCCGGCCCAGGACTGGTGAAGCCTTCGGGGACCCTGTCC | SEQ ID NO:85 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCCCTTGACTACTGGGGCCAGGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGGGTCTCCG | |
| GGGAGGACGGTAACCATCTCCTGCACCCGCAGCAGTGGCAGCATTGCCACCAACTAT | |
| GTGCAGTGGTACCAGCAGCGCCCGGGCAGTTCCCCCACCATTGTGATCTATGAAGAT | |
| AACCAAAGACCCTCTGGGGTCCCTGATCGCTTCTCTGGCTCCATCGACACCTCCTCC | |
| AACTCTGCCTCCCTCACCATCTCTGGACTGAAGACTGAGGACGAGGCTGACTACTAC | |
| TGTCAGTCTTATGATAGCAACAATCTGGGGGTGGTATTTGGCGGAGGGACCCAGCTC | |
| ACCGTTTTAAGT, | |
| PGIA-02-A12 | |
| CAGGTACAGCTGCAGCAGTCAGGGGCTGAGGTGAGGAAGCCTGGGGCCTCAGTGAAG | SEQ ID NO:86 |
| ATCTCCTGCAAGACTTCTGGATACACCTTCATGGACTACTACATACACTGGGTGCGA | |
| CAGGCCCCTGGACAAGGGCTTGAGTGGATGGGCTGGAGCAACCCTGTCACTGGTACG | |
| TCAGGCTCTTCACCTAAATTTCGGGGCAGGGTCACCTTGACCACTGACACGTCCGGC | |
| AACACAGCCTATTTGGACCTGAGGAGCCTTAGATCTGACGACACGGCCGTATTTTAC | |
| TGTGCGAGGCGTCACCAACAGAGCTTGGATTATTGGGGCCAAGGCACCCTGGTCACC | |
| GTCTCGAGTGGAGGCGGCGGTTCAGGCGGAGGTGGCTCTGGCGGTGGCGGAAGTGCA | |
| CAGGCTGTGCTGACTCAGCCGTCTTCCCTCTCTGCATCTCCTGGAGCATCAGCCAGT | |
| CTCACCTGCACCTTACGCAGTGACATCAATGTTGGTTCCTACAGTATAAACTGGTAC | |
| CAGCAGAAGCCAGGGAGTCCTCCCCAATATCTCCTGAACTACAGATCAGACTCAGAT | |
| AAGCAGCAGGGCTCTGGAGTCCCCAGCCGCTTCTCTGGATCGAAGGATGCTTCGGCC | |
| AATGCAGGGATTTTACTCATCTCTGGTCTCCAGTCTGAGGATGAGGCTGACTATTAC | |
| TGTATGATTTGGTACAGGACCGCTTGGGTGTTCGGCGGAGGGACCAAGGTCACCGTC | |
| CTAGGT, | |
| PGIA-02-B1 | |
| CAGGTCCAGCTGGTACAGTCTGGAGCTGAGGTGAGGAAGCCTGGGGCCTCAGTGAAG | SEQ ID NO:87 |
| GTCTCCTGCAAGACTTCTGGATACACCTTCATCGAATACTACATACACTGGGTGCGA | |
| CAGGCCCCTGGACAAGGGCTTGAGTGGATGGGCTGGAGCAACCCTGTCACTGGTACG | |
| TCAGGCTCTTCACCTAAGTTTCGGGGCAGGGTCACCTTGACCACTGACACGTCCGGC | |
| AACACAGCCTATTTGGACCTGAGGAGCCTTAGATCTGACGACACGGCCGTCTTTTAC | |
| TGTGCGAGGCGTCACCAACAGAGCTTGGATTATTGGGGGCGGGGGACCACGGTCACC | |
| GTCTCGAGTGGAGGCGGCGGTTCAGGCGGAGGTGGCTCTGGCGGTGGCGGAAGTGCA | |
| CAGTCTGTGCTGACGCAGCCGCCCTCAGTGTCTGCGGCCCCAGGACAGAAGGTCACC | |
| ATCTCCTGCTCTGGAACCAACTCCAACATTGGAAATTATTATGTATCTTGGTACCAG | |
| CAACTCCCAGGAACAGCCCCCAAACTCCTCATTTATGACAATAATAAGCGACCCTCA | |
| GGGGTCCCTGACCGATTCTCTGGCTCCAAGTCTGGCACCTCAGCCTCCCTGGTCATC | |
| AGTGGGCTCCGGTCCGAGGATGAGGCTGATTATTACTGTGCAGCATGGGATGGCAGC | |
| CTGACTGCTTGGGTGTTCGGCGGAGGGACCAAGGTCACCGTCCTAGGT, | |
| PGIA-03-A1 | |
| CAGGTGCAGCTGCAGGAGTCCGGCCCAGGACTGGTGAAGCCTTCGGGGACCCTGTCC | SEQ ID NO:88 |
| CTCACCTGCGCTGTCTCTGGTGACTCCATCAGCAGTAGTAACTGGTGGACTTGGGTC | |
| CGCCAGCCCCCAGGGAAGGGGCTGGAGTGGATTGGGGAAATCTTTCATAGTGGGACC | |
| ACCAACTACAACCCGTCCCTCAACAATCGAGTCACCATATCACTAGACGAGTCCAGG | |
| AACCAGTTCTCCCTGGAGTTGAGCTCTGTGACCGCCGCGGACACGGCCATATATTAC | |
| TGTGCGAGAGATTCGGGGAATTACGATGATAATAGAGGCTACGACTACTGGGGCCGA | |
| GGCACCCTGGTCACCGTCTCGAGTGGAGGCGGCGGTTCAGGCGGAGGTGGCTCTGGC | |
| GGTGGCGGAAGTGCACAGTCTGTGTTGACGCAGCCGCCCTCAGTGTCTGGGGCCCCA | |
| GGGCAGAGGGTCACCATCTCCTGCGCTGGGACCAGCTCCAACATCGGGGCAGGTTTT | |
| GATGTACACTGGTACCAGCTTCTTCCAGGAAGAGCCCCCAAACTCCTCATCTATGGT | |
| AACAACAATCGGCCCTCAGGGGTCCCTGACCGATTCTCTGGCTCCAAGTCTGGCACC | |
| TCAGCCTCCCTGGCCATCAGTGGTCTCCAGTCTGAGGACGAGGGTGACTATTACTGT | |
| GCAGCTTGGGATGACACCGTGGGTGGTCCGGTGTTCGGCGGAGGGACCAAGCTGACC | |
| GTCCTAGGT, | |
| PGIA-03-A2 | |
| CAGGTGCAGCTGCAGGAGTCGGGCCCAGGACTGGTGAAGCCTTCGGGGACCCTGTCC | SEQ ID NO:89 |
| CTCACCTGCGCTGTCTCTGGTGGCTCCATCAGCAGTACTAACTGGTGGAGTTGGGTC | |
| CGCCAGCCCCCAGGGAAGGGGCTGGAGTGGATTGGGGAAATCTATCATAGTGGGAGC | |
| ACCAACTACAACCCGTCCCTCAAGAGTCGAGTCACCATATCAGTAGACAAGTCCAAG | |
| AACCACTTCTCCCTGAACCTGAGCTCTGTGACCGCCGCGGACACGGCCGTGTATTAC | |
| TGTGCGAGAGATTCTATGGGAAGCACTGGCTGGCATTACGGTATGGACCTCTGGGGC | |
| AGGGGAACCCTGGTCACCGTCTCGAGTGGAGGCGGCGGTTCAGGCGGAGGTGGCTCT | |
| GGCGGTGGCGGAAGTGCACAGTCTGCCCTGACTCAGCCTGCCGCCGTGTCTGGGTCT | |
| CCTGGACAGTCGATCACCATCTCCTGCACTGGATCCAGCAGTGACGTTGGTGGTTAT | |
| AACTATGTCTCCTGGTACCAACAACACCCAGGCAAGGCCCCCAAACTCTTGATTTAT | |
| GATGTCAGTGATCGGCCCTCAGGGGTCTCTTATCGCTTCTCTGGCTCCAAGTCTGGC | |
| AACACGGCCTCCCTGACCATCTCTGGGCTCCAGGCTGAGGACGAGGCTGATTATTAC | |
| TGCAGCTCATATACAGCCACCGGCACTCTGGTATTCGGCGGAGGGACCAAGCTGACC | |
| GTCCTAGGT, | |
| PGIA-03-A3 | |
| CAGGTGCAGCTGCAGGAGTCGGGCCCAGGACTGGTGAAGCCTTCGGGGACCCTGTCC | SEQ ID NO:90 |
| CTCACCTGCGCTGTCTCTGGTGGCTCCATCAGCAGTACTAACTGGTGGAGTTGGGTC | |
| CGCCAGCCCCCAGGGAAGGGGCTGGAGTGGATTGGGGAAATCTATCATAGTGGGAGC | |
| ACCAACTACAACCCGTCCCTCAAGAGTCGAGTCACCATATCAGTAGACAAGTCCAAG | |
| AACCACTTCTCCCTGAACCTGAGCTCTGTGACCGCCGCGGACACGGCCGTGTATTAC | |
| TGTGCGAGAGATTCTATGGGAAGCACTGGCTGGCATTACGGTATGGACCTCTGGGGG | |
| CAGGGGACCACGGTCACCGTCTCGAGTGGAGGCGGCGGTTCAGGCGGAGGTGGCTCT | |
| GGCGGTGGCGGAAGTGCACAGTCTGCCCTGACTCAGCCTGCCTCCGTGTCTGGGTCT | |
| CCTGGACAGTCGATCACCATCTCCTGCACTGGAACCAGCAGTGACGTTGGTGGTTAT | |
| AACTATGTCTCCTGGTACCAACAGCACCCAGGCAAAGCCCCCAAACTCATGATTTAT | |
| GAGGTCAGTAATCGGCCCTTAGGGGTTTCTAATCGCTTCTCTGGCTCCAAGTCTGGC | |
| AACACGGCCTCCCTGACCATCTCTGGGCTCCAGGCTGAGGACGAGGGTGATTATTAC | |
| TGCAGCTCATATACAAGCAGCACCACTCTTATAGTATTCGGCGGAGGGACCAAGCTG | |
| ACCGTCCTAGGT, | |
| PGIA-03-A4 | |
| CAGGTGCAGCTGCAGGAGTCGGGCCCAGGACTGGTGAAGCCTTCGGGGACCCTGTCC | SEQ ID NO:91 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCAAGGC | |
| ACCCTGGTCACCGTCTCGAGTGGAGGCGGCGGTTCAGGCGGAGGTGGCTCTGGCGGT | |
| GGCGGAAGTGCACAGTCTGTGCTGACTCAGCCACCCTCAGTGTCTGGGACCACCGGG | |
| CAGAGGGTCATCCTCTCTTGTTCTGGAGGAAACTCCAACATCGGATATAATTCTGTA | |
| AACTGGTACCAGCAGCTCCCAGGAACGGCCCCCAAACTCCTCATCTATACTGATGAT | |
| CAGCGGCCCTCAGGGGTCCCTGACCGTTTCTCTGGCTCCAGGTCTGGCACCTCAGCC | |
| TCCCTGGCCATCAGTGGGCTCCAGTCTGAGGATGAGGCTGATTATTACTGTGCAACA | |
| TGGGATGACTCCCTGAATGCCGGGGTGTTCGGCGGCGGGACCAAGCTGACCGTCCTA | |
| GGT, | |
| PGIA-03-A5 | |
| CAGGTCCAGCTGGTGCAGTCTGGGGCTGAGGTGAGGAAGCCTGGGGCCTCAGTGAGG | SEQ ID NO:92 |
| GTCTCCTGTAAGACTTCTGGATACACCTTCTTGGAATACTACATACACTGGGTGCGA | |
| CAGGCCCCTGGACAAGGGCTTGAGTGGATGGCCTGGAGCAACCCTGTCACTGGAACG | |
| TCAGGCTCCTCACCTAAATTTCGGGGCAGAGTCACCCTGACCGCTGACACGTCCGGC | |
| AACACAGCCTATTTGGACCTGAAGAGCCTTACGTCTGACGACACGGCCATATTCTAC | |
| TGTGCGAGGCGTCACCAACAGAGCTTGGATTATTGGGGCCAAGGAACCCTGGTCACC | |
| GTCTCGAGTGGAGGCGGCGGTTCAGGCGGAGGTGGCTCTGGCGGTGGCGGAAGTGCA | |
| CAGTCTGTGCTGACTCAGCCACCCTCAGTGTCTGCGGCCCCAGGGCAGACGGTCACC | |
| ATCTCCTGCTCTGGAAGCAACTCCAACATTGGGAATAATCATGTATCTTGGTACCGA | |
| CAACTCCCGGAAACAGCCCCCAAACTCCTCATTTATGACAACAATAAGCGACCGTCA | |
| GGGATTCCTGACCGATTCTCTGGCTCCAAGTCTGGCACGTCAGCCACCCTGGACATC | |
| ACCGGACTCCAGACTGGGGACGAGGCCGATTATTACTGCGCGACATGGGATAACAGC | |
| CTGAGTGCCCCTTGGGTGTTCGGCGGCGGGACCAAGCTGACCGTCCTAGGT, | |
| PGIA-03-A6 | |
| CAGGTGCAGCTGCAGGAGTCGGGGGCTGAGGTGAAGAAGCCTGGGTCCTCGGTGAAG | SEQ ID NO:93 |
| GTCTCCTGCAAGGCTTCTGGAGGCACCTTCAGCAGCTCTGCTATCAGCTGGGTGCGA | |
| CAGGCCCCTGGACAAGGACTTGAGTGGATGGGAGGGATCATCCCTGTCTTTGGTACA | |
| GCAAATTACGCACAGAAGTTCCAGGACAGAGTCACTATTACCGCGGACGAGTCCACG | |
| AGCACAGCCTACCTGGAGCTGAGCAGGCTGACATCTGAGGACACGGCCGTGTATTAC | |
| TGTGCGTCGAGGGGGGAGTATGACTACGGTGACTACGACGTCTACTACTACTATATG | |
| GAGGTCTGGGGCCAGGGCACCCTGGTCACCGTCTCGAGTGGAGGCGGCGGTTCAGGC | |
| GGAGGTGGCTCTGGCGGTGGCGGAAGTGCACAGTCTGTGCTGACTCAGCCACCCTCG | |
| GTGTCAGTGGCCCCGGGACAGACGGCCAGGTTGACCTGTGGGGCAAACAACATTGGA | |
| AGTACAAGTGTTCACTGGTACCAGCAGAAGCCAGGCCAGGCCCCTGTGTTGGTCATA | |
| TATGATGATACTGACCGGCCCTCTGGTATCCCTGAGCGATTCTCTGGCTCCAACTCT | |
| GGGAACACGGCCACCCTGACCATCAGAAGGGTCGAAGCCGGGGATGAGGCCGACTAT | |
| TACTGTCAGGTGTGGGATACTAACAGTGATCATGTGATATTCGGCGGAGGGACCAAG | |
| CTGACCGTCCTAGGT | |
| PGIA-03-A7 | |
| GAGGTGCAGCTGGTGCAGTCTGGGGCTGAGGTGAAGAAGCCTGGGTCCTCGGTGAAG | SEQ ID NO:94 |
| GTCTCCTGCCAGGCTTCTGGAGGCACCTTCACAAGCCACGCTATGTACTGGGTGCGA | |
| CAGGCCCCTGGACAAGGACTTGAGTGGATGGGAGGGATCATCCCTATCTTTGGAAGA | |
| ACAAACTACGCACAGAAATTCCAGGGCAGAGTCACGTTTACCGCGGACATGTCCACG | |
| AGTACAGCCTATATGGAAATGACCAGCCTGAGATCTGACGACACGGCCGTATATTAC | |
| TGTGCGAGAGGCGATAATTGGAATGACCTTTACCCGATTGACTACTGGGGCCGAGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGCCACCACTTAC | |
| GTGCAGTGGTTCCAGCAGCGCCCGGGCAGTTCCCCCACCACTGTGATCTATGATGAT | |
| GACCAAAGACCGTCTGGGGTCCCTGATCGCTTCTCTGGATCCATCGACAGCTCCTCC | |
| AACTCTGCCTCCCTCACCATCTCTGGACTGATGCCTGAGGACGAGGCTGACTACTAC | |
| TGTCAGTCTTATGATAACACCGATCTGGTGTTCGGCGGTGGGACCCAGCTCACCGTT | |
| TTAAGT, | |
| PGIA-03-A8 | |
| GAGGTCCAGCTGGTACAGTCTGGGGCTGAGGTGAAGAAGCCTGGGGCCTCAGTGAAG | SEQ ID NO:95 |
| GTCTCCTGCAAGGTTTCCGGATACTCCCTCTCTGAATTATCCATGCACTGGGTGCGA | |
| CAGGCTCCTGGAAAAGGACTTGAGTGGATGGGAGGTTTTGATCCTCAAAATGGTTAC | |
| ACAATCTACGCACAGGAGTTCCAGGGCAGAATCACCATGACCGAGGACACATCTACA | |
| GACACAGTCTACATGGAACTGGGCAGCCTGAGATCTGAAGACACGGCCGTGTATTTC | |
| TGTGCAGCAATCGAAATAACTGGGGTGAACTGGTACTTCGATCTCTGGGGCAAAGGC | |
| ACCCTGGTCACCGTCTCGAGTGGAGGCGGCGGTTCAGGCGGAGGTGGCTCTGGCGGT | |
| GGCGGAAGTGCACTTTCTTCTGAGCTGACTCAGGACCCTGATGTGTCTGTGGCGTTG | |
| GGACAGACAGTCAGGATCACATGCCAAGGAGACAGCCTCAAAAAATTTTATCCAGGT | |
| TGGTACCAGCAGAAGCCAGGACAGGCCCCTCTACTTGTCCTATATGGTGAAAACATT | |
| CGGCCCTCAAGAATCCCCGACCGATTCTCTGGCTCCAGCTCCGGAAACACAGCTACC | |
| CTGACCATCACTGGGGCTCAGGCGGAGGATGAGGCTGTGTATTACTGTAATTCCCGG | |
| GAAGCCAGTGTTCACCATGTAAGGGTCTTCGGCGGAGGGACCAAGCTGACCGTCCTA | |
| GGT, | |
| PGIA-03-A9 | |
| CAGGTGCAGCTGCAGGAGTCGGGCCCAGGACTGGTGAAGCCTTCGGGGACCCTGTCC | SEQ ID NO:96 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCAAGGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGCCAGCAACTAT | |
| GTGCAGTGGTACCAGCAGCGCCCGGGCAGTTCCCCCACCACTGTGATCTATGAGGAT | |
| AACCAAAGACCCTCTGGGGTCCCTGATCGGTTCTCTGGCTCCATCGACAGCTCCTCC | |
| AACTCTGCCTCCCTCACCATCTCTGGACTGAAGACTGAGGACGAGGCTGACTACTAC | |
| TGTCAGTCTTATGATAGCAGCAATCAGGGGGTGGTCTTCGGCGGAGGGACCAAGCTG | |
| ACCGTCCTAGGT, | |
| PGIA-03-A10 | |
| CAGCTGCAGCTGCAGGAGTCGGGCCCAGGACTGGTGAAGCCTTCGGGGACCCTGTCC | SEQ ID NO:97 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCAAGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTCACCATCTCCTGCACCGGCAGCAGTGGCAGCATTGCCAGCAACTAT | |
| GTGCAGTGGTACCAGCAGCGCCCGGGCAGTGCCCCCACCACTCTGATCTATGAGGAT | |
| GACCAAAGACCCTCTGGGGTCCCTGATCGGTTCTCTGGCTCCGTCGACAGCTCCTCC | |
| AACTCTGCCTCCCTCACCATCTCTGGACTGAAGACTGAGGACGAGGCTGATTACTAT | |
| TGTCAGTCTTATGATAGGAGCAATCAGGCGGTGGTTTTCGGCGGAGGGACCAAGCTG | |
| ACCGTCCTAGGT, | |
| PGIA-03-A11 | |
| CAGGTCCAGCTGGTGCAGTCTGGGCCTGAGGTGAAGAAGCCTGGGGCCTCAGTGGAG | SEQ ID NO:98 |
| GTCTCCTGTAAGGCTTCTGGATACACCTTCACCGGCGACTATATGCACTGGGTGCGA | |
| CAGGCCCCTGGACAAGGACCTGAGTGGATGGGGTGGATCAACCCTCAGACTGGTGTC | |
| ACAAAGTATGCACAGAAGTTTCAGGGCAGGGTCACCATGGCCAGGGACACGTCCATC | |
| AACACAGCCTACATGGAACTGAGAGGGCTGAGATCCGACGACACGGCCGTGTATTAC | |
| TGTGTGCGAGAGGATCACAATTACGATTTGTGGAGTGCTTACAACGGTTTGGACGTC | |
| TGGGGCCAGGGCACCCTGGTCACCGTCTCGAGTGGAGGCGGCGGTTCAGGCGGAGGT | |
| GGCTCTGGCGGTGGCGGAAGTGCACAGTCTGTGCTGACGCAGCCGCCCTCAGTGTCT | |
| GCGGCCCCAGGACAGAAGGTCACCATCTCCTGCTCTGGAAGCAGCTCCAACATTGGG | |
| AATAATCATGTGTCGTGGTACCAGCAGCTCGCAGGAACAGCCCCCAAACTCCTCATT | |
| TTTGACAATGATAAGCGACCCTCAGGGATTCCTGACCGATTCTCTGGCTCCAAGTCT | |
| GGCACGTCAGCCACCCTGGGCATCACCGGACTCCAGACTGGGGACGAGGCCGATTAT | |
| TATTGCGGAACATGGGATAAGAGTCCGACTGACATTTATGTCTTCGGAAGTGGGACC | |
| AAGCTGACCGTCCTAGGT, | |
| PGIA-03-A12 | |
| CAGGTGCAGCTGCAGGAGTCCGGCCCAGGACTGGTGAAGCCTTCGGGGACCCTGTCC | SEQ ID NO:99 |
| CTCACCTGCGCTGTCTCTGGTGGCTCCATCAGCAGTAGTAACTGGTGGAGTTGGGTC | |
| CGCCAGGCCCCAGGGAAGGGGCTGGAGTGGATTGGGGAAATCTATTATGGTGGGAGC | |
| ACCAACTACAACCCGTCCCTCAAGAGTCGAGTCACCCTTTCAGTAGACAAGTCCAAG | |
| AACCAGTTCTCCCTGAGGCTGATTTCTGTGACCGCCGCGGACACGGCCGTCTATTAC | |
| TGTGCGAGAAGTAGTGGCCTCTACGGTGACTACGGGAACCTGTGGGGCCGAGGAACC | |
| CTGGTCACCGTCTCGAGTGGAGGCGGCGGTTCAGGCGGAGGTGGCTCTGGCGGTGGC | |
| GGAAGTGCACAGTCTGTCGTGACGCAGCCGCCCTCAGTGTCTGCGGCCCCAGGACAG | |
| AAGGTCACCATCTCCTGCTCTGGAAGCGCCTCCAACATTGGAGATCATTATATATCC | |
| TGGTACCAGCAGTTCCCAGGAACAGCCCCCAAACTCCTCATCTCTGACAATGATCAG | |
| CGACCCTCAGGGATTCCTGACCGGTTCTCTGGCTCCAAGTCTGGCACATCAGCCACC | |
| CTGGGCATCACCGGACTCCAGACTGGGGACGAGGCCGATTACTACTGCGGAACATGG | |
| GATAGCAACCTGAGTTCTTGGGTGTTTGGCAGTGGGACCAAGGTCACCGTCCTAGGT, | |
| PGIA-03-B1 | |
| GAAGTGCAGCTGGTGCAGTCTGGGGCTGAGGTGAAGAAGCCTGGGGCTACACTGAAA | SEQ ID NO:100 |
| GTCTCCTGCAAAGTTTCTGCATACACCTTCACCGACTACTCCATGCACTGGGTGCAA | |
| CAGGCCCCTGGAAAAGGGCTTAAGTGGATGGGACTTATTGATCTTGAAGATGGTAAT | |
| ACAATTTACGCAGAGGAGTTCCAGGACAGAGTCACCATAACCGCGGACACGTCTACA | |
| GACACAGCCTACATGGATCTGAGCAGCCTGAGATCTGAGGACACGGCCGTGTTTTAC | |
| TGTGCAATAAGTCCGCTTCGGGGACTTACCGCGGATGTTTTTGATGTCTGGGGCCAA | |
| GGAACCCTGGTCACCGTCTCGAGTGGAGGCGGCGGTTCAGGCGGAGGTGGCTCTGGC | |
| GGTGGCGGAAGTGCACAGTCTGCCCTGACTCAGCCTGCCTCCGCGTCTGGGTCTCCT | |
| GGACAGTCGATCACCATCTCCTGCACTGGAACCAGCAGTGACATTGGTCGTTATGAC | |
| TTTGTCTCTTGGTATCAACGACAACCAGGCAAAGCCCCCAAACTCATGATTTATGAT | |
| GTCATTAATCGGCCCTCAGGGGTTTCTAGTCGCTTCTCTGGCTCCAAGTCTGGCAAC | |
| ACGGCCTCCCTGACCATCTCTGGGCTCCAGGCTGAGGACGAGGCTGATTATTACTGC | |
| AGCTCATATGCAGGTTCCACCACTCTCTATGTCTTCGGCACTGGGACCAAGCTGACC | |
| GTCCTAGGT, | |
| PGIA-03-B2 | |
| CAGGTGCAGCTGCAGGAGTCGGGCCCAGGACTGGTGAAGCCTTCGGCGACCCTGTCC | SEQ ID NO:101 |
| CTCACCTGCGCTGTCTCTGGTGGCTCCATCAGCAGTAATCACTGGTGGAGTTGGGTC | |
| CGCCAGTCCCCCGGGAAGGGTCTGGAGTGGATTGGAGAAATCTATACTTATGGGGGC | |
| GCCAACTACAACCCGTCCCTCAAGAGTCGAGTCGACATATCAATGGACAAGTCCAAG | |
| AATCAGTTCTCCCTGCACTTGAGCTCTGTGACCGCCGCGGACACGGCCGTGTATTAC | |
| TGTGGGAGACACCTGACTGGTTACGATTGTTTTGATATCTGGGGCCAAGGAACCCTG | |
| GTCACCGTCTCGAGTGGAGGCGGCGGTTCAGGCGGAGGTGGCTCTGGCGGTGGCGGA | |
| AGTGCACAGGCTGTGCTGACTCAGCCGTCCTCAGTGTCTGGGGCCCCAGGGCAGAGG | |
| GTCACCATCTCCTGCACTGGGAGCAGCTCCAACATCGGGGCAGGTTATGATGTACAC | |
| TGGTACCAGCAGCTTCCAGGAACAGCCCCCAAACTCCTCATCTATGGTAACAGCAAT | |
| CGGCCCTCAGGGGTCCCTGACCGATTCTCTGGCTCCAAGTCTGGCACCTCAGCCTCC | |
| CTGGCCATCACTGGGCTCCAGGCTGAGGATGAGGCTGATTATTACTGCCAGTCCTAT | |
| GACAGCAGCCTGAGTGGTGTCTTCGGAACTGGGACCCAGCTCACCGTTTTAAGT, | |
| PGIA-03-B3 | |
| CAGGTGCAGCTGCAGGAGTCCGGCCCAGGACTGGTGAAGCCTTCGGGGACCCTGTCC | SEQ ID NO:102 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AACCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCAAGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGCCAGCAAGTAT | |
| GTGCAGTGGTACCAGCAGCGCCCGGGCAGTGCCCCCACCAGTGTCATCTATGAGGAT | |
| AACCAAAGACCCTCTGGGGTCCCTGATCGGTTCTCTGGCTCCATCGACAGCGCCTCC | |
| AACTCTGCCTCCCTCACCATCTCTGGACTGAAGACTGAGGACGAGGCTGACTACTAC | |
| TGTCAGTCTGATGATGGCAGCAGTGTGGTTTTCGGCGGAGGGACCAAGGTCACCGTC | |
| CTAGGT, | |
| PGIA-03-B4 | |
| GAGGTCCAGCTGGTGCAGTCTGGGGCTGAGGTGAAGAAGCCTGGGGCCTCAGTGAAG | SEQ ID NO:103 |
| GTCTCCTGCAAGGCTTCGGGATACAGCTTTCCCAGCTCTGGTCTCAGCTGGGTGCGA | |
| CAGGCCCCTGGACAAGGGCCTGAGTGGATGGGATGGATCGGCATTTACAATGGTAAC | |
| ACAGACTATGCACAGAAGTTCCAGGGCAGAGTCACCATGACCACAGACAAATCCACG | |
| AGCACAGCCTACATGGAGCTGAGGAGCCTGAGATCTGACGACACGGCCGTCTATTAC | |
| TGTGCGAGAGATTCCGTGGGGAGTATATCAGTGGCTGGTACGATGCAATACTACTAC | |
| TTCGCTATGGACGTCTGGGGCCAAGGAACCCTGGTCACCGTCTCGAGTGGAGGCGGC | |
| GGTTCAGGCGGAGGTGGCTCTGGCGGTGGCGGAAGTGCACAGTCTGTGTTGACGCAG | |
| CCGCCCTCCGCGTCCGGGTCTCCTGGACAGTCAGTCACCATCTCCTGCGCTGGAACC | |
| AGGTATGACATTGGTACTTATAATTATGTCTCGTGGTACCAACAACACCCAGCCAAA | |
| GGCCCCAAACTCATCATTTATGCGGTCAGTGAGCGGCCCTCAGGTGTCCCTAATCGA | |
| TTCTCTGGCTCCAAGTCTGGCAACACGGCCTCCCTGACCGTCTCCGGGCTCCGGGCT | |
| GAGGATGAGGCTCATTATTATTGCAGCTCATACGCAGGCAACAACAATGTGATTTTC | |
| GGCGGAGGGACCAAGGTCACCGTCCTAGGT, | |
| PGIA-03-B5 | |
| CAGGTGCAGCTGCAGGAGTCCGGCCCAGGACTGGTGAAGCCTTCGGGGACCCTGTCC | SEQ ID NO:104 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCGAGGG | |
| ACAATGGTCACCGTCTCGAGTGGAGGCGGCGGTTCAGGCGGAGGTGGCTCTGGCGGT | |
| GGCGGAAGTGCACAGTCTGTGCTGACGCAGCCGCCCTCAGCGTCTGGGACCCCCGGA | |
| CAGAGGGTCACCATCTCTTGTTCTGGAAGCTTCTCCAATATCGGAGGTAATTATGTG | |
| AACTGGTACCAGCAGCTCCCAGGAACGGCCCCCAAACTCCTCATCTATGGGAATAAT | |
| CAGCGGCCCTCAGGGGTCCCTGACCGATTCTCTAGTTTTAAGTCGGGCACCTCAGCC | |
| TCCCTGGCCATCAGTGGGCTCCGGTCCGAGGATGAGGCTGATTATTACTGTGCAACA | |
| TGGGATGACAGCCAGACTGTTTTATTCGGCGGAGGGACCAAGCTGACCGTCCTAGGT, | |
| PGIA-03-B6 | |
| GAGGTGCAGCTGTTGGAGTCTGGGGGAGGCTTGGTACAGCCTGGGGGGTCCCTGAGA | SEQ ID NO:105 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAGATGGAATGGTTTCCTGACAGCTCATGACTCCTGGGGCCGAGGGACAATG | |
| GTCACCGTCTCGAGTGGAGGCGGCGGTTCAGGCGGAGGTGGCTCTGGCGGTGGCGGA | |
| AGTGCACAGTCTGTGCTGACTCAGCCACCCTCAGCGTCTGGGACCCCCGGGCAGAGG | |
| GTCACCATCTCTTGTTCTGGAAGCAGTTCCAACATCGGAACTAATTATGTGTACTGG | |
| TACCAACAATTCCCAGGAACGGCCCCCAAACTCCTCATCTATAGGAGTAATCGGCGG | |
| CCCTCAGGGGTCCCTGACCGATTCTCTGCCTCCAAGTCTGGCACCTCAGCCTCCCTG | |
| GTCATCAGTGGGCTCCGGTCCGAAGATGAGGCTGACTATTACTGTGCAGCATGGGAT | |
| GACAGACTGAATGGCGAGATGTTCGGCGGAGGGACCAAGGTCACCGTCCTAGGT, | |
| PGIA-03-B7 | |
| GAGGTGCAGCTGTTGGAGTCTGGGGGAGGCTTGGTACAGCCTGGGGGGTCCCTGAGA | SEQ ID NO:106 |
| CTCTCNTGTGCAGCCTCTGGATTCACCTTTAGCAGCTATGCCATGAGCTGGGTTCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAGATGGTCCGGGCGGTTTTATGACTTCTGGGGGCAAGGGACCACGGTCACC | |
| GTCTCGAGTGGAGGCGGCGGTTCAGGCGGAGGTGGCTCTGGCGGTGGCGGACGTGCA | |
| CAGTCTGTGCTGACTCAGCCACCCTCAGCGTCTGGGACCCCCGGGCAGAGGATCACC | |
| ATCTCTTGTTCCGGAAGCAGCTCCAACATCGGAAGTAATTATGTATACTGGTACCAG | |
| CAACTCCCAGGAACGGCCCCCAAAATCCTCATCTATAGGAATAATCAGCGGCCCTCA | |
| GGGGTCCCTGAGCGATTCTCTGGCTCCAAGTCTGGCACCTCAGCCTCCCTGGCCATC | |
| AGTGGGCTCCGGTCCGAGGATGAGGCTGACTACTATTGTGCAGCATGGGATGACAGC | |
| CTGAGTGAAGTGTTCGGCGGAGGGACCAAGGTCACCGTCCTAGGT, | |
| PGIA-03-B8 | |
| GAGGTGCAGCTGTTGGAGTCTGGGGGAGGCTTGGTACAGCCTGGGGGGTCCCTGAGA | SEQ ID NO:107 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCCCG | |
| AACACGCTGTATCTGCAAATGAACAGCCTGAGAGCCGAGGACACGGCCGTGTATTAC | |
| TGTGCGAGAGATAAGGGTTATAGTGGCTTTGACTACTGGGGCCGGGGAACCCTGGTC | |
| ACCGTCTCGAGTGGAGGCGGCGGTTCAGGCGGAGGTGGCTCTGGCGGTGGCGGAAGT | |
| GCACAGTCTGTGTTGACGCAGCCGCCCTCAGCGTCTGGGACCCCCGGGCAGAGGGTC | |
| ACCATCTCTTGCTCTGGAAGCAGCTCCAACATCGGACGTCATACTGTTAACTGGTAC | |
| CAGCAACTCCCAGGAACGGCCCCCAAACTGCTCATCTATAGCAATCCTCAGCGGCCC | |
| TCAGGGGTCCCTGACCGATTCTCTGGCTCCAAGTCTGGCACCTCAGCCTCCCTGGCC | |
| ATCAGTGGGCTCCAGTCTGAAGATGAGGGTCATTATCACTGTGCAGCATGGGATGAC | |
| ACCCTGAATGGTGATGTGGTATTCGGCGGAGGGACCAAGGTCACCGTCCTAGGT, | |
| PGIA-04-A1 | |
| CAGCTGCAGCTGCAGGAGTCCGGCCCAGGACTGGTGAAGCCTTCGGGGACCCTGTCC | SEQ ID NO:108 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCAAGGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGCCAGCAACTAT | |
| GTGCAGTGGTACCAGCAGCGCCCGGGCAGTTCCCCCACCACTGTGATCTATGAGGAT | |
| AACCAAAGACCCTCTGGGGTCCCTGATCGGTTCTCTGGCTCCATCGACAGCTCCTCC | |
| AACTCTGCCTCCCTCACCATCTCTGGACTGAAGACTGAGGACGAGGCTGACTACTAC | |
| TGTCAGTCTTATGATAGCAGCAACCCTTATGTGGTATTCGGCGGAGGGACCAAGCTG | |
| ACCGTCCTAGGT, | |
| PGIA-04-A2 | |
| CAGGTGCAGCTGCAGGAGTCCGGCCCAGGACTGGTGAAGCCTTCGGGGACCCTGTCC | SEQ ID NO:109 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCCCTTGACTACTGGGGCCAGGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGGGTCTCCG | |
| GGGAGGACGGTAACCATCTCCTGCACCCGCAGCAGTGGCAGCATTGCCACCAACTAT | |
| GTGCAGTGGTACCAGCAGCGCCCGGGCAGTTCCCCCACCATTGTGATCTATGAAGAT | |
| AACCAAAGACCCTCTGGGGTCCCTGATCGCTTCTCTGGCTCCATCGACACCTCCTCC | |
| AACTCTGCCTCCCTCACCATCTCTGGACTGAAGACTGAGGACGAGGCTGACTACTAC | |
| TGTCAGTCTTATGATAGCAACAATCTGGGGGTGGTATTTGGCGGAGGGACCCAGCTC | |
| ACCGTTTTAAGT | |
| PGIA-04-A3 | |
| CAGCTGCAGCTGCAGGAGTCGGGCCCAGGACTGGTGAAGCCTTCGGGGACCCTGTCC | SEQ ID NO:110 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCAGGGC | |
| ACCCTGGTCACCGTCTCGAGTGGAGGCGGCGGTTCAGGCGGAGGTGGCTCTGGCGGT | |
| GGCGGAAGTGCACAGTCTGTCGTGACGCAGCCGCCCTCAGTGTCTGCGGCCCCAGGA | |
| CAGAAGGTCACCATCTCCTGCTCTGGAAGCAGCTCCAACATTGGGAATAATTATGTA | |
| TCCTGGTATAAACAACTCCCAGGAACAGCCCCCAAACTCCTCATCTATGACAATAAT | |
| AAGCGACCCTCTGGGATTCCTGACCGATTCTCTGGCTCCAAGTCTGGCACGTCAGCC | |
| ACCCTGGGCATAACCGGACTCCAGACTGGGGACGAGGCCGATTATTACTGCGGAACT | |
| TGGGATAGCAGCCTGAGTGGCGTGGTGTTCGGCGGAGGGACCAAGCTGACCGTCCTA | |
| GGT | |
| PGIA-04-A4 | |
| CAGCTGCAGCTGCAGGAGTCGGGCCCAGGACTGGTGAAGCCTTCGGGGACCCTGTCC | SEQ ID NO:111 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCGAGGA | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGCCAGCAACTAT | |
| GTGCAGTGGTACCAACAGCGCCCGGGCAGTTCCCCCACCACTTTGATCTATGACGAT | |
| AACCAGAGACCCTCTGGGGTCCCTGATCGGTTCTCTGGCTCCATCGACAGCTCCTCC | |
| AACTCTGCCTCCCTCACCATCTCTGGACTGAAGACTGAGGACGAGGCTGACTACTAC | |
| TGTCAGTCTTATGACAGCAGCAATCTGGGGGTGGTCTTCGGCGGAGGGACCAAGCTG | |
| ACCGTCCTAGGT | |
| PGIA-04-A5 | |
| CAGGTGCAGCTGCAGGAGTCGGGCCCAGGACTGGTGAAGCCTTCGGGGACCCTGTCC | SEQ ID NO:112 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCGGGGA | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGCAACCATCTCCTGCACCGGCAGCGGTGGCAGCATTGCCAGAAGCTAT | |
| GTGCAGTGGTACCAGCAGCGCCCGGGCCGTGCCCCCAGCATCGTTATCTATGAGGAT | |
| TATCAAAGGCCCTCTGGCGTCCCTGATCGGTTCTCTGGCTCCATCGACAGCTCCTCC | |
| AATTCTGCCTCTCTCACCATCACTGGGCTGAAGACTGACGACGAGGCTGACTACTAC | |
| TGTCAGTCCTCTGACGACAACAACAATGTCGTCTTCGGCGGAGGGACCAAGGTCACC | |
| GTCCTAGGT | |
| PGIA-04-A6 | |
| CAGGTGCAGCTGCAGGAGTCCGGCCCAGGACTGGTGAAGCCTTCGGGGACCCTGTCC | SEQ ID NO:113 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCAGGGGA | |
| ACCCTGGTCACCGTCTCGAGTGGAGGCGGCGGTTCAGGCGGAGGTGGCTCTGGCGGT | |
| GGCGGAAGTGCACAGGCTGTGCTGACTCAGCCGTCCTCAGTGTCTGCGGCCCCAGGA | |
| CAGAAGGTCACCATCTCCTGCTCTGGAAGCAGCTCCAACATTGGGAATAATTATGTA | |
| TCCTGGTACCAGCAGCTCCCAGGAACAGCCCCCAAACTCCTCATTTATGACAATAAT | |
| GAGCGACCCTCAGGGATTCCTGACCGATTCTCTGGCTCCAAGTCTGGCACGTCAGCC | |
| ACCCTGGGCATCACCGGACTCCAGACTGGGGACGAGGCCGATTATTACTGCGGAACA | |
| TGGGATAGCAGCCTGAGTACTGTGGTCTTCGGAACTGGGACCAAGGTCACCGTCCTA | |
| GGT, | |
| PGIA-04-A7 | |
| CAGCTGCAGCTGCAGGAGTCGGGCCCAGGACTGGTGAAGCCTTCGGGGACCCTGTCC | SEQ ID NO:114 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCAGGGA | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTGACCGTTTCCTGCACCGGCAGCGGTGGCAACATTGCCAGCAATTAT | |
| GTACAGTGGTACCAGCAGCGCCCGGACAGTGCCCCCACCCTTGTGATCTTTGAGGAT | |
| ACCCAAAGGCCCTCTGGGGTCCCTGCTCGGTTCTCTGGCTCCATCGACAGCTCCTCC | |
| AACTCTGCCTCCCTCATCATCTCCTCACTGAGGACTGAGGACGAGGCTGATTACTAT | |
| TGTCAATCTTCTGATTCCAACAGGGTGGTGTTCGGCGGAGGGACCAAGGTCACCGTC | |
| CTAGGT, | |
| PGIA-04-A8 | |
| CAGGTGCAGCTGCAGGAGTCGGGCCCAGGACTGGTGAAGCCTTCGGAGACCCTGTCC | SEQ ID NO:115 |
| CTCACCTGCAATGTCTCTGGTGGCTCCATCAGGAATTACTTCTGGAGTTGGATCCGG | |
| CAGCCCCCAGGGCAGGGACTGGAGTACATTGGGTATATCTATTACAGTGGGACCACC | |
| GACTACAACCCCTCCCTCAAGGGTCGAGTCACCATATCACTAGACACGTCCAAGACC | |
| CAGTTCTCCTTGAAGCTGAACTCTGTGACCGCTGCGGACACGGCCTTCTATTACTGT | |
| GTGAGAGGCCCGAATAAGTATGCGTTCGACCCCTGGGGCCAAGGCACCCTGGTCACC | |
| GTCTCGAGTGGAGGCGGCGGTTCAGGCGGAGGTGGCTCTGGCGGTGGCGGAAGTGCA | |
| CTTTCCTATGAGCTGACTCAGCCACCCTCAGTGTCCGTGTCCCCCGGACAGACAGCC | |
| AGCATCACCTGCTCTGGAGATAAATTGGGGGATAAATTTGCTTCCTGGTATCAACAG | |
| AAGGCAGGCCAGTCCCCTGTGCTGGTCATCTATCGAGATACCAAGCGCCCCTCAGGG | |
| ATCCCTGAGCGATTCTCTGGCTCCAACTCTGGGAACACAGCCACTCTCACCATCAGC | |
| GGGACCCAGGCTATGGATGAGGCTGATTATTACTGTCAGGCGTGGGACAGCAGCACG | |
| GCGGTCTTCGGAACTGGGACCAAGGTCACCGTCCTAGGT, | |
| PGIA-04-A9 | |
| CAGCTGCAGCTGCAGGAGTCGGGCCCAGGACTGGTGAAGCCTTCGGGGACCCTGTCC | SEQ ID NO:116 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCAAGGA | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCACCCGCAGCAGTGGCAGCATTGACAACAACTAT | |
| GTCCAGTGGTACCAGCAGCGCCCGGGCAGTTCCCCCACTACTGTGATCTTTGAGGAT | |
| AACCAAAGACCCTCTGGGGTCCCTGATCGCTTCTCTGGCTCCATCGACAGCTCCTCC | |
| AACTCTGCCTCCCTCACCATCTCTGGACTGAAGACTGAGGACGAGGCTGACTACTAC | |
| TGTCAGTCTTATGATAGCCACAATCAGGGGGTGGTCTTCGGCGGAGGGACCAAGCTG | |
| ACCGTCCTAGGT, | |
| PGIA-04-A10 | |
| CAGCTGCAGCTGCAGGAGTCCGGCCCAGGACTGGTGAAGCCTTCGGGGACCCTGTCC | SEQ ID NO:117 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCGAGGA | |
| ACCCTGGTCACCGTCTCGAGTGGAGGCGGCGGTTCAGGCGGAGGTGGCTCTGGCGGT | |
| GGCGGAAGTGCACAGTCTGTGCTGACGCAGCCGCCCTCAGTGTCTGCGGCCCCAGGA | |
| CAGAAGGTCACCATCTCCTGCTCTGGAAGTAGCTCCAACATTGGGAATAGTTATGTA | |
| TCGTGGTACAAGCAGCTCCCAGGTACAGCCCCCAAAGTCCTCATTTATGACAACCAG | |
| AAGCGATCCTCAGGGATCCCTGACCGATTCTCTGCCTCCAAGTCTGGCACGTCAGCC | |
| ACCCTGGGCATCACCGGACTCCGGACTGAGGACGAGGCCGATTATTACTGCGGAACA | |
| TGGGATACCAGCCTGAGTGCGGTGGTGTTCGGCGGAGGGACCAAGCTGACCGTCCTA | |
| GGT, | |
| PGIA-04-A11 | |
| GAGGTGCAGCTGGTGGAGTCTGGCCCAGGACTGGTGAAGCCTTCGGGGACCCTGTCC | SEQ ID NO:118 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCCGGGGA | |
| ACCCTGGTCACCGTCTCGAGTGGAGGCGGCGGTTCAGGCGGAGGTGGCTCTGGCGGT | |
| GGCGGAAGTGCACAGTCTGTCGTGACGCAGCCGCCCTCAGTATCTGCGGCCCCAGGA | |
| CAGAAGGTCACCATCTCCTGCTCTGGAAACTTCTCCAACATTGAATATAATTATGTA | |
| TCGTGGTACCAGCACCTCCCAGGAACAGCCCCCAAACTCCTCATTTTTGACAATAAT | |
| CAGCGACCCTCATGGATTCCTGACCGATTCTCTGGCTCCAAGTCTGGCACGTCAGCC | |
| ACCCTGGGCATCACCGGGCTCCAGACTGGGGACGAGGCCGATTACTACTGCGGAACA | |
| TGGGATAGCAGCCTGAATGCTGGGGTGTTCGGCGGAGGGACCAAGGTCACCGTCCTA | |
| GGT, | |
| PGIA-04-A12 | |
| GAGGTGCAGCTGTTGGAGTCTGGGGGAGGCTTGGTACGGCCTGGGGGGTCCCTGAGA | SEQ ID NO:119 |
| CTCTCCTGTGCAGCCTCTGGATTCACCTTTAGCAGCTATGCCATGAGCTGGGTCCGC | |
| CAGGCTCCAGGGAAGGGGCTGGAGTGGGTCTCAGCTATTAGTGGTAGTGGTGGTAGC | |
| ACATACTACGCAGACTCCGTGAAGGGCCGGTTCACCATCTCCAGAGACAATTCCAAG | |
| AACACGCTGTATCTGCAAATGAACAGCCTGAGAGCCGAGGACACGGCCGTGTATTAC | |
| TGTGCGAAAGATCGAAGGGGTGTCCTCGACCCCTGGGGCAAAGGGACAATGGTCACC | |
| GTCTCGAGTGGAGGCGGCGGTTCAGGCGGAGGTGGCTCTGGCGGTGGCGGAAGTGCA | |
| CAGTCTGTGCTGACGCAGCCGCCCTCAGTGTCTGGGGCCCCAGGGCAGAGGGTCACC | |
| ATCTCCTGCACTGGGAGCAGCTCCAACATCGGGGCAGGCTATGATGTACACTGGTAC | |
| CAGCACCTTCCAGGAACAGCCCCCAGACTCCTCATCTATGGTAACAGCAATCGGCCC | |
| TCAGGGGTCCCTGACCGATTCTCTGGCTCCAAGTCTGGCACCTCAGCCTCCCTGGCC | |
| ATCTCTGGCCTCCAGGCTGAGGATGAGGCTGATTATTACTGCCAGTCCTATGACAGC | |
| AGCCTGAGTGATTGGGTGTTCGGCGGAGGGACCAAGGTCACCGTCCTAGGTC, | |
| and | |
| PGIA-05-A1 | |
| CAGCTGCAGCTGCAGGAGTCCGGCCCAGGACTGGTGAAGCCTTCGGGGACCCTGTCC | SEQ ID NO:120 |
| CTCACCTGCGCTGTCTCTGGTGGCTCCATCAGCACTAGTGACTGGTGGAGTTGGGTC | |
| CGCCGGCCCCCAGGGAAGGGGCTGGAGTGGATTGGGGAAATCTATCATAGTGGGAGC | |
| ACCAACTACCACCCGTCACTCAAGAGTCGAGTCACCATATCACTTGACAAATCGAAG | |
| AATCAGTTCTCCCTGAAACTGAGCTCTGTGACCGCCGCGGACACGGCCGTGTATTAC | |
| TGTGCGAGAGAGGGGGGCCATAGTGGGAGTTACCCTCTTGACTACTGGGGCAGGGGC | |
| ACCCTGGTCACCGTCTCGAGTGGAGGCGGCGGTTCAGGCGGAGGTGGCTCTGGCGGT | |
| GGCGGAAGTGCACTTAATTTTATGCTGACTCAGCCCCACTCTGTGTCGGAGTCTCCG | |
| GGGAAGACGGTAACCATCTCCTGCGCCCGCAGCAGTGGCAGCATTGCCAGCAACTAT | |
| GTGCAGTGGTACCAGCAGCGCCCGGGCAGTTCCCCCACCACTTTGATCTATGAGGAT | |
| AGGCAAAGACCCTCTGGGGTCCCTGATCGGTTCTCTGGCTCCATCGACAGCTCCTCC | |
| AACTCTGCCTCCCTCACCATCTCTGGACTGAAGACTGAGGACGAGGCTGACTACTAC | |
| TGTCAGTCTTATGATAGCAGCGATCATGTGGTCTTCGGCGGAGGGACCAAGCTGACC | |
| GTCCTAGGT. | |

### Inhibition of c-Met Activity by c-Met Antibody

Inhibition of HGF Binding to c-Met

In another embodiment, the invention provides c-Met antibodies that inhibit the binding of HGF to c-Met. In a preferred embodiment, the c-Met is of human origin. In another preferred embodiment, the c-Met antibody is a human antibody. In another embodiment, the antibody or portion thereof inhibits binding between c-Met and HGF with an IC₅₀ of no more than 100 nM. In a preferred embodiment, the IC₅₀ is no more than 10 nM. In a more preferred embodiment, the IC₅₀ is no more than 5 nM. The IC₅₀ can be measured by any of a number of methods known in the art. Typically, an IC₅₀ can be measured by ELISA or RIA. In a preferred embodiment, the IC₅₀ is measured by RIA.

In another embodiment, the invention provides a c-Met antibody that prevents activation of c-Met in the presence of HGF. In a preferred embodiment, the c-Met antibody inhibits c-Met-induced tyrosine phosphorylation of the kinase domain following receptor autophosphorylation. The c-Met antibody inhibits downstream cellular events from occurring. For instance, the c-Met antibody can inhibit serine phosphorylation of Akt that is normally phosphorylated and activated when cells are treated with HGF. One can determine whether a c-Met antibody can prevent activation of c-Met in the presence of HGF by determining the levels of tyrosine phosphorylation for c-Met, or serine phosphorylation at Ser 473 on Akt by Western blot, immunoprecipitation, or ELISA assay.

In another aspect of the invention, the antibody causes the downregulation of c-Met from a cell treated with the antibody. In one embodiment, the c-Met is internalized into the endosomal pathway of the cell. After the c-Met antibody binds to c-Met, the antibody bound to c-Met is internalized. One may measure the downregulation of c-Met by any method known in the art including immunoprecipitation, confocal microscopy, or Western blot. In a preferred embodiment, the antibody is selected PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-Al1, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, or comprises a heavy chain, light chain or antigen-binding region thereof.

### Activation of c-Met by c-Met Antibody Binding

Another aspect of the present invention involves activating c-Met antibodies. An activating antibody differs from an inhibiting antibody because it amplifies or substitutes for the effects of HGF on c-Met. In one embodiment, the activating antibody is able to bind to c-Met and cause it to be activated in the absence of HGF. This type of activating antibody is essentially a partial or complete mimetic of HGF. In another embodiment, the activating antibody amplifies the effect of HGF on c-Met.

This type of antibody does not activate c-Met by itself, but rather increases the activation of c-Met in the presence of HGF. A mimic anti c-Met antibody may be easily distinguished from an amplifying c-Met antibody by treating cells in vitro with an antibody in the presence or absence of low levels of HGF. If the antibody is able to cause c-Met activation in the absence of HGF, e.g., it increases c-Met tyrosine phosphorylation, and then the antibody is a mimic antibody. If the antibody cannot cause c-Met activation in the absence of HGF but is able to amplify the amount of c-Met activation, then the antibody is an amplifying antibody.

### Inhibition of c-Met Tyrosine Phosphorylation, c-Met Levels, and Tumor Cell Growth in vivo by c-Met Antibodies

Another embodiment of the invention provides a c-Met antibody that inhibits c-Met tyrosine phosphorylation and receptor levels in vivo. In one embodiment, administration of c-Met antibody to an animal causes a reduction in c-Met phosphotyrosine signal in c-Met-expressing tumors. In a preferred embodiment, the c-Met antibody causes a reduction in phosphotyrosine signal by at least 20%. In a more preferred embodiment, the c-Met antibody causes a decrease in phosphotyrosine signal by at least 50%, more preferably 60%. In an even more preferred embodiment, the antibody causes a decrease in phosphotyrosine signal of at least 70%, more preferably 80%, even more preferably 90%. In a preferred embodiment, the antibody is administered approximately 24 hours before the levels of tyrosine phosphorylation are measured.

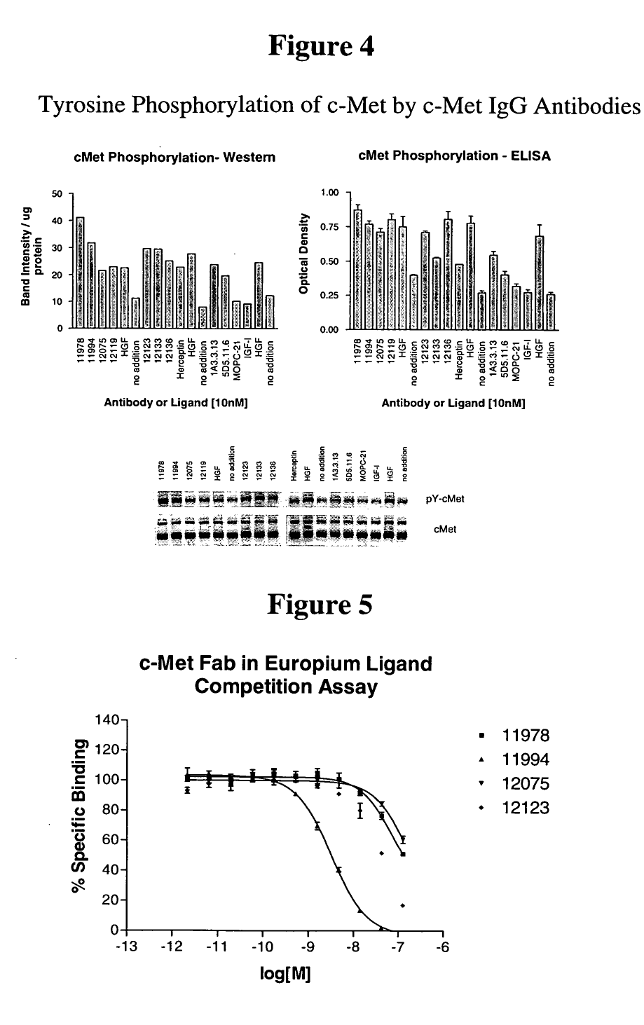

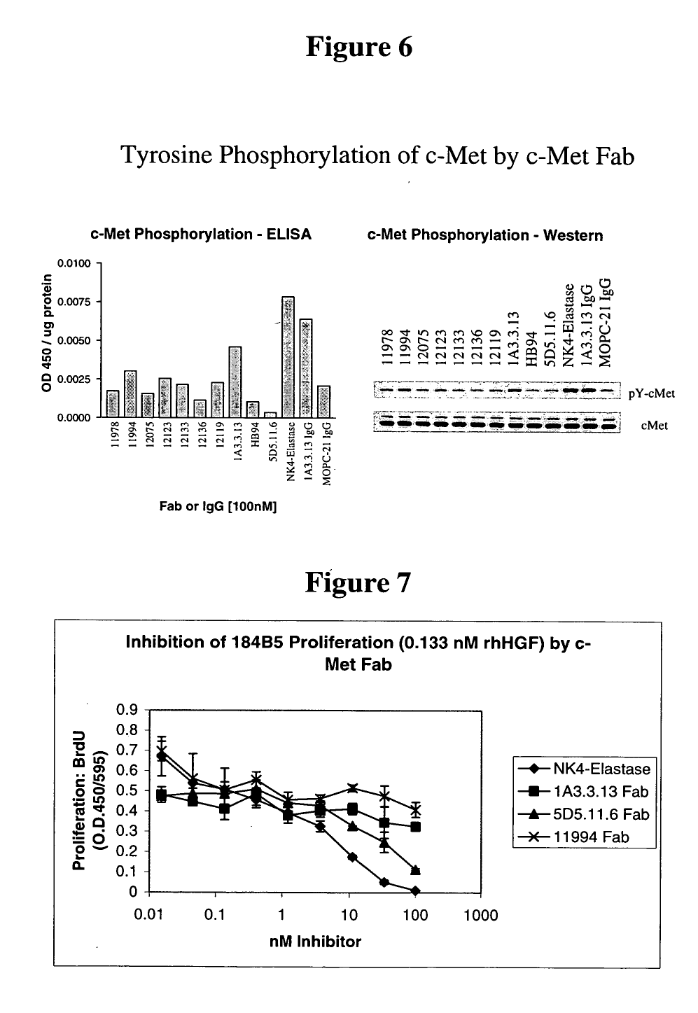

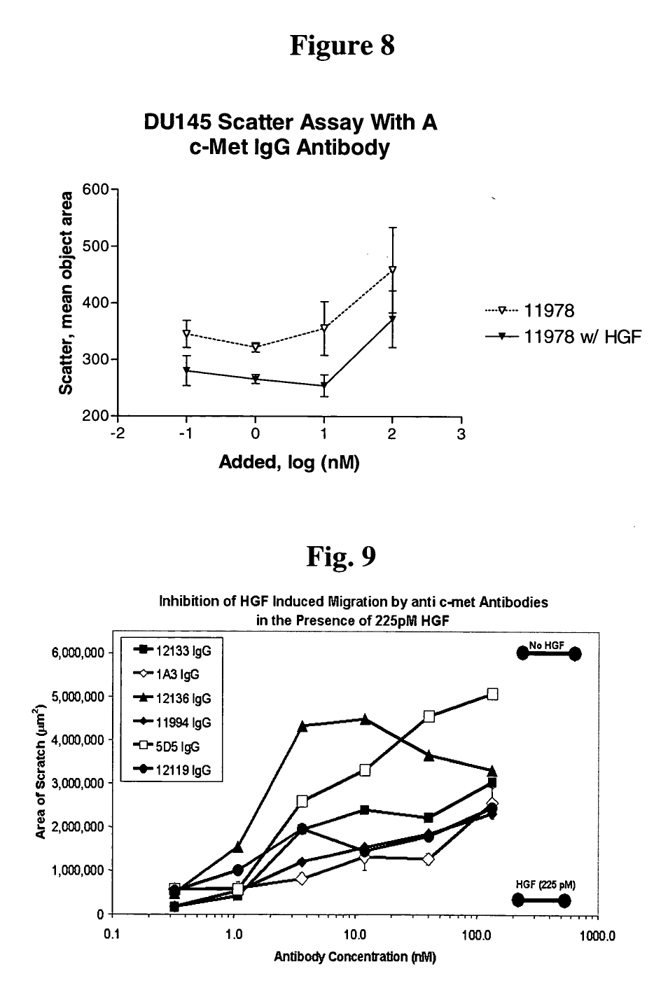

The levels of tyrosine phosphorylation may be measured by any method known in the art, such as those described infra. See, e.g., Example 5 and FIGS. 4 & 6. In a preferred embodiment, the antibody is selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, or comprises a heavy chain, light chain or antigen-binding portion thereof.

In another embodiment, administration of c-Met antibody to an animal causes a reduction in c-Met levels in c-Met-expressing tumors. In a preferred embodiment, the c-Met antibody causes a reduction in receptor levels by at least 20% compared to an untreated animal. In a more preferred embodiment, the c-Met antibody causes a decrease in receptor levels to at least 60%, more preferably 50% of the receptor levels in an untreated animal. In an even more preferred embodiment, the antibody causes a decrease in receptor levels to at least 40%, more preferably 30%. In a preferred embodiment, the antibody is administered approximately 24 hours before the c-Met levels are measured. The c-Met levels may be measured by any method known in the art, such as those described infra. In a preferred embodiment, the antibody is selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1 or comprises a heavy chain, light chain or antigen-binding portion thereof.

In another embodiment, a c-Met antibody inhibits tumor cell growth in vivo. The tumor cell may be derived from any cell type including, without limitation, epidermal, epithelial, endothelial, leukemia, sarcoma, multiple myeloma, or mesodermal cells. Examples of common tumor cell lines for use in xenograft tumor studies include A549 (non-small cell lung carcinoma) cells, DU-145 cells, HCT-116 cells, MCF-7 cells, Colo 205 cells, 3T3/c-Met cells, 184B5 cells, NCI H441 cells, HEP G2 cells, MDA MB 231 cells, HT-29 cells, MDA-MB-435 cells, GTL-16 cells, B×PC3 cells, S114 cells, MDCK cells, A549 cells, U0118 MG cells, B16 cells, U-87 MG cells, and A431 cells. In a preferred embodiment, the antibody inhibits tumor cell growth as compared to the growth of the tumor in an untreated animal. In a more preferred embodiment, the antibody inhibits tumor cell growth by 50%. In an even more preferred embodiment, the antibody inhibits tumor cell growth by 60%, 65%, 70%, or 75%. In one embodiment, the inhibition of tumor cell growth is measured at least 7 days after the animals have started treatment with the antibody. In a more preferred embodiment, the inhibition of tumor cell growth is measured at least 14 days after the animals have started treatment with the antibody. In another preferred embodiment, another antineoplastic agent is administered to the animal with the c-Met antibody. In a preferred embodiment, the antineoplastic agent is able to further inhibit tumor cell growth. In an even more preferred embodiment, the antineoplastic agent is Adriamycin, taxol, tamoxifen, 5-fluorodeoxyuridine (5-FU) or CP-358,774. In a preferred embodiment, the co-administration of an antineoplastic agent and the c-Met antibody inhibits tumor cell growth by at least 50%, more preferably 60%, 65%, 70% or 75%, more preferably 80%, 85% or 90% after a period of 22-24 days.

### Induction of Apoptosis by c-Met Antibodies

Another aspect of the invention provides a c-Met antibody that induces cell death. In one embodiment, the antibody causes apoptosis. The antibody may induce apoptosis either in vivo or in vitro. In general, tumor cells are more sensitive to apoptosis than normal cells, such that administration of a c-Met antibody causes apoptosis of a tumor cell preferentially to that of a normal cell. In another embodiment, the administration of a c-Met antibody effects the activation of a kinase Akt, which is involved in the phosphatidyl inositol (PI) kinase pathway.

The PI kinase pathway, in turn, is involved in the cell proliferation and prevention of apoptosis. Thus, inhibition of Akt can cause apoptosis. In a more preferred embodiment, the antibody is administered in vivo to cause apoptosis of a HGF expressing cell. In a preferred embodiment, the antibody is selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, or comprises a heavy chain, light chain, or antigen-binding portion thereof.

### Methods of Producing Antibodies and Antibody-Producing Cell Lines

Immunization

In one embodiment of the instant invention, human antibodies are produced by immunizing a non-human animal comprising some or the entire human immunoglobulin locus with a c-Met antigen. In a preferred embodiment, the non-human animal is a XENOMOUSE™, which is an engineered mouse strain that comprises large fragments of the human immunoglobulin loci and is deficient in mouse antibody production. See, e.g. Green et al. Nature Genetics 7: 13-21(1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00/09560, published Feb. 24, 2000 and WO 00/037504, published Jun. 29, 2000. The XENOMOUSE™ produces an adult-like human repertoire of fully human antibodies, and generates antigen specific human Mabs. A second generation XENOMOUSE™ contains approximately 80% of the human antibody repertoire through introduction of megabase sized, germline configuration YAC fragments of the human heavy chain loci and κ light chain loci. See Mendez et al. Nature Genetics 15:146-156 (1997), Green and Jakobovits J. Exp. Med. 188:483-495 (1998), the disclosures of which are hereby incorporated by reference.

The invention also provides a method for making c-Met antibodies from non-human, non-mouse animals by immunizing non-human transgenic animals that comprise human immunoglobulin loci. One may produce such animals using the methods described immediately above. The methods disclosed in these patents may be modified as described in U.S. Pat. No. 5,994,619. In a preferred embodiment, the non-human animals may be rats, sheep, pigs, goats, cattle, or horses. In another embodiment, the non-human animal comprising human immunoglobulin gene loci are animals that have a “minilocus” of human immunoglobulins. In the minilocus approach, an exogenous Ig locus is mimicked through the inclusion of individual genes from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described, inter alia, in U.S. Pat. Nos. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

An advantage of the minilocus approach is the rapidity with which constructs including portions of the Ig locus can be generated and introduced into animals. However, a potential disadvantage of the minilocus approach is that there may not be sufficient immunoglobulin diversity to support full B-cell development, such that there may be lower antibody production.

In order to produce a human c-Met antibody, a non-human animal comprising some or all of the human immunoglobulin loci is immunized with a c-Met antigen and the antibody or the antibody-producing cell is isolated from the animal. The c-Met antigen may be isolated and/or purified c-Met and is preferably a human c-Met. In another embodiment, the c-Met antigen is a fragment of c-Met, preferably the extracellular domain of c-Met. In another embodiment, the c-Met antigen is a fragment that comprises at least one epitope of c-Met. In another embodiment, the c-Met antigen is a cell that expresses c-Met on its cell surface, preferably a cell that overexpresses c-Met on its cell surface.

Immunization of animals may be done by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow, Lane supra, and U.S. Pat. No. 5,994,619. In a preferred embodiment, the c-Met antigen is administered with an adjuvant to stimulate the immune response.

Such adjuvants include complete or incomplete Freund's adjuvant, RIBI (muramyl dipeptides), or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

Production of Antibodies and Antibody-Producing Cell Lines

After immunization of an animal with a c-Met antigen, antibodies and/or antibody-producing cells may be obtained from the animal. A c-Met antibody-containing serum is obtained from the animal by bleeding or sacrificing the animal. The serum may be used as it is obtained from the animal, an immunoglobulin fraction may be obtained from the serum, or the c-Met antibodies may be purified from the serum. Serum or immunoglobulins obtained in this manner are polyclonal, which are disadvantageous because the amount of antibodies that can be obtained is limited and the polyclonal antibody has a heterogeneous array of properties. In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using c-Met, a portion thereof, or a cell expressing c-Met. In a preferred embodiment, the initial screening is performed using an enzyme-linked immunoassay (ELISA) or a radioimmunoassay (RIA), preferably an ELISA. An example of ELISA screening is provided in WO 00/37504, herein incorporated by reference.

In another embodiment, antibody-producing cells may be prepared from a human who has an autoimmune disorder and who expresses c-Met antibodies. Cells expressing the c-Met antibodies may be isolated by isolating white blood cells and subjecting them to fluorescence activated cell sorting (FACS) or by panning on plates coated with c-Met or a portion thereof. These cells may be fused with a human non-secretory myeloma to produce human hybridomas expressing human c-Met antibodies. In general, this is a less preferred embodiment because it is likely that the c-Met antibodies will have a low affinity for c-Met.

C-Met antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro.

Methods of selecting, cloning and expanding hybridomas are well known to those of ordinary skill in the art.

Preferably, the immunized animal is a non-human animal that expresses human immunoglobulin genes and the splenic B cells are fused to a myeloma derived from the same species as the non-human animal. More preferably, the immunized animal is a XENOMOUSE™ and the myeloma cell line is a non-secretory mouse myeloma, such as the myeloma cell line is NSO-bcl-2.

In one aspect, the invention provides hybridomas are produced that produce human c-Met antibodies. In a preferred embodiment, the hybridomas are mouse hybridomas, as described above. In another preferred embodiment, the hybridomas are produced in a non-human, non-mouse species such as rats, sheep, pigs, goats, cattle, or horses. In another embodiment, the hybridomas are human hybridomas, in which a human non-secretory myeloma is fused with a human cell expressing a c-Met antibody.

### Nucleic Acids, Vectors, Host Cells, and Recombinant Methods of Making Antibodies

Nucleic Acids

Nucleic acid molecules encoding c-Met antibodies of the invention are provided. In one embodiment, the nucleic acid molecule encodes a heavy and/or light chain of a c-Met immunoglobulin. In a preferred embodiment, a single nucleic acid molecule encodes a heavy chain of a c-Met immunoglobulin and another nucleic acid molecule encodes the light chain of a c-Met immunoglobulin. In a more preferred embodiment, the encoded immunoglobulin is a human immunoglobulin, preferably a human IgG. The encoded light chain may be a λ chain or a κ chain, preferably a λ chain.

The nucleic acid molecule encoding the variable region of the light chain may be derived from the A30, A27, or O12 Vκ gene. In another preferred embodiment, the nucleic acid molecule encoding the light chain comprises the joining region derived from Jκ1, Jκ2, or Jκ4. In an even more preferred embodiment, the nucleic acid molecule encoding the light chain contains no more than ten amino acid changes from the germline, preferably no more than six amino acid changes, and even more preferably no more than three amino acid changes.

The invention provides a nucleic acid molecule that encodes a variable region of the light chain (VL) containing at least three amino acid changes compared to the germline sequence, wherein the amino acid changes are identical to the amino acid changes from the germline sequence from the VL of one of the antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. The invention also provides a nucleic acid molecule comprising a nucleic acid sequence that encodes the amino acid sequence of the variable region of the light chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. The invention also provides a nucleic acid molecule comprising a nucleic acid sequence that encodes the amino acid sequence of one or more of the CDRs of any one of the light chains of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In a preferred embodiment, the nucleic acid molecule comprises a nucleic acid sequence that encodes the amino acid sequence of all of the CDRs of any one of the light chains of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another embodiment, the nucleic acid molecule comprises a nucleic acid sequence that encodes the VL amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60 or comprises a nucleic acid sequence of one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112,SEQ ID NO:113,SEQ ID NO:114,SEQ ID NO:115,SEQ ID NO:116, SEQ ID NO:117, SEQ ID NO:118, SEQ ID NO:119, or SEQ ID NO:120 or a fragment thereof.

In another preferred embodiment, the nucleic acid molecule comprises a nucleic acid sequence that encodes the amino acid sequence of one or more of the CDRs of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60 or comprises a nucleic acid sequence of one or more of the CDRs of any one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, or SEQ ID NO:120. In a more preferred embodiment, the nucleic acid molecule comprises a nucleic acid sequence that encodes the amino acid sequence of all of the CDRs of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60 or comprises a nucleic acid sequence of all the CDRs of any one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO: 105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO: 119, or SEQ ID NO:120. The invention also provides a nucleic acid molecules that encodes an amino acid sequence of a VL that has an amino acid sequence that is at least 70%, 75%, 80%, 85%, 90%, 95%, 96%, 97%, 98% or 99% identical to a VL described above, particularly to a VL that comprises an amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60. The invention also provides a nucleic acid sequence that is at least 70%, 75%, 80%, 85%, 90%, 95%, 96%, 97%, 98% or 99% identical to a nucleic acid sequence of one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ BD NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, or SEQ ID NO:120 or a fragment thereof. In another embodiment, the invention provides a nucleic acid molecule encoding a VL that hybridizes under highly stringent conditions to a nucleic acid molecule encoding a VL as described above, particularly a nucleic acid molecule that comprises a nucleic acid sequence encoding a VL amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. The invention also provides a nucleic acid sequence encoding an VL that hybridizes under highly stringent conditions to a nucleic acid molecule comprising a nucleic acid sequence of one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:10, SEQ ID NO:102, SEQ ID NO: 103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120 or a nucleic acid sequence that would hybridize except for the degeneracy of the genetic code.

The invention also provides a nucleic acid molecule encoding the variable region of the heavy chain (VH) is derived from the DP-35, DP-47, DP-71, or VIV-4/4.35 VH gene. In another embodiment, the nucleic acid molecule encoding the VH comprises the joining region derived from JH6 or JH5. In another preferred embodiment, the D segment is derived from 3-3, 6-19 or 4-17. In an even more preferred embodiment, the nucleic acid molecule encoding the VH contains no more than ten amino acid changes from the germline gene, preferably no more than six amino acid changes, and even more preferably no more than three amino acid changes. In a highly preferred embodiment, the nucleic acid molecule encoding the VH contains at least one amino acid change compared to the germline sequence, wherein the amino acid change is identical to the amino acid change from the germline sequence from the heavy chain of one of the antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In an even more preferred embodiment, the VH contains at least three amino acid changes compared to the germline sequences, wherein the changes are identical to those changes from the germline sequence from the VH of one of the antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1.

In one embodiment, the nucleic acid molecule comprises a nucleic acid sequence that encodes the amino acid sequence of the VH of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1 or a fragment of any one thereof. In a preferred embodiment, the nucleic acid molecule comprises a nucleic acid sequence that encodes the amino acid sequence of PGIA-01-A8, PGIA-03-A9, PGIA-03-A11, PGIA-03-B2, PGIA-04-A5, PGIA-04-A8, and PGIA-05-A1 or a fragment of any one thereof. In a preferred embodiment, the nucleic acid molecule comprises a nucleic acid sequence that encodes the amino acid sequence of PGIA-03-A9, PGIA-04-A5, and PGIA-04-A8 or a fragment of any one thereof. Table 1 shows the amino acid sequences of the scFvs PGIA-01-A1 through PGIA-05-A1 above.

In another embodiment, the nucleic acid molecule comprises a nucleic acid sequence that encodes the amino acid sequence of one or more of the CDRs of the heavy chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In a preferred embodiment, the nucleic acid molecule comprises a nucleic acid sequence that encodes the amino acid sequences of all of the CDRs of the heavy chain of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, or PGIA-05-A1. In another preferred embodiment, the nucleic acid molecule comprises a nucleic acid sequence that encodes the VH amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO: 18, SEQ ID NO:19, SEQ ID NO:20, SEQ ID NO:2 1, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60 or that comprises a nucleic acid sequence of one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, or SEQ ID NO:120. In another preferred embodiment, the nucleic acid molecule comprises a nucleic acid sequence that encodes the amino acid sequence of one or more of the CDRs of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60 or comprises a nucleic acid sequence of one or more of the CDRs of any one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ED NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120. In a preferred embodiment, the nucleic acid molecule comprises a nucleic acid sequence that encodes the amino acid sequences of all of the CDRs of any one SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60 or comprises a nucleic acid sequence of all of the CDRs of any one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120.

In another embodiment, the nucleic acid molecule encodes an amino acid sequence of a VH that is at least 70%, 75%, 80%, 85%, 90%, 95%, 96%, 97%, 98% or 99% identical to one of the amino acid sequences encoding a VH as described immediately above, particularly to a VH that comprises an amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60. The invention also provides a nucleic acid sequence that is at least 70%, 75%, 80%, 85%, 90%, 95%, 96%, 97%, 98% or 99% identical to a nucleic acid sequence of one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, or SEQ ID NO:120. In another embodiment, the nucleic acid molecule encoding a VH is one that hybridizes under highly stringent conditions to a nucleic acid sequence encoding a VH as described above, particularly to a VH that comprises an amino acid sequence of one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60. The invention also provides a nucleic acid sequence encoding a VH that hybridizes under highly stringent conditions to a nucleic acid molecule comprising a nucleic acid sequence of one of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120 or a nucleic acid sequence that would hybridize except for the degeneracy of the genetic code.

The nucleic acid molecule encoding either or both of the entire heavy and light chains of an c-Met antibody or the variable regions thereof may be obtained from any source that produces an c-Met antibody. Methods of isolating mRNA encoding an antibody are well known in the art. See, e.g., Sambrook et al. The mRNA may be used to produce cDNA for use in the polymerase chain reaction (PCR) or cDNA cloning of antibody genes. In one embodiment of the invention, the nucleic acid molecules may be obtained from a hybridoma that expresses an c-Met antibody, as described above, preferably a hybridoma that has as one of its fusion partners a transgenic animal cell that expresses human immunoglobulin genes, such as a XENOMOUSE™, non-human mouse transgenic animal or a nonhuman, non-mouse transgenic animal. In another embodiment, the hybridoma is derived from a non-human, non-transgenic animal, which may be used, e.g., for humanized antibodies.

A nucleic acid molecule encoding the entire heavy chain of a c-Met antibody may be constructed by fusing a nucleic acid molecule encoding the variable domain of a heavy chain or an antigen-binding domain thereof with a constant domain of a heavy chain. Similarly, a nucleic acid molecule encoding the light chain of a c-Met antibody may be constructed by fusing a nucleic acid molecule encoding the variable domain of a light chain or an antigen-binding domain thereof with a constant domain of a light chain. The nucleic acid molecules encoding the VH and VL chain may be converted to full-length antibody genes by inserting them into expression vectors already encoding heavy chain constant and light chain constant regions, respectively, such that the VH segment is operatively linked to the heavy chain constant region (CH) segment(s) within the vector and the VL segment is operatively linked to the light chain constant region (CL) segment within the vector.

Alternatively, the nucleic acid molecules encoding the VH or VL chains are converted into full-length antibody genes by linking, e.g., ligating the nucleic acid molecule encoding a VH chain to a nucleic acid molecule encoding a CH chain using standard molecular biological techniques. The same may be achieved using nucleic acid molecules encoding VL and CL chains. The sequences of human heavy and light chain constant region genes are known in the art. See, e.g., Kabat et al., Sequences of Proteins of Immunological Interest, 5th Ed., NIH Publ. No. 91-3242, 1991. Nucleic acid molecules encoding the full-length heavy and/or light chains may then be expressed from a cell into which they have been introduced and the c-Met antibody isolated.

In a preferred embodiment, the nucleic acid encoding the variable region of the heavy chain encodes the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60, and the nucleic acid molecule encoding the variable region of the light chains encodes the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60.

In another embodiment, a nucleic acid molecule encoding either the heavy chain of an c-Met antibody or an antigen-binding domain thereof, or the light chain of an c-Met antibody or an antigen-binding domain thereof may be isolated from a non-human, non-mouse animal that expresses human immunoglobulin genes and has been immunized with an c-Met antigen. In other embodiment, the nucleic acid molecule may be isolated from a c-Met antibody-producing cell derived from a non-transgenic animal or from a human patient who produces c-Met antibodies. Methods of isolating mRNA from the c-Met antibody producing cells may be isolated by standard techniques, cloned and/or amplified using PCR and library construction techniques, and screened using standard protocols to obtain nucleic acid molecules encoding c-Met heavy and light chains.

The nucleic acid molecules may be used to recombinantly express large quantities of c-Met antibodies, as described below. The nucleic acid molecules may also be used to produce chimeric antibodies, single chain antibodies, immunoadhesins, diabodies, mutated antibodies and antibody derivatives, as described further below. If the nucleic acid molecules are derived from a non-human, non-transgenic animal, the nucleic acid molecules may be used for antibody humanization, also as described below.

In another embodiment, the nucleic acid molecules of the invention may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of c-Met antibodies. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

Vectors

The invention provides vectors comprising the nucleic acid molecules of the invention that encode the heavy chain or the antigen-binding portion thereof. The invention also provides vectors comprising the nucleic acid molecules of the invention that encode the light chain or antigen-binding portion thereof. The invention also provides vectors comprising nucleic acid molecules encoding fusion proteins, modified antibodies, antibody fragments, and probes thereof.

To express the antibodies, or antibody portions of the invention, DNAs encoding partial or full-length light and heavy chains, obtained as described above, are inserted into expression vectors such that the genes are operatively linked to transcriptional and translational control sequences. Expression vectors include plasmids, retroviruses, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present). A convenient vector is one that encodes a functionally complete human CH or CL immunoglobulin sequence, with appropriate restriction sites engineered so that any VH or VL sequence can be easily inserted and expressed, as described above.

In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding 10 regions. The recombinant expression vector can also encode a signal peptide that facilitates secretion of the antibody chain from a host cell. The antibody chain gene may be cloned into the vector such that the signal peptide is linked inframe to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399 216, 4,634,665, and 5,179,017, all by Axel et al.). For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

Non-Hybridoma Host Cells and Methods of Recombinantly Producing Protein

Nucleic acid molecules encoding the heavy chain or an antigen binding portion thereof and/or the light chain or an antigen-binding portion thereof of a c-Met antibody, and vectors comprising these nucleic acid molecules, can be used for transformation of a suitable mammalian host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, and encapsulation of the polynucleotide(s) in liposomes, biolistic injection, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (which patents are hereby incorporated herein by reference).

Mammalian cell lines available as hosts for expression are well known in the art and include many immortalized cell lines available from the American Type Culture Collection (ATCC). These include, inter aria, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and a number of other cell lines. Mammalian host cells include human, mouse, rat, dog, monkey, pig, goat, bovine, horse, and hamster cells. Cell lines of particular preference are selected through determining which cell lines have high expression levels. Other cell lines that may be used are insect cell lines, such as Sf9 cells, amphibian cells, bacterial cells, plant cells, and fungal cells. When recombinant expression vectors encoding the heavy chain or antigen-binding portion thereof, the light chain and/or antigen-binding portion thereof are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

Transgenic Animals

The invention also provides transgenic non-human animals comprising one or more nucleic acid molecules of the invention that may be used to produce antibodies of the invention. Antibodies can be produced in and recovered from tissue or bodily fluids, such as milk, blood or urine, of goats, cows, horses, pigs, rats, mice, rabbits, hamsters or other mammals. See, e.g., U.S. Pat. Nos. 5,827,690, 5,756,687, 5,750,172, and 5,741,957. As described above, non-human transgenic animals that comprise human immunoglobulin loci can be produced by immunizing with c-Met or a portion thereof.

In another embodiment, non-human transgenic animals are produced by introducing one or more nucleic acid molecules of the invention into the animal by standard transgenic techniques. See Hogan, sierra. The transgenic cells used for making the transgenic animal can be embryonic stem cells or somatic cells. The transgenic non-human organisms can be chimeric, non-chimeric heterozygotes, and non-chimeric homozygotes. See, e.g., Hogan et al., Manipulating the Mouse Embryo: A Laboratory Manual 2 ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999). In another embodiment, the transgenic non-human organisms may have a targeted disruption and replacement that encodes a heavy chain and/or a light chain of interest. In a preferred embodiment, the transgenic animals comprise and express nucleic acid molecules encoding heavy and light chains that bind specifically to c-Met, preferably human c-Met. In another embodiment, the transgenic animals comprise nucleic acid molecules encoding a modified antibody such as a single-chain antibody, a chimeric antibody or a humanized antibody. The c-Met antibodies may be made in any transgenic animal. In a preferred embodiment, the nonhuman animals are mice, rats, sheep, pigs, goats, cattle, or horses. The non-human transgenic animal expresses said encoded polypeptides in blood, milk, urine, saliva, tears, mucus, and other bodily fluids.

Phage Display Libraries

The invention provides a method for producing an c-Met antibody or antigen-binding portion thereof comprising the steps of synthesizing a library of human antibodies on phage, screening the library with a c-Met or a portion thereof, isolating phage that bind c-Met, and obtaining the antibody from the phage. One method to prepare the library of antibodies comprises the steps of immunizing a non-human host animal comprising a human immunoglobulin locus with c-Met or an antigenic portion thereof to create an immune response, extracting cells from the host animal the cells that are responsible for production of antibodies; isolating RNA from the extracted cells, reverse transcribing the RNA to produce cDNA, amplifying the cDNA using a primer, and inserting the cDNA into phage display vector such that antibodies are expressed on the phage. Recombinant c-Met antibodies of the invention may be obtained in this way.

Recombinant c-Met human antibodies of the invention in addition to the c-Met antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurZAP™ phage display kit, catalog no. 240612). There are also other methods and reagents that can be used in generating and screening antibody display libraries (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibody. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9: 1373-1377; Hoogenboom et al. (1991) Nuc Acid Res 19:4133-4137; and Barbas et al. (1991) Proc. Natl. Acad. Sci. USA 88:7978-7982.

In a preferred embodiment, to isolate human c-Met antibodies with the desired characteristics, a human c-Met antibody as described herein is first used to select human heavy and light chain sequences having similar binding activity toward c-Met, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552554; and Griffiths et al., (1993) EMBO J 12:725-734. The scFv antibody libraries preferably are screened using human c-Met as the antigen.

Once initial human VL and VH segments are selected, “mix and match” experiments, in which different pairs of the initially selected VL and VH segments are screened for c-Met binding, are performed to select preferred VL/VH pair combinations. Additionally, to further improve the quality of the antibody, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to c-Met.

Following screening and isolation of a c-Met antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

Class Switching

Another aspect of the instant invention is to provide a mechanism by which the class of a c-Met antibody may be switched with another. In one aspect of the invention, a nucleic acid molecule encoding VL or VH is isolated using methods well known in the art such that it does not include any nucleic acid sequences encoding CL or CH. The nucleic acid molecule encoding VL or VH are then operatively linked to a nucleic acid sequence encoding a CL or CH from a different class of immunoglobulin molecule. This may be achieved using a vector or nucleic acid molecule that comprises a CL or CH chain, as described above. For example, a c-Met antibody that was originally IgM may be class switched to an IgG. Further, the class switching may be used to convert one IgG subclass to another, e.g., from IgG1 to IgG2. A preferred method for producing an antibody of the invention comprising a desired isotypes comprises the steps of isolating a nucleic acid encoding the heavy chain of an c-Met antibody and a nucleic acid encoding the light chain of an c-Met antibody, obtaining the variable region of the heavy chain, ligating the variable region of the heavy chain with the constant domain of a heavy chain of the desired isotype, expressing the light chain and the ligated heavy chain in a cell, and collecting the c-Met antibody with the desired isotype.

### Antibody Derivatives

One may use the nucleic acid molecules described above to generate antibody derivatives using techniques and methods known to one of ordinary skill in the art.

Humanized Antibodies

As was discussed above in connection with human antibody generation, there are advantages to producing antibodies with reduced immunogenicity. This can be accomplished to some extent using techniques of humanization and display techniques using appropriate libraries. It will be appreciated that marine antibodies or antibodies from other species can be humanized or primatized using techniques well known in the art. See e.g. Winter and Harris Immunol Today 14:43-46 (1993) and Wright et al. Crit. Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). In a preferred embodiment, the c-Met antibody can be humanized by substituting the CH1, CH2, CH3, hinge domains, and/or the framework domain with the corresponding human sequence while maintaining all of the CDRS of the heavy chain, the light chain or both the heavy and light chains.

Mutated Antibodies

In another embodiment, the nucleic acid molecules, vectors, and host cells may be used to make mutated c-Met antibodies. The antibodies may be mutated in the variable domains of the heavy and/or light chains to alter a binding property of the antibody. For example, a mutation may be made in one or more of the CDR regions to increase or decrease the K_{d} of the antibody for c-Met, to increase or decrease K_{off}, or to alter the binding specificity of the antibody. Techniques in site directed mutagenesis are well known in the art. See, e.g., Sambrook et al. and Ausubel et al., supra. In a preferred embodiment, mutations are made at an amino acid residue that is known to be changed compared to germline in a variable region of a c-Met antibody. In a more preferred embodiment, one or more mutations are made at an amino acid residue that is known to be changed compared to the germline in a variable region or CDR region of one of the c-Met antibodies PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another embodiment, one or more mutations are made at an amino acid residue that is known to be changed compared to the germline in a variable region or CDR region whose amino acid sequence is presented in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or whose nucleic acid sequence is presented in SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120.

In another embodiment, the nucleic acid molecules are mutated in one or more of the framework regions. A mutation may be made in a framework region or constant domain to increase the half-life of the c-Met antibody. See, e.g., WO 00/09560, published Feb. 24, 2000, herein incorporated by reference. In one embodiment, there may be one, three, or five point mutations and no more than ten point mutations. A mutation in a framework region or constant domain may also be made to alter the immunogenicity of the antibody, to provide a site for covalent or non-covalent binding to another molecule, or to alter such properties as complement fixation. Mutations may be made in each of the framework regions, the constant domain, and the variable regions in a single mutated antibody. Alternatively, mutations may be made in only one of the framework regions, the variable regions, or the constant domain in a single mutated antibody.

In one embodiment, there are no greater than ten amino acid changes in either the VH or VL regions of the mutated c-Met antibody compared to the c-Met antibody prior to mutation. In a more preferred embodiment, there are no more than five amino acid changes in either the VH or VL regions of the mutated c-Met antibody, more preferably no more than three amino acid changes. In another embodiment, there are no more than fifteen amino acid changes in the constant domains, more preferably, no more than ten amino acid changes, even more preferably, no more than five amino acid changes.

Modified Antibodies

In another embodiment, a fusion antibody or immunoadhesin may be made which comprises all or a portion of an anti- c-Met antibody linked to another polypeptide. In a preferred embodiment, only the variable regions of the c-Met antibody are linked to the polypeptide. In another preferred embodiment, the VH domain of an c-Met antibody are linked to a first polypeptide, while the VL domain of an c-Met antibody are linked to a second polypeptide that associates with the first polypeptide in a manner in which the VH and VL domains can interact with one another to form an antibody binding site. In another preferred embodiment, the VH domain is separated from the VL domain by a linker such that the VH and VL domains can interact with one another (see below under Single Chain Antibodies). The VH-linker-VL antibody is then linked to the polypeptide of interest. The fusion antibody is useful to directing a polypeptide to a c-Met expressing cell or tissue. The polypeptide may be a therapeutic agent, such as a toxin, growth factor, or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

To create a single chain antibody, (scFv) the VH- and VL-encoding DNA fragments are operatively linked to another fragment encoding a flexible linker, e.g., encoding the amino acid sequence (Gly₄-Ser)₃ (SEQ ID NO: 121), such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554). The single chain antibody may be monovalent, if only a single VH and VL are used, bivalent, if two VH and VL are used, or polyvalent, if more than two VH and VL are used.

In another embodiment, other modified antibodies may be prepared using c-Met-encoding nucleic acid molecules. For instance, “Kappa bodies” (Ill et al., Protein Eng 10: 949-57 (1997)), “Minibodies” (Martin et al., EMBO J 13: 5303 9 (1994)), “Diabodies” (Holliger et al., PNAS USA 90: 6444-6448 (1993)), or “Janusins” (Traunecker et al., EMBO J 10: 3655-3659 (1991) and Traunecker et al. “Janusin: new molecular design for bispecific reagents” Int J Cancer Suppl 7:51-52 (1992)) may be prepared using standard molecular biological techniques following the teachings of the specification.

In another aspect, chimeric and bispecific antibodies can be generated. A chimeric antibody may be made that comprises CDRs and framework regions from different antibodies. In a preferred embodiment, the CDRs of the chimeric antibody comprises all of the CDRs of the variable region of a light chain or heavy chain of an c-Met antibody, while the framework regions are derived from one or more different antibodies. In a more preferred embodiment, the CDRs of the chimeric antibody comprise all of the CDRs of the variable regions of the light chain and the heavy chain of a c-Met antibody. The framework regions may be from another species and may, in a preferred embodiment, be humanized. Alternatively, the framework regions may be from another human antibody.

A bispecific antibody can be generated that binds specifically to c-Met through one binding domain and to a second molecule through a second binding domain. The bispecific antibody can be produced through recombinant molecular biological techniques, or may be physically conjugated together. In addition, a single chain antibody containing more than one VH and VL may be generated that binds specifically to c-Met and to another molecule. Such bispecific antibodies can be generated using techniques that are well known for example, in connection with (i) and (ii) see e.g. Fanger et al. Immunol Methods 4: 72-81 (1994) and Wright and Harris, supra, and in connection with (iii) see e.g. Traunecker et al. Int. J. Cancer (Suppl.) 7: 51-52 (1992). In a preferred embodiment, the bispecific antibody binds to c-Met and to another molecule expressed at high level on cancer or tumor cells. In a more preferred embodiment, the other molecule is RON, IGF-1R, erbB2 receptor, VEGF-2 or 3, CD20, or EGF-R.

In another embodiment, the modified antibodies described above are prepared using one or more of the variable regions or one or more CDR regions from one of the antibodies selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1. In another embodiment, the modified antibodies are prepared using one or more of the variable regions or one or more CDR regions whose amino acid sequence is presented in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or whose nucleic acid sequence is presented in SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120.

Derivatized and Labeled Antibodies

An antibody or antibody portion of the invention can be derivatized or linked to another molecule (e.g., another peptide or protein). In general, the antibodies or portion thereof is derivatized such that the c-Met binding is not affected adversely by the derivatization or labeling. Accordingly, the antibodies and antibody portions of the invention are intended to include both intact and modified forms of the human c-Met antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antibody portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody may also be labeled with enzymes that are useful for detection, such as horseradish peroxidase, β-galactosidase, luciferase, alkaline phosphatase, glucose oxidase, and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a brown reaction product, which is detectable. An antibody may also be labeled with biotin, and detected through indirect measurement of avidin or streptavidin binding. An antibody may be labeled with a magnetic agent, such as gadolinium. An antibody may also be labeled with a predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

A c-Met antibody may also be labeled with a radiolabeled amino acid. The radiolabel may be used for both diagnostic and therapeutic purposes. For instance, the radiolabel may be used to detect c-Met-expressing tumors by x-ray or other diagnostic techniques. Further, the radiolabel may be used therapeutically as a toxin for cancerous cells or tumors. Examples of labels for polypeptides include, but are not limited to, the following radioisotopes or radionuclides—³H, ¹⁴C, ¹⁵N, ³⁵S, ⁹⁰Y ⁹⁹Tc ¹¹¹In, ¹²⁵I, and ¹³¹I.

A c-Met antibody may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

### Pharmaceutical Compositions and Kits

The invention also relates to a pharmaceutical composition for the treatment of a hyperproliferative disorder in a mammal, which comprises a therapeutically effective amount of a compound of the invention and a pharmaceutically acceptable carrier. In one embodiment, said pharmaceutical composition is for the treatment of cancer such as brain, lung, squamous cell, bladder, gastric, pancreatic, breast, head, neck, renal, kidney, ovarian, prostate, colorectal, esophageal, gynecological or thyroid cancer. In another embodiment, said pharmaceutical composition relates to non-cancerous hyperproliferative disorders such as, without limitation, restenosis after angioplasty and psoriasis. In another embodiment, the invention relates to pharmaceutical compositions for the treatment of a mammal that requires activation of c-Met, wherein the pharmaceutical composition comprises a therapeutically effective amount of an activating antibody of the invention and a pharmaceutically acceptable carrier. Pharmaceutical compositions comprising activating antibodies ma′ be used to treat animals that lack sufficient HGF, or may be used to treat osteoporosis, frailty or disorders in which the mammal secretes too little active growth hormone or is unable to respond to growth hormone. The c-Met antibodies of the invention can be incorporated into pharmaceutical compositions suitable for administration to a subject. Typically, the pharmaceutical composition comprises an antibody of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid, and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the c-Met antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts, and gelatin.

The antibodies of the present invention can be administered by a variety of methods known in the art, although for many therapeutic applications, the preferred route/mode of administration is intraperitoneal, subcutaneous, intramuscular, intravenous, or infusion. As will be appreciated by the skilled artisan, the route and/or mode of administration will vary depending upon the desired results. In one embodiment, the antibodies of the present inventor can be administered as a single dose or may be administered as multiple doses.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the c-Met of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, a c-Met antibody of the invention is coformulated with and/or coadministered with one or more additional therapeutic agents, such as a chemotherapeutic agent, an antineoplastic agent, or an anti-tumor agent. For example, a c-Met antibody may be coformulated and/or coadministered with one or more additional therapeutic agents. These agents include, without limitation, antibodies that bind other targets (e.g., antibodies that bind one or more growth factors or cytokines, their cell surface receptors or HGF), HGF binding proteins, antineoplastic agents, chemotherapeutic agents, antitumor agents, antisense oligonucleotides against c-Met or HGF, peptide analogues that block c-Met activation, soluble c-Met, and/or one or more chemical agents that inhibit HGF production or activity, which are known in the art, e.g., octreotide. For a pharmaceutical composition comprising an activating antibody, the c-Met antibody may be formulated with a factor that increases cell proliferation or prevents apoptosis. Such factors include growth factors such as HGF, and/or analogues of HGF that activate c-Met. Such combination therapies may require lower dosages of the c-Met antibody as well as the co-administered agents, thus avoiding possible toxicities or complications associated with the various monotherapies. In one embodiment, composition comprises the antibody and one or more additional therapeutic agent.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. Pharmaceutical composition comprising the antibody or comprising a combination therapy comprising the antibody and one or more additional therapeutic agents may be formulated for single or multiple doses. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals. A particularly useful formulation is 5 mg/ml c-Met antibody in a buffer of 20 mM sodium citrate, pH 5.5, 140 mM NaCl, and 0.2 mg/ml polysorbate 80.

An exemplary, non-limiting range for a therapeutically or prophylactically effective amount of an antibody or antibody portion of the invention is 0.1-100 mg/kg, more preferably 0.5-50 mg/kg, more preferably 1-20 mg/kg, and even more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. In one embodiment, the therapeutically or prophylactically effective amount of an antibody or antigen-binding portion thereof is administered along with one or more additional therapeutic agents.

Another aspect of the present invention provides kits comprising the c-Met antibodies and the pharmaceutical compositions comprising these antibodies. A kit may include, in addition to the antibody or pharmaceutical composition, diagnostic or therapeutic agents. A kit may also include instructions for use in a diagnostic or therapeutic method. In a preferred embodiment, the kit includes the antibody or a pharmaceutical composition thereof and a diagnostic agent that can be used in a method described below. In another preferred embodiment, the kit includes the antibody or a pharmaceutical composition thereof and one or more therapeutic agents, such as an additional antineoplastic agent, anti-tumor agent, or chemotherapeutic agent, which can be used in a method described below.

This invention also relates to pharmaceutical compositions for inhibiting abnormal cell growth in a mammal which comprise an amount of a compound of the invention in combination with an amount of a chemotherapeutic agent, wherein the amounts of the compound, salt, solvate, or prodrug, and of the chemotherapeutic agent are together effective in inhibiting abnormal cell growth. Many chemotherapeutic agents are presently known in the art. In one embodiment, the chemotherapeutic agents is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti angiogenesis agents.

Anti-angiogenic agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with a compound of the invention. Examples of useful COX-II inhibitors include CELEBREX™ (celecoxib), BEXTRA™ (valdecoxib), and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP inhibitors are those that do not demonstrate arthralgia. More preferred, are those that selectively inhibit MMP-2 And/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list:3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa bicyclo[3.2.1]octane-3-carboxylic acid hydroxyamide; (2R, 3R) 1-[4-(2-chloro-4 fluoro-benzyloxy)benzenesulfonyl]-3-hydroxy-3-methyl-piperidine-2-carboxylic acid hydroxyamide; 4-[4-(4-fluoro-phenoxy)-benzenesulfonylamino]-tetrahydro pyran-4-carboxylic acid hydroxyamide; 3-[[4-(4-fluoro-phenoxy)benzenesulfonyl] (1-hydroxycarbamoyl-cyclobutyl)-amino]-propionic acid; 4[4-(4-chloro-phenoxy) benzenesulfonylamino]-tetrahydro-pyran-4-carboxylic acid hydroxyamide; (R) 3-[4 (4-chloro-phenoxy)-benzenesulfonylamino]tetrahydro-pyran-3-carboxylic acid hydroxyamide; (2R, 3R) 1-[4-(4-fluoro-2-methyl-benzyloxy)-benzenesulfonyl]-3 hydroxy-3-methyl-piperidine-2-carboxylic acid hydroxyamide; 3-[[4-(4-fluoro phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro pyran-4-yl)-amino]-propionic acid; 3-exo-3-[4-(4-chloro-phenoxy)-benzenesulfonylamino]-8-oxa-icyclo[3.2.1]octane-3-carboxylic acid hydroxyamide; 3-endo-3-[4-(4-fluoro-phenoxy)-benzenesulfonylamino]-8-oxaicyclo[3.2.1]octane-3 carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of said compounds.

A compound of the invention can also be used with signal transduction inhibitors, such as agents that can inhibit EGF-R (epidermal growth factor receptor) responses, such as EGF-R antibodies, EGF antibodies, and molecules that are EGF-R inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc.). EGF-R inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated), ABX-EGF (Abgenix/Cell Genesys), EMD-7200 (Merck KgaA), EMD-5590 (Merck KgaA), MDX-447/H-477 (Medarex Inc. and Merck KgaA), and the compounds ZD 1834, ZD-1838 and ZD-1839 (AstraZeneca), PKI-166 (Novartis), PKI-166/CGP 75166 (Novartis), PTK 787 (Novartis), CP 701 (Cephalon), leflunomide (Pharmacia/Sugen), CI-1033 (Warner Lambert Parke Davis), CI-1033/PD 183,805 (Warner Lambert Parke Davis), CL-387,785 (Wyeth-Ayerst), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co.), VRCTC 310 (Ventech Research), EGF fusion toxin (Seragen Inc.), DAB-389 (Seragen/Ligand), ZM-252808 (Imperial Cancer Research Fund), RG-50864 (INSEAM), LFM-A12 (Parker Hughes Cancer Center), WHI-P97 (Parker Hughes Cancer Center), GW-282974 (Glaxo), KT-8391 (Kyowa Hakko) and EGF-R Vaccine (York Medical/Centro de Immunologia Molecular (CIM)). These and other EGF-R inhibiting agents can be used in the present invention.

VEGF inhibitors, for example SU-11248 (Sugen Inc.), SH-268 (Schering), and NX-1838 (NeXstar) can also be combined with the compound of the present invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc.); anti-VEGF monoclonal antibody of Genentech, Inc.; and angiozyme, a synthetic ribozyme from Ribozyme and Chiron. These and other VEGF inhibitors can be used in the present invention as described herein.

ErbB2 receptor inhibitors, such as GW-282974 (Glaxo Wellcome plc), and the monoclonal antibodies AR-209 (Aronex Pharmaceuticals Inc.) and 2B-I (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with the compound of the present invention in accordance with the present invention.

IGF-1 receptor inhibitors, such as the anti-IGF-1R antibodies of WO 02/053596 can be used in combination with the antibodies of the present invention.

Another component of the combination of the present invention is a cycloxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

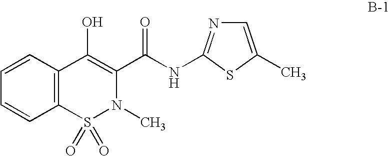

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

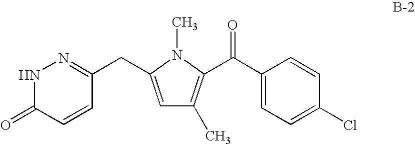

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes. Benzopyrans that can serve as a cyclooxygenase-2 selective inhibitor of the present invention include substituted benzopyran derivatives that are described in U.S. Pat. No. 6,271,253. Other benzopyran Cox-2 selective inhibitors useful in the practice of the present invention are described in U.S. Pat. Nos. 6,034,256 and 6,077,850.

In a further preferred embodiment of the invention the cyclooxygenase inhibitor can be selected from the class of tricyclic cyclooxygenase-2 selective inhibitors represented by the general structure of formula I:

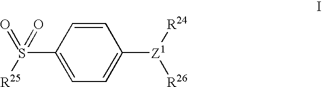

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a prodrug thereof.

In a preferred embodiment of the invention the cyclooxygenase-2 selective inhibitor represented by the above Formula I is selected from the group of compounds, illustrated in Table 3, which includes celecoxib (B-3), valdecoxib (B-4), deracoxib (B-5), rofecoxib (B-6), etoricoxib (MK-663; B-7), JTE-522 (B-8), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 3

| Compound | |
|---|---|
| Number | Structural Formula |
| B-3 | 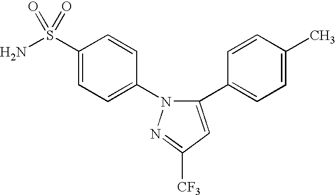 |
| B-4 | 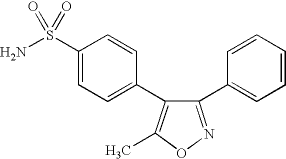 |
| B-5 | 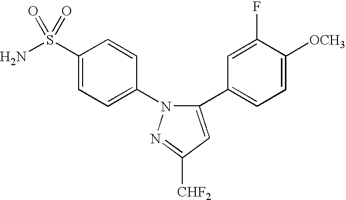 |
| B-6 | 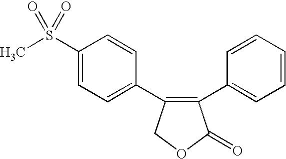 |
| B-7 | 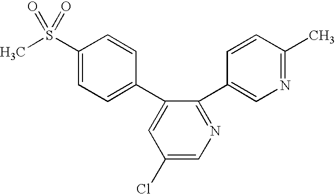 |
| B-8 | 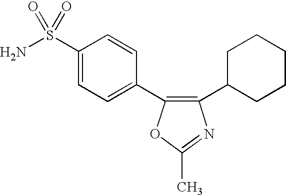 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-9, which is a therapeutically effective prodrug of the tricyclic cyclooxygenase-2 selective inhibitor valdecoxib, B-4, (See, e.g., U.S. Pat. No. 5,633,272), may be advantageously employed as a source of a cyclooxygenase inhibitor.

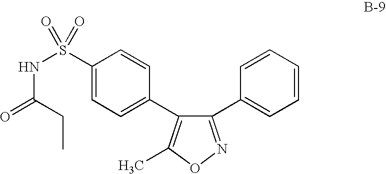

A preferred form of parecoxib is sodium parecoxib.

In another embodiment of the invention, the compound ABT-963 having the formula B-10 that has been previously described in International Publication number WO 00/24719, is another tricyclic cyclooxygenase-2 selective inhibitor which may be advantageously employed.

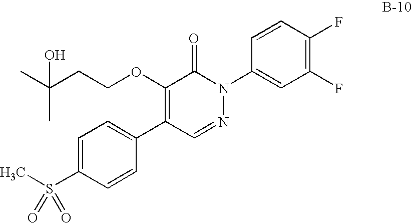

In a further embodiment of the invention, the cyclooxygenase inhibitor can be selected from the class of phenylacetic acid derivative cyclooxygenase-2 selective inhibitors described in WO 99/11605 WO 02/20090 is a compound that is referred to as COX-189 (also termed lumiracoxib), having CAS Reg. No. 220991-20-8.

Compounds that have a structure similar can serve as the Cox-2 selective inhibitor of the present invention, are described in U.S. Pat. Nos. 6,310,099, 6,291,523, and 5,958,978.

Further information on the applications of the Cox-2 selective inhibitor N-(2-cyclohexyloxynitrophenyl) methane sulfonamide (NS-398, CAS RN 123653-11-2), having a structure as shown in formula B-11, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (Jun. 06, 2001); and Iwata, K. et al., in Jpn. J. Pharmacol., 75(2):191-194 (1997).

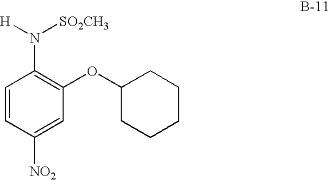

An evaluation of the anti-inflammatory activity of the cyclooxygenase-2 selective inhibitor, RWJ 63556, in a canine model of inflammation, was described by Kirchner et al., in J Pharmacol Exp Ther 282, 1094-1101 (1997).

Materials that can serve as the cyclooxygenase-2 selective inhibitor of the present invention include diarylmethylidenefuran derivatives that are described in U.S. Pat. No. 6,180,651.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

Compounds that may act as cyclooxygenase-2 selective inhibitors include multibinding compounds containing from 2 to 10 ligands covanlently attached to one or more linkers, as described in U.S. Pat. No. 6,395,724. Compounds that may act as cyclooxygenase-2 inhibitors include conjugated linoleic acid that is described in U.S. Pat. No. 6,077,868. Materials that can serve as a cyclooxygenase-2 selective inhibitor of the present invention include heterocyclic aromatic oxazole compounds that are described in U.S. Pat. Nos. 5,994,381 and 6,362,209. Cox-2 selective inhibitors that are useful in the subject method and compositions can include compounds that are described in U.S. Pat. Nos. 6,080,876 and 6,133,292. Materials that can serve as cyclooxygenase-2 selective inhibitors include pyridines that are described in U.S. Pat. Nos. 6, 369,275, 6,127,545, 6,130,334, 6,204,387, 6,071,936, 6,001,843 and 6,040,450. Materials that can serve as the cyclooxygenase-2 selective inhibitor of the present invention include diarylbenzopyran derivatives that are described in U.S. Pat. No. 6,340,694. Materials that can serve as the cyclooxygenase-2 selective inhibitor of the present invention include 1-(4-sulfamylaryl)-3-substituted-5-aryl-2-pyrazolines that are described in U.S. Pat. No. 6,376,519.

Materials that can serve as the cyclooxygenase-2 selective inhibitor of the present invention include heterocycles that are described in U.S. Pat. No. 6,153,787. Materials that can serve as the cyclooxygenase-2 selective inhibitor of the present invention include 2,3,5-trisubstituted pyridines that are described in U.S. Pat. No. 6,046,217. Materials that can serve as the cyclooxygenase-2 selective inhibitor of the present invention include diaryl bicyclic heterocycles that are described in U.S. Pat. No. 6,329,421. Compounds that may act as cyclooxygenase-2 inhibitors include salts of 5-amino or a substituted amino 1,2,3-triazole compound that are described in U.S. Pat. No. 6,239,137.

Materials that can serve as a cyclooxygenase-2 selective inhibitor of the present invention include pyrazole derivatives that are described in U.S. Pat. No. 6,136,831. Materials that can serve as a cyclooxygenase-2 selective inhibitor of the present invention include substituted derivatives of benzosulphonamides that are described in U.S. Pat. No. 6,297,282. Materials that can serve as a cyclooxygenase-2 selective inhibitor of the present invention include bicycliccarbonyl indole compounds that are described in U.S. Pat. No. 6,303,628. Materials that can serve as a cyclooxygenase-2 selective inhibitor of the present invention include benzimidazole compounds that are described in U.S. Pat. No. 6,310,079. Materials that can serve as a cyclooxygenase-2 selective inhibitor of the present invention include indole compounds that are described in U.S. Pat. No. 6,300,363. Materials that can serve as a cyclooxygenase-2 selective inhibitor of the present invention include aryl phenylhydrazides that are described in U.S. Pat. No. 6,077,869. Materials that can serve as a cyclooxygenase-2 selective inhibitor of the present invention include 2-aryloxy, 4-aryl furan-2-ones that are described in U.S. Pat. No. 6,140,515. Materials that can serve as a cyclooxygenase-2 selective inhibitor of the present invention include bisaryl compounds that are described in U.S. Pat. No. 5,994,379. Materials that can serve as a cyclooxygenase-2 selective inhibitor of the present invention include 1,5-diarylpyrazoles that are described in U.S. Pat. No. 6,028,202. Materials that can serve as a cyclooxygenase-2 selective inhibitor of the present invention include 2-substituted imidazoles that are described in U.S. Pat. No. 6,040,320. Materials that can serve as a cyclooxygenase-2 selective inhibitor of the present invention include 1,3- and 2,3-diarylcycloalkano and cycloalkeno pyrazoles that are described in U.S. Pat. No. 6,083,969. Materials that can serve as a cyclooxygenase-2 selective inhibitor of the present invention include esters derived from indolealkanols and novel amides derived from indolealkylamides that are described in U.S. Pat. No. 6,306,890. Materials that can serve as a cyclooxygenase-2 selective inhibitor of the present invention include pyridazinone compounds that are described in U.S. Pat. No. 6,307,047. Materials that can serve as a cyclooxygenase-2 selective inhibitor of the present invention include benzosulphonamide derivatives that are described in U.S. Pat. No. 6,004,948. Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

Anti-survival agents include c-Met antibodies and anti-integrin agents, such as anti-integrin antibodies.

### Diagnostic Methods of Use

The c-Met antibodies may be used to detect c-Met in a biological sample if in vitro or in vivo. The c-Met antibodies may be used in a conventional immunoassay, including, without limitation, an ELISA, an RIA, FACS, tissue immunohistochemistry, Western blot, or immunoprecipitation. The c-Met antibodies of the invention may be used to detect c-Met from humans. In another embodiment, the c-Met antibodies may be used to detect c-Met from Old World primates such as cynomolgus and rhesus monkeys, chimpanzees and apes.

The invention provides a method for detecting c-Met in a biological sample comprising contacting a biological sample with an c-Met antibody of the invention and detecting the bound antibody bound to c-Met, to detect the c-Met in the biological sample. In one embodiment, the c-Met antibody is directly labeled with a detectable label. In another embodiment, the c-Met antibody (the first antibody) is unlabeled and a second antibody or other molecule that can bind the c-Met antibody and is labeled. As is well known to one of skill in the art, a second antibody is chosen that is able to specifically bind the specific species and class of the first antibody. For example, if the c-Met antibody is a human IgG, then the secondary antibody may be an anti-human-IgG. Other molecules that can bind to many antibodies include, without limitation, Protein A and Protein G, both of which are available commercially, e.g., Amersham Pharmacia Biotech. Suitable labels for the antibody or secondary detection antibodies have been disclosed supra, and include various enzymes, prosthetic groups, fluorescent materials, luminescent materials, magnetic agents and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; an example of a magnetic agent includes gadolinium; and examples of suitable radioactive material include ¹²⁵I, ¹³¹I, ³⁵S or ³H.

In an alternative embodiment, c-Met can be assayed in a biological sample by a competition immunoassay utilizing c-Met standards labeled with a detectable substance and an unlabeled c-Met antibody. In this assay, the biological sample, the labeled c-Met standards, and the c-Met antibody are combined and the amount of labeled c-Met standard bound to the unlabeled antibody is determined. The amount of c-Met in the biological sample is inversely proportional to the amount of labeled c-Met standard bound to the c-Met antibody.

One may use the immunoassays disclosed above for a number of purposes. In one embodiment, the c-Met antibodies may be used to detect c-Met present in cells in cell culture. In a preferred embodiment, the c-Met antibodies may be used to determine the level of tyrosine phosphorylation, tyrosine autophosphorylation of c-Met, and/or the amount of c-Met on the cell surface after treatment of the cells with various compounds. This method can be used to test compounds that may be used to activate or inhibit c-Met, or result in a redistribution of c-Met on the cell surface or intracellularly. In this method, one sample of cells is treated with a test compound for a period of time while another sample is left untreated. If tyrosine autophosphorylation is to be measured, the cells are lysed and tyrosine phosphorylation of the c-Met is measured using an immunoassay described above or as described in Example III, which uses an ELISA. If the total level of c-Met is to be measured, the cells are lysed and the total c-Met level is measured using one of the immunoassays described above. The level of cell-surface c-Met may be determined using antibodies of the invention staining tissue culture cells following fixation of the cells. Standard practices of those skilled in the art allow fluorescence-activated cell sorting (FACS) to be used with a secondary detection antibody to determine the amount of binding of the primary (c-Met) antibody to the cell surface. Cells may also be permeabilized with detergents or toxins to allow the penetration of normally impermeant antibodies to now label intracellular sites where c-Met is localized.

A preferred immunoassay for determining c-Met tyrosine phosphorylation or for measuring total c-Met levels is an ELISA or Western blot. If only the cell surface level of c-Met is to be measured, the cells are not lysed, and the cell surface levels of c-Met are measured using one of the immunoassays described above (e.g., FACS). A preferred immunoassay for determining cell surface levels of c-Met includes the steps of labeling exclusively the cell surface proteins with a detectable label, such as biotin or ¹²⁵I, immunoprecipitating a detergent-soluble fraction of the cells containing integral membrane proteins with a c-Met antibody, and then detecting the fraction of total c-Met containing the detectable label. Another preferred immunoassay for determining the localization of c-Met, e.g., cell surface levels is by using immunofluorescence or immunohistochemistry. Methods such as ELISA, RIA, Western blot, immunohistochemistry, cell surface labeling of integral membrane proteins and immunoprecipitation are well known in the art. See, e.g., Harlow and Lane, supra. In addition, the immunoassays may be scaled up for high throughput screening in order to test a large number of compounds for either activation or inhibition of c-Met.

The c-Met antibodies of the invention may also be used to determine the levels of c-Met in a tissue or in cells derived from the tissue. In a preferred embodiment, the tissue is a diseased tissue. In a more preferred embodiment, the tissue is a tumor or a biopsy thereof. In a preferred embodiment of the method, a tissue or a biopsy thereof is excised from a patient. The tissue or biopsy is then used in an immunoassay to determine, e.g., c-Met levels, cell surface levels of c-Met, levels of tyrosine phosphorylation of c-Met, or localization of c-Met by the methods discussed above. The method can be used to determine if a tumor expresses c-Met at a high level.

The above-described diagnostic method can be used to determine whether a tumor expresses high levels of c-Met, which may he indicative that the tumor will respond well to treatment with c-Met antibody. The diagnostic method may also be used to determine whether a tumor is potentially cancerous, if it expresses high levels of c-Met, or benign, if it expresses low levels of c-Met. Further, the diagnostic method may also be used to determine whether treatment with c-Met antibody (see below) is causing a tumor to express lower levels of c-Met and/or to express lower levels of tyrosine autophosphorylation, and thus can be used to determine whether the treatment is successful. In general, a method to determine whether an c-Met antibody decreases tyrosine phosphorylation comprises the steps of measuring the level of tyrosine phosphorylation in a cell or tissue of interest, incubating the cell or tissue with an c-Met antibody or antigen-binding portion thereof, then re-measuring the level of tyrosine phosphorylation in the cell or tissue. The tyrosine phosphorylation of c-Met or of another protein(s) may be measured. The diagnostic method may also be used to determine whether a tissue or cell is not expressing high enough levels of c-Met or high enough levels of activated c-Met, which may be the case for individuals with dwarfism, osteoporosis, or diabetes. A diagnosis that levels of c-Met or active c-Met are too low could be used for treatment with activating c-Met antibodies, HGF or other therapeutic agents for increasing c-Met levels or activity.

The antibodies of the present invention may also be used in vivo to localize tissues and organs that express c-Met. In a preferred embodiment, the c-Met antibodies can be used to localize c-Met expressing tumors. The advantage of the c-Met antibodies of the present invention is that they will not generate an immune response upon administration. The method comprises the steps of administering an c-Met antibody or a pharmaceutical composition thereof to a patient in need of such a diagnostic test and subjecting the patient to imaging analysis determine the location of the c-Met expressing tissues. Imaging analysis is well known in the medical art, and includes, without limitation, x-ray analysis, magnetic resonance imaging (MRI), or computed tomography (CE). In another embodiment of the method, a biopsy is obtained from the patient to determine whether the tissue of interest expresses c-Met rather than subjecting the patient to imaging analysis. In a preferred embodiment, the c-Met antibodies may be labeled with a detectable agent that can be imaged in a patient. For example, the antibody may be labeled with a contrast agent, such as barium, which can be used for x-ray analysis, or a magnetic contrast agent, such as a gadolinium chelate, which can be used for MRI or CE. Other labeling agents include, without limitation, radioisotopes, such as ⁹⁹Tc. In another embodiment, the c-Met antibody will be unlabeled and will be imaged by administering a second antibody or other molecule that is detectable and that can bind the c-Met antibody.

### Therapeutic Methods of Use

In another embodiment, the invention provides a method for inhibiting c-Met activity by administering a c-Met antibody to a patient in need thereof. Any of the types of antibodies described herein may be used therapeutically. In a preferred embodiment, the c-Met antibody is a human, chimeric, or humanized antibody. In another preferred embodiment, the c-Met is human and the patient is a human patient. Alternatively, the patient may be a mammal that expresses a c-Met that the c-Met antibody cross-reacts with. The antibody may be administered to a nonhuman mammal expressing a c-Met with which the antibody cross-reacts (i. e. a primate, or a cynomolgus or rhesus monkey) for veterinary purposes or as an animal model of human disease. Such animal models may be useful for evaluating the therapeutic efficacy of antibodies of this invention.

As used herein, the term “a disorder in which c-Met activity is detrimental” is intended to include diseases and other disorders in which the presence of high levels of c-Met in a subject suffering from the disorder has been shown to be or is suspected of being either responsible for the pathophysiology of the disorder or a factor that contributes to a worsening of the disorder. Accordingly, a disorder in which high levels of c-Met activity is detrimental is a disorder in which inhibition of c-Met activity is expected to alleviate the symptoms and/or progression of the disorder. Such disorders may be evidenced, for example, by an increase in the levels of c-Met on the cell surface or in increased tyrosine autophosphorylation of c-Met in the affected cells or tissues of a subject suffering from the disorder. The increase in c-Met levels may be detected, for example, using a c-Met antibody as described above.

In a preferred embodiment, a c-Met antibody may be administered to a patient who has a c-Met-expressing tumor. A tumor may be a solid tumor or may be a non-solid tumor, such as a lymphoma. In a more preferred embodiment, an anti-IGF-antibody may be administered to a patient who has a c-Met-expressing tumor that is cancerous. In an even more preferred embodiment, the c-Met antibody is administered to a patient who has a tumor of the lung, breast, prostate, or colon. In a highly preferred embodiment, the method causes the tumor not to increase in weight or volume or to decrease in weight or volume. In another embodiment, the method causes the c-Met on the tumor to be internalized. In a preferred embodiment, the antibody is selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, or comprises a heavy chain, light chain or antigen-binding region thereof.

In another preferred embodiment, a c-Met antibody may be administered to a patient who expresses inappropriately high levels of HGF. It is known in the art that high level expression of HGF can lead to a variety of common cancers. In a more preferred embodiment, the c-Met antibody is administered to a patient with prostate cancer, glioma, or fibrosarcoma. In an even more preferred embodiment, the method causes the cancer to stop proliferating abnormally, or not to increase in weight or volume or to decrease in weight or volume.

In one embodiment, said method relates to the treatment of cancer such as brain, squamous cell, bladder, gastric, pancreatic, breast, head, neck, esophageal, prostate, colorectal, lung, renal, kidney, ovarian, gynecological or thyroid cancer. Patients that can be treated with a compounds of the invention according to the methods of this invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The antibody may be administered once, but more preferably is administered multiple times. The antibody may be administered from three times daily to once every six months. The administering may be on a schedule such as three times daily, twice daily, once daily, once every two days, once every three days, once weekly, once every two weeks, once every month, once every two months, once every three months and once every six months. The antibody may be administered via an oral, mucosal, buccal, intranasal, inhalable, intravenous, subcutaneous, intramuscular, parenteral, intratumor, or topical route. The antibody may be administered at a site distant from the site of the tumor. The antibody may also be administered continuously via a minipump. The antibody may be administered once, at least twice or for at least the period of time until the condition is treated, palliated, or cured. The antibody generally will be administered for as long as the tumor is present provided that the antibody causes the tumor or cancer to stop growing or to decrease in weight or volume. The antibody will generally be administered as part of a pharmaceutical composition as described supra. The dosage of antibody will generally be in the range of 0.1-100 mg/kg, more preferably 0.5-50 mg/kg, more preferably 1-20 mg/kg, and even more preferably 1-10 mg/kg. The serum concentration of the antibody may be measured by any method known in the art. The antibody may also be administered prophylactically in order to prevent a cancer or tumor from occurring. This may be especially useful in patients that have a “high normal” level of HGF because these patients have been shown to have a higher risk of developing common cancers. See Rosen et al., supra.

In another aspect, the c-Met antibody may be co-administered with other therapeutic agents, such as antineoplastic drugs or molecules, to a patient who has a hyperproliferative disorder, such as cancer or a tumor. In one aspect, the invention relates to a method for the treatment of the hyperproliferative disorder in a mammal comprising administering to said mammal a therapeutically effective amount of a compound of the invention in combination with an anti-tumor agent selected from the group consisting of, but not limited to, mitotic inhibitors, alkylating agents, anti-metabolites, intercalating agents, growth factor inhibitors, cell cycle inhibitors, enzymes, topoisomerase inhibitors, biological response modifiers, anti-hormones, kinase inhibitors, matrix metalloprotease inhibitors, genetic therapeutics and anti androgens. In a more preferred embodiment, the antibody may be administered with an antineoplastic agent, such as Adriamycin or taxol. In another preferred embodiment, the antibody or combination therapy is administered along with radiotherapy, chemotherapy, photodynamic therapy, surgery, or other immunotherapy. In yet another preferred embodiment, the antibody will be administered with another antibody. For example, the c-Met antibody may be administered with an antibody or other agent that is known to inhibit tumor or cancer cell proliferation, e.g., an antibody or agent that inhibits erbB2 receptor, EGF-R, CD20, or VEGF.

Co-administration of the antibody with an additional therapeutic agent (combination therapy) encompasses administering a pharmaceutical composition comprising the c-Met antibody and the additional therapeutic agent and administering two or more separate pharmaceutical compositions, one comprising the c-Met antibody and the other(s) comprising the additional therapeutic agent(s). Further, although co-administration or combination therapy generally means that the antibody and additional therapeutic agents are administered at the same time as one another, it also encompasses instances in which the antibody and additional therapeutic agents are administered at different times. For instance, the antibody may be administered once every three days, while the additional therapeutic agent is administered once daily. Alternatively, the antibody may be administered prior to or subsequent to treatment of the disorder with the additional therapeutic agent. Similarly, administration of the c-Met antibody may be administered prior to or subsequent to other therapy, such as radiotherapy, chemotherapy, photodynamic therapy, surgery, or other immunotherapy

The antibody and one or more additional therapeutic agents (the combination therapy) may be administered once, twice or at least the period of time until the condition is treated, palliated or cured. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months and once every six months, or may be administered continuously via a minipump. The combination therapy may be administered via an oral, mucosal, buccal, intranasal, inhalable, intravenous, subcutaneous, intramuscular, parenteral, intratumor or topical route. The combination therapy may be administered at a site distant from the site of the tumor. The combination therapy generally will be administered for as long as the tumor is present provided that the antibody causes the tumor or cancer to stop growing or to decrease in weight or volume.

In a still further embodiment, the c-Met antibody is labeled with a radiolabel, an immunotoxin, or a toxin, or is a fusion protein comprising a toxic peptide. The c-Met antibody or c-Met antibody fusion protein directs the radiolabel, immunotoxin, toxin, or toxic peptide to the c-Met-expressing tumor or cancer cell. In a preferred embodiment, the radiolabel, immunotoxin, toxin, or toxic peptide is internalized after the c-Met antibody binds to the c-Met on the surface of the tumor or cancer cell.

In another aspect, the c-Met antibody may be used therapeutically to induce apoptosis of specific cells in a patient in need thereof. In many cases, the cells targeted for apoptosis are cancerous or tumor cells. Thus, in a preferred embodiment, the invention provides a method of inducing apoptosis by administering a therapeutically effective amount of a c-Met antibody to a patient in need thereof. In a preferred embodiment, the antibody is selected from PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1, or comprises a heavy chain, light chain, or antigen-binding region thereof.

In another aspect, the c-Met antibody may be used to treat noncancerous states in which high levels of HGF and/or c-Met have been associated with the noncancerous state or disease. In one embodiment, the method comprises the step of administering a c-Met antibody to a patient who has a noncancerous pathological state caused or exacerbated by high levels of HGF and/or c-Met levels or activity. In a preferred embodiment, the noncancerous pathological state is psoriasis, atherosclerosis, smooth muscle restenosis of blood vessels or inappropriate microvascular proliferation, such as that found as a complication of diabetes, especially of the eye. In a more preferred embodiment, the c-Met antibody slows the progress of the noncancerous pathological state. In a more preferred embodiment, the c-Met antibody stops or reverses, at least in part, the noncancerous pathological state.

The antibodies of the present would also be useful in the treatment or prevention of ophthalmic diseases, for example glaucoma, retinitis, retinopathies (e.g., diabetic retinopathy), uveitis, ocular photophobia, macular degeneration (e.g., age related macular degeneration, wet-type macular degeneration, and dry-type macular degeneration) and of inflammation and pain associated with acute injury to the eye tissue. The compounds would be further useful in treatment or prevention of postsurgical ophthalmic pain and inflammation.

In another aspect, the invention provides a method of administering an activating c-Met antibody to a patient in need thereof. In one embodiment, the activating antibody or pharmaceutical composition is administered to a patient in need thereof in an amount effective to increase c-Met activity. In a more preferred embodiment, the activating antibody is able to restore normal c-Met activity. In another preferred embodiment, the activating antibody may be administered to a patient who has small stature, neuropathy, a decrease in muscle mass or osteoporosis. In another preferred embodiment, the activating antibody may be administered with one or more other factors that increase cell proliferation, prevent apoptosis, or increase c-Met activity. Such factors include growth factors such as HGF, and/or analogues of HGF that activate c-Met.

### Gene Therapy

The nucleic acid molecules of the instant invention may be administered to a patient in need thereof via gene therapy. The therapy may be either in vivo or ex viva. In a preferred embodiment, nucleic acid molecules encoding both a heavy chain and a light chain are administered to a patient. In a more preferred embodiment, the nucleic acid molecules are administered such that they are stably integrated into the chromosome of B cells because these cells are specialized for producing antibodies. In a preferred embodiment, precursor B cells are transfected or infected ex vivo and retransplanted into a patient in need thereof. In another embodiment, precursor B cells or other cells are infected in vivo using a virus known to infect the cell type of interest. Typical vectors used for gene therapy include liposomes, plasmids, or viral vectors, such as retroviruses, adenoviruses, and adeno associated viruses. After infection either in viva or ex vivo, levels of antibody expression may be monitored by taking a sample from the treated patient and using any immunoassay known in the art and discussed herein.

In a preferred embodiment, the gene therapy method comprises the steps of administering an effective amount of an isolated nucleic acid molecule encoding the heavy chain or the antigen-binding portion thereof of the human antibody or portion thereof and expressing the nucleic acid molecule. In another embodiment, the gene therapy method comprises the steps of administering an effective amount of an isolated nucleic acid molecule encoding the light chain or the antigen-binding portion thereof of the human antibody or portion thereof and expressing the nucleic acid molecule. In a more preferred method, the gene therapy method comprises the steps of administering an effective amount of an isolated nucleic acid molecule encoding the heavy chain or the antigen binding portion thereof of the human antibody or portion thereof and an effective amount of an isolated nucleic acid molecule encoding the light chain or the antigen-binding portion thereof of the human antibody or portion thereof and expressing the nucleic acid molecules. The gene therapy method may also comprise the step of administering another anti cancer agent, such as taxol, tamoxifen, 5-FU, Adriamycin or CP-358,774.

In order that this invention may be better understood, the following examples are set forth. These examples are for purposes of illustration only and are not to be construed as limiting the scope of the invention in any manner.

### EXAMPLES

### Example 1

### Selection of c-Met Binding ScFv's

An scFv phagemid library, which is an expanded version of the 1.38×10¹⁰ library described by Vaughan et al. (Nature Biotech. (1996) 14: 309-314) was used to select antibodies specific for human c-Met. Two selection methodologies were employed; panning selections and soluble selections.

For the panning method, soluble c-Met fusion protein (at 10 μg/ml in phosphate buffered saline (PBS)) or control fusion protein (at 10 μg/ml in PBS) was coated onto wells of a microtitre plate overnight at 4° C. Wells were washed in PBS and blocked for 1 hour at 37° C. in MPBS (3% milk powder in PBS). Purified phage (10¹² transducing units (tu)) was blocked for 1 hour in a final volume of 10 μl of 3% MPBS. Blocked phage was added to blocked control fusion protein wells and incubated for 1 hour. The blocked and deselected phage was then transferred to the blocked wells that were coated with the c-Met fusion protein and were incubated for an additional hour. Wells were washed 5 times with PBST (PBS containing 0.1% v/v Tween 20), then 5 times with PBS. Bound phage particles were eluted and used to infect 10 ml of exponentially growing E. coli TG1. Infected cells were grown in 2TY broth for 1 hour at 37° C., then spread onto 2TYAG plates and incubated overnight at 30° C. Colonies were scraped off the plates into 10 ml 2TY broth and 15% glycerol added for storage at −70° C.

Glycerol stock cultures from the first round panning selection were superinfected with helper phage and rescued to give scFv antibody-expressing phage particles for the second round of panning. A total of three rounds of panning were carried out in this way for isolation of antibody-expressing phage particles specific for human c-Met.

For the soluble selection method, biotinylated human c-Met fusion protein at a final concentration of 50 nM was used with scFv phagemid library, as described above. Purified scFv phage (10¹² tu) in 1 ml 3% MPBS were blocked for 30 minutes, then biotinylated antigen was added and incubated at room temperature for 1 hour. Phage/antigen was added to 50 μl of Dynal M280 Streptavidin magnetic beads that had been blocked for 1 hour at 37° C. in 1 ml of 3% MPBS and incubated for a further 15 minutes at room temperature. Beads were captured using a magnetic rack and washed 5× in 1 ml of 3% MPBS/0.1% (v/v) Tween 20 followed by 2 washes in PBS. After the last PBS wash, beads were resuspended in 100 μl PBS and used to infect 5 ml of exponentially growing E. coli TG-1 cells. Infected cells were incubated for 1 hour at 37° C. (30 minutes stationary, 30 minutes shaking at 250 rpm), then spread on 2TYAG plates and incubated overnight at 30° C. Output colonies were scraped off the plates and phage rescued as described above. Two further rounds of soluble selection were performed as described above.

The nomenclature used to refer to the single-chain (scFv) antibodies was “PGIA” followed by the microtiter plate number and well number. For Example the c-Met scFv antibody from plate 1, well A1 was designated “PGIA-01-A1”.

### Example 2

### c-Met Protein Expression and Purification

Conversion to IgG

Clones were converted into the IgG format as described below. Reformatting involves the subcloning of the VH domain from the scFv into a vector containing the human heavy chain constant domains, and regulatory elements for the appropriate expression in mammalian cells. Similarly, the VL domain is subcloned into an expression vector containing the human light chain constant domain (lambda or kappa class) along with the appropriate regulatory elements

The nucleic acid sequence encoding the appropriate domain from the scFv clone was amplified, followed by restriction enzyme digestion and ligation into the appropriate expression vector. Heavy Chain (IgG1 constant domain) were cloned into pEU1, Light Chain (lambda class) were cloned into pEU4, and Light Chain (kappa class) were cloned into pEU3 (Persic, L. et al., Gene 187:9-18 (1997))

Site Directed Mutagenesis

Prior to reformatting, it was observed that several scFvs (including PGIA-03-A11) contained an internal BstEII restriction site within the VH domain that would interfere with cloning of the VH into the IgG1 heavy chain vector. The internal restriction site was removed by Quikchange™ (Invitrogen)site-directed mutagenesis using the method as described in the kit. Oligos MUTF QFRVTM (CAGGGCAGGGTCACAATGGCCAG SEQ ID NO:121) and MUTR QFRVTM (CTGGCCATTGTGACCCTGCCCTG SEQ ID NO:122) were designed to remove the restriction site but maintaining the same amino acid sequence. Sequencing was carried out to ensure that the site had been mutated correctly.

VH/VL Cloning PCR

Once all sequences were checked for the absence of restriction sites, the nucleic acid sequence encoding the VH and VL domains were amplified in separate PCR reactions.

100 ul PCR reactions were set up for each VH and VL domain using 50 ul 2×PCR master mix, 5 ul forward primer (@ 10 uM), 5 ul reverse primer (@ 10 uM), and 40 ul water. Primers were allocated according to the scFv sequence, and are shown in Table 4

TABLE 4

| | | VH | VH | VL | VL |
|---|---|---|---|---|---|
| IgG | | Forward | reverse | forward | reverse |
| Clone | scFv Clone | primer | primer | primer | primer |
| 11978 | PGIA-1-A8 | AF14 | H-Link | AF42 | AF23 |
| 11994 | PGIA-3-A9 | AF11 | H-Link | AF42 | AF23 |
| 12075 | PGIA-3-A11 | AF18 | H-Link | AF31 | AF28 |
| 12119 | PGIA-5-A1 | RH55 | H-Link | AF42 | AF23 |
| 12123 | PGIA-3-B2 | AF11 | H-Link | AF21 | RH62 |
| 12133 | PGIA-4-A5 | AF11 | H-Link | AF42 | AF47 |
| 12136 | PGIA-4-A8 | AF11 | H-Link | AF40 | AF29 |

A single bacterial colony containing the appropriate nucleic acid encoding the scFv in pCANTAB6 (WO 94/13804, FIGS. 19 and 20) was picked into each PCR reaction and the sample was amplified using the following parameters: 94° C. for 5 minutes, 94° C. for 1 min., 30 cycles of 55° C. for 1 min. and 72° C. 1 min., and 72° C. 5 min.

Digestion

The PCR products were cleaned up using a QIAquick™ 8-well purification kit (Catalog # 28144, Qiagen, Valencia Calif.) according to the manufacturer's directions. A 25 ul aliquot of the amplified VH PCR products was digested with BssHII and BstEII. A 25 ul aliquot of the amplified VL PCR products was digested with ApaLI and PacI.

The digested VH and VL PCR products were cleaned up using a QIAquick purification kit.

Ligation and Transformation

An aliquot of the cleaned up, digested PCR product was ligated into the appropriate vector digested with the same restriction enzymes. VH domains were ligated into pMON27816 (pEU1), and VL domains were ligated into either pMON27820 (pEU3) or pMON27819 (pEU4), depending on light chain class (Persic et al., Gene 187: 9-18, 1997). A portion of each of the ligation reactions was transformed into previously prepared chemically competent DH5α E. coli by heat shock and grown overnight on 2×TY agar plates containing Ampicillin.

Screening

Individual ampicillin resistant colonies were picked into liquid 2TY media (containing Ampicillin) in a 96-well plate and grown overnight. Once cultured, the colonies were screened by PCR to determine whether the vectors contained the appropriate domains. VH-containing plasmids were screened using the primers, PECSEQ1 and p95, and VL-containing plasmids were screened using the primers, PECSEQ1 and p156.

Colonies containing inserts were analyzed by DNA sequencing using the same primers as used for the screening PCR.

Table 5 shows the oligonucleotide primers used to amplify the VH and VL domains.

TABLE 5

| | | |
|---|---|---|
| Oligo Name | Oligo Sequence (5′-3′) | Function of Oligo |

| | | | |
|---|---|---|---|
| AF11 | CTCTCCACAGGCGCGCACTCCCAGGTGCAGCTG | SEQ ID NO: 123 | VH forward PCR cloning |
| | CAGGAG | | primer |
| AF14 | CTCTCCACAGGCGCGCACTCCGAGGTGCAGCTG | SEQ ID NO: 124 | VH forward PCR cloning |
| | TTGGAG | | primer |
| AF18 | CTCTCCACAGGCGCGCACTCCCAGGT(GC)CAG | SEQ ID NO: 125 | VH forward PCR cloning |
| | CTGGTGCA | | primer |
| RH55 | CTCTCCACAGGCGCGCACTCCCAGCTGCAGCTG | SEQ ID NO: 126 | VH forward PCR cloning |
| | CAGGAGTCGGGC | | primer |
| HLINK | ACCGCCAGAGCCACCTCCGCC | SEQ ID NO: 127 | VH reverse PCR cloning |
| | | | primer |
| AF21 | CTCCACAGGCGTGCACTCCCAGGCTGTGCTGAC | SEQ ID NO: 128 | VL forward PCR cloning |
| | TCAGCC | | primer |
| AF31 | CTCTCCACAGGCGTGCACTCCCAGTCTGTGCTG | SEQ ID NO: 129 | VL forward PCR cloning |
| | ACTCAGCC | | primer |
| AF40 | CCACAGGCGTGCACTCCTCCTATGAGCTGACTC | SEQ ID NO: 130 | VL forward PCR cloning |
| | AG | | primer |
| AF42 | CTCCACAGGCGTGCACTCCAATTTTATGCTGAC | SEQ ID NO: 131 | VL forward PCR cloning |
| | TCAG | | primer |
| AF23 | CTATTCCTTAATTAAGTTAGATCTATTCTGACT | SEQ ID NO: 132 | VL reverse PCR cloning |
| | CACCTAGGACGGTCAGCTTGGTCCCTC | | primer |
| AF47 | CTATTCCTTAATTAAGTTAGATCTATTCTGACT | SEQ ID NO: 133 | VL reverse PCR cloning |
| | CACCTAGGACGGTGACCTTGGTCCC | | primer |
| AF28 | CTATTCCTTAATTAAGTTAGATCTATTCTGACT | SEQ ID NO: 134 | VL reverse POR cloning |
| | CACCTAGGACGGTCAGCTTGGTCCCACT | | primer |
| AF29 | CTATTCCTTAATTAAGTTAGATCTATTCTGACT | SEQ ID NO: 135 | VL reverse PCR cloning |
| | CACCTAGGACGGTGACCTTGGTCCCAGT | | primer |
| RH62 | CTATTCCTTAATTAAGTTAGATCTATTCTGACT | SEQ ID NO: 136 | VL reverse PCR cloning |
| | CACCTAGGACGGTGAGCTGGGTCCC | | primer |
| PECSEQ1 | GCAGGCTTGAGGTCTGGAC | SEQ ID NO: 137 | VH/VL forward screening |
| | | | Primer |
| P156 | TAATTATAGCAAGGAGACCAAGAAG | SEQ ID NO: 138 | VL reverse screening primer |
| P95 | CAGAGGTGCTCTTGGAGGAGGGTGC | SEQ ID NO: 139 | VH reverse screening primer |

After the scFvs were converted to IgGs or Fabs a different naming convention was used. Table 6 shows the correlation between the scFv nomenclature and the corresponding IgG or Fab nomenclature. For example scFv “PGIA-01-A2” was converted to an IgG designated “12118 IgG” and the Fab designated “12118 Fab”.

TABLE 6

| scFv | IgG and |
|---|---|
| Clone ID | Fab |
| PGIA-1-A1 | * |
| PGIA-1-A2 | 12118 |
| PGIA-1-A3 | 11987 |
| PGIA-1-A4 | * |
| PGIA-1-A5 | 12122 |
| PGIA-1-A6 | 12129 |
| PGIA-1-A7 | * |
| PGIA-1-A8 | 11978 |
| PGIA-1-A9 | 12126 |
| PGIA-1-A10 | * |
| PGIA-1-A11 | * |
| PGIA-1-A12 | * |
| PGIA-1-B1 | 11988 |
| PGIA--1-B2 | * |
| PGIA-2-A1 | 11989 |
| PGIA-2-A2 | 12068 |
| PGIA-2-A3 | 11990 |
| PGIA-2-A4 | 12069 |
| PGIA-2-A5 | 12070 |
| PGIA-2-A6 | 11979 |
| PGIA-2-A7 | 12071 |
| PGIA-2-A8 | 12072 |
| PGIA-2-A9 | 11980 |
| PGIA-2-A10 | 11981 |
| PGIA-2-A11 | 11991 |
| PGIA-2-A12 | 12073 |
| PGIA-2-B1 | 12074 |
| PGIA-3-A1 | 11982 |
| PGIA-3-A2 | 12130 |
| PGIA-3-A3 | 11983 |
| PGIA-3-A4 | 11984 |
| PGIA-3-A5 | 11992 |
| PGIA-3-A6 | 11985 |
| PGIA-3-A7 | 12127 |
| PGIA-3-A8 | 11993 |
| PGIA-3-A9 | 11994 |
| PGIA-3-A10 | 11995 |
| PGIA-3-A11 | 12075 |
| PGIA-3-A12 | 11997 |
| PGIA-3-B1 | 11986 |
| PGIA-3-B2 | 12123 |
| PGIA-3-B3 | 12076 |
| PGIA-3-B4 | 12077 |
| PGIA-3-B5 | 12128 |
| PGIA-3-B6♦ | 12078 |
| PGIA-3-B6♦ | 12124 |
| PGIA-3-B7♦ | 12079 |
| PGIA-3-B7♦ | 12125 |
| PGIA-3-B8 | 12080 |
| PGIA-4-A1 | 12131 |
| PGIA-4-A2 | * |
| PGIA-4-A3 | 12132 |
| PGIA-4-A4 | 12139 |
| PGIA-4-A5 | 12133 |
| PGIA-4-A6 | 12134 |
| PGIA-4-A7 | 12135 |
| PGIA-4-A8 | 12136 |
| PGIA-4-A9 | 12137 |
| PGIA-4-A10 | 12138 |
| PGIA-4-A11 | 12120 |
| PGIA-4-A12 | 12121 |
| PGIA-5-A1 | 12119 |
| PGIA-3-B4 | 12077 |

* = not converted to IgG and Fab
♦= two isolates selected

Expression of c-Met MAb

Expression of the functional heavy chain gene cassette was driven by the GV promoter and terminated by the SV40 poly adenylation signal. The GV promoter is a synthetic promoter comprised of five repeats of the yeast Gal4 upstream activation sequence plus a minimal CMV promoter (Carey, M. et al., Nature 345 (1990), 361-364). The vector also contained the dhfr expression cassette from pSV2dhfr. Chinese hamster ovary (CHO/GV) cells transformed to express a chimeric transactivator (GV) derived from the fusion of the yeast Gal4 DNA binding domain and the VP16 transactivation domain (Carey, M. et al., Nature 345 (1990), 361-364) were transfected simultaneously with heavy-chain and light chain expression vectors using Lipofectamine 2000 (Gibco) according to the manufacturers instructions. Cell were grown at 37° C., 5% CO₂ in IMDM (Invitrogen)+10% FBS (Invitrogen)+1×HT supplement (Invitrogen) for forty-eight hours after transfection and then the cells were placed under selection by removing hypoxanthine and thymidine from the media (IMDM+10% dialyzed FBS (Invitrogen)). After 10 days the pool of cells was cloned in 96-well plates and after 14 days in culture the 96-well plates were screened and the highest expressing clones were expanded. Expression was done in roller bottles by plating one confluent T75 flask into one 1700 cm² roller bottle containing 400 ml of IMDM+10% dialyzed FBS media.

Purification of c-Met MAb

Purification of c-Met immunoglobulins was accomplished by affinity chromatography utilizing 1 ml Amersham Fast Flow recombinant protein A columns. The columns were equilibrated with 20 mls of GIBCO PBS pH 7.4(#12388-013) at 1 ml per minute. Conditioned media containing anti c-Met IgG was 0.2 micron filtered then applied to the equilibrated column at 0.5 ml per minute. Unbound protein was washed from the column with 60 ml of PBS at 1 ml per minute. The IgG was eluted with 20 ml of 0.1 M glycine plus 0.15 M NaCl pH 2.8 at 1 ml per minute. The eluate was collected into 2 ml of 1 M Tris Cl pH 8.3 with stirring. Amicon Centriprep YM-30 filtration units were used to concentrate the eluates (22 ml) to approximately 1.5 ml. The concentrates were dialyzed in Pierce 10K MWCO Slide-A-lyzer cassettes versus 2×1 L of PBS. Following dialysis the IgG was passed through a 0.2 micron filter, aliquoted and stored frozen at −80 C. IgG was characterized by reducing and non-reducing SDS PAGE, size exclusion chromatography and quantitated by absorbance at 280 nm using a calculated extinction coefficient of 1.45 OD units equals 1 mg/ml. A subset was additionally characterized by N-terminal amino acid sequencing and amino acid compositional analysis.

c-Met Fab Production

Fabs of selected c-Met IgG were generated and purified by papain cleavage and protein A separation utilizing the Pierce ImmunoPure Fab Kit # 44885 following the protocols supplied with the kit. Fabs were characterized by reducing and non-reducing SDS PAGE and size exclusion chromatography. For the c-Met 11978 Fab which bound to protein A after papain cleavage, anion exchange chromatography on a TosoHaas Q-5PW HPLC column of dimensions 7.5 mm×7.5 CM, particle size 10μ, catalog #18257 was utilized for the purification process. The separation was achieved using a binary buffer system, with the primary buffer 20 mM Tris, pH9.0 the counter ion buffer was 20 mM Tris, pH9.0, 1M NaCl. The c-Met 11978 Fab was buffer exchanged into 20 mM Tris, pH9.0 then injected onto the anion exchange column. The column was then washed with 30 ml of primary buffer. The c-Met 11978 Fab was purified by a linear gradient of 0-60% counter ion buffer over 40 minutes. The c-Met 11978 Fab eluted at 0.3M NaCl. The purity was >95%.

### Example 3

### Expression and Purification of Recombinant NK4 Proteins

The CHO DG44 cell line was transfected with pPHA27965 [A cDNA encoding NK4-6His was synthesized by PCR as described (Kuba et al., BBRC 279: 846, 2000) and inserted by standard cloning techniques into pCMV1 (pEU1) with the CMV promoter (Stinski et al., J Virol 46: 1-14, 1983) substituted for the elongation factor promoter]. Forty-eight hours after transfection the cells were placed under selection and expanded. After 7-10 days the cells were then amplified with methotrexate. Once amplified the CHO DG44/pPHA27965 cells were cloned, screened and expanded. The highest expressing clone was further expanded and the protein was expressed in roller bottles.

Purification of Recombinant NK4-6His

Conditioned medium harvested from the roller bottle cultures of NK4-6His, was pooled and adjusted to 50 mM Hepes (pH 6.8). Gross particulates were removed by centrifuging at 28,000 g for 1 hour, and the supernatant fractions were adjusted to 0.02% sodium azide. The NK4-6His was purified by a two-stage chromatographic procedure. The first stage was nickel agarose affinity purification. The NK4-6His was eluted by a linear gradient of imidazole from 5-250 mM. The nickel agarose elution fractions containing NK4-6His were determined by SDS-PAGE and the relevant fractions were pooled. The first stage pool was then dialyzed against 20 mM sodium citrate (pH 6.5), containing 0.01% Tween-80. The adjusted pool was then loaded onto heparin agarose resin. The heparin agarose resin was eluted by a linear sodium chloride gradient from 0-1.8M. The NK4-6His eluted from the resin at approximately 1.3 M sodium chloride. The finished sample was >99% pure by analytical GPC and SDS-PAGE and had a molecular weight of 55 kDa.

### Example 4

### c-Met Ligand Competition ELISA

ELISA Plate Preparation

96-well Fluoronunc plates were coated with 50 ul of 0.5 ug/ml c-Met/Fc Chimera (R&D Systems, Minneapolis Minn., catalog # 358-MT-100) in phosphate buffered saline (PBS) and the plates were incubated overnight at room temperature. Wells were washed three times with washing buffer (PBS+0.1% Tween 20), blotting the plates on paper towels between each wash. Nonspecific binding in the wells was blocked by the addition of 250 ul of blocking buffer (3.0% milk (Carnation) in PBS) to each well and incubated at room temperature for two hours.

ELISA for Detecting Inhibition of Binding of Biotin-HGF to c-Met/Fc Chimera

The c-Met antibodies were diluted in reagent buffer (PBS, 0.5% BSA, 0.05% Tween-20) and titrations were performed in 96 well polypropylene plates. Biotinylated HGF (0.4 ug/ml) (R&D Systems, biotinylated with Pierce #21335 as per manufacturer's instructions) was added to each well. 50 ul of the dilutions were transferred into the Fluoronunc plates containing human c-Met-Fc fusion protein (R&D Systems, #) and the plates were incubated for two hours at room temperature. The plates were washed three times with wash buffer and blotted onto paper towels. 50 ul of europium-labeled Streptavidin (Wallac Perkin Elmer) diluted 1:1000 in Delfia assay buffer (Wallac Perkin Elmer) was added per well and the plates were incubated for one hour at room temperature. The plates were washed seven times with Delfia wash buffer (Tris buffered saline (TBS) supplemented with 0.1% Tween-20) and blotted onto paper towels. 100 ul Delfia enhancer solution (Wallac Perkin Elmer) was added to each well and the plates incubated for 5 minutes on a plate shaker at room temperature. Plates were read on a fluorescence plate reader and analyzed using GraphPad Prism software (GraphPad Software, San Diego, Calif.).

Table 7 shows the IC50 values for the c-Met IgG antibodies and Fab fragments. C-Met antibodies 1A3.3.13 (#HB-11894, ATCC Hybridoma) and 5D5.11.6 (#HB-11895, ATCC Hybridoma) were used as positive controls. MOPC-21 (#M-7894, Sigma) was used as an IgG isotype control and HB-94 (#HB-94, ATCC Hybridoma) was converted into a Fab fragment and used as a Fab isotype control. NK4-Elastase is a kringle to kringle 4 fragment resulting from digesting intact HGF purified from S-114 cells with elastase (Date et al., FEBS Lett. 420:1-6 (1977).

TABLE 7

| | IgG (n = 2) | Fab (n = 2) |
|---|---|---|
| Sample ID | IC50 (nM) | IC50 (nM) |
| 11978 | 0.84 | 65.9, >125 |
| 11994 | 0.58 | 24.57 |
| 12075 | 2.55 | >125 |
| 12119 | 0.64 | 10.65 |
| 12123 | 0.50 | |
| 12133 | 0.58 | |
| 12136 | 1.00 | |
| 11986 | 0.52 | 80.00 |
| 1A3.3.13 _{(+mAb control)} | 0.50 | 6.83 |
| 5D5.11.6 _{(+mAb control)} | | 9.72 |
| MOPC-21 _{(-mAb control)} | >125 | |
| HB94 _{(-Fab control)} | | >125 |
| NK4-Elastase | | 900, 551.4 |
| NK4-His | | >125 |

### Example 5

### Inhibition of HGF-induced Cellular Proliferation by c-Met Antibodies

c-Met antibodies in the IgG and Fab formats were assayed to evaluate their ability to inhibit HGF-induced DNA synthesis. Human mammary epithelial 184B5 cels (ATTCC #CRL-8799) were plated at a cell density of 2.5×10⁴/well into 96-well flat bottom cell culture cluster plates (Corning #3596) in 80 μl per well of starvation media containing RPMI-1640 (Gibco, #21870-084) supplemented with 2mM L-glutamine (Gibco #25030-081), 10 mM 4-(2-hydroxyethyl)-1-piperazineethanesulfonic acid (Gibco #15630-080; Hepes), 50 U/ml penicillin-streptomycin (Gibco #15070-063), and 0.1% protease-free bovine serum albumin (Equitech-Bio, Kerrville, Tex.). Plates were incubated at 37° C./5% CO₂ for 24 hours. 10 μl of assay media or 10 μl of 10× the final concentration for the test monoclonal antibodies was added per well. Plates were incubated at 37° C./5% CO₂ for 30 minutes. 10 μl of 10× the final concentration (130 pM) of rhHGF (R&D Systems #294-HGN/CF) diluted in assay media was added to each well and incubated 16-20 hours at 37° C./5% CO₂. During the last 2 hrs of this incubation 10 μl of diluted BrdU labeling solution, 10 μM final concentration (Roche, #1647229, Cell Proliferation Elisa, BrdU, colorimetric) was added to all wells. The media was decanted by inverting the plates and blotting gently onto a paper towel. Plates were then dried at 60° C. for 1 hour. Fix denaturing solution (Roche, #1647229) was then added at 200 μl per well and incubated 30-45 minutes at room temperature. Plates were decanted again onto a paper towel and 200 μl of Dulbecco's PBS (Gibco, #14040-117) containing 2% BSA (Equitech-Bio) was added to each well to block for 30 minutes at room temperature. PBS was decanted and 100 μl of anti-BrdU-POD (monoclonal antibody, clone BMG 6H8, Fab fragment conjugated with peroxidase) was added per well and incubated for 90 minutes at room temperature. The antibody conjugate was removed by decanting and tapping the plate onto a paper towel. The plates were rinsed 3× with 275 μl/well washing solution (Roche, #1647229). 100 μl/well of TMB substrate solution (tetramethyl-benzidine, Roche, #1647229) was added to the wells and incubated at room temperature for 5-30 minutes. 25 μl of 1M H₂SO₄ (VWR, #VW3232-1) was added and incubated approximately 1 minute with thorough mixing to stop further plate development. The optical density was measured on an ELISA plate reader (Bio-Rad, Model #3550) at 450 nm against a reference wavelength 595 nm.

Table 8 indicates the ability of several IgG antibodies, Fab fragments of these antibodies, or compounds to inhibit HGF dependent proliferation of these cells under assay conditions.

TABLE 8

| Sample ID | IgG (n = 3) | Fab (n = 2) |
|---|---|---|
| 11978 | + | − |
| 11994 | ++ | + |
| 12075 | + | − |
| 12119 | + | + |
| 12123 | + | − |
| 12133 | ++ | + |
| 12136 | + | + |
| 1A3.3.13 | ++ | + |
| MOPC-21 IgG | − | |
| anti-HGF Ab | +++++ | |
| 5D5.11.6 | +++ | + |
| HB94 | | − |
| NK4-Elastase | +++ | |
| NK4-His | +++ | |
| Media alone | − | − |
| ovalbumin | − | |

*Number of + = Degree of Inhibition
− = No Inhibition

### Example 6

### Enhancement of c-Met IgGs and Fabs on c-Met Tyrosine Phosphorylation

To evaluate whether addition of IgG or Fab versions of c-Met antibodies could enhance the phosphorylation of c-Met protein kinase domain HCT-116 human colon carcinoma cells (ATCC #CCL-247) were plated at a cell density of 5×E4/well into six well tissue culture clusters with 2 ml per well of McCoy's medium (Gibco, #16600-082) supplemented with 2 mM L-glutamine (Gibco, #25030-081), 20 mM 4-(2-hydroxyethl)-1-piperazineethanesulfonic acid (Gibco, #15630-080; Hepes), and 10% fetal bovine serum (heat-inactivated; HyClone, #SH30070.03). Cells were incubated at 37° C./5% CO₂ until 70-80% confluent, and the culture media was replaced with 2 ml of the above medium containing 0.2% bovine serum albumin (Equitech-Bio, protease-free, Kerrville, Tex.) instead of FBS. After overnight incubation, the starvation media was replaced with 2.5 ml per well of fresh starvation media pre-warmed to 37° C., and containing 10 nM or 100 nM of selected ligands or test monoclonal antibodies. Dishes were incubated at 37° C. in a circulating water bath for 10 minutes, the media was aspirated, dishes were placed on ice-water, and the cell monolayer was washed three times with 2 ml per well of ice-cold Dulbecco's PBS (Gibco #14040-117). All subsequent operations were conducted at 4° C. Cells were removed from the dishes by addition of 0.3 ml per well of cell lysis buffer. Cell lysis buffer is 1% (v/v) Nonidet P40 (Boehringer Mannheim #1332473), 0.15M NaCl, 25 mM Tris-HCl, (pH 7.5) containing 10% (v/v) glycerol, 5 mM EDTA, 2 mM sodium fluoride, and a 1/100 dilution of stock protease (Sigma P-8340), and phosphatase (Sigma P-2850 and P-5726) inhibitor cocktails. Dishes were shaken in lysis buffer for 5 min, and the contents of each well containing 1.17×E6 cell equivalents were transferred to microfuge tubes, vortexed briefly, and allowed to stand for 30 minutes. The lysate was clarified by centrifuging at 10,000 g for 20 min (Sorvall Legend RT) at 5° C., and 2 ul of the supernatant fraction was assayed for total protein by the method of Bradford (Bradford, Anal. Biochem. 72:248-254, 1976) using the dye reagent obtained from BioRad (# 500-0006) and bovine serum albumin as a protein standard. Equivalent volumes of the supernatant fraction (with a known amount of protein) were mixed with SDS-PAGE sample buffer (Novex ) containing 5% (v/v) 2-mercaptoethanol, heated at 90° C. for 5 minutes, and analyzed by SDS-PAGE on 4-12% Nu-PAGE Bis-Tris gels (Novex # NP0322) in MOPS buffer (Novex # NP0001). For Western blot analysis, proteins were transferred to nitrocellulose (Schleicher and Schuell, BA-85) overnight at 4° C. at 0.2A in Nu-PAGE transfer buffer (Novex # NP0006-1) containing 10% (v/v) methanol. Membranes were blocked for 1 hour at room temperature with blotto (5% (w/v) non-fat dry milk (Carnation), 25 mM Tris-HCl (pH 7.5), 0.15M NaCl, 0.1% (v/v) Tween20, 0.01% thimerosol), then incubated for three hours at room temperature in 1/5000 dilution of rabbit c-Met (Santa Cruz Biotechnology, #sc-161) in 25 mM Tris-HCl, (pH 7.5), 0.15M NaCl, 0.05% (v/v) Tween-20 (TBST) supplemented with 5% bovine serum albumin. Alternatively, that portion of c-Met containing phosphotyrosine within the kinase domain activation loop was determined by incubation of membranes prepared in an identical manner as above in 1/5000 dilution of rabbit anti-pY c-Met IgG (Biosource, #44-888). Peroxidase-conjugated secondary antibody (Jackson Immunoresearch, #111-035-144) was applied at 1/7500 dilution for 45 minutes at room temperature, and then the membranes were washed twice for 30 minutes with TBS containing 0.2% Tween-20, and developed with Supersignal (Pierce #34080) as per manufacturer's instructions. Exposures were captured for 10 or 20 seconds on Bio-Max MR-1 film (Sigma, Z35, 039-7) and band intensity was quantitated by laser densitometry (Molecular Dynamics, Personal Densitometer SI) and analyzed using ImageQuant software. Band intensity was normalized for the total protein contained in each sample, and the fold increase versus control (no addition) signal intensity was determined. FIG. 4 shows that both HGF and multiple c-Met antibodies enhanced the phosphorylation of the c-Met kinase domain over this time period under these conditions, whereas isotype control irrelevant monoclonal antibody (MOPC-21) or irrelevant ligand (IGF-1) did not significantly enhance the endogenous level of phosphotyrosine-containing c-Met. The total amount of c-Met protein subjected to analysis (detected as both the 170 kDa precursor and 145 kDa mature versions of the receptor) was found to be comparable in each analyzed sample.

### Example 7

### c-Met Phosphorylation ELISA

The ability of c-Met monoclonal antibodies to induce tyrosine phosphorylation of c-Met upon binding was also determined using an ELISA format. For this purpose, 96 well plates (VWR, #62409-002) were coated overnight at 4° C. with 100 ng per well of mouse c-Met monoclonal antibody (1A3.3.13 IgG1; ATCC #HB-11894) or isotype-control monoclonal antibody (Sigma, M-5284) in 50 ul of 50 mM sodium borate (pH 8.3; Pierce, #28384). Residual capture antibody was removed and unreacted binding sites were blocked by addition of 180 ul per well of Superblock-TBS (Pierce, #37535). After standing five minutes at room temperature, the blocking step was repeated, then the wells were rinsed twice with Tris-buffered saline (TBS, Sigma, T-5912) supplemented with 0.05% Tween-20 (Sigma P-1379) (TBST), and once with distilled water. Dilutions of cell lysates were added to wells in a final volume of 50 ul of TBS containing 0.1% Tween-20 and 0.2% BSA (Equitech-Bio, 30% solution, protease-free, Kerrville, Tex.) (ELISA buffer), and capture of c-Met protein was allowed to proceed overnight at 4° C. Wells were rinsed twice with TBST and once with distilled water, then 50 ul/well of a 1/2000 dilution of rabbit anti-phosphotyrosine c-Met (Biosource, #44-888) was added to each well in ELISA buffer and incubated for one hour at room temperature. Wells were washed twice with TBST and once with distilled water. 100 ul per well of a 1/20,000 dilution of horseradish peroxidase-conjugated goat anti-rabbit IgG—(Jackson Immunoresearch, #111-035-144) in ELISA buffer was added and the plates incubated for one hour at room temperature. Wells were rinsed three times with TBST and once with distilled water, then developed by addition of 100 ul per well of TMB solution (Sigma, T-4444). Development was allowed proceed at room temperature for 2-5 minutes, then the signal was quenched by addition of 100 ul per well of 7.7% (v/v) phosphoric acid. Optical density was then recorded at 450 nm versus 595 nm reference wavelength using an ELISA reader (Bio-Rad). The results shown in FIG. 4 on duplicate samples obtained with this ELISA assay were comparable to those observed with Western blotting analysis, and confirmed the ability of the tested c-Met monoclonal antibodies to enhance tyrosine phosphorylation of c-Met when compared to MOPC-21 control isotype antibody or untreated control samples.

### Example 8

### Scatter Assay

The agonistic potential of the c-Met antibodies in the absence of HGF as well as the antagonistic potential of c-Met antibodies in the presence of HGF was evaluated using a scatter assay. DU-145 cells were plated at 1000 cells/well in 96-well Perkin Elmer view plates (catalog no. 6005182), or 2500 cells/well in 48-well Greiner Cellstar plates (catalog no. 677180), in RPMI-1640 Media supplemented with 10% Fetal Bovine Serum and Gibco non-essential amino acids. After the cells were allowed to settle down for two hours in a humidified cell culture chamber at 37 C and 5% CO2, HGF and/or inhibitors are added to the wells in triplicates. The cells were kept in the cell culture chamber above for 48 to 72 hours. Subsequently, the cells were fixed with 2% paraformaldehyde (Electron Microscopy Sciences, catalog no. 15713-S). Cytoplasmic and nucleic areas of the cells were stained with propidium iodide (Molecular Probes, catalog no. P-3566) and Hoechst dye, respectively. Levels of scattering were measured in a Cellomics ArrayScan II, and expressed as mean object areas.

Table 9 shows the agonistic potential of several c-Met antibodies and Fab fragments and compounds in the absence of HGF as well as the antagonistic potential of c-Met antibodies and compounds in the presence of HGF.

TABLE 9

| | IgG | | Fab | |
|---|---|---|---|---|
| | Agonist | Antagonist | Agonist | Antagonist |
| Sample ID | (alone) | (w/30 pM HGF) | (alone) | (w/30 pM HGF) |
| 11978 | 459 +/− 130 | 372 +/− 87 | | |
| 11994-50 | 272 +/− 30* | 294 +/− 27* | 738 +/− 145 | 404 +/− 19 |
| 11986 | | | 501 +/− 82 | 557 +/− 201 |
| 12075 | 318 +/− 98 | 289 +/− 61 | | |
| 12119 | 285 +/− 15 | 293 +/− 20 | | |
| 12123 | 234 +/− 0.007 | 226 +/− 2 | | |
| 12133 | 241 +/− 23 | 230 +/− 38 | | |
| 12136 | 249 +/− 27* | 296 +/− 64* | | |
| 1A3.3.13 | 305 +/− 66 | 254 +/− 24 | 597 +/− 45 | 400 (n = 1) |
| 5D5.11.6 | 239 +/− 16 | 241 +/− 13 | 632 +/− 74 | 592 +/− 76 |
| HB94 | 365 +/− 38 | 199 +/− 14 | 592 +/− 84 | 478 +/− 91 |
| NK4-Elastase | 335 +/− 17 | 471 +/− 130 | 740 +/− 129 | 697 +/− 208 |
| HGF | | 324 +/− 37 | | |
| media alone | | | 685 +/− 445 | 352 +/− 56 |
| | | | (No HGF) | |

Cellomics measurement at 100 nM IgG or Fab except * at 50 nM.
The smaller the number, the more scattering.

### Example 9

### Scratch Assay with c-Met Antibodies

To evaluate the ability of the c-Met IgG and Fab antibodies to inhibit recombinant human HGF (R&D Systems, # 249-HG)-induced cell motility a scratch assay was used that incorporated robotic-induced scratches. Visualization using a fluorogenic intracellular substrate, Vybrant CFDA (Molecular Probes, #V-12883) was used to maximize invasion visibility and produce images with a high signal/noise ratio. Analysis of the migration into the scratch area was performed using AnalySIS Software (Soft Imaging Systems, Lakewood Colo.).

Plate Setup

NCI H441 (ATCC #HTB-174) adenocarinoma cells from a 70-90% confluent T-162 cm² flask were washed with PBS and harvested with trypsin/EDTA. Released cells were suspended in 10 ml RPMI-1640 (Gibco, #11875-085) supplemented with 10% fetal bovine serum (Gibco, #26140-079) and dispensed into 48-well tissue culture plates containing 0.5 ml of RPMI-1640 supplemented with 10% fetal bovine serum. Scratches were induced in confluent monolayers by a pipette tip using a Biomek 2001 robot (Beckman Coulter, Fullerton Calif.), producing a single scratch per well. A fresh tip was used for each row. The wounded cell monolayers were gently washed twice with 0.5 ml RPMI-1640, once with PBS, and then treated with 0.5 ml per well RPMI-1640 with 0.1% BSA (Sigma, #A8327) containing test antibodies or controls at concentrations ranging from 0.1-30 ug/ml. After a 20 minute pre-incubation, 50 ul of HGF (final concentration=225 pM) was added to each well and the plates were incubated 20-24 hours at 37° C./5% CO₂.

Plate Staining and Analysis

Vybrant Dye Solution was prepared by dissolving 90 ul of DMSO in one vial of dye and then transferring to 37 ml of HBSS (Gibco, #14025-092). Media from the wells was aspirated and 0.5 ml of Vybrant Dye solution was added. After 30 minutes incubation at 37° C./5% CO₂, the dye solution was replaced with 0.5 ml HBSS. After 20 minutes at 37° C./5% CO₂ image analysis was performed. Cell monolayers were then fixed with 1% freshly prepared formaldehyde in PBS.

Fluorescence images were captured on a Nikon TE300 inverted fluorescence microscope with a 2× objective and a FITC filter pack. The microscope has a motorized stage controlled by AnalySIS well navigator software (Soft Imaging System GMBH) and was used to automate the data collection. Analysis of the area of the scratch was done using this software. Area of the scratch was reported in um². Data was processed and plotted using Excel Software. When replicates were tested, SEM was used for error bars.

Table 10 Displays data of the inhibition of the c-Met IgG antibodies and Fab fragments compared with that observed with 1A3.3.13 and 5D5.11.6 IgGs and Fabs, or recombinant NK4.

TABLE 10

| | Scratch Assay* | (Cell Motility) (n = 3) |
|---|---|---|
| Sample ID | IgG | Fab |
| 11978 | + | +/− |
| 11994 | ++ | +/− |
| 12075 | +/− | +/− |
| 12119 | ++ | +/− |
| 12123 | + | +/− |
| 12133 | ++ | +/−(2/3); +(1/3) |
| 12136 | ++ | +/− |
| 1A3.3.13 | + | +/− |
| 5D5.11.6 | ++ | +/− |
| NK4-His | ++ | NR |
| MOPC-21 | +/− | +/− |
| HB94 | +/− | +/− |

*Inhibition > 1A3 IgG (++)
Inhibition < 1A3 IgG (+)
No Activity (+/−)
NR—Not Relevant

### Example 10

### Biacore Assay

The binding properties (on-rate, off-rate and affinity) of c-Met monoclonal antibodies (IgG or Fab versions) with human c-Met extracellular domain was determined using surface plasmon resonance, or BLAcore, technology. For the binding studies with IgG, a low density (<1 ng/mm2) of c-Met-Fc (R&D Systems, #358-MT-100/CF) containing 5.1 biotin per c-Met molecule (prepared with Pierce #21335 as per manufacturer's instructions) was captured onto a SA chip precoated with Streptavidin (BIAcore Inc.). A streptavidin flow cell without adsorbed c-Met-Fc was used as a control cell for non-specificbinding. The antibody sample to be analyzed was prepared in HEPES buffer (0.15M NaCl, 10 mM HEPES, 3.4 mM EDTA, 0.005% surfactant P-20, pH 7.4) to form a set of solutions varied in concentration from 0.78 nM to 100 nM. The HEPES buffer used as the running solution was set at a flow rate of 50 ul/min. Each sample solution was injected over the two flow cells for three minutes, followed by 5 minutes of dissociation. The flow cells were then regenerated with 4.5M MgCl₂ for one minute to remove the bound antibody for the next cycle of binding study. The net sensorgrams (subtraction of sensorgrams from the negative control flow cell as well as that from the buffer blank) obtained for each set of samples were processed simultaneously in a global fitting using a bivalent binding model of the BIAevaluation software program equipped with the system. The on-rate (Kₐ), off-rate (K_{d}) and binding affinity (K_{D}) were determined from the fitting with K_{D} equal to k_{d}/kₐ.

For the binding study of Fab fragments derived from antibodies of the invention, a high density (>2 ng/mm²) of protein A was first immobilized covalently onto a CM5 sensorchip using EDC/NHS amine coupling chemistry [. The flow cell containing c-Met-Fc captured by the protein A was used as the positive control while a flow cell containing only protein A was used as the negative control. The Fab sample to be analyzed was as above for antibodies to form a set of solutions with concentration ranged from 3.9 nM to 500 nM. The HEPES buffer used as the running solution was set at a flow rate of 50 ul/min. For each cycle of binding study, low density (<1 ng/mm²) of c-Met-Fc was captured first onto the positive flow cell. Each sample solution was then injected over the two flow cells (one negative and one positive in series) for three minutes followed by 5 minutes of dissociation. The flow cells were then regenerated with 4.5M MgCl₂ for one minute to remove the bound c-Met-Fc/Fab complexes for the next cycle of binding. The net sensorgrams (subtraction of sensorgrams from the negative control flow cell as well as that from the buffer blank) obtained from the set of samples were fitted simultaneously in a global fitting using a Langmuir 1:1 binding model of the BIAevaluation program equipped with the system. The on-rate (Kₐ), off-rate (K_{d}) and binding affinity (K_{D}) were determined from the fitting with K_{D} equal to k_{d}/kₐ.

Tables 11 and 12 show the binding kinetics of several c-Met IgG antibodies and Fab fragments respectively.

TABLE 11
c-Met IgGs

| Sample ID | on-rate(1/sM) | off-rate(1/s) | KD(nM) |
|---|---|---|---|
| 11978 | ND | ND | ND |
| 11994 | 9.06E+04 | 7.59E−04 | 8.4 |
| 12075 | 1.53E+04 | 8.45E−03 | 552 |
| 12119 | 8.60E+04 | 1.12E−03 | 13 |
| 12123 | 3.38E+05 | 3.29E−03 | 9.7 |
| 12133 | 9.89E+04 | 5.98E−04 | 6 |
| 12136 | 2.94E+05 | 2.29E−04 | 0.8 |
| 1A3.3.13 | 2.10E+05 | 2.89E−04 | 1.4 |
| 5D5.11.6 | 6.88E+04 | 4.06E−04 | 5.9 |

TABLE 12
c-Met Fabs

| Sample ID | on-rate(1/sM) | off-rate(1/s) | Kd(nM) |
|---|---|---|---|
| 11994 | 3.83E+05 | 5.28E−03 | 13.8 |
| 12133 | 2.80E+05 | 2.45E−03 | 8.8 |
| 12136 | 1.68E+05 | 1.01E−03 | 6 |
| 1A3.3.13 | 4.97E+05 | 3.13E−03 | 6.3 |
| 5D5.11.6 | 1.26E+05 | 1.29E−04 | 1 |

What is claimed is:

1. An antibody or antigen binding portion thereof that specifically binds to c-MET, wherein said antibody comprises a c-MET antibody selected from the group consisting of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1 or fragment of any one thereof.

2. The antibody or antigen binding portion thereof according to claim 1 wherein said c-Met antibody is selected from the group consisting of PGIA-01-A8, PGIA-03-A9, PGIA-03-A11, PGIA-03-B2, PGIA-04-A5, PGIA-04-A8, PGIA-05-A1 or a fragment of any one thereof.

3. The antibody or antigen binding portion thereof according to claim 1 wherein said c-Met antibody is selected from the group consisting of PGIA-03-A9, PGIA-04-A5, and PGIA-04-A8 or a fragment of any one thereof.

4. The antibody or antigen binding portion thereof of claim 1, wherein said antibody comprises at least one light chain of said c-Met antibody.

5. The antibody or antigen binding portion thereof of claim 1, wherein said antibody comprises at least one heavy chain of said c-Met antibody.

6. The antibody or antigen binding portion thereof of claim 4 or 5, wherein said antibody comprises at least one CDR of said c-Met antibody.

7. The antibody or antigen binding portion thereof of claim 6, wherein said antibody comprises all of the CDRs of at least one heavy chain of said c-Met antibody.

8. The antibody or antigen binding portion thereof of claim 6, wherein said antibody comprises all of the CDRs of at least one light chain of said c-Met antibody.

9. The antibody or antigen binding portion thereof of claim 6, wherein said antibody comprises all of the CDRs of a heavy chain and a light chain of said c-Met antibody.

10. The antibody or antigen binding portion thereof of claim 6, wherein said antibody comprises CDRs from different light chains of said c-Met antibody.

11. The antibody or antigen binding portion thereof of claim 6, wherein said antibody comprises CDRs from different heavy chains of said c-Met antibody.

12. The antibody or antigen binding portion thereof of claim 6, wherein said antibody comprises a V_{L} and/or V_{H} variable region of said c-Met antibody.

13. The antibody or antigen binding portion thereof according to claim 1, wherein said c-Met antibody comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or a fragment of any one thereof.

14. The antibody or antigen binding portion thereof of claim 13, wherein said antibody comprises at least one light chain of said c-Met antibody.

15. The antibody or antigen binding portion thereof of claim 13, wherein said antibody comprises at least one heavy chain of said c-Met antibody.

16. The antibody or antigen binding portion thereof of claim 14 or 15, wherein said antibody comprises at least one CDR of said c-Met antibody.

17. The antibody or antigen binding portion thereof of claim 16, wherein said antibody comprises all the CDRs of at least one heavy chain of said c-Met antibody.

18. The antibody or antigen binding portion thereof of claim 16, wherein said antibody comprises all the CDRs of at least one light chain of said c-Met antibody.

19. The antibody or antigen binding portion thereof of claim 16, wherein said antibody comprises all of the CDRs of a heavy chain and a light chain of said c-Met antibody.

20. The antibody or antigen binding portion thereof of claim 16, wherein said antibody comprises CDRs from different light chains of said c-Met antibody.

21. The antibody or antigen binding portion thereof of claim 16, wherein said antibody comprises CDRs from different heavy chains of said c-Met antibody.

22. The antibody or antigen binding portion thereof of claim 16, wherein said antibody comprises at least one V_{L} and/or V_{H} variable region of said c-Met antibody.

23. The antibody or antigen-binding portion thereof according to any one of claims 1 or 13, wherein the antibody or portion thereof has at least one property selected from the group consisting of:
a) cross-competes for binding to human c-Met with the c-Met antibody selected from the group consisting of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1;
b) binds to the same epitope of human c-Met as the c-Met antibody selected from the group consisting of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1;
c) binds to human c-Met with substantially the same K_{d} as the c-Met antibody selected from the group consisting of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1; and
d) binds to human c-MET with substantially the same off rate as the c-Met antibody selected from the group consisting of PGIA-01-A1, PGIA-01-A2, PGIA-01-A3, PGIA-01-A4, PGIA-01-A5, PGIA-01-A6, PGIA-01-A7, PGIA-01-A8, PGIA-01-A9, PGIA-01-A10, PGIA-01-A11, PGIA-01-A12, PGIA-01-B1, PGIA-01-B2, PGIA-02-A1, PGIA-02-A2, PGIA-02-A3, PGIA-02-A4, PGIA-02-A5, PGIA-02-A6, PGIA-02-A7, PGIA-02-A8, PGIA-02-A9, PGIA-02-A10, PGIA-02-A11, PGIA-02-A12, PGIA-02-B1, PGIA-03-A1, PGIA-03-A2, PGIA-03-A3, PGIA-03-A4, PGIA-03-A5, PGIA-03-A6, PGIA-03-A7, PGIA-03-A8, PGIA-03-A9, PGIA-03-A10, PGIA-03-A11, PGIA-03-A12, PGIA-03-B1, PGIA-03-B2, PGIA-03-B3, PGIA-03-B4, PGIA-03-B5, PGIA-03-B6, PGIA-03-B7, PGIA-03-B8, PGIA-04-A1, PGIA-04-A2, PGIA-04-A3, PGIA-04-A4, PGIA-04-A5, PGIA-04-A6, PGIA-04-A7, PGIA-04-A8, PGIA-04-A9, PGIA-04-A10, PGIA-04-A11, PGIA-04-A12, and PGIA-05-A1.

24. The antibody or antigen-binding portion thereof according to claim 1 or 13, wherein said antibody or antigen-binding portion thereof comprises a variable region of a light chain, wherein the sequence of said variable region of said light chain comprises no more than ten amino acid changes from the amino acid sequence encoded by a germline gene thereof.

25. The antibody or antigen-binding portion thereof according to any one of claims 1 or 13 that is
a) an immunoglobulin G (IgG), an IgM, an IgE, an IgA or an IgD molecule;
b) an Fab fragment, an F(ab′)2 fragment, an Fv fragment, a single chain antibody; or
c) a humanized antibody, a human antibody, a chimeric antibody or a bispecific antibody.

26. The antibody of claim 25 a) wherein said c-Met antibody is an IgG selected from the group consisting of 11978, 11994, 12075, 12119, 12123, 12133, and 12136.

27. The antibody of claim 26 selected from the group consisting 11994, 12133, and 12136.

28. The antibody of claim 25 b) wherein said c-Met antibody is a Fab selected from the group consisting of 11978, 11994, 12075, 12119, 12123, 12133, and 12136.

29. The antibody of claim 28 selected from the group consisting 11994, 12133, and 12136.

30. A pharmaceutical composition comprising the antibody or portion thereof according to claim 1 and a pharmaceutically acceptable carrier.

31. An isolated cell line that produces the antibody according to claim 1.

32. A method of diagnosing the presence or location of an HGF expressing tumor in a subject in need thereof, comprising the steps of
a) injecting the antibody according to claim 1 into the subject,
b) determining the expression of c-MET in the subject by localizing where the antibody has bound,
c) comparing the expression in part (b) with that of a normal reference subject or standard, and
d) diagnosing the presence or location of the tumor.

33. A method of treating cancer in a human with the antibody or antigen-binding portion thereof according to claim 1, comprising the step of administering to said human an effective amount of said antibody.

34. An isolated nucleic acid molecule that comprises a nucleic acid sequence that encodes a heavy chain or antigen-binding portion thereof or a light chain or antigen-binding portion thereof of an antibody according to claim 1.

35. The nucleic acid sequence according to claim 34 wherein said nucleic acid sequences is selected from the group consisting of: SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:100, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, and SEQ ID NO:120 or a fragment thereof.

36. A vector comprising the nucleic acid molecule according to claim 34 or 35, wherein the vector optionally comprises an expression control sequence operably linked to the nucleic acid molecule.

37. A host cell transformed or transfected with the nucleic acid sequence of claim 34 or 35.

38. The antibody or antigen binding portion thereof of claim 1, wherein said antibody or antigen binding portion is a partial agonist against c-MET.

39. The antibody or antigen binding portion thereof of claim 1, wherein said antibody or antigen binding portion blocks HGF driven proliferation.

40. The antibody or antigen binding portion thereof of claim 1, wherein said antibody or antigen binding portion blocks HGF binding to human c-MET.


SEQUENCE LISTING (161 sequences)

SEQ ID NO: 1 - protein, 238 aa, artificial
  phage display generated human antibody
EVQLLESGRGLVQPGGSLRLSCAASGFTFSSYAMSWVRQAPGKGLEWVSAISGSGGSTYYADSVKGRFTISRDNSKNTLYLQMNSLRAEDTAVYYCARWGQGTTVTVSSGGGGSGGGGSGGGGSAQAVLTQPSSVSGAPGQRVTISCTGSSSNIGADYDVHWYQQLPGTAPKLLIYGNNNRPSGVPDRFSGSKSGTSASLAITGLQAEDEADYYCQSYDNSPDAYVVFGGGTKLTVLS

SEQ ID NO: 2 - protein, 244 aa, artificial
  phage display generated human antibody
QVQLVQSGAEVRKPGASVKVSCKTSGYTFIDYYIHWVRQAPGQGLEWMGWVNPVTGTSGSSPNFRGRVTMTTDTSGNTAYMELRSLRSDDTAVFYCARRHQQSLDYWGQGTLVTVSSGGGGSGGGGSGGGGSAQSVLTQPPSVSAPPGQKVTISCSGSSSNIGTNYVSWYQQLPGTAPKLLIYDNHKRPSVIPDRFSGSKSGTSATLGISGLQTGDEADYYCGTWDYSLSTWVFGGGTKLTVLG

SEQ ID NO: 3 - protein, 240 aa, artificial
  phage display generated human antibody
QLQLQESGPGLVKPSGTLSLTCAVSGDSVSSYYWWSWVRQPPGKGLEWIGEIFRDGSSNYNRSLKSRVTISPDKPKNQFSLRLSSVTAADTAIYYCARHIRGYDAFDIWGRGTLVTVSSGGGGSGGGGSGGGGSAQSVLTQPPSVSGAPGQRVTISCTGSSSNIGAGYDVHWYQQFPGRAPKLLIYGNTNRPSGVPDRFSGSKSDISASLAITGLQAEDEADYYCQSYDSNLTGVFGGGT

SEQ ID NO: 4 - protein, 244 aa, artificial
  phage display generated human antibody
QVQLVQSGAEVRKPGASVKVSCKTSGYTFMDYYIHWVRQAPGQGLEWMGWSNPVTGTSGSSPKFRGRVTLTTDTSGNTAYLDLRSLRSDDTAVFYCARRHQQSLDYWGQGTMVTVSSGGGGSGGGGSGGGGSAQSVLTQPPSVSAAPGQKVTISCSGSSSNIGNNYVSWYQQLPGTAPKLLMYENSKRPSGIPDRFSGSKSGTSGTLGITGLQTGDEADYYCGTWDTSLRAWVFGGGTKVTVLG

SEQ ID NO: 5 - protein, 244 aa, artificial
  phage display generated human antibody
QVQLQQSGAEVRKPGASAKVSCKTSGYTFIDYYIHWVRQAPGQGLEWMGWINPVTGASGSSPNFRGRVTLTTDTSGNTAYMELRSLRSDDTAVFYCARRHQQSLDYWGRGTTVTVSSGGGGSGGGGSGGGGSAQSVVTQPPSVSAAPGQKVTISCSGRTSNIGNNYVSWYQQVPGAPPKLLIFDNNKRPSGTPARFSGSKSGTSATLAISGLQTGDEADYYCGTWDTTLRGFVFGPGTKVTVLG

SEQ ID NO: 6 - protein, 250 aa, artificial
  phage display generated human antibody
QLQLQESGPGLVKPSGTLSLTCAVSGGSISSTNWWSWVRQPPGKGLEWIGEIYHSGSTNYNPSLKSRVTISVDKSKNHFSLNLSSVTAADTAVYYCARDSMGSTGWHYGMDLWGRGTLVTVSSGGGGSGGGGSGGGGSAQSALTQPPSASGSPGQSVTISCSGSSSDIGDYNHVSWYQQHPGKAPKLMIYDVNKWPSGVPDRFSGSKSGNTASLTVSGLQAEDEADYYCSSYSGIYNLVFGGGTKVTVLG

SEQ ID NO: 7 - protein, 251 aa, artificial
  phage display generated human antibody
EVQLVQSGAEVKKPGSSVKVSCKASGGTFKTYAINWVRQAPGQGLEWMGGIIPVLGTANYVQKFQGRVTITADESTTTAYMELRGLRSEDTAVYYCARGEGSGWYDHYYGLDVWGQGTLVTVSSGGGGSGGGGSGGGGSAQSVLTQPPSASGTPGQRVTISCSGSSSNIGSNTVNWYRQLPGTAPKLLIFGDDQRPSGVPDRFSGSRSGTSVSLAISGLQSEDEADYYCAAWDDSLNGGVFGGGTKLTVLG

SEQ ID NO: 8 - protein, 250 aa, artificial
  phage display generated human antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDHYYDSSGYLDYWGQGTLVTVSSGGGGSGGGGSGGGGSALNFMLTQPHSVSESPGKTVTISCTRSSGSIAFDYVQWYQQRPGSAPTTVIYEDNQRPSGVPDRFSASIDSSSNSASLTISALKTEDEADYYCQSYDNSNSWVFGGGTKLTVLG

SEQ ID NO: 9 - protein, 242 aa, artificial
  phage display generated human antibody
KVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDVRNAFDIWGRGTTVTVSSGGGGSGGGGSGGGGSAQSVLTQPPSVSVSPGQTTSITCSRDKLGEQYVYWYQQRPGQSPILLLYQDSRRPSWIPERFSGSNSGDTATLTISGTQALDEADYYCQAWDNSSYVAFGGGTKVTVLG

SEQ ID NO: 10 - protein, 245 aa, artificial
  phage display generated human antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGELWNPYLDYWGQGTLVTVSSGGGGSGGGGSGGGGSALPVLTQPPSVSVAPGKTARITCGGNDIASKSVQWFQQKPGQAPVLVIYYDSDRPSGIPERFSGSNSENTATLTISRVEAGDEADYYCQVWDSSSDHPVFGGGTKLTVLG

SEQ ID NO: 11 - protein, 250 aa, artificial
  phage display generated human antibody
QVQLVQSGAEVKKPGESLKISCKGSGYTFTNYWIAWVRQMPGKGLEWMGIIYPDDSDTRYNPSFQGQVTMSADKSIDTAYLQWSSLKASDTAIYYCARPSGWNDNGYFDYWGRGTTVTVSSGGGGSGGGGSGGGGSALNFMLTQPHSVSASPGKTVTLSCTGSSGSIASNYVQWYRQRPGSAPTTVIYDDNQRPSGVPDRFSGSIDSSSNSASLTISGLKTEDEADYYCQSFDNDNHWVFGGGTKLTVLG

SEQ ID NO: 12 - protein, 247 aa, artificial
  phage display generated human antibody
QVQLQESGPGLVRSSGILSLTCSVSGVSVSSNNWWSWVRQTPGKGLEWIGEIYQTGTTNYNPSLKSRVAISLDKSRNQFSLILKSVTAADTAVYYCARTSSAWSNADWGKGTMVTVSSGGGGSGGGGSGGGGSALSSELTQDPSASGSPGQSVSISCTGTSSDVGGYNYVSWYQQHPGKAPKLMISEVTKRPSGVPDRFSGSKSGNTASLTVSGLQAEDEADYYCSSFGANNNYLVFGGGTKLTVLG

SEQ ID NO: 13 - protein, 251 aa, artificial
  phage display generated human antibody
QVQLQESGPRLVKPSQTLSLTCTVSNDSIISGDYFWSWIRQPPGKGLEWIGNIFYTGSTSYNPSLKSRLTMSLDTSKNQFSLRLSSVTAADTAVYFCARGRQGMNWNSGTYFDSWGRGTLVTVSSGGGGSGGGGSGGGGSALSYVLTQPPSVSVAPGKTANITCGGKNIGNKSVQWYQQKPGQAPVVVMYYDSDRPSGIPERFSGSNAGNTATLTIDRVEAGDEADYYCQVWDKSSDRPVFGGGTKLTVLG

SEQ ID NO: 14 - protein, 245 aa, artificial
  phage display generated human antibody
QVQLVQSGAEVKKPGASVKVSCKTSGYTFMEYYIHWVRQAPGQGLEWMGWSNPVTGTSGSSPKFRGRVTLTTDTSGNTAYLDLRSLRSDDTAVFYCARRHQQSLDYWGQGTLVTVSSGGGGSGGGGSGGGGSAQSVVTQPPSASGSPGQSVTISCSGYSSSNIGNNAVSWYQQLPGTAPKLLIFDNNKRPSGIPARFSGSQSGTTATLGITGLQTGDEADYFCGTWDSSLSAFVFGSGTKVTVLG

SEQ ID NO: 15 - protein, 246 aa, artificial
  phage display generated human antibody
EVQLVQSGAEVKKPGSSVKVSCKASGGSFSNYDFSWVRQAPGQGLEWMGEIINAFGSSRYAQKFQDRVTITADESASTAYMELRGLTSEDTATYYCARAERWELNMAFDMWGRGTLVTVSSGGGGSGGGGSGGGGSAQSVLTQPPSVSVAPGQTARITCGGDNIGRKNVHWYQQRPGLAPVLVVYDDTDRPSGIPERFSGSNSGDTATLTITWVEAGDEADYYCQLWDSDTYDVLFGGGTKLTVLG

SEQ ID NO: 16 - protein, 247 aa, artificial
  phage display generated human antibody
EVQLVQSGAEVKKPGSSVKVSCKSSGGPFSSYGISWVRQAPGQGLEWMGGISPIFGTANYAQKFQGRVTITADESTETAYMELSSLRSEDTAVYYCARDESPVGFYALDIWGRGTTVTVSSGGGGSGGGGSGGGGSALSYELTQPPSVSVAPGQTARINCGGDKIGSRSVHWYQQKPGQAPVMVVYDDSDRPSGIPERFSGSNSGNTATLTISSVEAGDEADYYCQVWDGSTDPWVFGGGTKVTVLG

SEQ ID NO: 17 - protein, 255 aa, artificial
  phage display generated human antibody
EVQLVQSGAEMKKPGSSVKVSCKASGGTFSSYAVNWVRQAPGQGLEWMGGIIPIFDTSNYAQKFQGRLTMTADDSTNTAYMELRSLRSEDTAVYYCARGAPRGTVMAFSSYYFDLWGQGTLVTVSSGGGGSGGGGSGGGGSALNFMLTQPHSVSESPGKTVIISCAGSGGNIATNYVQWYQHRPGSAPITVIYEDNQRPSGVPDRFSGSVDSSSNSASLTISGLQTEDEADYYCHSYDNTDQGVFGTGTKVTVLG

SEQ ID NO: 18 - protein, 253 aa, artificial
  phage display generated human antibody
EVQLVESGGGLVQPGRSLRLSCAASGFTFDDYDMHWVRQAPGKGLEWVSSISWSGGTIGYADSVKGRFTVSRDNAKNSLYLQMNSVRAEDTALYYCAKDRGAVAALPDYQYGMDVWGRGTLVTVSSGGGGSGGGGSGGGGSAQSALTQPASVSGSPGQSITISCTGTSSDIGSYNLVSWYQQHPGKAPKLMIYEDYKRASGVSNHFSGSKSGNTASLTISGLQAEDEADYYCSSYAGSSAWVFGGGTKVTVLG

SEQ ID NO: 19 - protein, 245 aa, artificial
  phage display generated human antibody
EVQLVQSGAEVRKPGSSMKVSCKASGDTFRNFAFSWVRQAPGQGLEWMGGVIPLVGPPKYAQKFQGRLTITADESTSTSYMDLTSLTLEDTAVYFCARGGVYAPFDKWGQGTLVTVSSGGGGSGGGGSGGGGSAQSVVTQPPSVSEAPRQRVTISCSGSSSNIGNNAVNWYQQLPGKAPKLLIYYNDLLPSGVSDRFSGSKSGTSASLAISGLQSEDEADYYCAAWDDSLNGWVFGGGTKVTVLG

SEQ ID NO: 20 - protein, 251 aa, artificial; identical to SEQ ID NO: 7
  phage display generated human antibody

SEQ ID NO: 21 - protein, 248 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGKGTLVTVSSGGGGSGGGGSGGGGSAQAVLTQPSSVSAAPGQKVTISCSGSSSNIGNNYVSWYQQLPGTAPKLLIYDNNKRPSGIPDRFSGSRSGTSATLGITGLQTGDEADYYCGTWDSSLSAVVFGTGTKLTVLG

SEQ ID NO: 22 - protein, 250 aa, artificial
  phage display generated human antibody
QLQLQESGPGLVKPSGTLSLTCAVSGGSISSTNWWSWVRQPPGKGLEWIGEIYHSGSTNYNPSLKSRVTISVDKSKNHFSLNLSSVTAADTAVYYCARDSMGSTGWHYGMDLWGKGTLVTVSSGGGGSGGGGSGGGGSAQSALTQPASVSGSPGQSIAISCTGTSSDVGGYNYVSWYQQHPGKAPKLMIYAVTNRPSGVSDRFSGSKSGNTASLTISGLQADDEADYYCSSYTSSSSLVFGGGTKLTVLG

SEQ ID NO: 23 - protein, 240 aa, artificial
  phage display generated human antibody
GVQLVESGGGLVKPGGSLRLSCAASGFTFSSYTMNWVRQAPGKGLEWVSYISSSGSATYYADSVKGRFTISRDNANNSLYLQMNSLRAEDTAVYYCARGYRYGMDVWGQGTLVTVSSGGGGSGGGGSGGGGSGIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKR

SEQ ID NO: 24 - protein, 245 aa, artificial
  phage display generated human antibody
EVQLLESGGGLVQPGGSLRLTCAASGFTFSSYAMSWVRQAPGKGLEWVSAISGSGGSTYYADSVKGRFTISRDNSKNTLYLQMNSLRAEDTAVYYCARDLAVAGIDYWGRGTMVTVSSGGGGSGGGGSGGGGSAQSVLTQPPSASGTPGQRVTISCSGSSSNIRSNYVYWYQQFPGTAPKLLIYRNNQRPSGVPDRFSGSKSGTSASLAISGLRSEDEADYYCAAWDDTLDAYVFAAGTKLTVLG

SEQ ID NO: 25 - protein, 251 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSALNFMLTQPHSVSGSPGRTVTISCTRSSGSIATNYVQWYQQRPGSSPTIVIYEDNQRPSGVPDRFSGSIDTSSNSASLTISGLKTEDEADYYCQSYDSNNLGVVFGGGTQLTVLS

SEQ ID NO: 26 - protein, 249 aa, artificial
  phage display generated human antibody
QVQLQQSGAEVRKPGASVKISCKTSGYTFMDYYIHWVRQAPGQGLEWMGWSNPVTGTSGSSPKFRGRVTLTTDTSGNTAYLDLRSLRSDDTAVFYCARRHQQSLDYWGQGTLVTVSSGGGGSGGGGSGGGGSAQAVLTQPSSLSASPGASASLTCTLRSDINVGSYSINWYQQKPGSPPQYLLNYRSDSDKQQGSGVPSRFSGSKDASANAGILLISGLQSEDEADYYCMIWYRTAWVFGGGTKVTVLG

SEQ ID NO: 27 - protein, 244 aa, artificial
  phage display generated human antibody
QVQLVQSGAEVRKPGASVKVSCKTSGYTFIEYYIHWVRQAPGQGLEWMGWSNPVTGTSGSSPKFRGRVTLTTDTSGNTAYLDLRSLRSDDTAVFYCARRHQQSLDYWGRGTTVTVSSGGGGSGGGGSGGGGSAQSVLTQPPSVSAAPGQKVTISCSGTNSNIGNYYVSWYQQLPGTAPKLLIYDNNKRPSGVPDRFSGSKSGTSASLVISGLRSEDEADYYCAAWDGSLTAWVFGGGTKVTVLG

SEQ ID NO: 28 - protein, 250 aa, artificial
  phage display generated human antibody
QVQLQESGPGLVKPSGTLSLTCAVSGDSISSSNWWTWVRQPPGKGLEWIGEIFHSGTTNYNPSLNNRVTISLDESRNQFSLELSSVTAADTAIYYCARDSGNYDDNRGYDYWGRGTLVTVSSGGGGSGGGGSGGGGSAQSVLTQPPSVSGAPGQRVTISCAGTSSNIGAGFDVHWYQLLPGRAPKLLIYGNNNRPSGVPDRFSGSKSGTSASLAISGLQSEDEGDYYCAAWDDTVGGPVFGGGTKLTVLG

SEQ ID NO: 29 - protein, 250 aa, artificial
  phage display generated human antibody
QVQLQESGPGLVKPSGTLSLTCAVSGGSISSTNWWSWVRQPPGKGLEWIGEIYHSGSTNYNPSLKSRVTISVDKSKNHFSLNLSSVTAADTAVYYCARDSMGSTGWHYGMDLWGRGTLVTVSSGGGGSGGGGSGGGGSAQSALTQPAAVSGSPGQSITISCTGSSSDVGGYNYVSWYQQHPGKAPKLLIYDVSDRPSGVSYRFSGSKSGNTASLTISGLQAEDEADYYCSSYTATGTLVFGGGTKLTVLG

SEQ ID NO: 30 - protein, 251 aa, artificial
  phage display generated human antibody
QVQLQESGPGLVKPSGTLSLTCAVSGGSISSTNWWSWVRQPPGKGLEWIGEIYHSGSTNYNPSLKSRVTISVDKSKNHFSLNLSSVTAADTAVYYCARDSMGSTGWHYGMDLWGQGTTVTVSSGGGGSGGGGSGGGGSAQSALTQPASVSGSPGQSITISCTGTSSDVGGYNYVSWYQQHPGKAPKLMIYEVSNRPLGVSNRFSGSKSGNTASLTISGLQAEDEGDYYCSSYTSSTTLIVFGGGTKLTVLG

SEQ ID NO: 31 - protein, 248 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSAQSVLTQPPSVSGTTGQRVILSCSGGNSNIGYNSVNWYQQLPGTAPKLLIYTDDQRPSGVPDRFSGSRSGTSASLAISGLQSEDEADYYCATWDDSLNAGVFGGGTKLTVLG

SEQ ID NO: 32 - protein, 245 aa, artificial
  phage display generated human antibody
QVQLVQSGAEVRKPGASVRVSCKTSGYTFLEYYIHWVRQAPGQGLEWMAWSNPVTGTSGSSPKFRGRVTLTADTSGNTAYLDLKSLTSDDTAIFYCARRHQQSLDYWGQGTLVTVSSGGGGSGGGGSGGGGSAQSVLTQPPSVSAAPGQTVTISCSGSNSNIGNNHVSWYRQLPETAPKLLIYDNNKRPSGIPDRFSGSKSGTSATLDITGLQTGDEADYYCATWDNSLSAPWVFGGGTKLTVLG

SEQ ID NO: 33 - protein, 252 aa, artificial
  phage display generated human antibody
QVQLQESGAEVKKPGSSVKVSCKASGGTFSSSAISWVRQAPGQGLEWMGGIIPVFGTANYAQKFQDRVTITADESTSTAYLELSRLTSEDTAVYYCASRGEYDYGDYDVYYYYMEVWGQGTLVTVSSGGGGSGGGGSGGGGSAQSVLTQPPSVSVAPGQTARLTCGANNIGSTSVHWYQQKPGQAPVLVIYDDTDRPSGIPERFSGSNSGNTATLTIRRVEAGDEADYYCQVWDTNSDHVIFGGGTKLTVLG

SEQ ID NO: 34 - protein, 249 aa, artificial
  phage display generated human antibody
EVQLVQSGAEVKKPGSSVKVSCQASGGTFTSHAMYWVRQAPGQGLEWMGGIIPIFGRTNYAQKFQGRVTFTADMSTSTAYMEMTSLRSDDTAVYYCARGDNWNDLYPIDYWGRGTLVTVSSGGGGSGGGGSGGGGSALNFMLTQPHSVSESPGKTVTISCTRSSGSIATTYVQWFQQRPGSSPTTVIYDDDQRPSGVPDRFSGSIDSSSNSASLTISGLMPEDEADYYCQSYDNTDLVFGGGTQLTVLS

SEQ ID NO: 35 - protein, 248 aa, artificial
  phage display generated human antibody
EVQLVQSGAEVKKPGASVKVSCKVSGYSLSELSMHWVRQAPGKGLEWMGGFDPQNGYTIYAQEFQGRITMTEDTSTDTVYMELGSLRSEDTAVYFCAAIEITGVNWYFDLWGKGTLVTVSSGGGGSGGGGSGGGGSALSSELTQDPDVSVALGQTVRITCQGDSLKKFYPGWYQQKPGQAPLLVLYGENIRPSRIPDRFSGSSSGNTATLTITGAQAEDEAVYYCNSREASVHHVRVFGGGTKLTVLG

SEQ ID NO: 36 - protein, 251 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGKGTLVTVSSGGGGSGGGGSGGGGSALNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNQGVVFGGGTKLTVLG

SEQ ID NO: 37 - protein, 251 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSALNFMLTQPHSVSESPGKTVTISCTGSSGSIASNYVQWYQQRPGSAPTTLIYEDDQRPSGVPDRFSGSVDSSSNSASLTISGLKTEDEADYYCQSYDRSNQAVVFGGGTKLTVLG

SEQ ID NO: 38 - protein, 253 aa, artificial
  phage display generated human antibody
QVQLVQSGPEVKKPGASVEVSCKASGYTFTGDYMHWVRQAPGQGPEWMGWINPQTGVTKYAQKFQGRVTMARDTSINTAYMELRGLRSDDTAVYYCVREDHNYDLWSAYNGLDVWGQGTLVTVSSGGGGSGGGGSGGGGSAQSVLTQPPSVSAAPGQKVTISCSGSSSNIGNNHVSWYQQLAGTAPKLLIFDNDKRPSGIPDRFSGSKSGTSATLGITGLQTGDEADYYCGTWDKSPTDIYVFGSGTKLTVLG

SEQ ID NO: 39 - protein, 247 aa, artificial
  phage display generated human antibody
QVQLQESGPGLVKPSGTLSLTCAVSGGSISSSNWWSWVRQAPGKGLEWIGEIYYGGSTNYNPSLKSRVTLSVDKSKNQFSLRLISVTAADTAVYYCARSSGLYGDYGNLWGRGTLVTVSSGGGGSGGGGSGGGGSAQSVVTQPPSVSAAPGQKVTISCSGSASNIGDHYISWYQQFPGTAPKLLISDNDQRPSGIPDRFSGSKSGTSATLGITGLQTGDEADYYCGTWDSNLSSWVFGSGTKVTVLG

SEQ ID NO: 40 - protein, 250 aa, artificial
  phage display generated human antibody
EVQLVQSGAEVKKPGATLKVSCKVSAYTFTDYSMHWVQQAPGKGLKWMGLIDLEDGNTIYAEEFQDRVTITADTSTDTAYMDLSSLRSEDTAVFYCAISPLRGLTADVFDVWGQGTLVTVSSGGGGSGGGGSGGGGSAQSALTQPASASGSPGQSITISCTGTSSDIGRYDFVSWYQRQPGKAPKLMIYDVINRPSGVSSRFSGSKSGNTASLTISGLQAEDEADYYCSSYAGSTTLYVFGTGTKLTVLG

SEQ ID NO: 41 - protein, 246 aa, artificial
  phage display generated human antibody
QVQLQESGPGLVKPSATLSLTCAVSGGSISSNHWWSWVRQSPGKGLEWIGEIYTYGGANYNPSLKSRVDISMDKSKNQFSLHLSSVTAADTAVYYCGRHLTGYDCFDIWGQGTLVTVSSGGGGSGGGGSGGGGSAQAVLTQPSSVSGAPGQRVTISCTGSSSNIGAGYDVHWYQQLPGTAPKLLIYGNSNRPSGVPDRFSGSKSGTSASLAITGLQAEDEADYYCQSYDSSLSGVFGTGTQLTVLS

SEQ ID NO: 42 - protein, 249 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSALNFMLTQPHSVSESPGKTVTISCTRSSGSIASKYVQWYQQRPGSAPTSVIYEDNQRPSGVPDRFSGSIDSASNSASLTISGLKTEDEADYYCQSDDGSSVVFGGGTKVTVLG

SEQ ID NO: 43 - protein, 257 aa, artificial
  phage display generated human antibody
EVQLVQSGAEVKKPGASVKVSCKASGYSFPSSGLSWVRQAPGQGPEWMGWIGIYNGNTDYAQKFQGRVTMTTDKSTSTAYMELRSLRSDDTAVYYCARDSVGSISVAGTMQYYYFAMDVWGQGTLVTVSSGGGGSGGGGSGGGGSAQSVLTQPPSASGSPGQSVTISCAGTRYDIGTYNYVSWYQQHPAKGPKLIIYAVSERPSGVPNRFSGSKSGNTASLTVSGLRAEDEAHYYCSSYAGNNNVIFGGGTKVTVLG

SEQ ID NO: 44 - protein, 247 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTMVTVSSGGGGSGGGGSGGGGSAQSVLTQPPSASGTPGQRVTISCSGSFSNIGGNYVNWYQQLPGTAPKLLIYGNNQRPSGVPDRFSSFKSGTSASLAISGLRSEDEADYYCATWDDSQTVLFGGGTKLTVLG

SEQ ID NO: 45 - protein, 246 aa, artificial
  phage display generated human antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARWNGFLTAHDSWGRGTMVTVSSGGGGSGGGGSGGGGSAQSVLTQPPSASGTPGQRVTISCSGSSSNIGTNYVYWYQQFPGTAPKLLIYRSNRRPSGVPDRFSASKSGTSASLVISGLRSEDEADYYCAAWDDRLNGEMFGGGTKVTVLG

SEQ ID NO: 46 - protein, 243 aa, artificial
  phage display generated human antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARWSGRFYDFWGQGTTVTVSSGGGGSGGGGSGGGGSAQSVLTQPPSASGTPGQRITISCSGSSSNIGSNYVYWYQQLPGTAPKILIYRNNQRPSGVPERFSGSKSGTSASLAISGLRSEDEADYYCAAWDDSLSEVFGGGTKVTVLG

SEQ ID NO: 47 - protein, 246 aa, artificial
  phage display generated human antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDKGYSGFDYWGRGTLVTVSSGGGGSGGGGSGGGGSAQSVLTQPPSASGTPGQRVTISCSGSSSNIGRHTVNWYQQLPGTAPKLLIYSNNQRPSGVPDRFSGSKSGTSASLAISGLQSEDEGHYHCAAWDDTLNGDVVFGGGTKVTVLG

SEQ ID NO: 48 - protein, 251 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGKGTLVTVSSGGGGSGGGGSGGGGSALNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNPYVVFGGGTKLTVLG

SEQ ID NO: 49 - protein, 251 aa, artificial; identical to SEQ ID NO: 25
  phage display generated human antibody

SEQ ID NO: 50 - protein, 248 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSAQSVVTQPPSVSAAPGQKVTISCSGSSSNIGNNYVSWYKQLPGTAPKLLIYDNNKRPSGIPDRFSGSKSGTSATLGITGLQTGDEADYYCGTWDSSLSGVVFGGGTKLTVLG

SEQ ID NO: 51 - protein, 251 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSSGGGGSGGGGSGGGGSALNFMLTQPHSVSESPGKTVTISCTRSSGSIASNYVQWYQQRPGSSPTTLIYDDNQRPSGVPDRFSGSIDSSSNSASLTISGLKTEDEADYYCQSYDSSNLGVVFGGGTKLTVLG

SEQ ID NO: 52 - protein, 250 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSSGGGGSGGGGSGGGGSALNFMLTQPHSVSESPGKTATISCTGSGGSIARSYVQWYQQRPGRAPSIVIYEDYQRPSGVPDRFSGSIDSSSNSASLTITGLKTDDEADYYCQSSDDNNNVVFGGGTKVTVLG

SEQ ID NO: 53 - protein, 248 aa, artificial
  phage display generated human antibody
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSSGGGGSGGGGSGGGGSAQAVLTQPSSVSAAPGQKVTISCSGSSSNIGNNYVSWYQQLPGTAPKLLIYDNNERPSGIPDRFSGSKSGTSATLGITGLQTGDEADYYCGTWDSSLSTVVFGTGTKVTVLG

SEQ ID NO: 54 - protein, 249 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSALNFMLTQPHSVSESPGKTVTVSCTGSGGNIASNYVQWYQQRPDSAPTLVIFEDTQRPSGVPARFSGSIDSSSNSASLIISSLRTEDEADYYCQSSDSNRVVFGGGTKVTVLG

SEQ ID NO: 55 - protein, 241 aa, artificial
  phage display generated human antibody
QVQLQESGPGLVKPSETLSLTCNVSGGSIRNYFWSWIRQPPGQGLEYIGYIYYSGTTDYNPSLKGRVTISLDTSKTQFSLKLNSVTAADTAFYYCVRGPNKYAFDPWGQGTLVTVSSGGGGSGGGGSGGGGSALSYELTQPPSVSVSPGQTASITCSGDKLGDKFASWYQQKAGQSPVLVIYRDTKRPSGIPERFSGSNSGNTATLTISGTQAMDEADYYCQAWDSSTAVFGTGTKVTVLG

SEQ ID NO: 56 - protein, 251 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGQGTLVTVSSGGGGSGGGGSGGGGSALNFMLTQPHSVSESPGKTVTISCTRSSGSIDNNYVQWYQQRPGSSPTTVIFEDNQRPSGVPDRFSGSIDSSSNSASLTISGLKTEDEADYYCQSYDSHNQGVVFGGGTKLTVLG

SEQ ID NO: 57 - protein, 248 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSSGGGGSGGGGSGGGGSAQSVLTQPPSVSAAPGQKVTISCSGSSSNIGNSYVSWYKQLPGTAPKVLIYDNQKRSSGIPDRFSASKSGTSATLGITGLRTEDEADYYCGTWDTSLSAVVFGGGTKLTVLG

SEQ ID NO: 58 - protein, 248 aa, artificial
  phage display generated human antibody
EVQLVESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSSGGGGSGGGGSGGGGSAQSVVTQPPSVSAAPGQKVTISCSGNFSNIEYNYVSWYQHLPGTAPKLLIFDNNQRPSWIPDRFSGSKSGTSATLGITGLQTGDEADYYCGTWDSSLNAGVFGGGTKVTVLG

SEQ ID NO: 59 - protein, 245 aa, artificial
  phage display generated human antibody
EVQLLESGGGLVRPGGSLRLSCAASGFTFSSYAMSWVRQAPGKGLEWVSAISGSGGSTYYADSVKGRFTISRDNSKNTLYLQMNSLRAEDTAVYYCAKDRRGVLDPWGKGTMVTVSSGGGGSGGGGSGGGGSAQSVLTQPPSVSGAPGQRVTISCTGSSSNIGAGYDVHWYQHLPGTAPRLLIYGNSNRPSGVPDRFSGSKSGTSASLAISGLQAEDEADYYCQSYDSSLSDWVFGGGTKVTVLG

SEQ ID NO: 60 - protein, 250 aa, artificial
  phage display generated human antibody
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSSGGGGSGGGGSGGGGSALNFMLTQPHSVSESPGKTVTISCARSSGSIASNYVQWYQQRPGSSPTTLIYEDRQRPSGVPDRFSGSIDSSSNSASLTISGLKTEDEADYYCQSYDSSDHVVFGGGTKLTVLG

SEQ ID NO: 61 - DNA, 741 bp, artificial
  phage display generated human antibody
gaggtgcagctgttggagtctgggcgaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagatttgccgtaactggggagtttgactactgggggcaggggaccacggtcaccgtctcgagtggaggcggcggttcaggcggaggtggctctggcggtggcggaagtgcacaggctgtgctgactcagccgtcctcagtgtctggggccccagggcagagggtcaccatctcctgcactgggagcagctccaacatcggggcagattatgatgtacactggtaccagcagcttccaggaacagcccccaaactcctcatctatggtaacaacaatcggccctcaggggtccctgaccgattctctggctccaagtctggcacctcagcctccctggccatcactgggctccaggctgaggatgaggctgattattactgccagtcctatgacaacagcccggatgcctatgtggtcttcggcggagggaccaagctgaccgtcctaagt

SEQ ID NO: 62 - DNA, 732 bp, artificial
  phage display generated human antibody
caggtgcagctggtgcagtctggggctgaggtgagaaagcctggggcctcagtgaaggtctcctgcaagacttctggatacaccttcatcgactactatatacactgggtgcgacaggcccctggacaagggcttgagtggatgggctgggtcaaccctgtcactggaacctcaggctcttcacccaactttcggggcagggtcaccatgaccaccgacacgtccggcaacacagcctatatggaactgaggagccttagatctgacgacacggccgtattttactgtgcgaggcgtcaccaacagagcttggattattggggccagggaaccctggtcaccgtctcgagtggaggcggcggttcaggcggaggtggctctggcggtggcggaagtgcacagtctgtgttgacgcagccgccctcagtgtctgcgcccccgggacagaaggtcaccatctcctgctctggaagcagctccaacattgggactaattatgtatcctggtaccagcagctcccaggaacagcccccaaactcctcatttatgacaatcataagcgaccctcagtgattcctgaccgcttctctggctccaagtctggcacgtcagccaccctgggcatctccggactccagactggggacgaggccgattattactgcggaacatgggattacagcctgagtacttgggtgttcggcggagggaccaagctgaccgtcctaggt

SEQ ID NO: 63 - DNA, 720 bp, artificial
  phage display generated human antibody
cagttgcagctgcaggagtccggcccaggactggtgaagccttcggggaccctgtccctcacctgcgctgtctctggagactccgtcagcagttattactggtggagttgggtccgccagcccccagggaaggggctggagtggattggagaaatctttcgtgatgggagctccaactacaaccggtccctcaagagtcgggtcaccatatccccagacaagcccaagaatcagttctctctgaggctgagctctgtgaccgccgcggacacggccatttactactgtgcgaggcatatacgcggttatgatgcttttgacatctggggccggggaaccctggtcaccgtctcgagtggaggcggcggttcaggcggaggtggctctggcggtggcggaagtgcacagtctgtgttgacgcagccgccctcagtgtctggggccccagggcagagggtcaccatctcctgtactgggagcagctccaacatcggggcaggttatgatgtacactggtaccagcagtttccaggaagagcccccaagctcctcatctatggtaacaccaatcggccctcaggggtccctgaccgattctctggctccaagtctgacatctcagcctccctggccatcactgggctccaggctgaggatgaggctgattattactgtcagtcctatgacagcaacctgactggggtgttcggcggagggacc

SEQ ID NO: 64 - DNA, 732 bp, artificial
  phage display generated human antibody
caggtgcagctggtgcagtctggggctgaggtgaggaagcctggggcctcagtgaaggtctcctgcaagacttctggatacaccttcatggactactacatacactgggtgcgacaggcccctggacaagggcttgagtggatgggctggagcaaccctgtcactggtacgtcaggctcttcacctaaatttcggggcagggtcaccttgaccactgacacgtccggcaacacagcctatttggacctgaggagccttagatctgacgacacggccgtattttactgtgcgaggcgtcaccaacagagcttggattattggggccaagggacaatggtcaccgtctcgagtggaggcggcggttcaggcggaggtggctctggcggtggcggaagtgcacagtctgtgttgacgcagccgccctcagtgtctgcggccccaggacagaaggtcaccatctcctgctctggaagcagctccaacattgggaataattatgtatcctggtaccagcaactcccaggaacagcccccaaactcctcatgtatgaaaatagtaagcgaccctcagggattcctgaccggttctctggctccaagtctggcacgtcaggcaccctgggcatcaccggactccagactggggacgaggccgattattactgcggaacatgggataccagcctgagagcttgggtgttcggcggagggaccaaggtcaccgtcctaggt

SEQ ID NO: 65 - DNA, 732 bp, artificial
  phage display generated human antibody
caggtacagctgcagcagtcaggggctgaggtgaggaagcctggggcctcggcgaaggtctcctgcaagacttctggatacaccttcatcgactactatatacactgggtgcgacaggcccctggacaagggcttgagtggatgggctggatcaaccctgtcactggtgcctcaggctcttcacctaactttcggggcagggtcaccttgaccaccgacacgtccggcaacacagcctatatggagctgaggagccttagatctgacgacacggccgtgttttactgtgcgaggcgtcaccaacagagcttggattattgggggcgggggaccacggtcaccgtctcgagtggaggcggcggttcaggcggaggtggctctggcggtggcggaagtgcacagtctgtcgtgacgcagccgccctcagtgtctgcggctccaggacagaaggtcaccatctcctgctctgggaggacatccaacattgggaacaattatgtatcctggtatcagcaagtcccaggagcgccccccaaactactcatttttgacaataataagcgaccctcagggactcctgcccgattctctggctccaagtctggcacgtcagccaccctggccatctccggactccagaccggggacgaggccgattattactgcggaacatgggatactaccctgcgtggttttgtcttcgggcccgggaccaaggtcaccgtcctaggt

SEQ ID NO: 66 - DNA, 750 bp, artificial
  phage display generated human antibody
cagctgcagctgcaggagtcgggcccaggactggtgaagccttcggggaccctgtccctcacctgcgctgtctctggtggctccatcagcagtactaactggtggagttgggtccgccagcccccagggaaggggctggagtggattggggaaatctatcatagtgggagcaccaactacaacccgtccctcaagagtcgagtcaccatatcagtagacaagtccaagaaccacttctccctgaacctgagctctgtgaccgccgcggacacggccgtgtattactgtgcgagagattctatgggaagcactggctggcattacggtatggacctctggggccggggaaccctggtcaccgtctcgagtggaggcggcggttcaggcggaggtggctctggcggtggcggaagtgcacaatctgccctgactcagcctccctccgcgtccgggtctcctggacagtcagtcaccatctcctgcagtggaagcagtagtgacattggtgattataaccatgtctcctggtaccaacagcacccaggcaaagcccccaaactcatgatttatgacgtcaataagtggccctcaggggtccctgatcgcttctctggctccaagtctggcaacacggcctccctgaccgtctctgggctccaggctgaggatgaggctgattattattgcagctcatattcaggcatctacaatttggttttcggcggagggaccaaggtcaccgtcctaggt

SEQ ID NO: 67 - DNA, 753 bp, artificial
  phage display generated human antibody
gaggtgcagctggtgcagtctggggctgaagtgaagaagcctgggtcctcggtgaaggtctcctgtaaggcctctggaggcaccttcaagacctatgctatcaattgggtgcgacaggcccctggacaagggcttgagtggatgggaggaatcatccctgtcctgggaacagcaaattacgttcagaagttccagggcagagtcacgattaccgcggacgaatcgacgaccacagcctacatggagctgaggggcctgagatctgaggacacggccgtttattattgtgcgagaggagagggcagtggctggtacgatcactactacggattggacgtctggggccaaggaaccctggtcaccgtctcgagtggaggcggcggttcaggcggaggtggctctggcggtggcggaagtgcacagtctgtgctgacgcagccgccctcagcgtctgggacccccgggcagagggtcaccatctcttgttctggaagcagctccaacatcggaagtaatactgtaaactggtaccggcagctcccaggaacggcccccaaactcctcatctttggtgatgatcagcggccctcaggggtccctgaccgattctctggctccaggtctggcacctcagtctccctggccatcagtgggctccagtctgaggatgaggctgactattactgtgcagcatgggatgacagcctgaatggcggggtgttcggcggagggaccaagctgaccgtcctaggt

SEQ ID NO: 68 - DNA, 750 bp, artificial
  phage display generated human antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaagatcattactatgatagtagtggttatcttgactactggggccaaggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgccttcgactatgtgcagtggtaccagcagcgcccgggcagtgcccccaccactgtgatctatgaggataatcaaagaccctctggggtccctgatcggttctctgcctccatcgacagctcctccaactctgcctccctcaccatctctgcactgaagactgaggacgaggctgactactactgtcagtcttatgataacagcaattcttgggtcttcggcggagggaccaagctgaccgtcctaggt

SEQ ID NO: 69 - DNA, 726 bp, artificial
  phage display generated human antibody
aaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaagatgatgttcggaatgcttttgatatctgggggagggggaccacggtcaccgtctcgagtggaggcggcggttcaggcggaggtggctctggcggtggcggaagtgcacagtctgtgctgactcagccaccctcagtgtccgtgtccccaggacagacaaccagcatcacctgctctagagataagttgggagaacaatatgtttactggtatcaacagaggccaggccagtcccctattctactcctctatcaagattccaggcggccctcatggatccctgagcgattctctggctccaactctggggacacagccactctgaccatcagcgggacccaggctctggatgaggctgactactactgtcaggcgtgggacaacagttcctatgtagcattcggcggagggaccaaggtcaccgtcctaggt

SEQ ID NO: 70 - DNA, 735 bp, artificial
  phage display generated human antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaggggagctgtggaatccatatttagactactggggccagggcaccctggtcaccgtctcgagtggaggcggcggttcaggcggaggtggctctggcggtggcggaagtgcactgcctgtgctgactcagcccccctcagtgtcagtggccccagggaagacggccaggattacctgtgggggaaacgacattgcaagtaaaagtgtgcagtggtttcagcagaagccaggccaggcccctgtactggtcatctattatgatagcgaccggccctcagggatccctgagcgattctctggctccaactctgagaacacggccaccctgaccatcagcagggtcgaagcgggggatgaggccgactattattgtcaggtgtgggatagcagtagtgatcatccggtgttcggcggagggaccaagctgaccgtcctaggt

SEQ ID NO: 71 - DNA, 750 bp, artificial
  phage display generated human antibody
caggtccagctggtgcagtctggggcagaggtgaaaaagcccggggagtctctgaaaatctcctgtaagggttctggatacacttttaccaattactggatcgcctgggtgcgccagatgcccggaaaaggcctggagtggatgggaatcatttatcctgatgactctgataccagatacaacccgtccttccaaggccaggtcaccatgtcagccgacaagtccatcgacaccgcctatctgcagtggagcagcctgaaggcctcggacaccgccatatattactgtgcgagaccctcgggctggaacgacaatggctactttgactactgggggcgagggaccacggtcaccgtctcgagtggaggcggcggttcaggcggaggtggctctggcggtggcggaagtgcacttaattttatgctgactcagccccactctgtgtcggcgtctccggggaagacggtcaccctctcctgcaccggctccagtggcagcattgccagcaactatgtgcagtggtaccggcagcgcccgggcagtgcccccaccactgtgatctatgacgataatcaaagaccctctggggtccctgatcgtttctctggctccatcgacagctcctccaactctgcctccctcaccatctctggactgaagactgaggacgaggctgactactactgtcagtcttttgataacgacaatcattgggtgtttggcggagggaccaagctgaccgtcctaggt

SEQ ID NO: 72 - DNA, 741 bp, artificial
  phage display generated human antibody
caggtgcagctgcaggagtcgggcccaggactggtgaggtcttcggggatcctgtccctcacctgctctgtctctggtgtctccgtcagcagtaataactggtggagttgggtccgccagaccccagggaaggggctggagtggatcggggaaatctatcagaccgggaccaccaactacaacccgtctctcaagagccgagtcgccatatcactagacaagtccaggaatcagttctccctgattttgaagtctgtgaccgccgcggacacggccgtatattactgcgcgagaactagcagcgcctggtctaacgctgattggggcaaagggacaatggtcaccgtctcgagtggaggcggcggttcaggcggaggtggctctggcggtggcggaagtgcactttcttctgagctgactcaggacccctccgcgtccgggtctcctggacagtcagtcagcatctcttgcactggaaccagcagtgacgttggtggttataattatgtctcctggtaccaacagcacccaggcaaagcccccaaactcatgatttctgaggtcactaagcggccctcaggggtccctgatcgcttctctggctccaagtctggcaacacggcctccctgaccgtctctgggctccaggctgaagatgaggctgattattactgcagctcatttggagccaacaacaattatctcgtattcggcggagggaccaagctgaccgtcctaggt

SEQ ID NO: 73 - DNA, 753 bp, artificial
  phage display generated human antibody
caggtgcagctgcaggagtcgggcccaagactggtgaagccttcacagaccctgtccctcacctgcactgtctctaatgactccatcatcagtggcgattacttctggagttggatccgccagcccccagggaagggcctggagtggattgggaacatcttttatactgggagcacctcttacaatccgtccctcaagagtcgacttaccatgtccctagacacgtccaagaaccagttctccctgagattgagctctgtgactgccgcagacacggccgtatatttttgtgccagaggtcgacaggggatgaactggaattccgggacctacttcgactcctggggcagaggaaccctggtcaccgtctcgagtggaggcggcggttcaggcggaggtggctctggcggtggcggaagtgcactttcctatgtgctgactcagccaccctctgtgtccgtggccccaggaaagacggccaatataacttgtgggggaaagaacattggaaataaaagtgtgcagtggtatcagcagaagccaggccaggcccctgtggtagtcatgtattatgacagcgaccggccctcagggattcctgagcgattctctggctccaacgctgggaacacggccaccctgaccatcgacagggtcgaggccggggatgaggccgattattactgtcaggtgtgggataaaagtagtgatcgtccggtcttcggcggagggaccaagctgaccgtcctaggt

SEQ ID NO: 74 - DNA, 735 bp, artificial
  phage display generated human antibody
caggtccagctggtgcagtctggggctgaggtgaagaagcctggggcctcagtgaaggtctcctgcaagacttctggatacaccttcatggaatactacatacactgggtgcgacaggcccctggacaagggcttgagtggatgggctggagcaatcctgtcactggtacgtcaggctcttcacctaagtttcggggcagggtcaccttgaccactgacacgtccggcaacacagcctatttggacctgaggagccttagatctgacgacacggccgttttttactgcgcgaggcgtcatcaacagagcttggattattggggccaaggcaccctggtcaccgtctcgagtggaggcggcggttcaggcggaggtggctctggcggtggcggaagtgcacagtctgtcgtgacgcagccgccctccgcgtccgggtctcctggacagtcagtcaccatctcctgctctggatacagctcctccaacatcgggaataatgctgtctcctggtaccaacaactcccaggaacagcccccaaactcctcatttttgacaataataagcgaccctcagggattcctgcccgattctctggctcccagtctggcacgacagccaccctgggcatcaccggactccagactggggacgaggccgattatttctgcggaacatgggatagcagcctgagtgcttttgtcttcggatccgggaccaaggtcaccgtcctaggt

SEQ ID NO: 75 - DNA, 744 bp, artificial
  phage display generated human antibody
atggccgaggtgcagctggtgcagtctggggctgaggtgaagaagcctgggtcctcggtgaaggtctcctgcaaggcttctggaggcagcttcagcaactatgatttcagttgggtgcggcaggcccccggacaagggcttgagtggatgggagagatcatcaatgcctttggttcatcaagatacgcacagaaattccaggacagagtcaccattaccgcggacgaatccgcgagcacagcctacatggaactaagaggcctgacatctgaggacacggccacttattactgtgcgagggcggaaaggtgggaacttaatatggcttttgatatgtggggcagaggaaccctggtcaccgtctcgagtggaggcggcggttcaggcggaggtggctctggcggtggcggaagtgcacagtctgtgctgactcagccaccctcggtgtcagtggccccagggcagacggccaggatcacctgtgggggagacaatatagggagaaaaaatgtccactggtaccagcagcggccaggcctggcccctgttttagtcgtctatgatgacaccgaccggccctcagggatccctgagcgattctctggctccaactctggggacacggccaccctgaccatcacctgggtcgaggccggggatgaagccgactattactgtcaactttgggatagtgacacctatgatgttttattcggcggagggaccaagctgaccgtcctaggt

SEQ ID NO: 76 - DNA, 741 bp, artificial
  phage display generated human antibody
gaggtgcagctggtgcagtctggggctgaggtgaagaagcctgggtcctccgtgaaggtctcctgcaagtcttctggaggccccttcagcagctatggtatcagctgggtgcgacaggcccccggacaagggcttgagtggatgggagggatcagccctatctttggtacagcaaactacgcacagaagttccagggcagagtcaccattaccgcggacgaatccacagagacagcctacatggagctgagtagcctgaggtctgaggacacggccgtgtattactgtgcgagagacgagtcaccggtcgggttttatgctttggatatctgggggcgagggaccacggtcaccgtctcgagtggaggcggcggttcaggcggaggtggctctggcggtggcggaagtgcactttcctatgagctgactcagccaccctcggtgtcagtggccccaggacagacggccaggattaactgtgggggagacaaaattggaagtagaagtgtacactggtaccagcagaagccaggccaggcccctgtgatggtcgtctatgatgatagcgaccggccctcagggatccctgagcgattctctggctccaactctgggaacacggcaaccctgaccatcagcagtgtcgaagccggggatgaggccgactattattgtcaggtgtgggatggtagtactgatccctgggtattcggcggagggaccaaggtcaccgtcctaggt

SEQ ID NO: 77 - DNA, 765 bp, artificial
  phage display generated human antibody
gaagtgcagctggtgcagtctggggctgagatgaagaagcctgggtcctcggtgaaggtctcctgcaaggcatctggaggcaccttcagcagctatgctgtcaactgggtgcgacaggcccctggacaagggcttgaatggatgggaggaatcatccctatttttgatacttcgaactacgcacagaagttccagggcagactcacgatgaccgcggacgactccacgaacacagcctacatggaactgaggagcctgagatctgaggacacggccgtatattactgtgcgagaggggccccgaggggaacagttatggcattcagctcttactactttgacttatggggccagggcaccctggtcaccgtctcgagtggaggcggcggttcaggcggaggtggctctggcggtggcggaagtgcacttaattttatgctgactcagccccactctgtgtcggagtctccggggaagacagtaattatctcctgcgccggcagcggtggcaacattgccaccaactatgtgcagtggtaccaacatcgcccgggcagtgcccccattactgtgatctatgaggataatcaaagaccctctggagtccctgatcgcttctctggctccgtcgacagctcctccaactctgcctccctcaccatctctggactgcagactgaggacgaagctgactactactgtcactcttatgacaacaccgatcagggggtcttcggaactgggaccaaggtcaccgtcctaggt

SEQ ID NO: 78 - DNA, 759 bp, artificial
  phage display generated human antibody
gaggtgcagctggtggagtccgggggaggcttggtacagcctggcaggtccctgagactctcctgtgcagcctctggattcacctttgatgattacgacatgcactgggtccggcaagctccagggaagggcctggagtgggtctcaagtattagttggagtggtggaactatagggtatgcggactctgtgaagggccgattcaccgtctccagagacaacgccaagaactccctgtatctgcaaatgaacagtgtgagagctgaggacacggccttatattactgtgcaaaagacaggggcgctgtagcagctctccccgactatcagtacggtatggacgtctggggcaggggcaccctggtcaccgtctcgagtggaggcggcggttcaggcggaggtggctctggcggtggcggaagtgcacagtctgccctgactcagcctgcctccgtgtctgggtctcctggacagtcgatcaccatctcctgcactggaaccagcagtgatattgggagttataaccttgtctcctggtaccaacaacacccaggcaaagcccccaaactcatgatttatgaggactataagcgggcctcaggggtttctaatcacttctctggctccaagtctggcaacacggcctccctgacaatctctgggctccaggctgaggacgaggctgattattactgctcctcatatgcaggtagtagcgcttgggtgttcggcggagggaccaaggtcaccgtcctaggt

SEQ ID NO: 79 - DNA, 735 bp, artificial
  phage display generated human antibody
gaagtgcagctggtgcagtctggggctgaggtgaggaagcctggatcctcgatgaaggtctcctgcaaggcctctggcgacaccttcaggaactttgctttcagttgggtgcgacaggcccctggacaaggacttgaatggatggggggagtcatccctttggttggtccaccaaagtacgctcagaagttccagggcagactcaccattaccgcggacgagtccacgagcacctcctacatggacttgaccagcctgacactcgaagacacggccgtctatttctgtgcgcgagggggggtttatgctccctttgacaaatggggccaaggaaccctggtcaccgtctcgagtggaggcggcggttcaggcggaggtggctctggcggtggcggaagtgcacagtctgtcgtgacgcagccgccctcagtgtctgaagcccccaggcagagggtcaccatctcctgttctggaagcagctccaacatcggaaataatgctgtaaactggtaccagcagctcccaggaaaggctcccaaactcctcatctattataatgatctgctgccctcaggggtctctgaccgattctctggctccaagtctggcacctcagcctccctggccatcagtgggctccagtctgaggatgaggctgattattactgtgcagcatgggatgacagcctgaatggctgggtgttcggcggagggaccaaggtcaccgtcctaggt

SEQ ID NO: 80 - DNA, 753 bp, artificial; identical to SEQ ID NO: 67
  phage display generated human antibody

SEQ ID NO: 81 - DNA, 744 bp, artificial
  phage display generated human antibody
cagctgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggcaaaggaaccctggtcaccgtctcgagtggaggcggcggttcaggcggaggtggctctggcggtggcggaagtgcacaggctgtgctgactcagccgtcctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaggtctggcacgtcagccaccctgggcatcaccggactccagactggggacgaggccgattattactgcggaacatgggatagcagcctgagtgctgtagtcttcggaactgggaccaagctgaccgtcctaggt

SEQ ID NO: 82 - DNA, 750 bp, artificial
  phage display generated human antibody
cagctgcagctgcaggagtcgggcccaggactggtgaagccttcggggaccctgtccctcacctgcgctgtctctggtggctccatcagcagtactaactggtggagttgggtccgccagcccccagggaaggggctggagtggattggggaaatctatcatagtgggagcaccaactacaacccgtccctcaagagtcgagtcaccatatcagtagacaagtccaagaaccacttctccctgaacctgagctctgtgaccgccgcggacacggccgtgtattactgtgcgagagattctatgggaagcactggctggcattacggtatggacctctggggcaaaggcaccctggtcaccgtctcgagtggaggcggcggttcaggcggaggtggctctggcggtggcggaagtgcacagtctgccctgactcagcctgcctccgtgtctgggtctcctggacagtcgatcgccatctcctgcactggaaccagcagtgacgttggtggttataactatgtctcgtggtaccaacagcacccaggcaaagcccccaaactcatgatttatgctgtcactaatcggccctcaggggtttctgatcgcttctctggctccaagtctggcaacacggcctccctgaccatctctgggctccaggctgacgacgaggctgattattactgcagctcatatacaagcagcagctctctggtgttcggcggagggaccaagctgaccgtcctaggt

SEQ ID NO: 83 - DNA, 720 bp, artificial
  phage display generated human antibody
ggggtgcagctggtggagtctgggggaggcctggtcaagcctggggggtccctgagactctcctgtgcagcctctggattcaccttcagtagttataccatgaactgggtccgccaggctccagggaaggggctggagtgggtttcatacattagtagtagtggtagtgccacatactacgcagactctgtgaagggccgattcaccatctccagggacaacgccaacaactcactgtatctgcaaatgaacagcctgagagccgaggacacggccgtgtattactgtgcgagagggtaccgctacggcatggacgtctggggccaaggaaccctggtcaccgtctcgagtggtggaggcggttcaggcggaggtggcagcggcggtggcggatcgggcatcgtgatgacccagtctccttccaccctgtctgcatctgtaggagacagagtcaccatcacttgccgggccagtcagggtattagtagctggttggcctggtatcagcagaaaccagggagagcccctaaggtcttgatctataaggcatctactttagaaagtggggtcccatcaaggttcagcggcagtggatctgggacagatttcactctcaccatcagcagtctgcaacctgaagattttgcaacttactactgtcaacagagttacagtaccccgtggacgttcggccaagggaccaagctggagatcaaacgt

SEQ ID NO: 84 - DNA, 735 bp, artificial
  phage display generated human antibody
gaggtgcagctgttggagtctgggggaggcttggtacagcctggggggtccctgagactcacctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagatttagcagtggcaggtattgactactggggccgggggacaatggtcaccgtctcgagtggaggcggcggttcaggcggaggtggctctggcggtggcggaagtgcacagtctgtgctgacgcagccgccctcagcgtctgggacccccgggcagagggtcaccatatcttgttctgggagcagttccaacatcagaagtaattatgtttactggtaccagcagttcccaggaacggcccccaaactcctcatttatagaaataatcagcggccctcaggggtccctgaccgattctctggctccaagtctggcacctcagcctccctggccatcagtgggctccggtccgaggatgaggctgattattattgtgcagcatgggatgacaccctggatgcttatgtcttcgcagctgggaccaagctgaccgtcctaggt

SEQ ID NO: 85 - DNA, 753 bp, artificial
  phage display generated human antibody
caggtgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttacccccttgactactggggccagggcaccctggtcaccgtctcgagtggaggcggcggttcaggcggaggtggctctggcggtggcggaagtgcacttaattttatgctgactcagccccactctgtgtcggggtctccggggaggacggtaaccatctcctgcacccgcagcagtggcagcattgccaccaactatgtgcagtggtaccagcagcgcccgggcagttcccccaccattgtgatctatgaagataaccaaagaccctctggggtccctgatcgcttctctggctccatcgacacctcctccaactctgcctccctcaccatctctggactgaagactgaggacgaggctgactactactgtcagtcttatgatagcaacaatctgggggtggtatttggcggagggacccagctcaccgttttaagt

SEQ ID NO: 86 - DNA, 747 bp, artificial
  phage display generated human antibody
caggtacagctgcagcagtcaggggctgaggtgaggaagcctggggcctcagtgaagatctcctgcaagacttctggatacaccttcatggactactacatacactgggtgcgacaggcccctggacaagggcttgagtggatgggctggagcaaccctgtcactggtacgtcaggctcttcacctaaatttcggggcagggtcaccttgaccactgacacgtccggcaacacagcctatttggacctgaggagccttagatctgacgacacggccgtattttactgtgcgaggcgtcaccaacagagcttggattattggggccaaggcaccctggtcaccgtctcgagtggaggcggcggttcaggcggaggtggctctggcggtggcggaagtgcacaggctgtgctgactcagccgtcttccctctctgcatctcctggagcatcagccagtctcacctgcaccttacgcagtgacatcaatgttggttcctacagtataaactggtaccagcagaagccagggagtcctccccaatatctcctgaactacagatcagactcagataagcagcagggctctggagtccccagccgcttctctggatcgaaggatgcttcggccaatgcagggattttactcatctctggtctccagtctgaggatgaggctgactattactgtatgatttggtacaggaccgcttgggtgttcggcggagggaccaaggtcaccgtcctaggt

SEQ ID NO: 87 - DNA, 732 bp, artificial
  phage display generated human antibody
caggtccagctggtacagtctggagctgaggtgaggaagcctggggcctcagtgaaggtctcctgcaagacttctggatacaccttcatcgaatactacatacactgggtgcgacaggcccctggacaagggcttgagtggatgggctggagcaaccctgtcactggtacgtcaggctcttcacctaagtttcggggcagggtcaccttgaccactgacacgtccggcaacacagcctatttggacctgaggagccttagatctgacgacacggccgtcttttactgtgcgaggcgtcaccaacagagcttggattattgggggcgggggaccacggtcaccgtctcgagtggaggcggcggttcaggcggaggtggctctggcggtggcggaagtgcacagtctgtgctgacgcagccgccctcagtgtctgcggccccaggacagaaggtcaccatctcctgctctggaaccaactccaacattggaaattattatgtatcttggtaccagcaactcccaggaacagcccccaaactcctcatttatgacaataataagcgaccctcaggggtccctgaccgattctctggctccaagtctggcacctcagcctccctggtcatcagtgggctccggtccgaggatgaggctgattattactgtgcagcatgggatggcagcctgactgcttgggtgttcggcggagggaccaaggtcaccgtcctaggt

SEQ ID NO: 88 - DNA, 750 bp, artificial
  phage display generated human antibody
caggtgcagctgcaggagtccggcccaggactggtgaagccttcggggaccctgtccctcacctgcgctgtctctggtgactccatcagcagtagtaactggtggacttgggtccgccagcccccagggaaggggctggagtggattggggaaatctttcatagtgggaccaccaactacaacccgtccctcaacaatcgagtcaccatatcactagacgagtccaggaaccagttctccctggagttgagctctgtgaccgccgcggacacggccatatattactgtgcgagagattcggggaattacgatgataatagaggctacgactactggggccgaggcaccctggtcaccgtctcgagtggaggcggcggttcaggcggaggtggctctggcggtggcggaagtgcacagtctgtgttgacgcagccgccctcagtgtctggggccccagggcagagggtcaccatctcctgcgctgggaccagctccaacatcggggcaggttttgatgtacactggtaccagcttcttccaggaagagcccccaaactcctcatctatggtaacaacaatcggccctcaggggtccctgaccgattctctggctccaagtctggcacctcagcctccctggccatcagtggtctccagtctgaggacgagggtgactattactgtgcagcttgggatgacaccgtgggtggtccggtgttcggcggagggaccaagctgaccgtcctaggt

SEQ ID NO: 89 - DNA, 750 bp, artificial
  phage display generated human antibody
caggtgcagctgcaggagtcgggcccaggactggtgaagccttcggggaccctgtccctcacctgcgctgtctctggtggctccatcagcagtactaactggtggagttgggtccgccagcccccagggaaggggctggagtggattggggaaatctatcatagtgggagcaccaactacaacccgtccctcaagagtcgagtcaccatatcagtagacaagtccaagaaccacttctccctgaacctgagctctgtgaccgccgcggacacggccgtgtattactgtgcgagagattctatgggaagcactggctggcattacggtatggacctctggggcaggggaaccctggtcaccgtctcgagtggaggcggcggttcaggcggaggtggctctggcggtggcggaagtgcacagtctgccctgactcagcctgccgccgtgtctgggtctcctggacagtcgatcaccatctcctgcactggatccagcagtgacgttggtggttataactatgtctcctggtaccaacaacacccaggcaaggcccccaaactcttgatttatgatgtcagtgatcggccctcaggggtctcttatcgcttctctggctccaagtctggcaacacggcctccctgaccatctctgggctccaggctgaggacgaggctgattattactgcagctcatatacagccaccggcactctggtattcggcggagggaccaagctgaccgtcctaggt

SEQ ID NO: 90 - DNA, 753 bp, artificial
  phage display generated human antibody
caggtgcagctgcaggagtcgggcccaggactggtgaagccttcggggaccctgtccctcacctgcgctgtctctggtggctccatcagcagtactaactggtggagttgggtccgccagcccccagggaaggggctggagtggattggggaaatctatcatagtgggagcaccaactacaacccgtccctcaagagtcgagtcaccatatcagtagacaagtccaagaaccacttctccctgaacctgagctctgtgaccgccgcggacacggccgtgtattactgtgcgagagattctatgggaagcactggctggcattacggtatggacctctgggggcaggggaccacggtcaccgtctcgagtggaggcggcggttcaggcggaggtggctctggcggtggcggaagtgcacagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggcccttaggggtttctaatcgcttctctggctccaagtctggcaacacggcctccctgaccatctctgggctccaggctgaggacgagggtgattattactgcagctcatatacaagcagcaccactcttatagtattcggcggagggaccaagctgaccgtcctaggt

SEQ ID NO: 91 - DNA, 744 bp, artificial
  phage display generated human antibody
caggtgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccaaggcaccctggtcaccgtctcgagtggaggcggcggttcaggcggaggtggctctggcggtggcggaagtgcacagtctgtgctgactcagccaccctcagtgtctgggaccaccgggcagagggtcatcctctcttgttctggaggaaactccaacatcggatataattctgtaaactggtaccagcagctcccaggaacggcccccaaactcctcatctatactgatgatcagcggccctcaggggtccctgaccgtttctctggctccaggtctggcacctcagcctccctggccatcagtgggctccagtctgaggatgaggctgattattactgtgcaacatgggatgactccctgaatgccggggtgttcggcggcgggaccaagctgaccgtcctaggt

SEQ ID NO: 92 - DNA, 735 bp, artificial
  phage display generated human antibody
caggtccagctggtgcagtctggggctgaggtgaggaagcctggggcctcagtgagggtctcctgtaagacttctggatacaccttcttggaatactacatacactgggtgcgacaggcccctggacaagggcttgagtggatggcctggagcaaccctgtcactggaacgtcaggctcctcacctaaatttcggggcagagtcaccctgaccgctgacacgtccggcaacacagcctatttggacctgaagagccttacgtctgacgacacggccatattctactgtgcgaggcgtcaccaacagagcttggattattggggccaaggaaccctggtcaccgtctcgagtggaggcggcggttcaggcggaggtggctctggcggtggcggaagtgcacagtctgtgctgactcagccaccctcagtgtctgcggccccagggcagacggtcaccatctcctgctctggaagcaactccaacattgggaataatcatgtatcttggtaccgacaactcccggaaacagcccccaaactcctcatttatgacaacaataagcgaccgtcagggattcctgaccgattctctggctccaagtctggcacgtcagccaccctggacatcaccggactccagactggggacgaggccgattattactgcgcgacatgggataacagcctgagtgccccttgggtgttcggcggcgggaccaagctgaccgtcctaggt

SEQ ID NO: 93 - DNA, 756 bp, artificial
  phage display generated human antibody
caggtgcagctgcaggagtcgggggctgaggtgaagaagcctgggtcctcggtgaaggtctcctgcaaggcttctggaggcaccttcagcagctctgctatcagctgggtgcgacaggcccctggacaaggacttgagtggatgggagggatcatccctgtctttggtacagcaaattacgcacagaagttccaggacagagtcactattaccgcggacgagtccacgagcacagcctacctggagctgagcaggctgacatctgaggacacggccgtgtattactgtgcgtcgaggggggagtatgactacggtgactacgacgtctactactactatatggaggtctggggccagggcaccctggtcaccgtctcgagtggaggcggcggttcaggcggaggtggctctggcggtggcggaagtgcacagtctgtgctgactcagccaccctcggtgtcagtggccccgggacagacggccaggttgacctgtggggcaaacaacattggaagtacaagtgttcactggtaccagcagaagccaggccaggcccctgtgttggtcatatatgatgatactgaccggccctctggtatccctgagcgattctctggctccaactctgggaacacggccaccctgaccatcagaagggtcgaagccggggatgaggccgactattactgtcaggtgtgggatactaacagtgatcatgtgatattcggcggagggaccaagctgaccgtcctaggt

SEQ ID NO: 94 - DNA, 747 bp, artificial
  phage display generated human antibody
gaggtgcagctggtgcagtctggggctgaggtgaagaagcctgggtcctcggtgaaggtctcctgccaggcttctggaggcaccttcacaagccacgctatgtactgggtgcgacaggcccctggacaaggacttgagtggatgggagggatcatccctatctttggaagaacaaactacgcacagaaattccagggcagagtcacgtttaccgcggacatgtccacgagtacagcctatatggaaatgaccagcctgagatctgacgacacggccgtatattactgtgcgagaggcgataattggaatgacctttacccgattgactactggggccgaggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgccaccacttacgtgcagtggttccagcagcgcccgggcagttcccccaccactgtgatctatgatgatgaccaaagaccgtctggggtccctgatcgcttctctggatccatcgacagctcctccaactctgcctccctcaccatctctggactgatgcctgaggacgaggctgactactactgtcagtcttatgataacaccgatctggtgttcggcggtgggacccagctcaccgttttaagt

SEQ ID NO: 95 - DNA, 744 bp, artificial
  phage display generated human antibody
gaggtccagctggtacagtctggggctgaggtgaagaagcctggggcctcagtgaaggtctcctgcaaggtttccggatactccctctctgaattatccatgcactgggtgcgacaggctcctggaaaaggacttgagtggatgggaggttttgatcctcaaaatggttacacaatctacgcacaggagttccagggcagaatcaccatgaccgaggacacatctacagacacagtctacatggaactgggcagcctgagatctgaagacacggccgtgtatttctgtgcagcaatcgaaataactggggtgaactggtacttcgatctctggggcaaaggcaccctggtcaccgtctcgagtggaggcggcggttcaggcggaggtggctctggcggtggcggaagtgcactttcttctgagctgactcaggaccctgatgtgtctgtggcgttgggacagacagtcaggatcacatgccaaggagacagcctcaaaaaattttatccaggttggtaccagcagaagccaggacaggcccctctacttgtcctatatggtgaaaacattcggccctcaagaatccccgaccgattctctggctccagctccggaaacacagctaccctgaccatcactggggctcaggcggaggatgaggctgtgtattactgtaattcccgggaagccagtgttcaccatgtaagggtcttcggcggagggaccaagctgaccgtcctaggt

SEQ ID NO: 96 - DNA, 753 bp, artificial
  phage display generated human antibody
caggtgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggcaagggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcagggggtggtcttcggcggagggaccaagctgaccgtcctaggt

SEQ ID NO: 97 - DNA, 753 bp, artificial
  phage display generated human antibody
cagctgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccaaggcaccctggtcaccgtctcgagtggaggcggcggttcaggcggaggtggctctggcggtggcggaagtgcacttaattttatgctgactcagccccactctgtgtcggagtctccggggaagacggtcaccatctcctgcaccggcagcagtggcagcattgccagcaactatgtgcagtggtaccagcagcgcccgggcagtgcccccaccactctgatctatgaggatgaccaaagaccctctggggtccctgatcggttctctggctccgtcgacagctcctccaactctgcctccctcaccatctctggactgaagactgaggacgaggctgattactattgtcagtcttatgataggagcaatcaggcggtggttttcggcggagggaccaagctgaccgtcctaggt

SEQ ID NO: 98 - DNA, 759 bp, artificial
  phage display generated human antibody
caggtccagctggtgcagtctgggcctgaggtgaagaagcctggggcctcagtggaggtctcctgtaaggcttctggatacaccttcaccggcgactatatgcactgggtgcgacaggcccctggacaaggacctgagtggatggggtggatcaaccctcagactggtgtcacaaagtatgcacagaagtttcagggcagggtcaccatggccagggacacgtccatcaacacagcctacatggaactgagagggctgagatccgacgacacggccgtgtattactgtgtgcgagaggatcacaattacgatttgtggagtgcttacaacggtttggacgtctggggccagggcaccctggtcaccgtctcgagtggaggcggcggttcaggcggaggtggctctggcggtggcggaagtgcacagtctgtgctgacgcagccgccctcagtgtctgcggccccaggacagaaggtcaccatctcctgctctggaagcagctccaacattgggaataatcatgtgtcgtggtaccagcagctcgcaggaacagcccccaaactcctcatttttgacaatgataagcgaccctcagggattcctgaccgattctctggctccaagtctggcacgtcagccaccctgggcatcaccggactccagactggggacgaggccgattattattgcggaacatgggataagagtccgactgacatttatgtcttcggaagtgggaccaagctgaccgtcctaggt

SEQ ID NO: 99 - DNA, 741 bp, artificial
  phage display generated human antibody
caggtgcagctgcaggagtccggcccaggactggtgaagccttcggggaccctgtccctcacctgcgctgtctctggtggctccatcagcagtagtaactggtggagttgggtccgccaggccccagggaaggggctggagtggattggggaaatctattatggtgggagcaccaactacaacccgtccctcaagagtcgagtcaccctttcagtagacaagtccaagaaccagttctccctgaggctgatttctgtgaccgccgcggacacggccgtctattactgtgcgagaagtagtggcctctacggtgactacgggaacctgtggggccgaggaaccctggtcaccgtctcgagtggaggcggcggttcaggcggaggtggctctggcggtggcggaagtgcacagtctgtcgtgacgcagccgccctcagtgtctgcggccccaggacagaaggtcaccatctcctgctctggaagcgcctccaacattggagatcattatatatcctggtaccagcagttcccaggaacagcccccaaactcctcatctctgacaatgatcagcgaccctcagggattcctgaccggttctctggctccaagtctggcacatcagccaccctgggcatcaccggactccagactggggacgaggccgattactactgcggaacatgggatagcaacctgagttcttgggtgtttggcagtgggaccaaggtcaccgtcctaggt

SEQ ID NO: 100 - DNA, 750 bp, artificial
  phage display generated human antibody
gaagtgcagctggtgcagtctggggctgaggtgaagaagcctggggctacactgaaagtctcctgcaaagtttctgcatacaccttcaccgactactccatgcactgggtgcaacaggcccctggaaaagggcttaagtggatgggacttattgatcttgaagatggtaatacaatttacgcagaggagttccaggacagagtcaccataaccgcggacacgtctacagacacagcctacatggatctgagcagcctgagatctgaggacacggccgtgttttactgtgcaataagtccgcttcggggacttaccgcggatgtttttgatgtctggggccaaggaaccctggtcaccgtctcgagtggaggcggcggttcaggcggaggtggctctggcggtggcggaagtgcacagtctgccctgactcagcctgcctccgcgtctgggtctcctggacagtcgatcaccatctcctgcactggaaccagcagtgacattggtcgttatgactttgtctcttggtatcaacgacaaccaggcaaagcccccaaactcatgatttatgatgtcattaatcggccctcaggggtttctagtcgcttctctggctccaagtctggcaacacggcctccctgaccatctctgggctccaggctgaggacgaggctgattattactgcagctcatatgcaggttccaccactctctatgtcttcggcactgggaccaagctgaccgtcctaggt

SEQ ID NO: 101 - DNA, 738 bp, artificial
  phage display generated human antibody
caggtgcagctgcaggagtcgggcccaggactggtgaagccttcggcgaccctgtccctcacctgcgctgtctctggtggctccatcagcagtaatcactggtggagttgggtccgccagtcccccgggaagggtctggagtggattggagaaatctatacttatgggggcgccaactacaacccgtccctcaagagtcgagtcgacatatcaatggacaagtccaagaatcagttctccctgcacttgagctctgtgaccgccgcggacacggccgtgtattactgtgggagacacctgactggttacgattgttttgatatctggggccaaggaaccctggtcaccgtctcgagtggaggcggcggttcaggcggaggtggctctggcggtggcggaagtgcacaggctgtgctgactcagccgtcctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggtgtcttcggaactgggacccagctcaccgttttaagt

SEQ ID NO: 102 - DNA, 747 bp, artificial
  phage display generated human antibody
caggtgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaaccagttctccctgaaactgagctctgtgaccgccgcggacacggccgtgtattactgtgcgagagaggggggccatagtgggagttaccctcttgactactggggccaaggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgccagcaagtatgtgcagtggtaccagcagcgcccgggcagtgcccccaccagtgtcatctatgaggataaccaaagaccctctggggtccctgatcggttctctggctccatcgacagcgcctccaactctgcctccctcaccatctctggactgaagactgaggacgaggctgactactactgtcagtctgatgatggcagcagtgtggttttcggcggagggaccaaggtcaccgtcctaggt

SEQ ID NO: 103 - DNA, 771 bp, artificial
  phage display generated human antibody
gaggtccagctggtgcagtctggggctgaggtgaagaagcctggggcctcagtgaaggtctcctgcaaggcttcgggatacagctttcccagctctggtctcagctgggtgcgacaggcccctggacaagggcctgagtggatgggatggatcggcatttacaatggtaacacagactatgcacagaagttccagggcagagtcaccatgaccacagacaaatccacgagcacagcctacatggagctgaggagcctgagatctgacgacacggccgtctattactgtgcgagagattccgtggggagtatatcagtggctggtacgatgcaatactactacttcgctatggacgtctggggccaaggaaccctggtcaccgtctcgagtggaggcggcggttcaggcggaggtggctctggcggtggcggaagtgcacagtctgtgttgacgcagccgccctccgcgtccgggtctcctggacagtcagtcaccatctcctgcgctggaaccaggtatgacattggtacttataattatgtctcgtggtaccaacaacacccagccaaaggccccaaactcatcatttatgcggtcagtgagcggccctcaggtgtccctaatcgattctctggctccaagtctggcaacacggcctccctgaccgtctccgggctccgggctgaggatgaggctcattattattgcagctcatacgcaggcaacaacaatgtgattttcggcggagggaccaaggtcaccgtcctaggt

SEQ ID NO: 104 - DNA, 741 bp, artificial
  phage display generated human antibody
caggtgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccgagggacaatggtcaccgtctcgagtggaggcggcggttcaggcggaggtggctctggcggtggcggaagtgcacagtctgtgctgacgcagccgccctcagcgtctgggacccccggacagagggtcaccatctcttgttctggaagcttctccaatatcggaggtaattatgtgaactggtaccagcagctcccaggaacggcccccaaactcctcatctatgggaataatcagcggccctcaggggtccctgaccgattctctagttttaagtcgggcacctcagcctccctggccatcagtgggctccggtccgaggatgaggctgattattactgtgcaacatgggatgacagccagactgttttattcggcggagggaccaagctgaccgtcctaggt

SEQ ID NO: 105 - DNA, 738 bp, artificial
  phage display generated human antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagatggaatggtttcctgacagctcatgactcctggggccgagggacaatggtcaccgtctcgagtggaggcggcggttcaggcggaggtggctctggcggtggcggaagtgcacagtctgtgctgactcagccaccctcagcgtctgggacccccgggcagagggtcaccatctcttgttctggaagcagttccaacatcggaactaattatgtgtactggtaccaacaattcccaggaacggcccccaaactcctcatctataggagtaatcggcggccctcaggggtccctgaccgattctctgcctccaagtctggcacctcagcctccctggtcatcagtgggctccggtccgaagatgaggctgactattactgtgcagcatgggatgacagactgaatggcgagatgttcggcggagggaccaaggtcaccgtcctaggt

SEQ ID NO: 106 - DNA, 729 bp, artificial
  phage display generated human antibody
gaggtgcagctgttggagtctgggggaggcttggtacagcctggggggtccctgagactctcntgtgcagcctctggattcacctttagcagctatgccatgagctgggttcgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagatggtccgggcggttttatgacttctgggggcaagggaccacggtcaccgtctcgagtggaggcggcggttcaggcggaggtggctctggcggtggcggaagtgcacagtctgtgctgactcagccaccctcagcgtctgggacccccgggcagaggatcaccatctcttgttccggaagcagctccaacatcggaagtaattatgtatactggtaccagcaactcccaggaacggcccccaaaatcctcatctataggaataatcagcggccctcaggggtccctgagcgattctctggctccaagtctggcacctcagcctccctggccatcagtgggctccggtccgaggatgaggctgactactattgtgcagcatgggatgacagcctgagtgaagtgttcggcggagggaccaaggtcaccgtcctaggt

SEQ ID NO: 107 - DNA, 738 bp, artificial
  phage display generated human antibody
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagataagggttatagtggctttgactactggggccggggaaccctggtcaccgtctcgagtggaggcggcggttcaggcggaggtggctctggcggtggcggaagtgcacagtctgtgttgacgcagccgccctcagcgtctgggacccccgggcagagggtcaccatctcttgctctggaagcagctccaacatcggacgtcatactgttaactggtaccagcaactcccaggaacggcccccaaactgctcatctatagcaataatcagcggccctcaggggtccctgaccgattctctggctccaagtctggcacctcagcctccctggccatcagtgggctccagtctgaagatgagggtcattatcactgtgcagcatgggatgacaccctgaatggtgatgtggtattcggcggagggaccaaggtcaccgtcctaggt

SEQ ID NO: 108 - DNA, 753 bp, artificial
  phage display generated human antibody
cagctgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggcaagggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaacccttatgtggtattcggcggagggaccaagctgaccgtcctaggt

SEQ ID NO: 109 - DNA, 753 bp, artificial; identical to SEQ ID NO: 85
  phage display generated human antibody

SEQ ID NO: 110 - DNA, 744 bp, artificial
  phage display generated human antibody
cagctgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccagggcaccctggtcaccgtctcgagtggaggcggcggttcaggcggaggtggctctggcggtggcggaagtgcacagtctgtcgtgacgcagccgccctcagtgtctgcggccccaggacagaaggtcaccatctcctgctctggaagcagctccaacattgggaataattatgtatcctggtataaacaactcccaggaacagcccccaaactcctcatctatgacaataataagcgaccctctgggattcctgaccgattctctggctccaagtctggcacgtcagccaccctgggcataaccggactccagactggggacgaggccgattattactgcggaacttgggatagcagcctgagtggcgtggtgttcggcggagggaccaagctgaccgtcctaggt

SEQ ID NO: 111 - DNA, 753 bp, artificial
  phage display generated human antibody
cagctgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccgaggaaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgccagcaactatgtgcagtggtaccaacagcgcccgggcagttcccccaccactttgatctatgacgataaccagagaccctctggggtccctgatcggttctctggctccatcgacagctcctccaactctgcctccctcaccatctctggactgaagactgaggacgaggctgactactactgtcagtcttatgacagcagcaatctgggggtggtcttcggcggagggaccaagctgaccgtcctaggt

SEQ ID NO: 112 - DNA, 750 bp, artificial
  phage display generated human antibody
caggtgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccggggaaccctggtcaccgtctcgagtggaggcggcggttcaggcggaggtggctctggcggtggcggaagtgcacttaattttatgctgactcagccccactctgtgtcggagtctccggggaagacggcaaccatctcctgcaccggcagcggtggcagcattgccagaagctatgtgcagtggtaccagcagcgcccgggccgtgcccccagcatcgttatctatgaggattatcaaaggccctctggcgtccctgatcggttctctggctccatcgacagctcctccaattctgcctctctcaccatcactgggctgaagactgacgacgaggctgactactactgtcagtcctctgacgacaacaacaatgtcgtcttcggcggagggaccaaggtcaccgtcctaggt

SEQ ID NO: 113 - DNA, 744 bp, artificial
  phage display generated human antibody
caggtgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggcaggggaaccctggtcaccgtctcgagtggaggcggcggttcaggcggaggtggctctggcggtggcggaagtgcacaggctgtgctgactcagccgtcctcagtgtctgcggccccaggacagaaggtcaccatctcctgctctggaagcagctccaacattgggaataattatgtatcctggtaccagcagctcccaggaacagcccccaaactcctcatttatgacaataatgagcgaccctcagggattcctgaccgattctctggctccaagtctggcacgtcagccaccctgggcatcaccggactccagactggggacgaggccgattattactgcggaacatgggatagcagcctgagtactgtggtcttcggaactgggaccaaggtcaccgtcctaggt

SEQ ID NO: 114 - DNA, 747 bp, artificial
  phage display generated human antibody
cagctgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccagggaaccctggtcaccgtctcgagtggaggcggcggttcaggcggaggtggctctggcggtggcggaagtgcacttaattttatgctgactcagccccactctgtgtcggagtctccggggaagacggtgaccgtttcctgcaccggcagcggtggcaacattgccagcaattatgtacagtggtaccagcagcgcccggacagtgcccccacccttgtgatctttgaggatacccaaaggccctctggggtccctgctcggttctctggctccatcgacagctcctccaactctgcctccctcatcatctcctcactgaggactgaggacgaggctgattactattgtcaatcttctgattccaacagggtggtgttcggcggagggaccaaggtcaccgtcctaggt

SEQ ID NO: 115 - DNA, 723 bp, artificial
  phage display generated human antibody
caggtgcagctgcaggagtcgggcccaggactggtgaagccttcggagaccctgtccctcacctgcaatgtctctggtggctccatcaggaattacttctggagttggatccggcagcccccagggcagggactggagtacattgggtatatctattacagtgggaccaccgactacaacccctccctcaagggtcgagtcaccatatcactagacacgtccaagacccagttctccttgaagctgaactctgtgaccgctgcggacacggccttctattactgtgtgagaggcccgaataagtatgcgttcgacccctggggccaaggcaccctggtcaccgtctcgagtggaggcggcggttcaggcggaggtggctctggcggtggcggaagtgcactttcctatgagctgactcagccaccctcagtgtccgtgtcccccggacagacagccagcatcacctgctctggagataaattgggggataaatttgcttcctggtatcaacagaaggcaggccagtcccctgtgctggtcatctatcgagataccaagcgcccctcagggatccctgagcgattctctggctccaactctgggaacacagccactctcaccatcagcgggacccaggctatggatgaggctgattattactgtcaggcgtgggacagcagcacggcggtcttcggaactgggaccaaggtcaccgtcctaggt

SEQ ID NO: 116 - DNA, 753 bp, artificial
  phage display generated human antibody
cagctgcagctgcaggagtcgggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccaaggaaccctggtcaccgtctcgagtggaggcggcggttcaggcggaggtggctctggcggtggcggaagtgcacttaattttatgctgactcagccccactctgtgtcggagtctccggggaagacggtaaccatctcctgcacccgcagcagtggcagcattgacaacaactatgtccagtggtaccagcagcgcccgggcagttcccccactactgtgatctttgaggataaccaaagaccctctggggtccctgatcgcttctctggctccatcgacagctcctccaactctgcctccctcaccatctctggactgaagactgaggacgaggctgactactactgtcagtcttatgatagccacaatcagggggtggtcttcggcggagggaccaagctgaccgtcctaggt

SEQ ID NO: 117 - DNA, 744 bp, artificial
  phage display generated human antibody
cagctgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccgaggaaccctggtcaccgtctcgagtggaggcggcggttcaggcggaggtggctctggcggtggcggaagtgcacagtctgtgctgacgcagccgccctcagtgtctgcggccccaggacagaaggtcaccatctcctgctctggaagtagctccaacattgggaatagttatgtatcgtggtacaagcagctcccaggtacagcccccaaagtcctcatttatgacaaccagaagcgatcctcagggatccctgaccgattctctgcctccaagtctggcacgtcagccaccctgggcatcaccggactccggactgaggacgaggccgattattactgcggaacatgggataccagcctgagtgcggtggtgttcggcggagggaccaagctgaccgtcctaggt

SEQ ID NO: 118 - DNA, 744 bp, artificial
  phage display generated human antibody
gaggtgcagctggtggagtctggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggccggggaaccctggtcaccgtctcgagtggaggcggcggttcaggcggaggtggctctggcggtggcggaagtgcacagtctgtcgtgacgcagccgccctcagtatctgcggccccaggacagaaggtcaccatctcctgctctggaaacttctccaacattgaatataattatgtatcgtggtaccagcacctcccaggaacagcccccaaactcctcatttttgacaataatcagcgaccctcatggattcctgaccgattctctggctccaagtctggcacgtcagccaccctgggcatcaccgggctccagactggggacgaggccgattactactgcggaacatgggatagcagcctgaatgctggggtgttcggcggagggaccaaggtcaccgtcctaggt

SEQ ID NO: 119 - DNA, 736 bp, artificial
  phage display generated human antibody
gaggtgcagctgttggagtctgggggaggcttggtacggcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaagatcgaaggggtgtcctcgacccctggggcaaagggacaatggtcaccgtctcgagtggaggcggcggttcaggcggaggtggctctggcggtggcggaagtgcacagtctgtgctgacgcagccgccctcagtgtctggggccccagggcagagggtcaccatctcctgcactgggagcagctccaacatcggggcaggctatgatgtacactggtaccagcaccttccaggaacagcccccagactcctcatctatggtaacagcaatcggccctcaggggtccctgaccgattctctggctccaagtctggcacctcagcctccctggccatctctgggctccaggctgaggatgaggctgattattactgccagtcctatgacagcagcctgagtgattgggtgttcggcggagggaccaaggtcaccgtcctaggtc

SEQ ID NO: 120 - DNA, 750 bp, artificial
  phage display generated human antibody
cagctgcagctgcaggagtccggcccaggactggtgaagccttcggggaccctgtccctcacctgcgctgtctctggtggctccatcagcactagtgactggtggagttgggtccgccggcccccagggaaggggctggagtggattggggaaatctatcatagtgggagcaccaactaccacccgtcactcaagagtcgagtcaccatatcacttgacaaatcgaagaatcagttctccctgaaactgagctctgtgaccgccgcggacacggccgtgtattactgtgcgagagaggggggccatagtgggagttaccctcttgactactggggcaggggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcgcccgcagcagtggcagcattgccagcaactatgtgcagtggtaccagcagcgcccgggcagttcccccaccactttgatctatgaggataggcaaagaccctctggggtccctgatcggttctctggctccatcgacagctcctccaactctgcctccctcaccatctctggactgaagactgaggacgaggctgactactactgtcagtcttatgatagcagcgatcatgtggtcttcggcggagggaccaagctgaccgtcctaggt

SEQ ID NO: 121 - DNA, 23 bp, artificial
  mutagenesis primer
cagggcagggtcacaatggccag

SEQ ID NO: 122 - DNA, 23 bp, artificial
  mutagenesis primer
ctggccattgtgaccctgccctg

SEQ ID NO: 123 - DNA, 39 bp, artificial
  PCR Primer
ctctccacaggcgcgcactcccaggtgcagctgcaggag

SEQ ID NO: 124 - DNA, 39 bp, artificial
  PCR Primer
ctctccacaggcgcgcactccgaggtgcagctgttggag

SEQ ID NO: 125 - DNA, 39 bp, artificial
  PCR Primer
ctctccacaggcgcgcactcccaggtgccagctggtgca

SEQ ID NO: 126 - DNA, 45 bp, artificial
  PCR Primer
ctctccacaggcgcgcactcccagctgcagctgcaggagtcgggc

SEQ ID NO: 127 - DNA, 21 bp, artificial
  PCR Primer
accgccagagccacctccgcc

SEQ ID NO: 128 - DNA, 39 bp, artificial
  PCR Primer
ctccacaggcgtgcactcccaggctgtgctgactcagcc

SEQ ID NO: 129 - DNA, 41 bp, artificial
  PCR Primer
ctctccacaggcgtgcactcccagtctgtgctgactcagcc

SEQ ID NO: 130 - DNA, 35 bp, artificial
  PCR Primer
ccacaggcgtgcactcctcctatgagctgactcag

SEQ ID NO: 131 - DNA, 37 bp, artificial
  PCR Primer
ctccacaggcgtgcactccaattttatgctgactcag

SEQ ID NO: 132 - DNA, 60 bp, artificial
  PCR Primer
ctattccttaattaagttagatctattctgactcacctaggacggtcagcttggtccctc

SEQ ID NO: 133 - DNA, 58 bp, artificial
  PCR Primer
ctattccttaattaagttagatctattctgactcacctaggacggtgaccttggtccc

SEQ ID NO: 134 - DNA, 61 bp, artificial
  PCR Primer
ctattccttaattaagttagatctattctgactcacctaggacggtcagcttggtcccact

SEQ ID NO: 135 - DNA, 61 bp, artificial
  PCR Primer
ctattccttaattaagttagatctattctgactcacctaggacggtgaccttggtcccagt

SEQ ID NO: 136 - DNA, 58 bp, artificial
  PCR Primer
ctattccttaattaagttagatctattctgactcacctaggacggtgagctgggtccc

SEQ ID NO: 137 - DNA, 19 bp, artificial
  PCR Primer
gcaggcttgaggtctggac

SEQ ID NO: 138 - DNA, 25 bp, artificial
  PCR Primer
taattatagcaaggagaccaagaag

SEQ ID NO: 139 - DNA, 25 bp, artificial
  PCR Primer
cagaggtgctcttggaggagggtgc

SEQ ID NO: 140 - protein, 120 aa, artificial
  V_region
EVQLLESGGGLVQPGGSLRLSCAASGFTFSSYAMSWVRQAPGKGLEWVSISGSGGSTYYADSVKGRFTISRDNSKNTLYLQMNSLRAEDTAVYYCAKDHYYDSSGYLDYWGQGTLVTVSS

SEQ ID NO: 141 - protein, 111 aa, artificial
  V_region
NFMLTQPHSVSESPGKTVTISCTRSSGSIAFDYVQWYQQRPGSAPTTVIYEDNQRPSGVPDRFSASIDSSSNSASLTISALKTEDEADYYCQSYDNSNSWVFGGGTKLTVL

SEQ ID NO: 142 - protein, 119 aa, artificial
  V_region
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCARGHSGSYPLDYWGKGTLVTVSS

SEQ ID NO: 143 - protein, 112 aa, artificial
  V_region
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNQGVVFGGGTKLTVL

SEQ ID NO: 144 - protein, 125 aa, artificial
  V_region
QVQLVQSGPEVKKPGASVEVSCKASGYTFTGDYMHWVRQAPGQGPEWMGWINPQTGVTKYAQKFQGRVTMARDTSINTAYMELRGLRSDDTAVYYCVREDHNYDLWSAYNGLDVWGQGTLVTVSS

SEQ ID NO: 145 - protein, 111 aa, artificial
  V_region
QSVLTQPPSVSAAPGQKVTISCSGSSSNIGNNHVSWYQQLAGTAPKLLIFDNDKRPSGIPDRFSGSKSGTSATLGITGLQTGDEADYYCGTWDKSPTDIYVFGSGTKLTVL

SEQ ID NO: 146 - protein, 121 aa, artificial
  V_region
QLQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSS

SEQ ID NO: 147 - protein, 111 aa, artificial
  V_region
NFMLTQPHSVSESPGKTVTISCARSSGSIASNYVQWYQQRPGSSPTTLIYEDRQRPSGVPDRFSGSIDSSSNSASLTISGLKTEDEADYYCQSYDSSDHVVFGGGTKLTVL

SEQ ID NO: 148 - protein, 119 aa, artificial
  V_region
QVQLQESGPGLVKPSATLSLTCAVSGGSISSNHWWSWVRQSPGKGLEWIGEIYTYGGANYNPSLKSRVDISMDKSKNQFSLHLSSVTAADTAVYYCGRHLTGYDCFDIWGQGTLVTVSS

SEQ ID NO: 149 - protein, 110 aa, artificial
  V_region
QAVLTQPSSVSGAPGQRVTISCTGSSSNIGAGYDVHWYQQLPGTAPKLLIYGNSNRPSGVPDRFSGSKSGTSASLAITGLQAEDEADYYCQSYDSSLSGVFGTGTQLTVL

SEQ ID NO: 150 - protein, 121 aa, artificial
  V_region
QVQLQESGPGLVKPSGTLSLTCAVSGGSISTSDWWSWVRRPPGKGLEWIGEIYHSGSTNYHPSLKSRVTISLDKSKNQFSLKLSSVTAADTAVYYCAREGGHSGSYPLDYWGRGTLVTVSS

SEQ ID NO: 151 - protein, 111 aa, artificial
  V_region
NFMLTQPHSVSESPGKTATISCTGSGGSIARSYVQWYQQRPGRAPSIVIYEDYQRPSGVPDRFSGSIDSSSNSASLTITGLKTDDEADYYCQSSDDNNNVVFGGGTKVTVL

SEQ ID NO: 152 - protein, 117 aa, artificial
  V_region
QVQLQESGPGLVKPSETLSLTCNVSGGSIRNYFWSWIRQPPGQGLEYIGYIYYSGTTDYNPSLKGRVTISLDTSKTQFSLKLNSVTAADTAFYYCVRGPNKYAFDPWGQGTLVTVSS

SEQ ID NO: 153 - protein, 106 aa, artificial
  V_region
SYELTQPPSVSVSPGQTASITCSGDKLGDKFASWYQQKAGQSPVLVIYRDTKRPSGIPERFSGSNSGNTATLTISGTQAMDEADYYCQAWDSSTAVFGTGTKVTVL

SEQ ID NO: 154 - protein, 109 aa, homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGQGTLVTVSS

SEQ ID NO: 155 - protein, 109 aa, homo sapiens
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWGQGTLVTVSS

SEQ ID NO: 156 - protein, 109 aa, homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARWGQGTLVTVSS

SEQ ID NO: 157 - protein, 108 aa, homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARWGQGTLVTVSS

SEQ ID NO: 158 - protein, 108 aa, homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNFGGGTKLTVL

SEQ ID NO: 159 - protein, 108 aa, homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAFGTGTKVTVL

SEQ ID NO: 160 - protein, 109 aa, homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGFGGGTQLTVL

SEQ ID NO: 161 - protein, 105 aa, homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTAFGTGTKVTVL